SEQUENCE LISTING (75 sequences)

SEQ ID NO: 1 - protein, 12 aa, Homo sapiens
  spacer (IgG4hinge)
ESKYGPPCPPCP

SEQ ID NO: 2 - DNA, 36 bp, Homo sapiens
  spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28 (amino acids 153-179 of Accession No. P10747)
FWVLVVVGGVLACYSLLVTVAFIIFWV

SEQ ID NO: 9 - protein, 66 aa, Homo sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  CD28 (LL to GG)
RSKRSRGGHSDYMNMTPRRPGPTRKHYQPYAPPRDFAAYRS

SEQ ID NO: 12 - protein, 42 aa, Homo sapiens
  4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  Linker
  REPEAT (5)...(9): SGGGG is repeated 5 times
PGGGSGGGGP

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, sArtificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 59 - protein, 18 aa, Artificial Sequence
  Linker
GSTSGSGKPGSGEGSTKG

SEQ ID NO: 60 - protein, 5 aa, Artificial Sequence
  CDRH1
NSGMH

SEQ ID NO: 61 - protein, 17 aa, Artificial Sequence
  CDRH2
VIWYDGSKRYYADSVKG

SEQ ID NO: 62 - protein, 4 aa, Artificial Sequence
  CDRH3
NDDY

SEQ ID NO: 63 - protein, 11 aa, Artificial Sequence
  CDRL1
RASQSVSSYLA

SEQ ID NO: 64 - protein, 7 aa, Artificial Sequence
  CDRL2
DASNRAT

SEQ ID NO: 65 - protein, 9 aa, Artificial Sequence
  CDRL3
QQSSNWPRT

SEQ ID NO: 66 - protein, 113 aa, Artificial Sequence [sequence omitted]

SEQ ID NO: 67 - protein, 107 aa, Artificial Sequence [sequence omitted]

SEQ ID NO: 68 - protein, 5 aa, Artificial Sequence
  CDR-H1
DYYWN

SEQ ID NO: 69 - protein, 16 aa, Artificial Sequence
  CDR-H2
EINHRGSTNSNPSLKS

SEQ ID NO: 70 - protein, 12 aa, Artificial Sequence
  CDR-H3
GYSDYEYNWFDP

SEQ ID NO: 71 - protein, 11 aa, Artificial Sequence
  CDR-L1
RASQSISSYLA

SEQ ID NO: 72 - protein, 7 aa, Artificial Sequence
  CDR-L2
DASNRAT

SEQ ID NO: 73 - protein, 9 aa, Artificial Sequence
  CDR-L3
QQRSNWPLT

SEQ ID NO: 74 - protein, 120 aa, Artificial Sequence
  VH
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 75 - protein, 107 aa, Artificial Sequence
  VL
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

(19) United States
(12) Patent Application Publication
(10) Pub. No.: US 2024/0181052 A1
(43) Pub. Date: Jun. 6, 2024

(54) METHODS FOR DOSING AND TREATMENT WITH A COMBINATION OF A CHECKPOINT INHIBITOR THERAPY AND A CAR T CELL THERAPY

(71) Applicant: Juno Therapeutics, Inc., Seattle, WA (US)

(72) Inventors: Nikolaus Sebastian TREDE, Seattle, WA (US); Satyendra SURYAWANSHI, Seattle, WA (US); Shruti AGRAWAL, Seattle, WA (US); Shivani SRIVASTAVA, Seattle, WA (US); Tim PULHAM, Boudry (CH); Joe DESTEFANO, Summit, NJ (US); Nicolas BRAND, Boudry (CH); Ronald DUBOWY, Seattle, WA (US)

(73) Assignee: Juno Therapeutics, Inc., Seattle, WA (US)

(21) Appl. No.: 18/284,725

(22) PCT Filed: Mar. 29, 2022
(86) PCT No.: PCT/US2022/022377

§ 371 Date: Sep. 28, 2023

Related U.S. Application Data
(60) Provisional application No. 63/167,596, filed on Mar. 29, 2021.

(51) Int. Cl. A61K 39/00; A61P 35/00; C07K 14/705; C07K 14/725; C07K 16/28

(52) U.S. Cl. CPC A61K 39/4611; A61K 39/4631; A61K 39/464412; A61P 35/00; C07K 14/7051; C07K 14/70521; C07K 14/70578; C07K 16/2803; C07K 16/2818; A61K 2039/507; A61K 2039/545; C07K 2317/622; C07K 2319/02; C07K 2319/03

(57) ABSTRACT

Provided herein are methods and uses of combination therapies involving a T cell therapy, e.g., a CAR T cell therapy, and a checkpoint inhibitor therapy, e.g. an anti-PD-1 antibody and/or an anti-LAG3 antibody, for treating subjects with cancers such as lymphomas, and related methods, uses, and articles of manufacture.

### CROSS-REFERENCE TO RELATED APPLICATION

This application claims priority to U.S. Provisional Application No. 63/167,596, filed Mar. 29, 2021, entitled “METHODS FOR DOSING AND TREATMENT WITH A COMBINATION OF A CHECKPOINT INHIBITOR THERAPY AND A CAR T CELL THERAPY,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042024940SeqList.TXT, created Mar. 28, 2022, which is 41,457 bytes in size. The information in the electronic format of the Sequence Listing is incorporated by reference in its entirety.

### FIELD

The present disclosure relates in some aspects to methods and uses of combination therapies involving a T cell therapy, e.g., a CAR T cell therapy, and a checkpoint inhibitor therapy, e.g. an anti-PD-1 antibody and/or an anti-LAG3 antibody, for treating subjects with cancers such as lymphomas, and related methods, uses, and articles of manufacture. The T cell therapy includes cells that express recombinant receptors such as chimeric antigen receptors (CARs).

### BACKGROUND

Various strategies are available for cell therapy for treating cancers, for example, adoptive cell therapies, including those involving the administration of cells expressing chimeric receptors specific for a disease or disorder of interest, such as chimeric antigen receptors (CARs) and/or other recombinant antigen receptors, as well as other adoptive immune cell and adoptive T cell therapies. In some cases, endogenous T cells, engineered T cells, or both, may become exhausted and less efficacious. Improved methods are therefore needed, for example, to overcome such exhaustion and increase the efficacy of these methods. Provided are methods and uses that meet such needs.

### SUMMARY

Provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering a PD-1 inhibitor to the subject in a dosing regimen including administration of at least two doses, wherein: (i) administration of the first dose is between Day 2 and Day 20, inclusive; and (ii) a dose is administered about every two weeks (Q2W) or about every four weeks (Q4W) in an amount of between at or about 140 mg and at or about 580 mg, inclusive.

In some embodiments, the first dose is administered between Day 8 and Day 15, inclusive. In some embodiments, the first dose is administered on Day 8. In some embodiments, the first dose is administered on Day 15.

In some embodiments, the amount of the PD-1 inhibitor is between at or about 160 mg and 560 mg. In some embodiments, the amount of the PD-1 inhibitor is at or about 240 mg or at or about 480 mg. In some embodiments, the amount of the PD-1 inhibitor is 240 mg. In some embodiments, the amount of the PD-1 inhibitor is 360 mg. In some embodiments, the amount of the PD-1 inhibitor is 480 mg.

In some embodiments, a dose is administered about every two weeks (Q2W). In some embodiments, a dose is administered about every four weeks (Q4W). In some embodiments, the amount of the PD-1 inhibitor is 240 mg and a dose is administered about Q2W or the amount of the PD-1 inhibitor is 480 mg and a dose is administered about Q4W. In some embodiments, the amount of the PD-1 inhibitor is 240 mg and a dose is administered about Q2W. In some embodiments, the amount of the PD-1 inhibitor is 360 mg and a dose is administered about Q3W. In some embodiments, the amount of the PD-1 inhibitor is 480 mg and a dose is administered about Q4W.

In any of the provided embodiments, the method further includes administering a dose of a LAG3 inhibitor to the subject about every two weeks (Q2W). In some embodiments, the method further includes administering a dose of a LAG3 inhibitor to the subject about every three weeks (Q3W). In some embodiments, the method further includes administering a dose of a LAG3 inhibitor to the subject about every four weeks (Q4W).

In any of the provided embodiments, the method further includes administering a LAG3 inhibitor to the subject in a dosing regimen including administration of a dose of the LAG3 inhibitor on each of the same days on which a dose of the PD-1 inhibitor is administered.

In some embodiments, each dose of the LAG3 inhibitor is administered in an amount between at or about 60 mg and at or about 1040 mg. In some embodiments, each dose of the LAG3 inhibitor is administered in an amount at or about 120 mg. In some embodiments, each dose of the LAG3 inhibitor is administered in an amount between at or about 160 mg and at or about 1040 mg. In some embodiments, each dose of the LAG3 inhibitor is administered in an amount between at or about 160 mg and at or about 320 mg. In some embodiments, each dose of the LAG3 inhibitor is administered in an amount at or about 160 mg. In some embodiments, each dose of the LAG3 inhibitor is administered in an amount at or about 240 mg. In some embodiments, each dose of the LAG3 inhibitor is administered in an amount between at or about 400 mg and at or about 560 mg. In some embodiments, each dose of the LAG3 inhibitor is administered in an amount at or about 480 mg. In some embodiments, each dose of the LAG3 inhibitor is administered in an amount between at or about 880 mg and at or about 1040 mg. In some embodiments, each dose of the LAG3 inhibitor is administered in an amount at or about 960 mg.

In some embodiments, the amount of the PD-1 inhibitor is 120 mg and a dose is administered about Q2W. In some embodiments, the amount of the PD-1 inhibitor is 240 mg and a dose is administered about Q4W. In some embodiments, the amount of the PD-1 inhibitor is 360 mg and a dose is administered about Q3W. In some embodiments, the amount of the PD-1 inhibitor is 240 mg and a dose is administered about Q4W. In some embodiments, the amount of the PD-1 inhibitor is 480 mg and a dose is administered about Q4W.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering a PD-1 inhibitor to the subject in a dosing regimen including: (i) administration of a first amount of the PD-1 inhibitor of between at or about 140 mg and at or about 580 mg, inclusive, for a first cycle, wherein at least one dose of the first amount of the PD-1 inhibitor is administered in the first cycle and the first dose of the first cycle is administered between Day 2 and Day 20, inclusive; and (ii) administration of a second amount of the PD-1 inhibitor of between at or about 380 mg and at or about 580 mg, inclusive, about once every four weeks (Q4W) for a second cycle, wherein at least two doses of the second amount of the PD-1 inhibitor are administered in the second cycle and the first dose of the second cycle is administered between Day 50 and Day 65.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering to the subject a PD-1 inhibitor in a dosing regimen including: (i) administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and (ii) administration of a second amount of the PD-1 inhibitor of between at or about 140 mg and at or about 580 mg, inclusive, about once every four weeks (Q4W) for a second cycle, wherein at least two doses of the second amount are administered in the second cycle, and the first dose of the second cycle is administered between Day 50 and Day 65.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering to the subject a PD-1 inhibitor in a dosing regimen including: (i) administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and (ii) administration of a second amount of the PD-1 inhibitor of between at or about 380 mg and at or about 580 mg, inclusive, about once every four weeks (Q4W) for a second cycle, wherein at least two doses of the second amount are administered in the second cycle, and the first dose of the second cycle is administered between Day 50 and Day 65.

In some embodiments, the first dose of the first cycle is administered between Day 8 and Day 15, inclusive. In some embodiments, the first dose of the first cycle is administered on Day 8. In some embodiments, the dose of the first cycle is administered on Day 15.

In some embodiments, the first amount of the PD-1 inhibitor is between at or about 160 mg and at or about 320 mg, inclusive. In some embodiments, the first amount of the PD-1 inhibitor is between at or about 200 mg and at or about 280 mg, inclusive. In some embodiments, the first amount of the PD-1 inhibitor is at or about 240 mg. In some embodiments, the first amount of the PD-1 inhibitor is between at or about 400 mg and at or about 560 mg. In some embodiments, the first amount of the PD-1 inhibitor is between at or about 440 mg and at or about 520 mg. In some embodiments, the first amount of the PD-1 inhibitor is at or about 480 mg.

In some embodiments, the second amount of the PD-1 inhibitor is between at or about 400 mg and at or about 560 mg, inclusive. In some embodiments, the second amount of the PD-1 inhibitor is between at or about 440 mg and at or about 520 mg, inclusive. In some embodiments, the second amount of the PD-1 inhibitor is at or about 480 mg.

In some embodiments, the first cycle is for at least about four weeks after administration of the first dose of the first cycle. In some embodiments, the first cycle is for up to about five weeks after administration of the first dose of the first cycle. In some embodiments, the first cycle is for up to about six weeks after administration of the first dose of the first cycle. In some embodiments, the first cycle is for up to about seven weeks after administration of the first dose of the first cycle. In some embodiments, the first cycle is for up to about eight weeks after administration of the dose of engineered T cells.

In some embodiments, doses of the first amount of the PD-1 inhibitor are administered on Days 8, 22 and 36. In some embodiments, doses of the first amount of the PD-1 inhibitor are administered on Days 8 and 36. In some embodiments, doses of the first amount of the PD-1 inhibitor are administered on Days 15, 29 and 43.

In some embodiments, the at least one dose of the first amount of the PD-1 inhibitor is one dose that is administered on Day 15.

In some embodiments, the second cycle is for up to at least about three months after the administration of the dose of engineered T cells. In some embodiments, the second cycle is for up to about three months after the administration of the dose of engineered T cells.

In some embodiments, the first and second dose of the second amount of the PD-1 inhibitor are administered on Days 57 and 85, respectively.

In some embodiments, doses of the first amount of the PD-1 inhibitor are administered on Days 8, 22, and 36; and the first and second dose of the second amount of the PD-1 inhibitor are administered on Days 57 and 85, respectively. In some embodiments, doses of the first amount of the PD-1 inhibitor are administered on Days 8 and 36; and the first and second dose of the second amount of the PD-1 inhibitor are administered on Days 57 and 85, respectively. In some embodiments, doses of the first amount of the PD-1 inhibitor are administered on Days 15, 29, and 43; and the first and second dose of the second amount of the PD-1 inhibitor are administered on Days 57 and 85, respectively. In some embodiments, the at least one dose of the first amount of the PD-1 inhibitor is one dose that is administered on Day 15; and the first and second dose of the second amount of the PD-1 inhibitor are administered on Days 57 and 85, respectively.

In any of the provided embodiments, the method further includes administering a dose of a LAG3 inhibitor to the subject about every two weeks (Q2W). In some embodiments, the method further includes administering a dose of a LAG3 inhibitor to the subject about every three weeks (Q3W). In some embodiments, the method further includes administering a dose of a LAG3 inhibitor to the subject about every four weeks (Q4W). In any of the provided embodiments, the method further includes administering a LAG3 inhibitor to the subject in a dosing regimen including administration of a dose of the LAG3 inhibitor on each of the same days on which a dose of the PD-1 inhibitor is administered.

In some embodiments, each dose of the LAG3 inhibitor is administered in an amount between at or about 60 mg and at or about 1040 mg. In some embodiments, each dose of the LAG3 inhibitor is administered in an amount at or about 120 mg. In some embodiments, each dose of the LAG3 inhibitor is administered in an amount between at or about 160 mg and at or about 1040 mg. In some embodiments, each dose of the LAG3 inhibitor is administered in an amount between at or about 160 mg and at or about 320 mg. In some embodiments, each dose of the LAG3 inhibitor is administered in an amount at or about 160 mg. In some embodiments, each dose of the LAG3 inhibitor is administered in an amount at or about 240 mg. In some embodiments, each dose of the LAG3 inhibitor is administered in an amount between at or about 400 mg and at or about 560 mg. In some embodiments, each dose of the LAG3 inhibitor is administered in an amount at or about 480 mg. In some embodiments, each dose of the LAG3 inhibitor is administered in an amount between at or about 880 mg and at or about 1040 mg. In some embodiments, each dose of the LAG3 inhibitor is administered in an amount at or about 960 mg.

In some embodiments, each dose of the LAG3 inhibitor is administered in a first amount during the first cycle and in a second amount during the second cycle.

In some embodiments, the first amount of the LAG3 inhibitor is between at or about 60 mg and at or about 320 mg. In some embodiments, the first amount of the LAG3 inhibitor is at or about 120 mg. In some embodiments, the first amount of the LAG3 inhibitor is between at or about 160 mg and at or about 320 mg. In some embodiments, the first amount of the LAG3 inhibitor is at or about 160 mg. In some embodiments, the first amount of the LAG3 inhibitor is between at or about 200 mg and at or about 280 mg. In some embodiments, the first amount of the LAG3 inhibitor is at or about 240 mg. In some embodiments, the first amount of the LAG3 inhibitor is between at or about 400 mg and at or about 560 mg. In some embodiments, the first amount of the LAG3 inhibitor is between at or about 440 mg and at or about 520 mg. In some embodiments, the first amount of the LAG3 inhibitor is at or about 480 mg. In some embodiments, the first amount of the LAG3 inhibitor is between at or about 880 mg and at or about 1040 mg. In some embodiments, the first amount of the LAG3 inhibitor is between at or about 920 mg and at or about 1000 mg. In some embodiments, the first amount of the LAG3 inhibitor is at or about 960 mg.

In some embodiments, the second amount of the LAG3 inhibitor is between at or about 400 mg and at or about 560 mg. In some embodiments, the second amount of the LAG3 inhibitor is between at or about 440 mg and at or about 520 mg. In some embodiments, the second amount of the LAG3 inhibitor is at or about 480 mg. In some embodiments, the second amount of the LAG3 inhibitor is between at or about 880 mg and at or about 1040 mg. In some embodiments, the second amount of the LAG3 inhibitor is between at or about 920 mg and at or about 1000 mg. In some embodiments, the second amount of the LAG3 inhibitor is at or about 960 mg.

In some embodiments, the first amount of the LAG3 inhibitor is at or about 240 mg, and the second amount of the LAG3 inhibitor is at or about 480 mg. In some embodiments, the first amount of the LAG3 inhibitor is at or about 480 mg, and the second amount of the LAG3 inhibitor is at or about 480 mg. In some embodiments, the first amount of the LAG3 inhibitor is at or about 480 mg, and the second amount of the LAG3 inhibitor is at or about 960 mg. In some embodiments, the first amount of the LAG3 inhibitor is at or about 960 mg, and the second amount of the LAG3 inhibitor is at or about 960 mg.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85; and (3) administering to the subject a LAG3 inhibitor in a dosing regimen including: (i) administration of a first amount of the LAG3 inhibitor, wherein the first amount is 240 mg and is administered on Days 8, 22, and 36; and (ii) administration of a second amount of the LAG3 inhibitor, wherein the second amount is 480 mg and is administered on Days 57 and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85; and (3) administering to the subject a LAG3 inhibitor in a dosing regimen including: (i) administration of a first amount of the LAG3 inhibitor, wherein the first amount is 480 mg and is administered on Days 8, 22, and 36; and (ii) administration of a second amount of the LAG3 inhibitor, wherein the second amount is 960 mg and is administered on Days 57 and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering to the subject a PD-1 inhibitor in a dosing regimen including: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including administration of 480 mg of the PD-1 inhibitor on Days 8, 36, 57, and 85; and (3) administering to the subject a LAG3 inhibitor in a dosing regimen including administration of 480 mg of the LAG3 inhibitor on Days 8, 36, 57, and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including administration of 480 mg of the PD-1 inhibitor on Days 8, 36, 57, and 85; and (3) administering to the subject a LAG3 inhibitor in a dosing regimen including administration of 960 mg of the LAG3 inhibitor on Days 8, 36, 57, and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering to the subject a PD-1 inhibitor in a dosing regimen including administration of 480 mg of the PD-1 inhibitor on Days 8, 36, 57, and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85; and (3) administering to the subject a LAG3 inhibitor in a dosing regimen including: (i) administration of a first amount of the LAG3 inhibitor, wherein the first amount is 240 mg and is administered on Days 15, 29, and 43; and (ii) administration of a second amount of the LAG3 inhibitor, wherein the second amount is 480 mg and is administered on Days 57 and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85; and (3) administering to the subject a LAG3 inhibitor in a dosing regimen including: (i) administration of a first amount of the LAG3 inhibitor, wherein the first amount is 480 mg and is administered on Days 15, 29, and 43; and (ii) administration of a second amount of the LAG3 inhibitor, wherein the second amount is 960 mg and is administered on Days 57 and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering to the subject a PD-1 inhibitor in a dosing regimen including: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including administration of 480 mg of the PD-1 inhibitor on Days 15, 57, and 85; and (3) administering to the subject a LAG3 inhibitor in a dosing regimen including administration of 480 mg of the LAG3 inhibitor on Days 15, 57, and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; (2) administering to the subject a PD-1 inhibitor in a dosing regimen including administration of 480 mg of the PD-1 inhibitor on Days 15, 57, and 85; and (3) administering to the subject a LAG3 inhibitor in a dosing regimen including administration of 960 mg of the LAG3 inhibitor on Days 15, 57, and 85.

Also provided herein is a method of treating a cancer, the method including: (1) administering to a subject having a CD19-expressing cancer a cell therapy containing a dose of engineered cells containing T cells expressing a chimeric antigen receptor (CAR) that binds cluster of differentiation 19 (CD19) on Day 1; and (2) administering to the subject a PD-1 inhibitor in a dosing regimen including administration of 480 mg of the PD-1 inhibitor on Days 15, 57, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering a PD-1 inhibitor to the subject; and (3) administering a LAG3 inhibitor to the subject. In some embodiments, a first dose of the PD-1 inhibitor and a first dose of the LAG3 inhibitor are independently administered, each between Day 2 and Day 20, inclusive.

Also provided herein is a method of treating a cancer, the method including: (1) administering to a subject having a CD19-expressing cancer a cell therapy comprising a dose of engineered cells comprising T cells expressing a chimeric antigen receptor (CAR) directed against the cancer on Day 1; (2) administering a PD-1 inhibitor to the subject; and (3) administering a LAG3 inhibitor to the subject.

In some embodiments, the first dose of the PD-1 inhibitor and the first dose of the LAG3 inhibitor are administered on the same day.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each dose of the PD-1 inhibitor is between at or about 140 mg and at or about 580 mg, inclusive.

Also provided herein is a method of treating a cancer, the method including: (1) administering to a subject having a CD19-expressing cancer a cell therapy comprising a dose of engineered cells comprising T cells expressing a chimeric antigen receptor (CAR) directed against the cancer on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each dose of the PD-1 inhibitor is between at or about 140 mg and at or about 580 mg, inclusive.

In some embodiments, each subsequent dose of the PD-1 inhibitor is administered about two weeks, about three weeks, or about four weeks after the previous dose of the PD-1 inhibitor. In some embodiments, a dose of the PD-1 inhibitor is administered about two weeks after the previous dose of the PD-1 inhibitor. In some embodiments, a dose of the PD-1 inhibitor is administered about three weeks after the previous dose of the PD-1 inhibitor. In some embodiments, a dose of the PD-1 inhibitor is administered about four weeks after the previous dose of the PD-1 inhibitor.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each subsequent dose of the PD-1 inhibitor is administered about two weeks, about three weeks, or about four weeks after the previous dose of the PD-1 inhibitor.

Also provided herein is a method of treating a cancer, the method including: (1) administering to a subject having a CD19-expressing cancer a cell therapy comprising a dose of engineered cells comprising T cells expressing a chimeric antigen receptor (CAR) directed against the cancer on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each subsequent dose of the PD-1 inhibitor is administered about two weeks, about three weeks, or about four weeks after the previous dose of the PD-1 inhibitor

In some embodiments, each dose of the PD-1 inhibitor is between at or about 140 mg and at or about 580 mg, inclusive. In some embodiments, a first dose of the PD-1 inhibitor is administered between Day 8 and Day 15, inclusive. In some embodiments, a first dose of the PD-1 inhibitor is administered on Day 8. In some embodiments, a first dose of the PD-1 inhibitor is administered on Day 15.

In some embodiments, the PD-1 inhibitor is administered for no longer than about three months. In some embodiments, a final dose of the PD-1 inhibitor is administered between about Day 80 and about Day 90. In some embodiments, the final dose of the PD-1 inhibitor is administered at about Day 85.

In some embodiments, each dose of the PD-1 inhibitor is between at or about 160 mg and 560 mg. In some embodiments, each dose of the PD-1 inhibitor is at or about 240 mg, or at or about 480 mg. In some embodiments, each dose of the PD-1 inhibitor is 240 mg. In some embodiments, each dose of the PD-1 inhibitor is 480 mg. In some embodiments, at least one dose of the PD-1 inhibitor is 240 mg, and at least one dose of the PD-1 inhibitor is 480 mg.

In some embodiments, at least four doses of the PD-1 inhibitor are administered. In some embodiments, four doses, five doses, or six doses of the PD-1 inhibitor are administered. In some embodiments, four doses of the PD-1 inhibitor are administered. In some embodiments, five doses of the PD-1 inhibitor are administered. In some embodiments, six doses of the PD-1 inhibitor are administered.

In some embodiments, the first three doses of the PD-1 inhibitor are administered every two weeks (Q2W). In some embodiments, each dose of the PD-1 inhibitor is administered every two weeks (Q2W).

In some embodiments, the fourth dose of the PD-1 inhibitor is administered about three weeks or about four weeks after the previous dose of the PD-1 inhibitor. In some embodiments, the fourth dose of the PD-1 inhibitor is administered about three weeks after the previous dose of the PD-1 inhibitor. In some embodiments, the fourth dose of the PD-1 inhibitor is administered about four weeks after the previous dose of the PD-1 inhibitor.

In some embodiments, five doses of the PD-1 inhibitor are administered. In some embodiments, the fifth dose of the PD-1 inhibitor is administered about four weeks after the fourth dose of the PD-1 inhibitor.

In some embodiments, about 240 mg of the PD-1 inhibitor is administered on each of Days 8, 22, and 36. In some embodiments, about 240 mg of the PD-1 inhibitor is administered on each of Days 15, 29, and 43. In some embodiments, about 480 mg of the PD-1 inhibitor is administered on each of Days 8, 36, 64, and 85. In some embodiments, about 480 mg of the PD-1 inhibitor is administered on each of Days 15, 43, 64, and 85.

In some embodiments, the method further includes administering a LAG3 inhibitor to the subject. In some embodiments, a first dose of the LAG3 inhibitor is administered between Day 2 and Day 20, inclusive. In some embodiments, a first dose of the LAG3 inhibitor is administered between Day 8 and Day 15, inclusive. In some embodiments, a first dose of the LAG3 inhibitor is administered on Day 8. In some embodiments, a first dose of the LAG3 inhibitor is administered on Day 15.

In some embodiments, each dose of the LAG3 inhibitor is between about 60 mg and about 540 mg, inclusive. In some embodiments, each dose of the LAG3 inhibitor is between about 120 mg and about 480 mg. In some embodiments, each dose of the LAG3 inhibitor is about 120 mg. In some embodiments, each dose of the LAG3 inhibitor is about 240 mg. In some embodiments, each dose of the LAG3 inhibitor is about 480 mg.

In some embodiments, at least three doses of the LAG3 inhibitor are administered. In some embodiments, three doses, four doses, or six doses of the LAG3 inhibitor are administered. In some embodiments, three doses of the LAG3 inhibitor are administered. In some embodiments, four doses of the LAG3 inhibitor are administered. In some embodiments, six doses of the LAG3 inhibitor are administered.

In some embodiments, the first three doses of the LAG3 inhibitor are administered every two weeks (Q2W). In some embodiments, each dose of the LAG3 inhibitor is administered every two weeks (Q2W).

In some embodiments, the second dose of the LAG3 inhibitor is administered about four weeks after the first dose of the LAG3 inhibitor. In some embodiments,

In some embodiments, doses of the PD-1 inhibitor and doses of the LAG3 inhibitor are administered with the same frequency. In some embodiments, (i) each dose of the PD-1 inhibitor is administered on the same day as a dose of the LAG3 inhibitor; and/or (ii) each dose of the LAG3 inhibitor is administered on the same day as a dose of the PD-1 inhibitor. In some embodiments, (i) each dose of the PD-1 inhibitor is administered on the same day as a dose of the LAG3 inhibitor; or (ii) each dose of the LAG3 inhibitor is administered on the same day as a dose of the PD-1 inhibitor. In some embodiments, each dose of the PD-1 inhibitor is administered on the same day as a dose of the LAG3 inhibitor. In some embodiments, each dose of the LAG3 inhibitor is administered on the same day as a dose of the PD-1 inhibitor. In some embodiments, (i) each dose of the PD-1 inhibitor is administered on the same day as a dose of the LAG3 inhibitor; and (ii) each dose of the LAG3 inhibitor is administered on the same day as a dose of the PD-1 inhibitor.

In some embodiments, doses of the LAG3 inhibitor are administered half as frequently as doses of the PD-1 inhibitor.

In some embodiments, each dose of the PD-1 inhibitor is double the dose of the LAG3 inhibitor. In some embodiments, each dose of the PD-1 inhibitor is the same as the dose of the LAG3 inhibitor.

In some embodiments, the PD-1 inhibitor and the LAG3 inhibitor are formulated in a single composition. In some embodiments, the PD-1 inhibitor and the LAG3 inhibitor are formulated in a single composition for intravenous administration.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 8, 22, 36, 57, 71, and 85; and (3) administering to the subject a LAG3 inhibitor in a dosing regimen comprising administration of 240 mg of the LAG3 inhibitor on Days 8, 36, and 71.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 15, 29, 43, 57, 71, and 85; and (3) administering to the subject a LAG3 inhibitor in a dosing regimen comprising administration of 240 mg on Days 15, 43, and 71.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 8, 22, 36, 57, 71, and 85; and (3) administering to the subject a LAG3 inhibitor in a dosing regimen comprising administration of 240 mg of the LAG3 inhibitor on Days 8, 22, 36, 57, 71, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 15, 29, 43, 57, 71, and 85; and (3) administering to the subject a LAG3 inhibitor in a dosing regimen comprising administration of 240 mg of the LAG3 inhibitor on Days 815, 29, 43, 57, 71, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 8, 22, 36, 57, 71, and 85; and (3) administering to the subject a LAG3 inhibitor in a dosing regimen comprising administration of 120 mg of the LAG3 inhibitor on Days 8, 22, 36, 57, 71, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 15, 29, 43, 57, 71, and 85; and (3) administering to the subject a LAG3 inhibitor in a dosing regimen comprising administration of 120 mg of the LAG3 inhibitor on Days 15, 29, 43, 57, 71, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 8, 36, 64, and 85; and (3) administering to the subject a LAG3 inhibitor in a dosing regimen comprising administration of 240 mg of the LAG3 inhibitor on Days 8, 36, 64, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 15, 43, 64, and 85; and (3) administering to the subject a LAG3 inhibitor in a dosing regimen comprising administration of 240 mg of the LAG3 inhibitor on Days 15, 43, 64, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 8, 36, 64, and 85; and (3) administering to the subject a LAG3 inhibitor in a dosing regimen comprising administration of 480 mg of the LAG3 inhibitor on Days 8, 36, 64, and 85.

Also provided herein is a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 15, 43, 64, and 85; and (3) administering to the subject a LAG3 inhibitor in a dosing regimen comprising administration of 480 mg of the LAG3 inhibitor on Days 15, 43, 64, and 85.

Also provided herein is use of a PD-1 inhibitor and a LAG3 inhibitor in the manufacture of a medicament for treating a subject having a CD19-expressing cancer, wherein the subject was previously administered a cell therapy comprising a dose of engineered cells comprising T cells expressing a chimeric antigen receptor (CAR) that binds cluster of differentiation 19 (CD19) on Day 1.

Also provided herein is use of a PD-1 inhibitor and a LAG3 inhibitor in the manufacture of a medicament for treating a subject having a CD19-expressing cancer, wherein the subject was previously administered a cell therapy comprising a dose of engineered cells comprising T cells expressing a chimeric antigen receptor (CAR) directed against the cancer on Day 1.

In some embodiments, the medicament is to be administered to the subject between Day 2 and Day 20.

Also provided herein is use of a PD-1 inhibitor in the manufacture of a medicament for treating a subject having a CD19-expressing cancer, wherein: (1) at least two doses of the medicament are to be administered to the subject; (2) a first dose of the medicament is to be administered to the subject between Day 2 and Day 20, inclusive; (3) each dose of the medicament comprises between at or about 140 mg and at or about 580 mg of the PD-1 inhibitor, inclusive; and (4) the subject was previously administered a cell therapy comprising a dose of engineered cells comprising T cells expressing a chimeric antigen receptor (CAR) that binds cluster of differentiation 19 (CD19) on Day 1.

Also provided herein is use of a PD-1 inhibitor in the manufacture of a medicament for treating a subject having a CD19-expressing cancer, wherein: (1) at least two doses of the medicament are to be administered to the subject; (2) a first dose of the medicament is to be administered to the subject between Day 2 and Day 20, inclusive; (3) each dose of the medicament comprises between at or about 140 mg and at or about 580 mg of the PD-1 inhibitor, inclusive; and (4) the subject was previously administered a cell therapy comprising a dose of engineered cells comprising T cells expressing a chimeric antigen receptor (CAR) directed against the cancer on Day 1.

In some embodiments, each subsequent dose of the medicament is to be administered about two weeks, about three weeks, or about four weeks after the previous dose of the medicament.

Also provided herein is use of a PD-1 inhibitor in the manufacture of a medicament for treating a CD19-expressing cancer, wherein: (1) at least two doses of the medicament are to be administered to the subject; (2) a first dose of the medicament is to be administered to the subject between Day 2 and Day 20, inclusive; (3) each subsequent dose of the medicament is to be administered about two weeks, about three weeks, or about four weeks after the previous dose of the medicament; and (4) the subject was previously administered a cell therapy comprising a dose of engineered cells comprising T cells expressing a chimeric antigen receptor (CAR) that binds cluster of differentiation 19 (CD19) on Day 1.

Also provided herein is use of a PD-1 inhibitor in the manufacture of a medicament for treating a CD19-expressing cancer, wherein: (1) at least two doses of the medicament are to be administered to the subject; (2) a first dose of the medicament is to be administered to the subject between Day 2 and Day 20, inclusive; (3) each subsequent dose of the medicament is to be administered about two weeks, about three weeks, or about four weeks after the previous dose of the medicament; and (4) the subject was previously administered a cell therapy comprising a dose of engineered cells comprising T cells expressing a chimeric antigen receptor (CAR) directed against the cancer on Day 1.

In some embodiments, each dose of the medicament comprises between at or about 140 mg and at or about 580 mg of the PD-1 inhibitor, inclusive. In some embodiments, the subject is administered a LAG3 inhibitor following administration of the cell therapy.

Also provided herein is a combination of a PD-1 inhibitor and a LAG3 inhibitor for use in a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering a PD-1 inhibitor to the subject; and (3) administering a LAG3 inhibitor to the subject.

Also provided herein is a combination of a PD-1 inhibitor and a LAG3 inhibitor for use in a method of treating a cancer, the method including: (1) administering to a subject having a CD19-expressing cancer a cell therapy comprising a dose of engineered cells comprising T cells expressing a chimeric antigen receptor (CAR) directed against the cancer on Day 1; (2) administering a PD-1 inhibitor to the subject; and (3) administering a LAG3 inhibitor to the subject.

In some embodiments, a first dose of the PD-1 inhibitor and a first dose of the LAG3 inhibitor are independently administered, each between Day 2 and Day 20. In some embodiments, the first dose of the PD-1 inhibitor and the first dose of the LAG3 inhibitor are administered on the same day.

Also provided herein is a PD-1 inhibitor for use in a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each dose of the PD-1 inhibitor is between at or about 140 mg and at or about 580 mg, inclusive.

Also provided herein is a PD-1 inhibitor for use in a method of treating a cancer, the method including: (1) administering to a subject having a CD19-expressing cancer a cell therapy comprising a dose of engineered cells comprising T cells expressing a chimeric antigen receptor (CAR) directed against the cancer on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each dose of the PD-1 inhibitor is between at or about 140 mg and at or about 580 mg, inclusive.

In some embodiments, each subsequent dose of the PD-1 inhibitor is administered about two weeks, about three weeks, or about four weeks after the previous dose of the PD-1 inhibitor.

Also provided herein is a PD-1 inhibitor for use in a method of treating a cancer, the method including: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) a first dose of the PD-1 inhibitor is administered between Day 2 and Day 20, inclusive; and (ii) each subsequent dose of the PD-1 inhibitor is administered about two weeks, about three weeks, or about four weeks after the previous dose of the PD-1 inhibitor.

Also provided herein is a PD-1 inhibitor for use in a method of treating a cancer, the method including: (1) administering to a subject having a CD19-expressing cancer a cell therapy comprising a dose of engineered cells comprising T cells expressing a chimeric antigen receptor (CAR) directed against the cancer on Day 1; and (2) administering a PD-1 inhibitor to the subject in a dosing regimen comprising administration of at least two doses, wherein: (i) a first dose of the PD-1 inhibitor is administered between Day 2 and Day 20, inclusive; and (ii) each subsequent dose of the PD-1 inhibitor is administered about two weeks, about three weeks, or about four weeks after the previous dose of the PD-1 inhibitor.

In some embodiments, each dose of the PD-1 inhibitor is between at or about 140 mg and at or about 580 mg, inclusive. In some embodiments, the method further comprises administering a LAG3 inhibitor to the subject.

In some embodiments, the PD-1 inhibitor is an anti-PD-1 antibody. In some embodiments, the anti-PD-1 antibody contains a heavy chain variable (VH) region having a CDR1, a CDR2, and a CDR3 containing the amino acid sequences set forth in SEQ ID NOS: 60, 61, and 62 respectively, and a light chain variable (VL) region having a CDR1, a CDR2, and a CDR3 containing the amino acid sequences set forth in SEQ ID NOS: 63, 64, and 65 respectively. In some embodiments, the VH region contains the amino acid sequence set forth in SEQ ID NO: 66, and the VL region contains the amino acid sequence set forth in SEQ ID NO: 67. In some embodiments, the anti-PD-1 antibody is nivolumab.

In some embodiments, the LAG3 inhibitor is an anti-LAG3 antibody. In some embodiments, the anti-LAG3 antibody contains a heavy chain variable (VH) region having a CDR1, a CDR2, and a CDR3 containing the amino acid sequences set forth in SEQ ID NOS: 68, 69, and 70 respectively, and a light chain variable (VL) region having a CDR1, a CDR2, and a CDR3 containing the amino acid sequences set forth in SEQ ID NOS: 71, 72, and 73 respectively. In some embodiments, the VH region contains the amino acid sequence set forth in SEQ ID NO: 74, and the VL region contains the amino acid sequence set forth in SEQ ID NO: 75. In some embodiments, the anti-LAG3 antibody is relatlimab.

In some embodiments, administration of the PD-1 inhibitor and administration of the LAG3 inhibitor are simultaneous. In some embodiments, simultaneous administration of the PD-1 inhibitor and the LAG3 inhibitor to the subject comprises administration of a single composition comprising the PD-1 inhibitor and the LAG3 inhibitor. In some embodiments, the single composition comprises about 480 mg of the PD-1 inhibitor and about 160 mg of the LAG3 inhibitor. In some embodiments, the PD-1 inhibitor and the LAG3 inhibitor are administered together as a single composition over the course of at or about 15 minutes, at or about 30 minutes, at or about 45 minutes, at or about 60 minutes, at or about 75 minutes, or at or about 90 minutes. In some embodiments, the PD-1 inhibitor and the LAG3 inhibitor are administered together as a single composition over the course of at or about 15 minutes. In some embodiments, the PD-1 inhibitor and the LAG3 inhibitor are administered as a single composition over the course of at or about 30 minutes. In some embodiments, the PD-1 inhibitor and the LAG3 inhibitor are administered as a single composition over the course of at or about 45 minutes. In some embodiments, the PD-1 inhibitor and the LAG3 inhibitor are administered as a single composition over the course of at or about 60 minutes. In some embodiments, the PD-1 inhibitor and the LAG3 inhibitor are administered as a single composition over the course of at or about 75 minutes. In some embodiments, the PD-1 inhibitor and the LAG3 inhibitor are administered as a single composition over the course of at or about 90 minutes.

In some embodiments, the PD-1 inhibitor and the LAG3 inhibitor are administered sequentially as separate compositions. In some embodiments, separate administration of the PD-1 inhibitor and the LAG3 inhibitor comprises a first composition comprising one of the PD-1 inhibitor and the LAG3 inhibitor, and a second composition comprising the other of the PD-1 inhibitor and the LAG3 inhibitor. In some embodiments, administration of the second composition is initiated at or about 5 minutes, at or about 10 minutes, at or about 15 minutes, at or about 20 minutes, at or about 25 minutes, at or about 30 minutes, at or about 35 minutes, at or about 40 minutes, or at or about 45 minutes after the administration of the first composition is complete. In some embodiments, administration of the second composition is initiated at or about 15 minutes after the administration of the first composition is complete. In some embodiments, administration of the second composition is initiated at or about 30 minutes after the administration of the first composition is complete. In some embodiments, the first composition comprises the PD-1 inhibitor. In some embodiments, the second composition comprises at or about 960 mg of the LAG3 inhibitor and is administered at or about 30 minutes after administration of a first composition comprising the PD-1 inhibitor.

In some embodiments, the composition comprising the PD-1 inhibitor is administered over the course of at or about 15 minutes, at or about 30 minutes, at or about 45 minutes, or at or about 60 minutes. In some embodiments, the composition comprising the PD-1 inhibitor is administered over the course of at or about 15 minutes. In some embodiments, the composition comprising the PD-1 inhibitor is administered over the course of at or about 30 minutes. In some embodiments, the composition comprising the PD-1 inhibitor is administered over the course of at or about 45 minutes.

In some embodiments, the composition comprising the LAG3 inhibitor is administered over the course of at or about 45 minutes, at or about 60 minutes, or at or about 75 minutes. In some embodiments, the composition comprising the LAG3 inhibitor is administered over the course of at or about 45 minutes. In some embodiments, the composition comprising the LAG3 inhibitor is administered over the course of at or about 60 minutes. In some embodiments, the composition comprising the LAG3 inhibitor is administered over the course of at or about 75 minutes.

In some embodiments, the method further includes administering a lymphodepleting therapy to the subject prior to administration of the dose of engineered T cells. In some embodiments, the lymphodepleting therapy is completed within about 7 days prior to initiation of the administration of the dose of engineered T cells. In some embodiments, the administration of the lymphodepleting therapy is completed within about 2 to 7 days prior to initiation of the administration of the dose of engineered T cells. In some embodiments, the lymphodepleting therapy includes the administration of fludarabine and/or cyclophosphamide. In some embodiments, the lymphodepleting therapy includes administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days. In some embodiments, the lymphodepleting therapy includes administration of cyclophosphamide at or about 300 mg/m², inclusive, for 3 days. In some embodiments, the lymphodepleting therapy includes administration fludarabine at or about 30 mg/m², daily for 3 days. In some embodiments, the lymphodepleting therapy includes administration of cyclophosphamide at or about 300 mg/m² and fludarabine at or about 30 mg/m² daily concurrently for 3 days.

In some embodiments, CD19 is human CD19.

In some embodiments, the chimeric antigen receptor (CAR) contains an scFv containing the variable heavy chain region and the variable light chain region of the antibody FMC63, a spacer that is 15 amino acids or less and contains an immunoglobulin hinge region or a modified version thereof, a transmembrane domain, and an intracellular signaling domain containing a signaling domain of a CD3-zeta (CD3ζ) chain and a costimulatory signaling region that is a signaling domain of 4-1BB.

In some embodiments, the immunoglobulin hinge region or a modified version thereof contains the formula X₁PPX₂P, wherein X₁ is glycine, cysteine or arginine and X₂ is cysteine or threonine (SEQ ID NO:58). In some embodiments, the immunoglobulin hinge region or a modified version thereof is an IgG1 hinge or a modified version thereof. In some embodiments, the immunoglobulin hinge region or a modified version thereof is an IgG4 hinge or a modified version thereof. In some embodiments, the spacer contains the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34. In some embodiments, the spacer consists of the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

In some embodiments, the spacer is at or about 12 amino acids in length. In some embodiments, the spacer contains the sequence set forth in SEQ ID NO: 1. In some embodiments, the spacer consists of the sequence set forth in SEQ ID NO: 1.

In some embodiments, the transmembrane domain is a transmembrane domain of CD28. In some embodiments, the transmembrane domain contains the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8. In some embodiments, the transmembrane domain contains the sequence of amino acids set forth in SEQ ID NO: 8. In some embodiments, the transmembrane domain contains a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8.

In some embodiments, the costimulatory domain contains the sequence set forth in SEQ ID NO: 12 or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 12. In some embodiments, the costimulatory domain contains the sequence set forth in SEQ ID NO: 12. In some embodiments, the costimulatory domain contains a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some embodiments, the signaling domain of a CD3-zeta (CD3ζ) chain contains the sequence set forth in SEQ ID NO: 13, 14, or 15, or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 13, 14, or 15. In some embodiments, the signaling domain of a CD3-zeta (CD3ζ) chain contains the sequence set forth in SEQ ID NO: 13, 14, or 15. In some embodiments, the signaling domain of a CD3-zeta (CD3ζ) chain contains a sequence of amino acids that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 13, 14, or 15. In some embodiments, the signaling domain of a CD3-zeta (CD3ζ) chain contains the sequence set forth in SEQ ID NO: 13. In some embodiments, the signaling domain of a CD3-zeta (CD3ζ) chain contains a sequence of amino acids that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 13.

In some embodiments, the scFv contains a CDRL1 sequence of SEQ ID NO: 35, a CDRL2 sequence of SEQ ID NO: 55, and a CDRL3 sequence of SEQ ID NO: 56; and a CDRH1 sequence of SEQ ID NO: 38, a CDRH2 sequence of SEQ ID NO: 39, and a CDRH3 sequence of SEQ ID NO: 54. In some embodiments, the scFv contains a CDRL1 sequence of SEQ ID NO: 35, a CDRL2 sequence of SEQ ID NO: 36, and a CDRL3 sequence of SEQ ID NO: 37; and a CDRH1 sequence of SEQ ID NO: 38, a CDRH2 sequence of SEQ ID NO: 39, and a CDRH3 sequence of SEQ ID NO: 40. In some embodiments, the scFv contains, in order from N-terminus to C-terminus, a V_{L} containing the sequence set forth in SEQ ID NO: 42, and a V_{H}, containing the sequence set forth in SEQ ID NO: 41. In some embodiments, the scFv contains the sequence set forth in SEQ ID NO: 43.

In some embodiments, the CAR contains in order from N-terminus to C-terminus: an extracellular antigen-binding domain that is the scFv set forth in SEQ ID NO: 43, the spacer set forth in SEQ ID NO: 1, the transmembrane domain set forth in SEQ ID NO: 8, the 4-1BB costimulatory signaling domain set forth in SEQ ID NO: 12, and the signaling domain of a CD3-zeta (CD3ζ) chain set forth in SEQ ID NO: 13.

In some embodiments, the dose of the engineered T cells contains CD4+ T CAR-expressing cells and CD8+ CAR-expressing T cells. In some embodiments, the dose of engineered T cells contains between about 5×10⁷ CAR-expressing T cells and about 1.1×10⁸ CAR-expressing T cells, inclusive of each. In some embodiments, the dose of engineered T cells contains about 5×10⁷ CAR-expressing T cells. In some embodiments, the dose of engineered T cells contains about 1×10⁸ CAR-expressing T cells.

In some embodiments, administration of the dose of engineered T cells includes administering a plurality of separate compositions, wherein the plurality of separate compositions includes a first composition containing the CD8+ CAR-expressing T cells and a second composition containing the CD4+ CAR-expressing T cells. In some embodiments, the first composition and the second composition are administered 0 to 12 hours apart, 0 to 6 hours apart, or 0 to 2 hours apart, or wherein the administration of the first composition and the administration of the second composition are carried out on the same day, between about 0 and about 12 hours apart, between about 0 and about 6 hours apart, or between about 0 and 2 hours apart. In some embodiments, initiation of administration of the first composition and the initiation of administration of the second composition are carried out between about 1 minute and about 1 hour apart or between about 5 minutes and about 30 minutes apart. In some embodiments, the first composition and the second composition are administered no more than 2 hours, no more than 1 hour, no more than 30 minutes, no more than 15 minutes, no more than 10 minutes, or no more than 5 minutes apart. In some embodiments, the first composition and the second composition are administered less than 2 hours apart. In some embodiments, the first composition containing the CD8⁺ CAR-expressing T cells is administered prior to the second composition containing the CD4⁺ CAR-expressing T cells. In some embodiments, the cells of the dose of the engineered T cells are administered intravenously.

In some embodiments, the T cells are primary T cells obtained from a sample from the subject, optionally wherein the sample is a whole blood sample, an apheresis sample, or a leukapheresis sample. In some embodiments, the sample is an apheresis sample. In some embodiments, the sample is a leukapheresis sample. In some embodiments, the sample is obtained from the subject prior to administration of the lymphodepleting therapy to the subject. In some embodiments, the T cells are autologous to the subject. In some embodiments, the subject is human.

In some embodiments, the CD19-expressing cancer is a B cell malignancy. In some embodiments, the CD19-expressing cancer is a myeloma, a leukemia, or a lymphoma. In some embodiments, the CD19-expressing cancer is an acute lymphoblastic leukemia (ALL), adult ALL, chronic lymphoblastic leukemia (CLL), a small lymphocytic lymphoma (SLL), non-Hodgkin lymphoma (NHL), or a large B cell lymphoma. In some embodiments, the CD19-expressing cancer is a non-Hodgkin lymphoma (NHL). In some embodiments, the NHL is selected from the group consisting of: diffuse large B-cell lymphoma (DLBCL) not otherwise specified (NOS) including transformed indolent NHL, follicular lymphoma Grade 3B (FL3B), T cell/histiocyte-rich large B-cell lymphoma, Epstein-Barr virus (EBV) positive DLBCL NOS, primary mediastinal (thymic) large B-cell lymphoma, and high grade B-cell lymphoma with MYC and BCL2 and/or BCL6 rearrangements with DLBCL histology (double/triple-hit lymphoma). In some embodiments, the NHL is selected from the group consisting of: diffuse large B-cell lymphoma (DLBCL) not otherwise specified (NOS) including transformed indolent NHL, follicular lymphoma Grade 3B (FL3B), T cell/histiocyte-rich large B-cell lymphoma, Epstein-Barr virus (EBV) positive DLBCL NOS, primary mediastinal (thymic) large B-cell lymphoma, Richter's Transformation and high grade B-cell lymphoma with MYC and BCL2 and/or BCL6 rearrangements with DLBCL histology (double/triple-hit lymphoma).

In some embodiments, the NHL is a relapsed/refractory (R/R) NHL. In some embodiments, the subject is relapsed or refractory to at least two prior lines of systemic therapy for the CD19-expressing cancer. In some embodiments, at least one of the at least two prior lines of systemic therapy includes a CD20-targeted agent and an anthracycline.

In some embodiments, the subject has an ECOG performance status of 0 or 1. In some embodiments, the subject has an ECOG performance status of 0. In some embodiments, the subject has an ECOG performance status of 1. In some embodiments, the subject's ECOG performance status is determined at the time of the administration of the dose of engineered T cells. In some embodiments, the subject's ECOG performance status is determined at screening. In some embodiments, screening occurs between about one week and about two weeks prior to the administration of the dose of engineered T cells.

In some embodiments, the subject has positron-emission tomography (PET)-positive disease. In some embodiments, the PET-positive disease is determined at the time of the administration of the dose of engineered T cells. In some embodiments, the PET-positive disease is determined at the time of screening. In some embodiments, the subject has computed tomography (CT) measurable disease. In some embodiments, the CT measurable disease is determined at the time of the administration of the dose of engineered T cells. In some embodiments, the CT measurable disease is determined at the time of screenin. In some embodiments, the subject has PET-positive and CT measurable disease. In some embodiments, screening occurs between about one week and about two weeks prior to the administration of the dose of engineered T cells.

In some embodiments, the subject has a sum of product of perpendicular diameters (SPD) of up to 6 index lesions of greater than or equal to 25 cm². In some embodiments, SPD is measured by CT scan. In some embodiments, the subject has a sum of product of perpendicular diameters (SPD) of up to 6 index lesions of greater than or equal to 25 cm² by CT scan. In some embodiments, SPD is determined at the time of the administration of the dose of engineered T cells. In some embodiments, SPD is determined at the time of screening. In some embodiments, screening occurs between about one week and about two weeks prior to the administration of the dose of engineered T cells.

### BRIEF DESCRIPTION OF THE DRAWINGS

FIG. 1 shows the expression, indicated by mean fluorescence intensity (MFI), of LAG3 by endogenous CD3+ cells in subjects exhibiting either a complete response (CR) or progressive disease (PD) at 3 months post-treatment.

FIG. 2A shows the correlation of LAG3 gene expression by CAR T cells and the maximum concentration (Cₘₐₓ) of CD3+ CAR T cells (cells/μL). Pearson correlation: −0.70, p=1.3×10⁻⁷; Spearman correlation: −0.71, p=3.2×10⁻⁷.

FIG. 2B shows LAG3 gene expression between subjects exhibiting short (<90 days) or long (>180 days) progression-free survival (PFS) (p=0.012).

FIG. 3 shows PD-1 gene expression by endogenous T cells 2 months following treatment for subjects who exhibited either a complete response (CR) or progressive disease (PD) at 9 months post-treatment.

FIG. 4A shows PD-1 expression by CD3+ CAR+ cells 3 months following treatment for subjects exhibiting either a complete response (CR) or progressive disease (PD) at 3 months post-treatment, as analyzed the mean fluorescence intensity (MFI) of PD-1 expression.

FIG. 4B shows PD-1 expression by CD4+ CAR+ cells 3 months following treatment for subjects exhibiting either a complete response (CR) or progressive disease (PD) at 3 months post-treatment, as analyzed by the mean fluorescence intensity (MFI) of PD-1 expression.

FIG. 4C shows PD-1 expression by CD8+ CAR+ cells 3 months following treatment for subjects exhibiting either a complete response (CR) or progressive disease (PD) at 3 months post-treatment, as analyzed by or the mean fluorescence intensity (MFI) of PD-1 expression.

FIGS. 5A and 5B show PD-1 expression by CD3+ CAR+ cells at various time points following treatment for subjects exhibiting either a complete response (CR) or progressive disease (PD) at 3 months post-treatment.

FIG. 6 shows exemplary dosing regimens for treating a non-Hodgkin lymphoma (NHL) with a combination therapy of CAR-expressing T cells, an exemplary anti-PD-1 antibody, and optionally an exemplary anti-LAG3 antibody (D: Day; R: relatlimab; N: nivolumab; LDC: lymphodepleting chemotherapy).

FIG. 7A shows peripheral LAG3 receptor occupancy (RO) at various doses of relatlimab and nivolumab, dosed every four weeks (Q4W).

FIG. 7B shows predicted tumoral LAG3 receptor occupancy (RO) at various doses of relatlimab, dosed every four weeks (Q4W).

FIG. 7C shows predicted free soluble LAG3 (sLAG3) over time with doses of 800 mg relatlimab.

FIGS. 8A-8C show exemplary dosing regimens for treating a non-Hodgkin lymphoma (NHL) with a combination therapy of CAR-expressing T cells, an exemplary anti-PD-1 antibody, and optionally an exemplary anti-LAG3 antibody (D: Day; R: relatlimab; N: nivolumab; LDC: lymphodepleting chemotherapy). For each cohort, leukapheresis, LDC, and CAR T cell infusion occur on the days indicated.

### DETAILED DESCRIPTION

All of the references cited herein are incorporated herein by reference in their entireties.

Provided herein are combination therapies for treating a subject having a cancer involving administration of a T cell therapy (e.g. CAR-T cells) and a checkpoint inhibitor therapy (e.g. an anti-PD-1 antibody and/or an anti-LAG3 antibody) for treating a cancer or a tumor, such as a lymphoma, in particular a non-Hodgkin lymphoma (NHL). In some embodiments, the T cell therapy includes any such therapy that specifically binds to an antigen associated with, expressed by, or present on cells of the cancer. In particular embodiments, the T cell therapy is or involves T cells expressing a chimeric antigen receptor (CAR) that binds CD19. In particular embodiments, the T cell therapy is or involves T cells expressing a chimeric antigen receptor (CAR) that binds CD20. In particular embodiments, the T cell therapy is or involves T cells expressing a chimeric antigen receptor (CAR) that binds CD22. In some embodiments, the checkpoint inhibitor therapy includes one or more antibodies targeting a checkpoint inhibitor, such as PD-1 and/or LAG3. Among the provided embodiments are combination therapies involving administration of a T cell therapy (e.g., CAR-expressing T cells), and administration of a checkpoint inhibitor therapy (e.g. an anti-PD-1 antibody and/or an anti-LAG3 antibody). Thus, in some embodiments, the methods and uses include administering to a subject T cells expressing genetically engineered (recombinant) cell surface receptors in adoptive cell therapy, which generally are chimeric receptors such as chimeric antigen receptors (CAR) recognizing an antigen expressed by, associated with and/or specific to the cell type from which it is derived, in combination with an anti-PD-1 antibody, and optionally, an anti-LAG3 antibody.

In some embodiments, the combination of the T cell therapy and the checkpoint inhibitor therapy is administered to a subject having a particular disease or condition to be treated, e.g., via adoptive cell therapy, such as adoptive T cell therapy. In some embodiments, the methods involve treating a subject having a CD19-expressing cancer, such a B cell malignancy (e.g. NHL) with a dose of antigen receptor-expressing cells (e.g. CAR-expressing cells).

In some embodiments, the provided methods involve treating a specific group or subset of subjects, e.g., subjects identified as having high-risk disease. In some aspects, the methods treat subjects having a poor prognosis NHL, such as NHL that has relapsed or is refractory (R/R) to standard therapy and/or has a poor prognosis. In some cases, the overall response rate (ORR) to available therapies, to a standard of care, or to a reference therapy for the disease and/or patient population for which the therapy is indicated, is less than 40% and/or the complete response (CR) is less than 20%. In some embodiments, in chemorefractory DLBCL, the ORR with a reference or available treatment or standard-of-care therapy is about 26% and the CR is about 8% (Crump et al. Outcomes in refractory aggressive diffuse large B-cell lymphoma (DLBCL): Results from the international SCHOLAR study. ASCO 2016 [Abstract 7516]). In some aspects, the provided methods, compositions, uses and articles of manufacture achieve improved and superior responses to available therapies.

CD19 is a 95 kDa glycoprotein present on B cells from early development until differentiation into plasma cells (Stamenkovic et al., J Exp Med. 1988; 168(3):1205-10). It is a member of the immunoglobulin superfamily and functions as a positive regulator of the B-cell receptor by lowering the signaling threshold for B-cell activation (Brentjens et al., Blood. 2011; 118(18):4817-28; LeBien et al, Blood. 2008; 112(5):1570-80). CD19 is an attractive therapeutic target because it is expressed by most B-cell malignancies, including B-cell NHL (Davila et al., Oncoimmunology. 2012; (9):1577-83). Importantly, the CD19 is not expressed on hematopoietic stem cells or on any normal tissue apart from those of the B-cell lineage. Additionally, CD19 is not shed in the circulation, which limits off-target adverse effects (Shank et al., Pharmacotherapy. 2017; 37(3):334-45).

In particular embodiments, the methods provided herein are based on administration of a CD19-directed CAR T cell therapy in which the CAR contains a CD19-directed scFv antigen binding domain (e.g. from FMC63) in combination with a checkpoint inhibitor therapy. The CAR further contains an intracellular signaling domain containing a signaling domain from CD3zeta, and also incorporates a 4-1BB costimulatory domain, which has been associated with lower incidence of cytokine release syndrome (CRS) and neurotoxicity (NT; e.g. neurological events (NE)) compared with CD28-containing constructs (Lu et al. J Clin Oncol. 2018; 36:3041). In some embodiments, the methods provided herein include CD8+ and CD4+ T-cell subsets that are transduced and expanded separately in vitro, and administered at equal (about 1:1) target doses. In some embodiments, there is low variability in the administered total CD4+ and CD8+ CAR+ T-cell doses, two parameters associated with increased toxicity in previous studies (Neelapu et al., N Engl Med. 2017. 377; 2531-44; Turtle et al., Sci Transl Med. 2016; 8:355ra116; Hay et al., Blood. 2017; 130:2295-306).

In particular embodiments, the methods provided herein are based on administration of a CD19-directed CAR T cell therapy in which the CAR contains a CD19-directed scFv antigen binding domain (e.g. from FMC63) in combination with a checkpoint inhibitor therapy including an anti-PD-1 antibody, and optionally an anti-LAG3 antibody.

In some embodiments, the methods include administration of the combination therapy to a subject selected or identified as having a certain prognosis or risk of a B cell malignancy, such as a NHL. Lymphomas, such as NHL, can be a variable disease. Some subjects with NHL may survive without treatment while others may require immediate intervention. In some embodiments, the methods, uses and articles of manufacture involve, or are used for treatment of subjects involving, selecting or identifying a particular group or subset of subjects, e.g., based on specific types of disease, diagnostic criteria, prior treatments and/or response to prior treatments, such as any group of subjects as described. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy. In some embodiments, at least one of the one or more prior lines of therapy included an anti-CD20 agent and an anthracycline. In some embodiments, the methods involve treating a subject having relapsed following remission after treatment with, or become refractory to, two or more prior therapies; or a subject that has relapsed or is refractory (R/R) to two or more prior therapies, e.g., two or more lines of standard therapy. In some embodiments, at least one of the two or more prior lines of therapy included an anti-CD20 agent and an anthracycline.

In particular embodiments, the provided combination therapies and methods improve responses to the T cell therapy by activity of the checkpoint inhibitor therapy to reduce or prevent exhaustion of endogenous T cells, cells of the T cell therapy (e.g. CAR T cells), or both. In some embodiments, the endogenous T cells, cells of the T cell therapy (e.g., CAR T cells), or both, thereby exhibit improved efficacy, such as effector-mediated killing of antigen-expressing cells when provided with the checkpoint inhibitor therapy.

In some embodiments, the provided combination therapies and methods improve responses to the T cell therapy by activity of the checkpoint inhibitor therapy to increase the expansion and/or persistence of administered cells of the T cell therapy (e.g. CAR T cells). Thus, in some embodiments, the cells of the T cell therapy (e.g. CAR T cells) exhibit improved expansion and/or persistence when provided with the checkpoint inhibitor therapy.

In some aspects, the provided methods, compositions, uses and articles of manufacture achieve improved and superior responses to available therapies, including a T cell therapy alone or a checkpoint inhibitor therapy alone. In some embodiments, the improved or superior responses are compared to current standard of care (SOC).

Also provided are combinations and articles of manufacture, such as kits, that contain a composition comprising the T cell therapy and/or a composition comprising the checkpoint inhibitor therapy, e.g., an anti-PD-1 antibody and/or an anti-LAG3 antibody, and uses of such compositions and combinations to treat or prevent a CD19-expressing cancer, such as a B cell malignancy (e.g. non-Hodgkin lymphoma).

Cell therapies, such as T cell-based therapies, for example, adoptive T cell therapies (including those involving the administration of cells expressing chimeric receptors specific for a cancer of interest, such as chimeric antigen receptors (CARs) and/or other recombinant antigen receptors, as well as other adoptive immune cell and adoptive T cell therapies) can be effective in the treatment of diseases and disorders such as a B cell malignancies. The engineered expression of recombinant receptors, such as chimeric antigen receptors (CARs), on the surface of T cells enables the redirection of T cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17).

In certain contexts, available approaches to adoptive cell therapy may not always be entirely satisfactory. In some contexts, optimal efficacy can depend on the ability of the administered cells to recognize and bind to a target, e.g., target antigen, and to exert various effector functions, including cytotoxic killing of cancer cells and secretion of various factors such as cytokines. In some cases, however, the administered cells may become exhausted, and thereby become less effective in exerting various effector functions, including killing of cancer cells and secretion of cytokines. In particular, results herein demonstrate that a checkpoint inhibitor therapy may reduce or prevent the exhaustion of cells of a T cell therapy.

In some aspects, the provided combination methods and uses provide for or achieve improved or more durable responses or efficacy as compared to alternative methods, such as alternative methods involving only the administration of the T cell therapy but not in combination with the checkpoint inhibitor therapy (e.g. an anti-PD-1 antibody and optionally, an anti-LAG3 antibody). In some embodiments, the methods are advantageous by virtue of administering a checkpoint inhibitor therapy (e.g. an anti-PD-1 antibody and optionally, an anti-LAG3 antibody) after (e.g. within 8 or 15 days of) administration of a T cell therapy (e.g. CAR-T cells), thereby preventing or reducing exhaustion of the administered cells of the T cell therapy. In some embodiments of the provided methods, the T cell therapy is CAR-expressing T cells (CAR T cells), and it is further found that the advantageous effect of preventing or reducing exhaustion of the administered cells can be achieved by initiating administration of the checkpoint inhibitor therapy (e.g. an anti-PD-1 antibody and optionally, an anti-LAG3 antibody) in a window of time after initiation of administration of the T cell therapy to minimize or avoid a detrimental effect of the checkpoint inhibitor therapy on cells of the T cell therapy. In particular, administration of the checkpoint inhibitor therapy may exacerbate activation induced cell death (AICD) of cells of the T cell therapy. However, delaying administration of the checkpoint inhibitor therapy (e.g. an anti-PD-1 antibody and optionally, an anti-LAG3 antibody) until a time after AICD is near or has reached its peak, or has decreased after having peaked, can avoid detrimental effects on the cells of the T cell therapy while substantially improving, e.g. synergistically increasing, T cell-mediated killing of the tumor by cells of the T cell therapy (e.g. CAR-T cells).

The provided methods are based on observations blockade of PD-1 may improve the anti-tumor activity of CAR-expressing T cells, such as by blocking inhibitory signals and preventing or restoring T cells from exhaustion, and that such improvement may be further increased by the addition of LAG3 blockade. In some embodiments, the combination of PD-1 and LAG3 blockade exhibits synergistic effects.

LAG3 is a checkpoint receptor protein expressed on chronically exhausted T-cells and is frequently co-expressed with PD-1, another checkpoint protein, on tolerized tumor infiltrating lymphocytes (TILs) across many tumor types (Chauvin et al., (2015)125(5):2046-58; Speiser et al., Nat Rev Immunol (2014) 14(11):768-74; Sharma et al., Cell (2017) 168(4):707-723). Notably, it is observed herein that in subjects treated with CAR T cells, exhaustion markers including PD-1 and LAG3 are observed to be upregulated on both CAR T cells and untransduced T cells in subjects that do not respond or respond poorly to therapy (see Example 1). These results evidence that administration of a checkpoint inhibitor therapy may improve responses to T cell therapies, such as CAR T cells. In particular, dual checkpoint inhibition, such as with an anti-PD-1 antibody and an anti-LAG3 antibody may result in enhanced T-cell effector function that is greater than the effects of either antibody alone.

Programmed death-1 (PD-1) cluster of differentiation 279 (CD279) is a cell surface membrane receptor. PD-1 is a negative regulatory molecule expressed by activated T and B lymphocytes. Binding of PD-1 to its ligands, programmed death-ligands 1 (PD-L1) and 2 (PD-L2), results in the down-regulation of lymphocyte activation. Inhibition of the interaction between PD-1 and its ligands promotes immune responses and antigen-specific T-cell responses to both foreign antigens as well as self-antigens.

Subsequent to activation, T cells express PD-1 on their surface and secrete interferons that in turn induce expression of checkpoint inhibitors such as PD-L1 and PD-L2 (Garcia Diaz et al., Cell Rep (2017) 19(6):1189-1201) on tumor cells and bystander cells (Hoekstra et al., Nat Cancer (2020) 1(3):291-301). Binding of PD-L1 and/or PD-L2 on tumor cells (including lymphoma) and the tumor microenvironment to PD-1 on T cells (including CAR T cells) results in inhibitory checkpoint signaling that decreases cytotoxicity and leads to T cell exhaustion (Andorsky, Clin Cancer Res (2011) 17(13):4232-44).

Various strategies exist for blocking the PD-1/PD-L1 pathway. In some aspects, the PD-1/PD-L1 pathway is targeted by PD-1 blockade, including PD-1 pathway inhibitors such as anti-PD-1 antibodies. PD-1 pathway inhibitors have been shown to be safe and effective in subjects with various cancers, and may be useful in reversing the PD-L1 mediated immunosuppression in subjects treated with CAR T cells.

A PD-1 pathway inhibitor for use in the methods of the disclosure includes, but is not limited to, a PD-1 inhibitor and/or a PD-L1 inhibitor.

In some aspects, the PD-1 inhibitor and/or the PD-L1 inhibitor is a small molecule.

In some aspects, the PD-1 inhibitor and/or the PD-L1 inhibitor is a millamolecule.

In some aspects, the PD-1 inhibitor and/or the PD-L1 inhibitor is a macrocyclic peptide.

In certain aspects, the PD-1 inhibitor and/or the PD-L1 inhibitor is BMS-986189.

In some aspects, the PD-1 inhibitor is an inhibitor disclosed in International Publication No. WO2014/151634, which is incorporated by reference herein in its entirety.

In some aspects, the PD-1 inhibitor is INCMGA00012 (Insight Pharmaceuticals).

In some aspects, the PD-1 inhibitor comprises a combination of an anti-PD-1 antibody disclosed herein and a PD-1 small molecule inhibitor.

In some aspects, the PD-L1 inhibitor comprises a millamolecule having a formula set forth in formula (I):

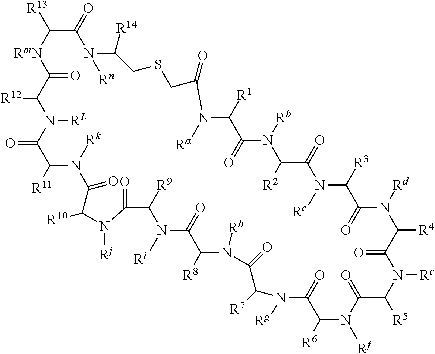

wherein R¹-R¹³ are amino acid side chains, R^{a}-Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In some aspects, the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224 (see, e.g., US 2013/0017199).

In some aspects, the PD-1 pathway inhibitor is an anti-PD-1 antibody and/or an anti-PD-L1 antibody.

In some aspects, the PD-1 pathway inhibitor is formulated for intravenous administration.

In some aspects, the PD-1 pathway inhibitor is administered at a flat dose.

In some aspects, the PD-1 pathway inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the PD-1 pathway inhibitor is administered at a dose of about 0.25 mg, about 0.5 mg, about 0.75 mg, about 1 mg, about 1.25 mg, about 1.5 mg, about 1.75 mg, about 2 mg, 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the PD-1 pathway inhibitor is administered at a weight-based dose.

In some aspects, the PD-1 pathway inhibitor is administered at a dose of from at least about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the PD-1 pathway inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the PD-1 pathway inhibitor is administered in a constant amount.

In some aspects, the dose of the PD-1 pathway inhibitor is administered in a varying amount. For example, in some aspects, a maintenance (or follow-on) dose of the PD-1 pathway inhibitor can be higher or the same as a loading dose that is first administered. In some aspects, the maintenance dose of the PD-1 pathway inhibitor can be lower or the same as the loading dose.

In some aspects, the dose of the PD-1 pathway inhibitor is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

### Anti-PD-1 Antibodies

Anti-PD-1 antibodies that are known in the art can be used in the methods of the disclosure. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a KD of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies that can be used in the methods of the disclosure have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Anti-PD-1 antibodies that can be used in the methods of the disclosure include nivolumab (also known as OPDIVO®, 5C4, BMS-936558, MDX-1106, and ONO-4538), pembrolizumab (Merck; also known as KEYTRUDA®, lambrolizumab, and MK3475; see WO 2008/156712), PDR001 (Novartis; also known as spartalizumab; see WO 2015/112900 and U.S. Pat. No. 9,683,048), MEDI-0680 (AstraZeneca; also known as AMP-514; see WO 2012/145493), TSR-042 (Tesaro Biopharmaceutical; also known as ANBO11 or dostarlimab; see WO 2014/179664), cemiplimab (Regeneron; also known as LIBTAYO® or REGN2810; see WO 2015/112800 and U.S. Pat. No. 9,987,500), JS001 (TAIZHOU JUNSHI PHARMA; also known as toripalimab; see Si-Yang Liu et al., J. Hematol. Oncol. 10:136 (2017)), PF-06801591 (Pfizer; also known as sasanlimab; US 2016/0159905), BGB-A317 (Beigene; also known as tislelizumab; see WO 2015/35606 and US 2015/0079109), BI 754091 (Boehringer Ingelheim; see Zettl M et al., Cancer. Res. (2018); 78(13 Suppl):Abstract 4558), INCSHR1210 (Jiangsu Hengrui Medicine; also known as SHR-1210 or camrelizumab; see WO 2015/085847; Si-Yang Liu et al., J. Hematol. Oncol. 10:136 (2017)), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), IBI308 (Innovent; also known as sintilimab; see WO 2017/024465, WO 2017/025016, WO 2017/132825, and WO 2017/133540), and SSI-361 (Lyvgen Biopharma Holdings Limited, US 2018/0346569).

Anti-PD-1 antibodies that can be used in the methods of the disclosure also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab.

In some aspects, the antibodies that cross-compete for binding to human PD-1 with, or bind to the same epitope region as, any anti-PD-1 antibody disclosed herein, e.g., nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies that can be used in the methods of the disclosure also include antigen-binding portions of any of the above full-length antibodies.

Anti-PD-1 antibodies that can be used in the methods of the disclosure are antibodies that bind to PD-1 with high specificity and affinity, block the binding of PD-L1 and/or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody is a full-length antibody. In some aspects, the anti-PD-1 antibody is a monoclonal, human, humanized, chimeric, or multispecific antibody. In some aspects, the multispecific antibody is a DART, a DVD-Ig, or bispecific antibody.

In some aspects, the anti-PD-1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-1 antibody is nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, 1B1308, or SSI-361, or comprises an antigen binding portion thereof.

In some aspects, the anti-PD-1 antibody is nivolumab. Nivolumab is a human monoclonal antibody that targets PD-1. Nivolumab (Opdivo®) is approved for the treatment of several types of cancer in multiple regions including the United States (US, December-2014), the European Union (EU, June-2015), and Japan (JP, July-2014). Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In some aspects, the anti-PD-1 antibody is pembrolizumab. Pembrolizumab is a humanized monoclonal IgG4 (S228P) antibody directed against human cell surface receptor PD-1. Pembrolizumab is described, for example, in U.S. Pat. Nos. 8,354,509 and 8,900,587.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 4 weeks. In some aspects, pembrolizumab is administered at a flat dose of about 400 mg once about every 6 weeks. In some aspects, pembrolizumab is administered at a flat dose of about 300 mg once about every 4-5 weeks.

In some aspects, pembrolizumab is administered intravenously at a dose of about 200 mg on Day 1, then once about every 3 weeks. In some aspects, pembrolizumab is administered for up to 35 cycles. In some aspects, pembrolizumab is administered intravenously at a dose of about 200 mg for about 30 minutes on Day 1 of a three-week cycle for up to 35 cycles.

In some aspects, the anti-PD-1 antibody is cemiplimab (REGN2810). Cemiplimab is described, for example, in WO 2015/112800 and U.S. Pat. No. 9,987,500.

In some aspects, cemiplimab is administered intravenously at a dose of about 3 mg/kg or about 350 mg once about every 3 weeks.

In some aspects, the anti-PD-1 antibody is spartalizumab (PDR001). Spartalizumab is described, for example, in WO 2015/112900 and U.S. Pat. No. 9,683,048.

In some aspects, spartalizumab is administered intravenously at a dose of about 300 mg once about every 3 weeks or 400 mg once about every 4 weeks.

### Anti-PD-L1 Antibodies

In some aspects, the PD-1/PD-L1 pathway is targeted by PD-1 blockade, including by anti-PD-L1 antibodies. Anti-PD-L1 antibodies that are known in the art can be used in the methods of the disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a KD of 1×10⁴ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

Anti-PD-L1 antibodies that can be used in the methods of the disclosure include BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), ICO 36, FAZ053 (Novartis), and CK-301 (Checkpoint Therapeutics; see Gorelik et al., AACR:Abstract 4606 (April 2016)).

Anti-PD-L1 antibodies that can be used in the methods of the disclosure also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. In certain aspects, the antibodies that cross-compete for binding to human PD-L1 with, or bind to the same epitope region as, any anti-PD-L1 antibody disclosed herein, e.g., atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies that can be used in the methods of the disclosure also include antigen-binding portions of any of the above full-length antibodies.

Anti-PD-L1 antibodies that can be used in the methods of the disclosure are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

In some aspects, an anti-PD-L1 antibody is substituted for the anti-PD-1 antibody in any of the methods disclosed herein.

In some aspects, the anti-PD-L1 antibody is a full-length antibody.

In some aspects, the anti-PD-L1 antibody is a monoclonal, human, humanized, chimeric, or multispecific antibody. In some aspects, the multispecific antibody is a DART, a DVD-Ig, or bispecific antibody.

In some aspects, the anti-PD-L1 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, FAZ053, or CK-301, or comprises an antigen binding portion thereof.

In some aspects, the PD-L1 antibody is atezolizumab. Atezolizumab is a fully humanized IgG1 monoclonal anti-PD-L1 antibody. In some aspects, atezolizumab is administered as a flat dose of about 800 mg once about every 2 weeks. In some aspects, atezolizumab is administered as a flat dose of about 840 mg once about every 2 weeks.

In some aspects, atezolizumab is administered intravenously at a dose of about 1,200 mg on Day 1 of a three-week cycle.

In some aspects, atezolizumab is administered intravenously at a dose of about 1,200 mg on Day 1 of a three-week cycle, and bevacizumab is administered at a dose of about 15 mg/kg on Day 1 of each cycle.

In some aspects, the PD-L1 antibody is durvalumab. Durvalumab is a human IgG1 kappa monoclonal anti-PD-L1 antibody. In some aspects, durvalumab is administered at a dose of about 10 mg/kg once about every 2 weeks. In some aspects, durvalumab is administered at a dose of about 10 mg/kg once about every 2 weeks for up to 12 months. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the PD-L1 antibody is avelumab. Avelumab is a human IgG1 lambda monoclonal anti-PD-L1 antibody. In some aspects, avelumab is administered as a flat dose of about 800 mg once about every 2 weeks.

Phase 1 studies using antibodies against PD-1 or PD-L1 in combination with CD19-directed CAR T therapies have demonstrated acceptable safety profiles and indicate potentially beneficial effect on CAR T pharmacokinetics, indicating that further evaluation of this approach is warranted (Siddiqi et al., Blood (2017) 129(8):1039-41; Tholouli et al., Ann Oncol (2020) 31 Suppl 4; 5651). However, some patients become resistant to therapies targeting PD-1 and/or PD-L1. While the exact mechanisms for such resistance are largely unknown, it is postulated that anti-PD-1 and/or anti-PD-L1 therapy may cause T cells to upregulate other inhibitory receptors, such as TIM3, LAG3, and/or CTLA4.

Addition of LAG3 blockade to PD-1 blockade in combination with a T cell therapy, e.g. CAR T cells, may further improve anti-tumor activity, such as in aggressive NHL, by blocking inhibitory signals and preventing or restoring T cells from exhaustion. Lymphocyte activation gene 3 (LAG3; also known as cluster of differentiation 223 (CD223) is a checkpoint receptor expressed on several immune cell types including activated CD4+ and CD8+ T cells, memory T cells, Treg cells, and natural killer cells (Andrews et al., Immunol Rev (2017) 276(1): 80-96). Activation of the LAG3 pathway occurs when LAG3 interacts with its ligands, such as MHC Class II or other emerging ligands (eg, FGL1), which triggers inhibitory activity that reduces the function of effector T cells (Andrews et al., Immunol Rev (2017) 276(1): 80-96; Wang et al., Cell (2019) 176(1-1): 334-347). Increased expression of LAG3 on TILs, especially in the context of PD-1 expression, further promotes T cell exhaustion, leading to an impaired ability to attack tumor cells and an increased potential for tumor growth (Andrews et al., (2017) 276(1): 80-96; Woo et al., Cancer Res (2012) 72(4):917-27). Inhibition of the LAG3 pathway may restore effector function of exhausted T cells, promoting proinflammatory cytokine signaling, and ultimately, an anti-tumor response.

Various anti-LAG3 strategies exist for LAG3 blockade, including LAG3 inhibitors, such as anti-LAG3 antibodies. LAG3 inhibitors for use in the methods of the disclosure include, but are not limited to, LAG3 antibodies and soluble LAG3 polypeptides. Anti-LAG3 antibodies include antibodies that specifically bind to LAG3 (i.e., an “anti-LAG3 antibody”).

In some aspects, the LAG3 inhibitor is an anti-LAG3 antibody.

Antibodies that bind to LAG3 have been disclosed, for example, in Int'l Publ. No. WO/2015/042246 and U.S. Publ. Nos. 2014/0093511 and 2011/0150892, each of which is incorporated by reference herein in its entirety.

An exemplary LAG3 antibody useful in the present disclosure is 25F7 (described in U.S. Publ. No. 2011/0150892). An additional exemplary LAG3 antibody useful in the present disclosure is BMS-986016 (relatlimab). In some aspects, an anti-LAG3 antibody useful in the present disclosure cross-competes with 25F7 or BMS-986016. In some aspects, an anti-LAG3 antibody useful in the present disclosure binds to the same epitope as 25F7 or BMS-986016. In some aspects, an anti-LAG3 antibody comprises six CDRs of 25F7 or BMS-986016.

Other art-recognized anti-LAG3 antibodies that can be used in the methods of the disclosure include IMP731 (H5L7BW) described in US 2011/007023, MK-4280 (28G-10, favezelimab) described in WO2016028672 and U.S. Publication No. 2020/0055938, REGN3767 (fianlimab) described in Burova E, et al., J. Immunother. Cancer (2016); 4(Supp. 1):P195 and U.S. Pat. No. 10,358,495, humanized BAP050 described in WO2017/019894, GSK2831781, IMP-701 (LAG-525; ieramilimab) described in U.S. Pat. No. 10,711,060 and U.S. Publ. No. 2020/0172617, aLAG3(0414), aLAG3(0416), Sym022, TSR-033, TSR-075, XmAb841 (previously XmAb22841), MGD013 (tebotelimab), B1754111, FS118, P 13B02-30, AVA-017, AGEN1746, RO7247669, INCAGN02385, IBI-110, EMB-02, IBI-323, LBL-007, and ABL501. These and other anti-LAG3 antibodies useful in the claimed invention can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

Anti-LAG3 antibodies that can be used in the methods of the disclosure also include isolated antibodies that bind specifically to human LAG3 and cross-compete for binding to human LAG3 with any anti-LAG3 antibody disclosed herein, e.g., relatlimab. In some aspects, the anti-LAG3 antibody binds the same epitope as any of the anti-LAG3 antibodies described herein, e.g., relatlimab.

In some aspects, the antibodies that cross-compete for binding to human LAG3 with, or bind to the same epitope region as, any anti-LAG3 antibody disclosed herein, e.g., relatlimab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

The ability of antibodies to cross-compete for binding to an antigen indicates that the antibodies bind to the same epitope region of the antigen and sterically hinder the binding of other cross-competing antibodies to that particular epitope region. These cross-competing antibodies are expected to have functional properties very similar those of the reference antibody, e.g., relatlimab, by virtue of their binding to the same epitope region. Cross-competing antibodies can be readily identified based on their ability to cross-compete in standard binding assays such as Biacore analysis, ELISA assays or flow cytometry (see, e.g., WO 2013/173223).

Anti-LAG3 antibodies that can be used in the methods of the disclosure also include antigen-binding portions of any of the above full-length antibodies. It has been amply demonstrated that the antigen-binding function of an antibody can be performed by fragments of a full-length antibody.

In some aspects, the anti-LAG3 antibody is a full-length antibody.

In some aspects, the anti-LAG3 antibody is a monoclonal, human, humanized, chimeric, or multispecific antibody. In some aspects, the multispecific antibody is a dual-affinity re-targeting antibody (DART), a DVD-Ig, or bispecific antibody.

In some aspects, the anti-LAG3 antibody is a F(ab′)₂ fragment, a Fab′ fragment, a Fab fragment, a Fv fragment, a scFv fragment, a dsFv fragment, a dAb fragment, or a single chain binding polypeptide.

In some aspects, the anti-LAG3 antibody is BMS-986016 (relatlimab), IMP731 (H5L7BW), MK4280 (28G-10, favezelimab), REGN3767 (fianlimab), GSK2831781, humanized BAP050, IMP-701 (LAG525, ieramilimab), aLAG3(0414), aLAG3(0416), Sym022, TSR-033, TSR-075, XmAb841 (XmAb22841), MGD013 (tebotelimab), B1754111, FS118, P 13B02-30, AVA-017, 25F7, AGEN1746, RO7247669, INCAGN02385, IBI-110, EMB-02, IBI-323, LBL-007, or ABL501, or comprises an antigen binding portion thereof.

In some aspects, the anti-LAG3 antibody is MGD013 (tebotelimab), which is a bispecific PD-1×LAG3 DART. In some aspects, tebotelimab is administered intravenously at a dose of about 300 mg or about 600 mg once about every 2 or 3 weeks. In some aspects, tebotelimab is administered intravenously at a dose of about 300 mg once about every 2 weeks. In some aspects, tebotelimab is administered intravenously at a dose of about 600 mg once about every 3 weeks.

In some aspects, the anti-LAG3 antibody is REGN3767 (fianlimab). In some aspects, fianlimab is administered intravenously at a dose of about 1 mg/kg, about 3 mg/kg, about 10 mg/kg, or about 20 mg/kg once about every 3 weeks. In some aspects, fianlimab is administered intravenously at a dose of about 1600 mg once about every 3 weeks.

In some aspects, the anti-LAG3 antibody is LAG525 (ieramilimab). In some aspects, ieramilimab is administered intravenously at a dose of about 300 mg, about 400 mg, about 500 mg, about 600 mg, about 700 mg, about 800 mg, about 900 mg, about 1000 mg, about 1100 mg, about 1200 mg, or about 1300 mg once about every 2, 3, or 4 weeks.

In some aspects, the anti-LAG3 antibody is MK4280 (favezelimab). In some aspects, favezelimab is administered intravenously at a dose of about 7 mg, about 21 mg, about 70 mg, about 210 mg, about 700 mg, or about 800 mg once about every 3 weeks or once about every 6 weeks. In some aspects, favezelimab is administered intravenously at a dose of about 200 mg once about every 3 weeks. In some aspects, favezelimab is administered intravenously at a dose of about 800 mg once about every 6 weeks. In some aspects, favezelimab is administered intravenously at a dose of about 800 mg on Day 1, then once about every 3 weeks. In some aspects, favezelimab is administered for up to 35 cycles. In some aspects, favezelimab is administered intravenously at a dose of about 800 mg for about 30 minutes on Day 1 of a three-week cycle for up to 35 cycles.

In some aspects, the LAG3 inhibitor is a soluble LAG3 polypeptide. In some aspects, the soluble LAG3 polypeptide is a fusion polypeptide, e.g., a fusion protein comprising the extracellular portion of LAG3. In some aspects, the soluble LAG3 polypeptide is a LAG3-Fc fusion polypeptide capable of binding to MHC Class II. In some aspects, the soluble LAG3 polypeptide comprises a ligand binding fragment of the LAG3 extracellular domain.

In some aspects, the LAG3 inhibitor is formulated for intravenous administration.

In some aspects, the LAG3 inhibitor is administered at a flat dose.

In some aspects, the LAG3 inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the LAG3 inhibitor is administered at a dose of about 0.25 mg, about 0.5 mg, about 0.75 mg, about 1 mg, about 1.25 mg, about 1.5 mg, about 1.75 mg, about 2 mg, 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG3 inhibitor is administered at a weight-based dose.

In some aspects, the LAG3 inhibitor is administered at a dose of from at least about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the LAG3 inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the LAG3 inhibitor is administered in a constant amount.

In some aspects, the dose of the LAG3 inhibitor is administered in a varying amount. For example, in some aspects, a maintenance (or follow-on) dose of the LAG3 inhibitor can be higher or the same as a loading dose that is first administered. In some aspects, the maintenance dose of the LAG3 inhibitor can be lower or the same as the loading dose.

In some aspects, the dose of the LAG3 inhibitor is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the anti-LAG3 antibody is relatlimab. Relatlimab (BMS-986016) is a fully human lymphocyte activation gene 3 (LAG3) specific antibody that was isolated following immunization of transgenic mice expressing human immunoglobulin (Ig) genes. Relatlimab binds to LAG3 receptors expressed on T-cells with high affinity and prevents binding of this receptor to cells bearing its ligands, major histocompatibility complex (MHC) Class II and fibrinogen-like protein 1 (FGL-1) (Andrews, Immunol Rev. 2017 March; 276(1): 80-96; Wang, Cell. 2019 Jan. 10; 176(1-1): 334-347). Relatlimab binding inhibits the negative regulatory function of LAG3 mediated through its interaction with ligands in vitro.

It is observed herein that resistance to immunotherapy, such as treatment with an anti-PD-1 antibody, may be overcome by additional blockade of LAG3. In particular, results herein demonstrate that near or complete engagement of the LAG3 receptor—in both its soluble and membrane-bound forms—may yield improved responses, including in patients that do not respond to anti-PD-1 therapies alone. For example, Opdualag™ is a fixed-dose dual immunotherapy combination treatment of nivolumab and relatlimab approved by the FDA for the treatment of adult and pediatric patients 12 years of age or older with unresectable or metastatic melanoma.

The combination of T cell therapy and a checkpoint inhibition therapy that includes both an anti-PD-1 antibody and an anti-LAG3 antibody may improve responses as compared to the combination of a T cell therapy and an anti-PD-1 antibody alone.

Observations herein indicate that the combination of a T cell therapy, e.g. a CAR-T cell therapy, with both a PD-1 inhibitor and a LAG3 inhibitor is advantageous. The results herein show that, relatively high doses of an anti-LAG3 antibody (e.g. 480 mg or 960 mg, such as 480 mg every two weeks (Q2W) or 960 mg every four weeks (Q4W)) increase the LAG3 receptor occupancy, both in peripheral blood and the tumor microenvironment. In particular, high doses of the anti-LAG3 antibody relatlimab (e.g. 960 mg Q4W) are observed herein to minimize the amount of soluble free LAG3 receptor protein in peripheral blood (see Example 2). In some aspects, maximized LAG3 receptor occupancy and/or minimized soluble free LAG3 may improve responses to a T cell therapy, e.g. CAR T cells. In some aspects, both maximization of LAG3 receptor occupancy and minimization of soluble free LAG3 receptors will improve responses to T cell therapy. In some aspects, near or complete engagement of LAG3 in the tumor microenvironment and the periphery improves clinical outcomes by reducing or eliminating the possibility of LAG3 expression by T cells.

In some aspects, such effects are observed despite a subject exhibiting resistance to a PD-1 inhibitor, or exhibiting high levels of PD-1. In particular, it is observed herein that the combination of a PD-1 inhibitor (e.g. an anti-PD-1 antibody, such as nivolumab) and a LAG3 inhibitor (e.g. an anti-LAG3 antibody) are synergistic. In some embodiments, treatment with an anti-PD-1 antibody and an anti-LAG3 antibody is more efficacious than treatment with either antibody alone, including in subjects who do not respond to treatment with an anti-PD-1 antibody. Thus, in some embodiments, the provided combination therapy achieves synergistic effects and activity compared to a therapy involving administration of a PD-1 inhibitor without administration of a LAG3 inhibitor.

Among the provided embodiments, the methods involve combination therapy of a therapy that targets or is directed to killing of cells of a cancer, e.g. a T cell therapy, such as a CAR T cell therapy, and a checkpoint inhibitor therapy. In some aspects, the checkpoint inhibitor therapy inhibits one or more checkpoints, e.g. immune checkpoint. In some embodiments, the immune checkpoint is PD-1. In some embodiments, checkpoint inhibitor therapy comprises or consists of an anti-PD-1 antibody. In some embodiments, the immune checkpoint is LAG3. In some embodiments, the immune checkpoint is PD-1. In some embodiments, checkpoint inhibitor therapy comprises an anti-LAG3 antibody. In some embodiments, the checkpoint inhibitor therapy targets PD-1 and/or LAG3. In some embodiments, the checkpoint inhibitor therapy targets PD-1 and LAG3. In some embodiments, the checkpoint inhibitor comprises or consists of an anti-PD-1 antibody and an anti-LAG3 antibody. Thus, in some embodiments, the method involve combination therapy of a CAR T cell therapy and a checkpoint inhibitor therapy comprising an anti-PD-1 antibody and an anti-LAG3 antibody. In some cases, the combination therapy is a CD19-targeting CAR T cell therapy, an anti-PD-1 antibody (e.g. nivolumab), and an anti-LAG3 antibody (e.g. relatlimab).

In some aspects, improvements in response to the combination therapy are observed despite that the tumor or disease or target cell itself is insensitive, resistant and/or otherwise not sufficiently responsive to the T cell therapy (e.g. CAR T cells) or to the checkpoint inhibitor therapy when each is administered alone. In some embodiments, the cells of the T cell therapy (e.g. CAR T cells) have decreased effector function and/or exhibit exhaustion. In some aspects, such exhaustion and/or decreased effector function of the T cell therapy is attributable to increased expression of one or more immune checkpoints by the T cells. For example, it is observed herein that the CAR T cells of subjects exhibiting worse clinical responses have higher expression of PD-1 and LAG3. Thus, a checkpoint inhibitor therapy may decrease expression of one or more immune checkpoints by the cells of the T cell therapy, thereby increasing the effector functions of the cells. In some embodiments, the provided combination therapy achieves synergistic effects and activity compared to a therapy involving only administration of the T cell therapy, or of the checkpoint inhibitor therapy given at the same dosing regimen, e.g. dose and frequency.

Further, in some aspect, endogenous T cells of a subject have decreased effector function and/or exhibit exhaustion. In some aspects, such exhaustion and/or decreased effector function of endogenous T cells is attributable to increased expression of one or more immune checkpoints by the endogenous T cells. For example, it is observed herein that the endogenous (i.e. CAR-negative) T cells of subjects exhibiting worse clinical responses have higher expression of PD-1 and LAG3. Thus, a checkpoint inhibitor therapy may decrease expression of one or more immune checkpoints by endogenous T cells, thereby increasing the effector functions of the cells. In some embodiments, the provided combination therapy achieves synergistic effects and activity compared to a therapy involving only administration of the T cell therapy, or of the checkpoint inhibitor therapy given at the same dosing regimen, e.g. dose and frequency.

In some embodiments, the provided embodiments involve initiating the administration of the checkpoint inhibitor therapy, e.g., an anti PD-1 antibody and optionally, an anti-LAG3 antibody, after administration of a T cell therapy (e.g. CAR T cell therapy) in a dosing regimen. In some embodiments, the initiation of the administration of the checkpoint inhibitor therapy is after administration of the T cell therapy, such as between about one week and two weeks after administration of the T cell therapy for treating the cancer. In some embodiments, the initiation of the administration of the checkpoint inhibitor therapy, is not until activation-induced cell death (AICD) of the cells of the T cell therapy has peaked. In some embodiments, administration of a checkpoint inhibitor therapy, such as an anti-PD-1 antibody, and optionally, an anti-LAG3 antibody, is not until about one week after administration of T cell therapy. In some cases, administration of the T cell therapy (e.g. CAR T cell therapy) occurs on Day 1, and administration of the checkpoint inhibitor therapy begins on Day 8. In some embodiments, administration of a checkpoint inhibitor therapy, such as an anti-PD-1 antibody, and optionally, an anti-LAG3 antibody, is not until about two weeks after administration of T cell therapy. In some cases, administration of the T cell therapy (e.g. CAR T cell therapy) occurs on Day 1, and administration of the checkpoint inhibitor therapy begins on Day 15.

In some embodiments, the methods include administering to the subject a PD-1 inhibitor (e.g. an anti-PD-1 antibody, such as nivolumab) and a LAG3 inhibitor (e.g. an anti-LAG3 antibody, such as relatlimab) after administration of a T cell therapy (e.g. anti-CD19 CAR T cells). In some embodiments, a first dose of the PD-1 inhibitor is administered between Day 2 and Day 20, inclusive. In some embodiments, a first dose of the LAG3 inhibitor is administered between Day 2 and Day 20, inclusive. In some embodiments, a first dose of the PD-1 inhibitor and a first dose of the LAG3 inhibitor are independently administered, each between Day 2 and Day 20, inclusive. In some embodiments, the first dose of the PD-1 inhibitor and the first dose of the LAG3 inhibitor are administered on the same day.

In some embodiments, the methods include administering to the subject a PD-1 inhibitor (e.g. an anti-PD-1 antibody, such as nivolumab) after administration of a T cell therapy (e.g. anti-CD19 CAR T cells). In some embodiments, the methods include administering at least two doses of the PD-1 inhibitor. In some embodiments, the PD-1 inhibitor is administered in a dosing regimen comprising administration of the PD-1 inhibitor about every two weeks (Q2W) or about every four weeks (Q4W). In some embodiments, the PD-1 inhibitor is administered in a dosing regimen comprising at least two doses, wherein the first dose is administered between Day 2 and Day 20, inclusive, wherein the T cell therapy is administered on Day 1. In some embodiments, a first dose of the PD-1 inhibitor is administered between Day 2 and Day 20, inclusive, wherein the T cell therapy is administered on Day 1. In some embodiments, each subsequent dose of the PD-1 inhibitor is administered about two weeks, about three weeks, or about four weeks after the previous dose of the PD-1 inhibitor. In some embodiments, between about 140 mg and 580 mg of the PD-1 inhibitor (e.g. an anti-PD-1 antibody such as nivolumab) is administered in each dose.

In some embodiments, the first dose of the PD-1 inhibitor is administered on Day 8. In some embodiments, the first dose of the PD-1 inhibitor is administered on Day 15. In some embodiments, about 240 mg of the PD-1 inhibitor (e.g. an anti-PD-1 antibody such as nivolumab) is administered in each dose. In some embodiments, about 480 mg of the PD-1 inhibitor (e.g. an anti-PD-1 antibody such as nivolumab) is administered in each dose.

In some embodiments, the method includes administering four doses of the PD-1 inhibitor. In some embodiments, each of the four doses of the PD-1 inhibitor are about 480 mg. In some embodiments, the second dose of the PD-1 inhibitor is administered about four weeks after the first dose of the PD-1 inhibitor. In some embodiments, the third dose of the PD-1 inhibitor is administered about three weeks or about four weeks after the second dose of the PD-1 inhibitor. In some embodiments, the third dose of the PD-1 inhibitor is administered about three weeks after the second dose of the PD-1 inhibitor. In some embodiments, the third dose of the PD-1 inhibitor is administered about four weeks after the second dose of the PD-1 inhibitor. In some embodiments, the fourth dose of the PD-1 inhibitor is administered about three weeks after the third dose of the PD-1 inhibitor.

In some embodiments, the method includes administering five doses of the PD-1 inhibitor. In some embodiments, at least one dose of the PD-1 inhibitor is about 240 mg, and at least one dose of the PD-1 inhibitor is about 480 mg. In some embodiments, three doses of the PD-1 inhibitor are 240 mg, and the subsequent two doses of the PD-1 inhibitor are 480 mg. In some embodiments, the second dose and the third dose of the PD-1 inhibitor are each administered about two weeks after the previous dose of the PD-1 inhibitor. In some embodiments, the fourth dose of the PD-1 inhibitor is administered about three weeks after the third dose of the PD-1 inhibitor. In some embodiments, each of the first four doses of the PD-1 inhibitor are administered about two weeks after the previous dose of the PD-1 inhibitor. In some embodiments, the fifth dose of the PD-1 inhibitor is administered about four weeks after the fourth dose of the PD-1 inhibitor.

In some embodiments, the method includes administering six doses of the PD-1 inhibitor. In some embodiments, the first dose and the second dose of the PD-1 inhibitor are each administered about two weeks after the previous dose of the PD-1 inhibitor. In some embodiments, each of the second, third, fifth and sixth doses of the PD-1 inhibitor are administered about two weeks after the previus dose of the PD-1 inhibitor. In some embodiments, the fifth dose of the PD-1 inhibitor is administered about three weeks after the fourth dose of the PD-1 inhibitor. In some embodiments, the PD-1 inhibitor is administered about every two weeks (Q2W).

In some embodiments, the PD-1 inhibitor is administered for no longer than about three months. In some embodiments, the dosing regimen lasts about 3 months. In some embodiments, the final dose of the PD-1 inhibitor is administered between about Day 80 and about Day 90. In some embodiments, the final dose of the PD-1 inhibitor is administered at about Day 85.

In some embodiments, the methods include administering a T cell therapy (e.g. anti-CD19 CAR T cells) to a subject on Day 1, followed by administration of a PD-1 inhibitor. In some embodiments, the PD-1 inhibitor is administered in a dosing regimen comprising a first cycle and a second cycle.

In some embodiments, the first dose of the first cycle of the PD-1 inhibitor (e.g. an anti-PD-1 antibody, such as nivolumab) is administered to the subject between about Day 2 and Day 20. In some embodiments, between about 140 mg and about 580 mg of nivolumab is administered in each dose of the first cycle. In some embodiments, about 240 mg of nivolumab is administered in each dose of the first cycle. In some embodiments, about 480 mg of nivolumab is administered in each dose of the first cycle. In some embodiments, at least one dose or at least two doses of the PD-1 inhibitor are administered during the first cycle. In some embodiments, one dose of the PD-1 inhibitor is administered during the first cycle. In some embodiments, two doses of the PD-1 inhibitor are administered during the first cycle. In some embodiments, three doses of the PD-1 inhibitor are administered during the first cycle.

In some embodiments, the first dose of the second cycle of the PD-1 inhibitor (e.g. an anti-PD-1 antibody, such as nivolumab) is administered to the subject between about Day 50 and Day 65. In some embodiments, between about 140 mg and about 580 mg of nivolumab is administered in each dose of the second cycle. In some embodiments, about 240 mg of nivolumab is administered in each dose of the second cycle. In some embodiments, about 480 mg of nivolumab is administered in each dose of the second cycle. In some embodiments, at least two doses of the PD-1 inhibitor are administered during the first cycle. In some embodiments, two doses of the PD-1 inhibitor are administered during the first cycle.

In some embodiments, the methods further comprise administering a LAG3 inhibitor (e.g. an anti-LAG3 antibody, such as relatlimab) to the subject. In some embodiments, a dose of a LAG3 inhibitor is administered about every two weeks (Q2W). In some embodiments, a dose of a LAG3 inhibitor is administered about every four weeks (Q4W). In some embodiments, a first dose of the LAG3 inhibitor is administered between Day 2 and Day 20, inclusive, wherein the T cell therapy is administered on Day 1. In some embodiments, a first dose of the LAG3 inhibitor is administered between Day 8 and Day 15, inclusive, wherein the T cell therapy is administered on Day 1. In some embodiments, the first dose of the LAG3 inhibitor is administered on Day 8. In some embodiments, the first dose of the LAG3 inhibitor is administered on Day 15. In some embodiments, each subsequent dose of the LAG3 inhibitor is administered about two weeks, about three weeks, about four weeks, or about five weeks after the previous dose of the LAG3 inhibitor. In some embodiments, a dose of a LAG3 inhibitor (e.g. an anti-LAG3 antibody, such as relatlimab) is administered on each of the same days on which a dose of the PD-1 inhibitor (e.g. an anti-PD-1 antibody, such as nivolumab) is administered.

In some embodiments, between about 60 mg and 1040 mg of the LAG3 inhibitor (e.g. an anti-LAG3 antibody such as relatlimab) is administered in each dose. In some embodiments, 120 mg of the relatlimab is administered in each dose. In some embodiments, between about 160 mg and 1040 mg of the LAG3 inhibitor (e.g. an anti-LAG3 antibody such as relatlimab) is administered in each dose. In some embodiments, 160 mg of the relatlimab is administered in each dose. In some embodiments, 240 mg of the relatlimab is administered in each dose. In some embodiments, 480 mg of relatlimab is administered in each dose. In some embodiments, 960 mg of relatlimab is administered in each dose.

In some embodiments, the method includes administering three doses of the LAG3 inhibitor. In some embodiments, the second dose is administered about four weeks after the first dose. In some embodiments, the third dose is administered about four weeks or about five weeks after the second dose. In some embodiments, the third dose is administered about four weeks after the second dose. In some embodiments, the third dose is administered about five weeks after the second dose. In some embodiments, each dose of the LAG3 inhibitor is about 240 mg.

In some embodiments, the method includes administering four doses of the LAG3 inhibitor. In some embodiments, the second dose is administered about four weeks after the first dose. In some embodiments, the third dose is administered about three weeks or about four weeks after the second dose. In some embodiments, the third dose is administered about three weeks after the second dose. In some embodiments, the third dose is administered about four weeks after the second dose. In some embodiments, the fourth dose is administered about three weeks after the third dose. In some embodiments, each dose of the LAG3 inhibitor is about 240 mg. In some embodiments, each dose of the LAG3 inhibitor is about 480 mg.

In some embodiments, the method includes administering six doses of the LAG3 inhibitor. In some embodiments, each of the second and third doses of the LAG3 inhibitor are administered about two weeks after the previous dose of the LAG3 inhibitor. In some embodiments, each of the second, third, fifth, and sixth doses are administered about two weeks after the previous dose. In some embodiments, the fourth dose of the LAG3 inhibitor is administered about three weeks after the third dose of the LAG3 inhibitor. In some embodiments, a dose of the LAG3 inhibitor is administered about every two weeks (Q2W).

In some embodiments, the LAG3 inhibitor (e.g. an anti-LAG3 antibody, such as relatlimab) is administered in a dosing regimen comprising the first cycle and the second cycle. In some embodiments, between about 160 mg and about 1040 mg of relatlimab is administered in each dose of the first cycle. In some embodiments, about 240 mg of relatlimab is administered in each dose of the first cycle. In some embodiments, about 480 mg of nivolumab is administered in each dose of the first cycle. In some embodiments, about 960 mg of nivolumab is administered in each dose of the first cycle. In some embodiments, at least one dose or at least two doses of the LAG3 inhibitor are administered during the first cycle. In some embodiments, one dose of the LAG3 inhibitor is administered during the first cycle. In some embodiments, two doses of the LAG3 inhibitor are administered during the first cycle. In some embodiments, three doses of the LAG3 inhibitor are administered during the first cycle.

In some embodiments, between about 400 mg and about 1040 mg of relatlimab is administered in each dose of the second cycle. In some embodiments, about 480 mg of nivolumab is administered in each dose of the second cycle. In some embodiments, about 960 mg of nivolumab is administered in each dose of the second cycle. In some embodiments, at least two doses of the LAG3 inhibitor are administered during the first cycle. In some embodiments, two doses of the LAG3 inhibitor are administered during the first cycle.

In some embodiments, about 480 mg nivolumab and about 160 mg relatlimab are administered Q4W.

In some embodiments, the provided methods can potentiate CAR-T cell therapy, which, in some aspects, can improve outcomes for treatment of subjects that have a cancer that is resistant or refractory to other therapies, is an aggressive or high-risk cancer, and/or that is or is likely to exhibit a relatively lower response rate to a CAR-T cell therapy when administered without the checkpoint inhibitor therapy. In some aspects, administering a checkpoint inhibitor therapy, e.g., an anti-PD-1 antibody, and optionally an anti-LAG3 antibody, according to the provided methods increases the activity of CAR-expressing cells for treating a cancer, e.g. B cell malignancy such as NHL, by reducing or preventing exhaustion of the CAR T cells. In some aspects, administering a checkpoint inhibitor therapy, e.g., an anti-PD-1 antibody, and optionally an anti-LAG3 antibody, according to the provided methods increases the activity of endogenous T cells for treating a cancer, e.g. B cell malignancy such as NHL, by reducing or preventing exhaustion of the endogenous T cells. In some aspects, the anti-tumor activity of administered CAR+ T cells against a CD19-expressing cancer (e.g. a B cell malignancy such as NHL) is improved. In some aspects, the anti-tumor activity of endogenous T cells against a CD19-expressing cancer (e.g. a B cell malignancy such as NHL) cell is improved.

### I. Combination Therapy

Provided herein are methods of treatment that involve administering a combination therapy of a T cell therapy, such as engineered cells or compositions containing engineered cells (e.g. CAR T cells) and a checkpoint inhibitor therapy (e.g. an anti-PD-1 antibody and optionally an anti-LAG3 antibody). In some embodiments, the T cell therapy (e.g. CAR T cells) specifically recognizes and/or binds to an antigen associated with, expressed by or present on cells of the cancer. In some embodiments, the antigen is CD19.

Also provided are methods and uses of a combination therapy of a T cell therapy, such as engineered cells (e.g., CAR T cells) and a checkpoint inhibitory therapy (e.g. an anti-PD-1 antibody and optionally an anti-LAG3 antibody), and/or compositions thereof, including methods for the treatment of subjects having a disease or condition. In some aspects, also provided are uses of a combination therapy of a T cell therapy, such as engineered cells (CAR T cells) and a checkpoint inhibitor therapy (e.g. an anti-PD-1 antibody and optionally an anti-LAG3 antibody) for treatment of a disease or condition. In some aspects, the uses of the combination therapy of a T cell therapy, such as engineered cells or compositions containing engineered cells (e.g. CAR T cells), and the checkpoint inhibitor therapy (e.g. an anti-PD-1 antibody and optionally an anti-LAG3 antibody) are in accord with any of the methods described herein.

Also provided are combinations and articles of manufacture, such as kits, that contain a composition comprising the T cell therapy and/or a composition comprising the checkpoint inhibitor therapy (e.g. an anti-PD-1 antibody and optionally, an anti-LAG3 antibody), and uses of such compositions and combinations to treat or prevent conditions, such as CD19-expressing cancers (e.g. B cell malignancies, such as NHL).

In some embodiments, the T cell therapy (e.g. CD19-targeting CAR T cells) is administered on Day 1. In some embodiments, methods can include administration of the checkpoint inhibitor therapy (e.g. an anti-PD-1 antibody such as nivolumab and optionally, an anti-LAG3 antibody such as relatlimab) subsequent to the administration (e.g., 2-20 days after initiation of the administration) of the T cell therapy on Day 1, wherein the T cell therapy specifically binds to an antigen associated with, expressed by or present on cells of the CD19-expressing cancer (e.g. a B cell malignancy such as NHL). In some embodiments, the checkpoint inhibitor therapy (e.g. an anti-PD-1 antibody such as nivolumab and optionally, an anti-LAG3 antibody such as relatlimab) is administered between about Day 2 and Day 20, such as on Day 8 or Day 15.

In some embodiments, the methods include administering the checkpoint inhibitor therapy (e.g. an anti-PD-1 antibody such as nivolumab and optionally, an anti-LAG3 antibody such as relatlimab) in a dosing regimen comprising administration of the checkpoint inhibitory therapy every two weeks (Q2W) or every four weeks (Q4W). In some embodiments, an anti-PD-1 antibody (e.g. nivolumab) is administered in an amount of between at or about 140 mg and at or about 580 mg. In some embodiments, an anti-PD-1 antibody (e.g. nivolumab) is administered in an amount of about 240 mg. In some embodiments, an anti-PD-1 antibody (e.g. nivolumab) is administered in an amount of about 480 mg.

In some embodiments, the methods include administering the checkpoint inhibitor therapy (e.g. an anti-PD-1 antibody such as nivolumab and optionally, an anti-LAG3 antibody such as relatlimab) in a dosing regimen comprising administration of the checkpoint inhibitory therapy every two weeks (Q2W). In some embodiments, an anti-PD-1 antibody (e.g. nivolumab) is administered about every two weeks in an amount of between at or about 60 mg and at or about 320 mg. In some embodiments, an anti-PD-1 antibody (e.g. nivolumab) is administered about every two weeks in an amount of at or about 120 mg. In some embodiments, an anti-PD-1 antibody (e.g. nivolumab) is administered about every two weeks in an amount of between at or about 140 mg and at or about 320 mg. In some embodiments, an anti-PD-1 antibody (e.g. nivolumab) is administered about every two weeks in an amount of at or about 240 mg.

In some embodiments, the methods include administering the checkpoint inhibitor therapy (e.g. an anti-PD-1 antibody such as nivolumab and optionally, an anti-LAG3 antibody such as relatlimab) in a dosing regimen comprising administration of the checkpoint inhibitory therapy every four weeks (Q4W). In some embodiments, an anti-PD-1 antibody (e.g. nivolumab) is administered about every four weeks in an amount of between at or about 380 mg and at or about 580 mg. In some embodiments, an anti-PD-1 antibody (e.g. nivolumab) is administered about every four weeks in an amount of at or about 480 mg.

In some embodiments, the methods include administering the checkpoint inhibitor therapy (e.g. an anti-PD-1 antibody such as nivolumab and an anti-LAG3 antibody such as relatlimab) in a dosing regimen comprising administration of the checkpoint inhibitory therapy every two weeks (Q2W) or every four weeks (Q4W). In some embodiments, an anti-LAG3 antibody (e.g. relatlimab) is administered in an amount of between at or about 60 mg and at or about 1040 mg. In some embodiments, an anti-LAG3 antibody (e.g. relatlimab) is administered in an amount of between at or about 140 mg and at or about 1040 mg.

In some embodiments, the methods include administering the checkpoint inhibitor therapy (e.g. an anti-PD-1 antibody such as nivolumab and an anti-LAG3 antibody such as relatlimab) in a dosing regimen comprising administration of the checkpoint inhibitory therapy every two weeks (Q2W). In some embodiments, an anti-LAG3 antibody (e.g. relatlimab) is administered about every two weeks in an amount of between at or about 60 mg and at or about 540 mg. In some embodiments, an anti-LAG3 antibody (e.g. relatlimab) is administered about every two weeks in an amount of at or about 120 mg. In some embodiments, an anti-LAG3 antibody (e.g. relatlimab) is administered about every two weeks in an amount of between at or about 140 mg and at or about 540 mg. In some embodiments, an anti-LAG3 antibody (e.g. relatlimab) is administered about every two weeks in an amount of at or about 160 mg. In some embodiments, an anti-LAG3 antibody (e.g. relatlimab) is administered about every two weeks in an amount of at or about 240 mg. In some embodiments, an anti-LAG3 antibody (e.g. relatlimab) is administered about every two weeks in an amount of at or about 360 mg. In some embodiments, an anti-LAG3 antibody (e.g. relatlimab) is administered about every two weeks in an amount of at or about 480 mg.

In some embodiments, the methods include administering the checkpoint inhibitor therapy (e.g. an anti-PD-1 antibody such as nivolumab and an anti-LAG3 antibody such as relatlimab) in a dosing regimen comprising administration of the checkpoint inhibitory therapy every four weeks (Q4W). In some embodiments, an anti-LAG3 antibody (e.g. relatlimab) is administered about every four weeks in an amount of between at or about 60 mg and at or about 1040 mg. In some embodiments, an anti-LAG3 antibody (e.g. relatlimab) is administered about every four weeks in an amount of between at or about 880 mg and at or about 1040 mg. In some embodiments, an anti-LAG3 antibody (e.g. relatlimab) is administered about every four weeks in an amount of at or about 120 mg. In some embodiments, an anti-LAG3 antibody (e.g. relatlimab) is administered about every four weeks in an amount of at or about 160 mg. In some embodiments, an anti-LAG3 antibody (e.g. relatlimab) is administered about every four weeks in an amount of at or about 240 mg. In some embodiments, an anti-LAG3 antibody (e.g. relatlimab) is administered about every four weeks in an amount of at or about 360 mg. In some embodiments, an anti-LAG3 antibody (e.g. relatlimab) is administered about every four weeks in an amount of at or about 480 mg. In some embodiments, an anti-LAG3 antibody (e.g. relatlimab) is administered about every four weeks in an amount of at or about 960 mg.

In some embodiments, the methods include administering the checkpoint inhibitor therapy (e.g. an anti-PD-1 antibody such as nivolumab and an anti-LAG3 antibody such as relatlimab) in a dosing regimen comprising administration of the checkpoint inhibitory therapy every three weeks (Q3W). In some embodiments, an anti-LAG3 antibody (e.g. relatlimab) is administered about every three weeks in an amount of between at or about 60 mg and at or about 1040 mg. In some embodiments, an anti-LAG3 antibody (e.g. relatlimab) is administered about every three weeks in an amount of between at or about 880 mg and at or about 1040 mg. In some embodiments, an anti-LAG3 antibody (e.g. relatlimab) is administered about every three weeks in an amount of at or about 120 mg. In some embodiments, an anti-LAG3 antibody (e.g. relatlimab) is administered about every threeweeks in an amount of at or about 160 mg. In some embodiments, an anti-LAG3 antibody (e.g. relatlimab) is administered about every three weeks in an amount of at or about 240 mg. In some embodiments, an anti-LAG3 antibody (e.g. relatlimab) is administered about every three weeks in an amount of at or about 360 mg. In some embodiments, an anti-LAG3 antibody (e.g. relatlimab) is administered about every threer weeks in an amount of at or about 480 mg. In some embodiments, an anti-LAG3 antibody (e.g. relatlimab) is administered about every three weeks in an amount of at or about 960 mg.

In some embodiments, the anti-PD-1 antibody (e.g. nivolumab) and the anti-LAG3 antibody (e.g. relatlimab) are administered on the same days. In some embodiments, about 240 mg of the anti-PD-1 antibody (e.g. nivolumab) and about 120 mg of the anti-LAG3 antibody (e.g. relatlimab) are administered on the same days. In some embodiments, about 240 mg of the anti-PD-1 antibody (e.g. nivolumab) and about 240 mg of the anti-LAG3 antibody (e.g. relatlimab) are administered on the same days. In some embodiments, about 480 mg of the anti-PD-1 antibody (e.g. nivolumab) and about 160 mg of the anti-LAG3 antibody (e.g. relatlimab) are administered on the same days. In some embodiments, about 480 mg of the anti-PD-1 antibody (e.g. nivolumab) and about 240 mg of the anti-LAG3 antibody (e.g. relatlimab) are administered on the same days. In some embodiments, about 480 mg of the anti-PD-1 antibody (e.g. nivolumab) and about 480 mg of the anti-LAG3 antibody (e.g. relatlimab) are administered on the same days.

In some embodiments, a PD-1 inhibitor (e.g. an anti-PD-1 antibody such as nivolumab) is administered to the subject as a single agent therapy (e.g. monotherapy) in combination with a T cell therapy (e.g. CAR T cells). In some embodiments administration as a monotherapy consists of a single type of treatment alone, to treat a disease or condition, except where otherwise provided. In some embodiments, a PD-1 inhibitor (e.g. an anti-PD-1 antibody) is provided as a monotherapy with a T cell therapy, such that no other treatment is provided to treat a disease or condition beyond provision of (1) the PD-1 inhibitor and (2) the immunotherapy or the cell therapy.

In some embodiments, the methods include administering the checkpoint inhibitor therapy (e.g. an anti-PD-1 antibody such as nivolumab) in a first cycle and a second cycle. In some embodiments, the first dose of the first cycle is administered between about Day 2 and Day 20. In some embodiments, the first dose of the first cycle is administered on about Day 8. In some embodiments, the first dose of the first cycle is administered on about Day 15. In some embodiments, at least one dose of the PD-1 inhibitor is administered in the first cycle. In some embodiments, one dose of the PD-1 inhibitor is administered in the first cycle. In some embodiments, the PD-1 inhibitor is administered on day 15 of the first cycle. In some embodiments, two doses of the PD-1 inhibitor are administered in the first cycle. In some embodiments, the PD-1 inhibitor is administered on Days 8 and 36 of the first cycle. In some embodiments, three doses of the PD-1 inhibitor are administered in the first cycle. In some embodiments, the PD-1 inhibitor is administered on Days 8, 22, and 36 of the first cycle. In some embodiments, the PD-1 inhibitor is administered on Days 15, 29, and 43 of the first cycle. In some embodiments, an anti-PD-1 antibody (e.g. nivolumab) is administered in an amount of between at or about 140 mg and at or about 580 mg. In some embodiments, an anti-PD-1 antibody (e.g. nivolumab) is administered in an amount of between at or about 140 mg and at or about 320 mg. In some embodiments, an anti-PD-1 antibody (e.g. nivolumab) is administered in an amount of at or about 240 mg. In some embodiments, an anti-PD-1 antibody (e.g. nivolumab) is administered in an amount of between at or about 380 mg and at or about 580 mg. In some embodiments, an anti-PD-1 antibody (e.g. nivolumab) is administered in an amount of at or about 480 mg. In some embodiments, at or about 240 mg of the PD-1 inhibitor is administered on Days 8, 22, and 36 of the first cycle. In some embodiments, at or about 480 mg of the PD-1 inhibitor is administered on Days 8 and 36 of the first cycle. In some embodiments, at or about 240 mg of the PD-1 inhibitor is administered on Days 15, 29, and 43 of the first cycle. In some embodiments, at or about 480 mg of the PD-1 inhibitor is administered on Day 15 of the first cycle.

In some embodiments, the first dose of the second cycle is administered between about Day 50 and Day 65. In some embodiments, the first dose of the second cycle is administered on about Day 57. In some embodiments, at least two doses of the PD-1 inhibitor are administered in the second cycle. In some embodiments, two doses of the PD-1 inhibitor are administered in the second cycle. In some embodiments, the PD-1 inhibitor is administered on Days 57 and 85 of the second cycle. In some embodiments, an anti-PD-1 antibody (e.g. nivolumab) is administered in an amount of between at or about 140 mg and at or about 580 mg. In some embodiments, an anti-PD-1 antibody (e.g. nivolumab) is administered in an amount of between at or about 380 mg and at or about 580 mg. In some embodiments, an anti-PD-1 antibody (e.g. nivolumab) is administered in an amount of at or about 480 mg. In some embodiments, at or about 480 mg of the PD-1 inhibitor is administered on Days 57 and 85 of the second cycle.

In some embodiments, a PD-1 inhibitor (e.g. an anti-PD-1 antibody such as nivolumab) and a LAG3 inhibitor (e.g. an anti-LAG3 antibody, such as relatlimab) are administered to the subject as a combination therapy in combination with a T cell therapy (e.g. CAR T cells). In some embodiments, a PD-1 inhibitor (e.g. an anti-PD-1 antibody such as nivolumab) and a LAG3 inhibitor (e.g. an anti-LAG3 antibody such as relatlimab) are provided as a combination therapy with a T cell therapy, such that no other treatment is provided to treat a disease or condition beyond provision of (1) the PD-1 and LAG3 inhibitors and (2) the T cell therapy.

In some embodiments, the methods include administering the checkpoint inhibitor therapy (e.g. an anti-PD-1 antibody such as nivolumab and an anti-LAG3 antibody such as relatlimab) in the first cycle and the second cycle. In some embodiments, the first dose of the first cycle is administered between about Day 2 and Day 20. In some embodiments, the first dose of the first cycle is administered on about Day 8. In some embodiments, the first dose of the first cycle is administered on about Day 15.

In some embodiments, an anti-LAG3 antibody (e.g. relatlimab) is administered in an amount of between at or about 140 mg and at or about 1040 mg. In some embodiments, an anti-LAG3 antibody (e.g. relatlimab) is administered about every two weeks in an amount of between at or about 140 mg and at or about 540 mg. In some embodiments, an anti-LAG3 antibody (e.g. relatlimab) is administered about every two weeks in an amount of at or about 240 mg. In some embodiments, an anti-LAG3 antibody (e.g. relatlimab) is administered about every two weeks in an amount of at or about 480 mg. In some embodiments, an anti-LAG3 antibody (e.g. relatlimab) is administered about every four weeks in an amount of between at or about 880 mg and at or about 1040 mg.

In some embodiments, the LAG3 inhibitor is administered on the same days on which the PD-1 inhibitor is administered. In some embodiments, at least one dose of the LAG3 inhibitor is administered in the first cycle. In some embodiments, one dose of the LAG3 inhibitor is administered in the first cycle. In some embodiments, the LAG3 inhibitor is administered on day 15 of the first cycle. In some embodiments, two doses of the LAG3 inhibitor are administered in the first cycle. In some embodiments, the LAG3 inhibitor is administered on Days 8 and 36 of the first cycle. In some embodiments, three doses of the LAG3 inhibitor are administered in the first cycle. In some embodiments, the LAG3 inhibitor is administered on Days 8, 22, and 36 of the first cycle. In some embodiments, the LAG3 inhibitor is administered on Days 15, 29, and 43 of the first cycle. In some embodiments, an anti-LAG3 antibody (e.g. relatlimab) is administered in an amount of between at or about 140 mg and at or about 1040 mg. In some embodiments, an anti-LAG3 antibody (e.g. relatlimab) is administered in an amount of between at or about 140 mg and at or about 320 mg. In some embodiments, an anti-LAG3 antibody (e.g. relatlimab) is administered in an amount of at or about 240 mg. In some embodiments, an anti-LAG3 antibody (e.g. relatlimab) is administered in an amount of between at or about 380 mg and at or about 580 mg. In some embodiments, an anti-LAG3 antibody (e.g. relatlimab) is administered in an amount of at or about 480 mg. In some embodiments, an anti-LAG3 antibody (e.g. relatlimab) is administered in an amount of between at or about 880 mg and at or about 1040 mg. In some embodiments, an anti-LAG3 antibody (e.g. relatlimab) is administered in an amount of at or about 960 mg. In some embodiments, at or about 240 mg of the LAG3 inhibitor is administered on Days 8, 22, and 36 of the first cycle. In some embodiments, at or about 480 mg of the LAG3 inhibitor is administered on Days 8, 22, and 36 of the first cycle. In some embodiments, at or about 480 mg of the LAG3 inhibitor is administered on Days 8 and 36 of the first cycle. In some embodiments, at or about 960 mg of the LAG3 inhibitor is administered on Days 8 and 36 of the first cycle. In some embodiments, at or about 240 mg of the LAG3 inhibitor is administered on Days 15, 29, and 43 of the first cycle. In some embodiments, at or about 480 mg of the LAG3 inhibitor is administered on Days 15, 29, and 43 of the first cycle. In some embodiments, at or about 480 mg of the LAG3 inhibitor is administered on Day 15 of the first cycle. In some embodiments, at or about 960 mg of the LAG3 inhibitor is administered on Day 15 of the first cycle.

In some embodiments, the first dose of the second cycle is administered between about Day 50 and Day 65. In some embodiments, the first dose of the second cycle is administered on about Day 57. In some embodiments, at least two doses of the LAG3 inhibitor are administered in the second cycle. In some embodiments, two doses of the LAG3 inhibitor are administered in the second cycle. In some embodiments, the LAG3 inhibitor is administered on Days 57 and 85 of the second cycle. In some embodiments, an anti-LAG3 antibody (e.g. relatlimab) is administered in an amount of between at or about 400 mg and at or about 1040 mg. In some embodiments, an anti-LAG3 antibody (e.g. relatlimab) is administered in an amount of between at or about 380 mg and at or about 580 mg. In some embodiments, an anti-LAG3 antibody (e.g. relatlimab) is administered in an amount of at or about 480 mg. In some embodiments, at or about 480 mg of the LAG3 inhibitor is administered on Days 57 and 85 of the second cycle. In some embodiments, an anti-LAG3 antibody (e.g. relatlimab) is administered in an amount of between at or about 880 mg and at or about 1040 mg. In some embodiments, an anti-LAG3 antibody (e.g. relatlimab) is administered in an amount of at or about 960 mg. In some embodiments, at or about 960 mg of the LAG3 inhibitor is administered on Days 57 and 85 of the second cycle.

In some embodiments, the T cell therapy is adoptive cell therapy. In some embodiments, the T cell therapy is or comprises a transgenic TCR therapy or a recombinant-receptor expressing cell therapy, which optionally is a chimeric antigen receptor (CAR)-expressing T cell therapy. In some embodiments, the therapy targets CD19. In some cases, the T cell therapy comprises T cells expressing a chimeric antigen-receptor (CAR), wherein the antigen-binding domain of the CAR binds to CD19. In some embodiments, the adoptive cell therapy comprises cells that are autologous to the subject. In some embodiments, the cells that are autologous to the subject are engineered to express a chimeric antigen receptor (CAR). In some embodiments, CAR-expressing autologous T cells are provided to the subject. In some embodiments, the cells and dosage regimens for administering the cells can include any as described in the Section II.

In some embodiments, the checkpoint inhibitor therapy reduces or prevents exhaustion of the cells of the cell therapy. In some embodiments, the checkpoint inhibitor therapy reduces or prevents exhaustion of endogenous T cells. In some ways, the checkpoint inhibitor therapy lowers resistance of cancer cells to the T cell therapy.

In some embodiments, the T cell therapy (e.g. CAR-expressing T cells) and the checkpoint inhibitor therapy are provided as pharmaceutical compositions for administration to the subject. In some embodiments, the pharmaceutical compositions contain therapeutically effective amounts of one or both of the agents for combination therapy, e.g., T cells for adoptive cell therapy and a checkpoint inhibitor therapy (e.g. an anti-PD-1 antibody and optionally, an anti-LAG3 antibody) as described. In some embodiments, the pharmaceutical compositions contain subtherapeutically effective amounts of one or both of the agents for combination therapy, T cells for adoptive cell therapy and a checkpoint inhibitor therapy (e.g. an anti-PD-1 antibody and optionally, an anti-LAG3 antibody) as described. In some embodiments, the agents are formulated for administration in separate pharmaceutical compositions. In some embodiments, any of the pharmaceutical compositions provided herein can be formulated in dosage forms appropriate for each route of administration.

In some embodiments, the combination therapy, which includes administering the T cell therapy, including engineered cells, such as CAR-T cell therapy, and the checkpoint inhibitor therapy, is administered to a subject or patient having a cancer (e.g. a NHL) or at risk for cancer, such as a CD19-expressing cancer. In some aspects, the methods treat, e.g., ameliorate one or more symptom of, the disease or condition, such as by lessening tumor burden in a cancer expressing an antigen recognized by the T cell therapy, e.g. recognized by an engineered T cell.

For the prevention or treatment of disease, the appropriate dosage of a PD-1 inhibitor, a LAG3 inhibitor, and/or a T cell therapy (e.g. CAR-expressing T cells) may depend on the type of disease to be treated, the particular inhibitor, cells and/or recombinant receptors expressed on the cells, the severity and course of the disease, route of administration, whether the PD-1 and/or LAG3 inhibitor and/or the T cell therapy, are administered for preventive or therapeutic purposes, previous therapy, frequency of administration, the subject's clinical history and response to the cells, and the discretion of the attending physician. The compositions and cells are in some embodiments suitably administered to the subject at one time or over a series of treatments. Exemplary dosage regimens and schedules for the provided combination therapy are described.

In some embodiments, the T cell therapy and the checkpoint inhibitor therapy are administered as part of a further combination treatment, which can be administered simultaneously with or sequentially to, in any order, another therapeutic intervention. In some contexts, the T cell therapy, such as CAR-expressing T cells, are co-administered with another therapy sufficiently close in time such that the immunotherapy enhances the effect of one or more additional therapeutic agents, or vice versa. In some embodiments, the cells are administered prior to the one or more additional therapeutic agents. In some embodiments, the T cell therapy, e.g. engineered T cells, such as CAR-expressing T cells, are administered after the one or more additional therapeutic agents. In some embodiments, the combination therapy methods further include a lymphodepleting therapy, such as administration of a chemotherapeutic agent. In some embodiments, the combination therapy further comprises administering another therapeutic agent, such as an anti-cancer agent, a checkpoint inhibitor, or another immune modulating agent. Uses include uses of the combination therapies in such methods and treatments, and uses of such compositions in the preparation of a medicament in order to carry out such combination therapy methods. In some embodiments, the methods and uses thereby treat the disease or condition or disorder, such as a cancer or proliferative disease, in the subject.

In some embodiments, the cell therapy and the checkpoint inhibitor therapy are administered without any other combination treatment. In some embodiments, the checkpoint inhibitor therapy is an anti-PD-1 antibody, and the cell therapy and the checkpoint inhibitor therapy are administered without any other combination treatment. In some embodiments, the checkpoint inhibitor therapy is an anti-PD-1 antibody and an anti-LAG3 antibody, and the cell therapy and the checkpoint inhibitor therapy are administered without any other combination treatment.

Prior to, during or following administration of the T cell therapy, such as CAR-T cell therapy and/or an checkpoint inhibitor therapy, the biological activity of the T cell therapy, e.g. the biological activity of the engineered cell populations, in some embodiments is measured, e.g., by any of a number of known methods. Parameters to assess include the ability of the engineered cells to destroy target cells, persistence and other measures of T cell activity, such as measured using any suitable method known in the art, such as assays described further below in Section II below. In some embodiments, the biological activity of the cells, e.g., T cells administered for the T cell based therapy, is measured by assaying cytotoxic cell killing, expression and/or secretion of one or more cytokines, proliferation or expansion, such as upon restimulation with antigen. In some aspects the biological activity is measured by assessing the disease burden and/or clinical outcome, such as reduction in tumor burden or load. In some aspects the biological activity is measured by assessing the presence of neutropenia in a subject. In some embodiments, administration of one or both agents of the combination therapy and/or any repeated administration of the therapy, can be determined based on the results of the assays before, during, during the course of or after administration of one or both agents of the combination therapy.

In some embodiments, the combined effect of the checkpoint inhibitor therapy in combination with the T cell therapy can be synergistic compared to treatments involving only the checkpoint inhibitor therapy or monotherapy with the cell therapy. For example, in some embodiments, the provided methods, compositions and articles of manufacture herein result in an increase or an improvement in a desired therapeutic effect, such as an increased or an improvement in the reduction or inhibition of one or more symptoms associated with cancer.

In some embodiments, the checkpoint inhibitor therapy increases the expansion, proliferation, or cytotoxicity of the engineered T cells, such as CAR T cells. In some embodiments, the increase in expansion, proliferation, or cytotoxicity is observed in vivo following administration of the checkpoint inhibitor therapy to a subject. In some embodiments, the increase in the number of engineered T cells, e.g. CAR-T cells, is increased by greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0 fold or more. In some embodiments, the increase in the cytotoxicity of the engineered T cells, e.g. CAR-T cells, against cancer cells is increased by greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0 fold or more.

In particular embodiments, the methods involve administering one or more doses of the cells to the subject that include a particular number or relative number of cells or of the engineered cells, such as a defined ratio or compositions of two or more sub-types within the composition, such as CD4 vs. CD8 T cells. In some embodiments, the engineered cells and the checkpoint inhibitor therapy, or compositions comprising the same, are administered in an effective amount to effect treatment of the disease or disorder. Uses include uses of the T cell therapy and the checkpoint inhibitor therapy, or compositions thereof, in such methods and treatments, and in the preparation of a medicament in order to carry out such therapeutic methods. In some embodiments, the methods are carried out by administering the T cell therapy and the checkpoint inhibitor therapy, or compositions comprising the same, to the subject having or suspected of having the disease or condition. In some embodiments, the methods thereby treat the disease or condition or disorder in the subject.

General methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

### A. Administration of a Checkpoint Inhibitor Therapy

Provided herein are combination therapy methods, combinations, kits and uses involving administration of a checkpoint inhibitory therapy (e.g. an anti-PD-1 antibody and/or an anti-LAG3 antibody), which can be administered subsequently to administration of the T cell therapy, e.g., CAR T cells. In some embodiments, expression of one or more checkpoint proteins, such as PD-1 and/or LAG3, can increase exhaustion of endogenous and/or administered T cells (e.g. engineered CAR T cells, e.g. CAR T cells). In some embodiments, expression of PD-1 has the ability to promote exhaustion of endogenous and/or administered CAR T cells, thereby allowing increased survival of cancer cells. In some embodiments, LAG3 protein has the ability to promote exhaustion of endogenous and/or administered CAR T cells, thereby allowing increased survival of cancer cells. In some embodiments, individuals may become immune to one or more checkpoint inhibitors, such as a PD-1 inhibitor. In some embodiments, immunity to PD-1 blockade can be overcome by LAG3 inhibition.

In some embodiments, the checkpoint inhibitor therapy in the combination therapy is an inhibitor of PD-1 (e.g. an anti-PD-1 antibody such as nivolumab), which, in some cases, is involved in the exhaustion of endogenous and/or administered T cells. In some embodiments, the checkpoint inhibitor therapy in the combination therapy is an inhibitor of PD-1 (e.g. an anti-PD-1 antibody, such as nivolumab) and an inhibitor of LAG3 (e.g. an anti-LAG3 antibody, such as relatlimab). In some cases, expression of PD-1 and/or LAG3 is involved in the exhaustion of endogenous and/or administered T cells.

In some embodiments, the checkpoint inhibitor therapy (e.g. an anti-PD-1 antibody and/or an anti-LAG3 antibody) reduces and/or prevents exhaustion of endogenous T cells. In some embodiments, the checkpoint inhibitor therapy (e.g. an anti-PD-1 antibody and/or an anti-LAG3 antibody) reduces and/or prevents exhaustion of administered T cells (e.g. engineered T cells, such as CAR T cells). In some cases, the inhibition of LAG3 overcomes resistance to PD-1 inhibition or sensitizes a subject to PD-1 inhibition.

In some embodiments, the checkpoint inhibitor comprises an anti-PD-1 antibody (e.g. nivolumab). In some embodiments, the anti-PD-1 antibody is nivolumab. In some embodiments, the anti-PD-1 antibody inhibits PD-1/PD-L1 signaling. In some embodiments, the checkpoint inhibitor comprises an anti-LAG3 antibody. In some embodiments, the anti-LAG3 antibody is relatlimab. In some embodiments, the anti-LAG3 antibody increases the receptor occupancy of LAG3 on T cells, such as in peripheral blood and/or in the tumor microenvironment. In some embodiments, the anti-LAG3 antibody decreases the amount of free soluble LAG3, such as in peripheral blood. In some embodiments, the anti-LAG3 antibody is provided in combination with the PD-1 antibody to overcome resistance to PD-1 blockade.

In some embodiments, the PD-1 inhibitor is an anti-PD-1 antibody, including but not limited to those described in PCT Patent Application Nos. WO2006121186 and WO2015112800; US Patent Application Nos. US20150203579, US20180346569, and US20130017199; U.S. Pat. Nos. 7,595,048, 8,008,449, 8,354,509, 8,609,089, 8,728,474, 8,735,553, 8,779,105, 8,900,587, 8,952,136, 9,067,999, 9,073,994, 9,683,048, 9,815,897, and 9,987,500; and European Patent Nos. 1537878, 2161336, 2170959, which are each incorporated by reference in their entireties.

Exemplary anti-PD-1 antibodies include, but are not limited to nivolumab, camrelizumab, cemiplimab, dostarlimab, MEDI-0680, pembrolizumab, spartalizumab, SSI-361, and tislelizumab. Other exemplary anti-PD-1 antibodies include, but are not limited to, AMP224, AMP-514, JTX-4014, retifanlimab, sintilimab, and toripalimab.

In some cases, the anti-PD-1 antibody comprises a heavy chain variable (VH) region comprising a CDR1, a CDR2, and a CDR3 comprising the amino acid sequences set forth in SEQ ID NOS: 60, 61, and 62, respectively, and a light chain variable (VL) region comprising a CDR1, a CDR2, and a CDR3 comprising the amino acid sequences set forth in SEQ ID NOS: 63, 64, and 65, respectively. In some embodiments, the VH region comprises the amino acid sequence set forth in SEQ ID NO 66, and the VL region comprises the amino acid sequence set forth in SEQ ID NO: 67. In some cases, the anti-PD-1 antibody is nivolumab.

In some embodiments, the LAG3 inhibitor is an anti-LAG3 antibody, including but not limited to those described in US Application No. US2110150892; PCT Application Nos. WO2010019570, WO2014008218, WO2015116539, and WO2015138920; and U.S. Pat. Nos. 9,505,839 and 9,908,936, which are each incorporated by reference in their entireties.

Exemplary anti-LAG3 antibodies include, but are not limited to relatlimab, favezelimab (MK-4280), and ieramilimab (LAG525).Other exemplary anti-LAG3 antibodies include, but are not limited to, REGN3767, TSR-033, Sym022, BI 754111, FS118.

In some cases, the anti-LAG3 antibody comprises a heavy chain variable (VH) region comprising a CDR1, a CDR2, and a CDR3 comprising the amino acid sequences set forth in SEQ ID NOS: 68, 69, and 70, respectively, and a light chain variable (VL) region comprising a CDR1, a CDR2, and a CDR3 comprising the amino acid sequences set forth in SEQ ID NOS: 71, 72, and 73, respectively. In some embodiments, the VH region comprises the amino acid sequence set forth in SEQ ID NO 74, and the VL region comprises the amino acid sequence set forth in SEQ ID NO: 75. In some cases, the anti-LAG3 antibody is relatlimab.

### 1. Anti-PD-1 Antibody

a. Compositions and Formulations

In some embodiments of the combination therapy methods, combinations, kits and uses provided herein, the combination therapy can be administered in one or more compositions, e.g., a pharmaceutical composition containing a PD-1 inhibitor (e.g. an anti-PD-1 antibody, such as nivolumab), and/or the T cell therapy, e.g., CAR T cell therapy.

In some embodiments, the composition, e.g., a pharmaceutical composition containing a PD-1 inhibitor, e.g., an anti-PD-1 antibody such as nivolumab, can include carriers such as a diluent, adjuvant, excipient, or vehicle with which the inhibitor(s), and/or the cells are administered. Examples of suitable pharmaceutical carriers are described in “Remington's Pharmaceutical Sciences” by E. W. Martin. Such compositions will contain a therapeutically effective amount of a PD-1 inhibitor, e.g., an anti-PD-1 antibody, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing a PD-1 inhibitor, e.g., an anti-PD-1 antibody such as nivolumab, can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, a PD-1 inhibitor, e.g., an anti-PD-1 antibody such as nivolumab, is typically formulated and administered in unit dosage forms or multiple dosage forms. Each unit dose contains a predetermined quantity of a therapeutically active PD-1 inhibitor, e.g., nivolumab, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of a PD-1 inhibitor, e.g., nivolumab. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

b. Dosing

In some embodiments, the provided combination therapy methods involve administering to the subject a checkpoint inhibitor therapy (e.g. a PD-1 inhibitor, and optionally a LAG3 inhibitor) and a T cell therapy (e.g. CAR T cells). In some embodiments, the provided combination therapy methods involve initiation administration of the checkpoint inhibitor therapy (e.g. a PD-1 inhibitor, and optionally a LAG3 inhibitor) subsequent to initiation of the T cell therapy (e.g. CAR T cells). In some embodiments, the provided combination therapy methods involve initiating administration of a checkpoint inhibitor therapy (e.g. a PD-1 inhibitor, and optionally a LAG3 inhibitor) between about 1 day and about 3 weeks after initiation of administration of the T cell therapy (CAR T cells). In some embodiments, initiation of administration of the checkpoint inhibitor therapy (e.g. a PD-1 inhibitor, and optionally a LAG3 inhibitor) is between about one week and about two weeks after initiation of administration of the T cell therapy (e.g. CAR T cells).

In some embodiments, the provided combination therapy methods involve initiating administration of the T cell therapy (e.g. CAR T cells) on Day 1. In some embodiments, the provided combination therapy methods involve initiating administration of a checkpoint inhibitor therapy (e.g. a PD-1 inhibitor, and optionally a LAG3 inhibitor) between about Day 2 and Day 20. In some embodiments, the provided combination therapy methods involve initiating administration of a checkpoint inhibitor therapy (e.g. a PD-1 inhibitor, and optionally a LAG3 inhibitor) on about Day 8 or Day 15. In some embodiments, the provided combination therapy methods involve initiating administration of a checkpoint inhibitor therapy (e.g. a PD-1 inhibitor, and optionally a LAG3 inhibitor) on about Day 8. In some embodiments, the provided combination therapy methods involve initiating administration of a checkpoint inhibitor therapy (e.g. a PD-1 inhibitor, and optionally a LAG3 inhibitor) on about Day 15.

In some embodiments, the method involves initiating administration of the checkpoint inhibitor therapy (e.g. a PD-1 inhibitor, and optionally a LAG3 inhibitor) after activation-induced cell death (AICD) of the cells of the T cell therapy (e.g. CAR T cells) has peaked.

In some embodiments, the provided combination therapy comprises: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) administration of the first dose is between Day 2 and Day 20, inclusive; and (ii) a dose is administered about every two weeks (Q2W) or about every four weeks (Q4W) in an amount of between at or about 140 mg and at or about 580 mg, inclusive.

In some embodiments, the provided combination therapy comprises: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii) each dose of the PD-1 inhibitor is between at or about 140 mg and at or about 580 mg, inclusive. In some embodiments, each subsequent dose of the PD-1 inhibitor is administered about two weeks about three weeks, or about four weeks after the previous dose of the PD-1 inhibitor.

In some embodiments, the provided combination therapy comprises: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor to the subject in a dosing regimen comprising administration of at least two doses, wherein: (i) administration of a first dose of the PD-1 inhibitor is between Day 2 and Day 20, inclusive; and (ii), each subsequent dose of the PD-1 inhibitor is administered about two weeks about three weeks, or about four weeks after the previous dose of the PD-1 inhibitor. In some embodiments, each dose of the PD-1 inhibitor is between at or about 140 mg and at or about 580 mg, inclusive.

In some embodiments, the first dose of the PD-1 inhibitor is administered between Day 8 and Day 15, inclusive. In some embodiments, the first dose of the PD-1 inhibitor is administered on Day 8. In some embodiments, the first dose is administered on Day 15. In some embodiments, the PD-1 inhibitor is administered for no longer than about three months. In some embodiments, a final dose of the PD-1 inhibitor is administered between about Day 80 and about Day 90. In some embodiments, the final dose of the PD-1 inhibitor is administered at about Day 85.

In some embodiments, each dose of wherein each dose of the PD-1 inhibitor is between at or about 160 mg and 560 mg. In some embodiments, each dose of the PD-1 inhibitor is at or about 240 mg, or at or about 480 mg. In some embodiments, each dose of the PD-1 inhibitor is 240 mg. In some embodiments, each dose of the PD-1 inhibitor is 480 mg.

In some embodiments, at least one dose of the PD-1 inhibitor is 240 mg, and at least one dose of the PD-1 inhibitor is 480 mg. In some embodiments, at least four doses of the PD-1 inhibitor are administered. In some embodiments, four doses, five doses, or six doses of the PD-1 inhibitor are administered.

In some embodiments, the first three doses of the PD-1 inhibitor are administered every two weeks (Q2W). In some embodiments, each dose of the PD-1 inhibitor is administered every two weeks (Q2W).

In some embodiments, the fourth dose of the PD-1 inhibitor is administered about three weeks or about four weeks after the previous dose of the PD-1 inhibitor. In some embodiments, the fourth dose of the PD-1 inhibitor is administered about three weeks after the previous dose of the PD-1 inhibitor. In some embodiments, the fourth dose of the PD-1 inhibitor is administered about four weeks after the previous dose of the PD-1 inhibitor.

In some embodiments, five doses of the PD-1 inhibitor are administered. In some embodiments, the first three doses of the PD-1 inhibitor are about 240 mg, and the fourth and fifth doses of the inhibitor are about 480 mg. In some cases, the five doses of the PD-1 inhibitor are administered on Days 8, 22, 36, 57, and 85. In some cases, the five doses of the PD-1 inhibitor are administered on Days 15, 29, 43, 57, and 85.

In some embodiments, five doses of the PD-1 inhibitor are administered. In some embodiments, the fifth dose of the PD-1 inhibitor is administered about four weeks after the fourth dose of the PD-1 inhibitor.

In some embodiments, about 240 mg of the PD-1 inhibitor is administered on each of Days 8, 22, and 36. In some embodiments, about 240 mg of the PD-1 inhibitor is administered on each of Days 15, 29, and 43. In some embodiments, about 480 mg of the PD-1 inhibitor is administered on each of Days 8, 36, 64, and 85. In some embodiments, about 480 mg of the PD-1 inhibitor is administered on each of Days 15, 43, 64, and 85.

In some embodiments, 240 mg of the PD-1 inhibitor is administered on each of Days 8, 22, 36, 57, 71, and 85. In some embodiments, 240 mg of the PD-1 inhibitor is administered on each of Days 15, 29, 43, 57, 71, and 85.

In some embodiments, 240 mg of the PD-1 inhibitor is administered on each of Days 8, 22, and 36; and 480 mg of the PD-1 inhibitor is administered on Days 57 and 85. In some embodiments, 240 mg of the PD-1 inhibitor is administered on each of Days 15, 29, and 43; and 480 mg of the PD-1 inhibitor is administered on 57, and 85.

In some embodiments, 480 mg of the PD-1 inhibitor is administered on each of Days 8, 36, 64, and 85. In some embodiments, 480 mg of the PD-1 inhibitor is administered on each of Days 15, 43, 64, and 85.

In some embodiments, the amount of the PD-1 inhibitor is between at or about 160 mg and 560 mg. In some embodiments, the amount of the PD-1 inhibitor is at or about 240 mg or at or about 480 mg. In some embodiments, the amount of the PD-1 inhibitor is 240 mg. In some embodiments, the amount of the PD-1 inhibitor is 480 mg. In some embodiments, a dose is administered about every two weeks (Q2W). In some embodiments, a dose is administered about every four weeks (Q4W).

In some embodiments, an amount of about 240 mg of the PD-1 inhibitor is administered Q2W. In some embodiments, an amount of about 480 mg of the PD-1 inhibitor is administered Q4W.

In some embodiments, the PD-1 inhibitor is administered in a first cycle and a second cycle, as described further below.

### 1) First Cycle

In some embodiments, the provided combination therapy methods involve administering to the subject a checkpoint inhibitor therapy (e.g. a PD-1 inhibitor, and optionally a LAG3 inhibitor) and a T cell therapy (e.g. CAR T cells), wherein the checkpoint inhibitory therapy is administered in a first cycle and a second cycle.

In some embodiments, at least one dose of the PD-1 inhibitor is administered in the first cycle. In some embodiments, at least two doses of the PD-1 inhibitor are administered in the first cycle. In some embodiments, at least three doses of the PD-1 inhibitor are administered in the first cycle. In some embodiments, two doses of the PD-1 inhibitor are administered in the first cycle. In some embodiments, three doses of the PD-1 inhibitor are administered in the first cycle.

In some embodiments, the first dose of the PD-1 inhibitor of the first cycle is administered subsequent to administration of the T cell therapy. In some embodiments, the first dose of the PD-1 inhibitor of the first cycle is administered between about 1 day and three weeks after initiation of administration of the T cell therapy. In some embodiments, the first dose of the PD-1 inhibitor of the first cycle is administered about one week after initiation of administration of the T cell therapy. In some embodiments, the first dose of the PD-1 inhibitor of the first cycle is administered about two weeks after initiation of administration of the T cell therapy.

In some embodiments, the T cell therapy is administered on Day 1. In some embodiments, the first dose of the PD-1 inhibitor of the first cycle is administered between about Day 2 and about Day 20. In some embodiments, the first dose of the PD-1 inhibitor of the first cycle is administered between on about Day 8 or on about Day 15. In some embodiments, the first dose of the PD-1 inhibitor of the first cycle is administered between on about Day 8. In some embodiments, the first dose of the PD-1 inhibitor of the first cycle is administered between on about Day 15.

In some embodiments, a dose of the PD-1 inhibitor is administered on Days 8, 15, 22, 29, 36, and/or 43. In some embodiments, a dose of the PD-1 inhibitor is administered on Day 15. In some embodiments, a dose of the PD-1 inhibitor is administered on Day 22. In some embodiments, a dose of the PD-1 inhibitor is administered on Day 29. In some embodiments, a dose of the PD-1 inhibitor is administered on Day 36. In some embodiments, a dose of the PD-1 inhibitor is administered on Day 43. In some embodiments, doses of the PD-1 inhibitor are administered on Days 8 and 36. In some embodiments, doses of the PD-1 inhibitor are administered on Days 8, 22, and 36. In some embodiments, a dose of the PD-1 is administered on Days 15, 29, and 43.

In some embodiments, a first amount of the PD-1 inhibitor is administered for each dose of the first cycle. In some embodiments, the first amount of the PD-1 inhibitor is between about 100 mg and 600 mg, between about 100 mg and about 550 mg, between about 100 mg and about 500 mg, between about 100 mg and about 450 mg, between about 100 mg and about 400 mg, between about 100 mg and about 350 mg, between about 100 mg and about 300 mg, between about 100 mg and about 250 mg, between about 100 mg and about 200 mg, between about 100 mg and about 150 mg, between about 150 mg and 600 mg, between about 150 mg and about 550 mg, between about 150 mg and about 500 mg, between about 150 mg and about 450 mg, between about 150 mg and about 400 mg, between about 150 mg and about 350 mg, between about 150 mg and about 300 mg, between about 150 mg and about 250 mg, between about 150 mg and about 200 mg, between about 200 mg and 600 mg, between about 200 mg and about 550 mg, between about 200 mg and about 500 mg, between about 200 mg and about 450 mg, between about 200 mg and about 400 mg, between about 200 mg and about 350 mg, between about 200 mg and about 300 mg, between about 200 mg and about 250 mg, between about 250 mg and 600 mg, between about 250 mg and about 550 mg, between about 250 mg and about 500 mg, between about 250 mg and about 450 mg, between about 250 mg and about 400 mg, between about 250 mg and about 350 mg, between about 250 mg and about 300 mg, between about 300 mg and 600 mg, between about 300 mg and about 550 mg, between about 300 mg and about 500 mg, between about 300 mg and about 450 mg, between about 300 mg and about 400 mg, between about 300 mg and about 350 mg, between about 350 mg and 600 mg, between about 350 mg and about 550 mg, between about 350 mg and about 500 mg, between about 350 mg and about 450 mg, between about 350 mg and about 400 mg, between about 400 mg and 600 mg, between about 400 mg and about 550 mg, between about 400 mg and about 500 mg, between about 400 mg and about 450 mg, between about 450 mg and 600 mg, between about 450 mg and about 550 mg, between about 450 mg and about 500 mg, between about 500 mg and about 600 mg, between about 500 mg and about 550 mg, or between about 550 mg and about 600 mg.

In some embodiments, the first amount of the PD-1 inhibitor is between about 140 mg and about 580 mg. In some embodiments, the first amount of the PD-1 inhibitor is between about 180 mg and about 540 mg. In some embodiments, the first amount of the PD-1 inhibitor is about 240 mg or about 480 mg. In some embodiments, the first amount of the PD-1 inhibitor is about 240 mg. In some embodiments, the first amount of the PD-1 inhibitor is about 480 mg.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20.

In some embodiments, at or about 240 mg of the PD-1 inhibitor is administered on Days 8, 22, and 36. In some embodiments, at or about 480 mg of the PD-1 inhibitor is administered on Days 8 and 36. In some embodiments, at or about 240 mg of the PD-1 inhibitor is administered on Days 15, 29, and 43. In some embodiments, at or about 480 mg of the PD-1 inhibitor is administered on Day 15.

In some embodiments, the PD-1 inhibitor is an anti-PD-1 antibody. In some embodiments, the anti-PD-1 antibody is selected from the group consisting of: nivolumab, camrelizumab, cemiplimab, dostarlimab, MEDI-0680, pembrolizumab, spartalizumab, SSI-361, tislelizumab, or a combination thereof. In some embodiments, the anti-PD-1 antibody is nivolumab.

### 2) Second Cycle

In some embodiments, the provided combination therapy methods involve administering to the subject a checkpoint inhibitor therapy (e.g. a PD-1 inhibitor, and optionally a LAG3 inhibitor) and a T cell therapy (e.g. CAR T cells), wherein the checkpoint inhibitory therapy is administered in a first cycle and a second cycle.

In some embodiments, at least one dose of the PD-1 inhibitor is administered in the second cycle. In some embodiments, at least two doses of the PD-1 inhibitor are administered in the second cycle. In some embodiments, two doses of the PD-1 inhibitor are administered in the second cycle.

In some embodiments, the first dose of the PD-1 inhibitor of the second cycle is administered between about six weeks and ten weeks after initiation of administration of the T cell therapy. In some embodiments, the first dose of the PD-1 inhibitor of the second cycle is administered about eight weeks after initiation of administration of the T cell therapy. In some embodiments, the first dose of the PD-1 inhibitor of the second cycle is administered about nine weeks after initiation of administration of the T cell therapy.

In some embodiments, the T cell therapy is administered on Day 1. In some embodiments, the first dose of the PD-1 inhibitor of the second cycle is administered between about Day 50 and about Day 65. In some embodiments, the first dose of the PD-1 inhibitor of the second cycle is administered between on about Day 50. In some embodiments, the second dose of the PD-1 inhibitor of the second cycle is administered between about Day 78 and Day 92. In some embodiments, the second dose of the PD-1 inhibitor of the second cycle is administered on about Day 85.

In some embodiments, a dose of the PD-1 inhibitor is administered on Day 57. In some embodiments, a dose of the PD-1 inhibitor is administered on Day 85. In some embodiments, the PD-1 inhibitor is administered on Days 57 and 85.

In some embodiments, a second amount of the PD-1 inhibitor is administered for each dose of the second cycle. In some embodiments, the second amount of the PD-1 inhibitor is between about 100 mg and 600 mg, between about 100 mg and about 550 mg, between about 100 mg and about 500 mg, between about 100 mg and about 450 mg, between about 100 mg and about 400 mg, between about 100 mg and about 350 mg, between about 100 mg and about 300 mg, between about 100 mg and about 250 mg, between about 100 mg and about 200 mg, between about 100 mg and about 150 mg, between about 150 mg and 600 mg, between about 150 mg and about 550 mg, between about 150 mg and about 500 mg, between about 150 mg and about 450 mg, between about 150 mg and about 400 mg, between about 150 mg and about 350 mg, between about 150 mg and about 300 mg, between about 150 mg and about 250 mg, between about 150 mg and about 200 mg, between about 200 mg and 600 mg, between about 200 mg and about 550 mg, between about 200 mg and about 500 mg, between about 200 mg and about 450 mg, between about 200 mg and about 400 mg, between about 200 mg and about 350 mg, between about 200 mg and about 300 mg, between about 200 mg and about 250 mg, between about 250 mg and 600 mg, between about 250 mg and about 550 mg, between about 250 mg and about 500 mg, between about 250 mg and about 450 mg, between about 250 mg and about 400 mg, between about 250 mg and about 350 mg, between about 250 mg and about 300 mg, between about 300 mg and 600 mg, between about 300 mg and about 550 mg, between about 300 mg and about 500 mg, between about 300 mg and about 450 mg, between about 300 mg and about 400 mg, between about 300 mg and about 350 mg, between about 350 mg and 600 mg, between about 350 mg and about 550 mg, between about 350 mg and about 500 mg, between about 350 mg and about 450 mg, between about 350 mg and about 400 mg, between about 400 mg and 600 mg, between about 400 mg and about 550 mg, between about 400 mg and about 500 mg, between about 400 mg and about 450 mg, between about 450 mg and 600 mg, between about 450 mg and about 550 mg, between about 450 mg and about 500 mg, between about 500 mg and about 600 mg, between about 500 mg and about 550 mg, or between about 550 mg and about 600 mg.

In some embodiments, the second amount of the PD-1 inhibitor is between about 140 mg and about 580 mg. In some embodiments, the second amount of the PD-1 inhibitor is between about 180 mg and about 540 mg. In some embodiments, the second amount of the PD-1 inhibitor is about 240 mg or about 480 mg. In some embodiments, the second amount of the PD-1 inhibitor is about 240 mg. In some embodiments, the second amount of the PD-1 inhibitor is about 480 mg.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and (ii) administration of a second amount of the PD-1 inhibitor of between at or about 140 mg and at or about 580 mg, inclusive, about once every four weeks (Q4W) for a second cycle, wherein at least two doses of the second amount are administered in the second cycle, and the first dose of the second cycle is administered between Day 50 and Day 65.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and (ii) administration of a second amount of the PD-1 inhibitor of between at or about 380 mg and at or about 580 mg, inclusive, about once every four weeks (Q4W) for a second cycle, wherein at least two doses of the second amount are administered in the second cycle, and the first dose of the second cycle is administered between Day 50 and Day 65.

In some embodiments, at or about 480 mg of the PD-1 inhibitor is administered on Day 57. In some embodiments, at or about 480 mg of the PD-1 inhibitor is administered on Day 85. In some embodiments, at or about 480 mg of the PD-1 inhibitor is administered on Days 57 and 85.

In some embodiments, at or about 240 mg of the PD-1 inhibitor is administered on Days 8, 22, and 36, and at or about 480 mg of the PD-1 inhibitor is administered on Days 57 and 85. In some embodiments, at or about 480 mg of the PD-1 inhibitor is administered on Days 8 and 36, and at or about 480 mg of the PD-1 inhibitor is administered on Days 57 and 85. In some embodiments, at or about 240 mg of the PD-1 inhibitor is administered on Days 15, 29, and 43, and at or about 480 mg of the PD-1 inhibitor is administered on Days 57 and 85. In some embodiments, at or about 480 mg of the PD-1 inhibitor is administered on Day 15, and at or about 480 mg of the PD-1 inhibitor is administered on Days 57 and 85.

In some embodiments, the PD-1 inhibitor is an anti-PD-1 antibody. In some embodiments, the anti-PD-1 antibody is selected from the group consisting of: nivolumab, camrelizumab, cemiplimab, dostarlimab, MEDI-0680, pembrolizumab, spartalizumab, SSI-361, tislelizumab, or a combination thereof. In some embodiments, the anti-PD-1 antibody is nivolumab.

### 2. Anti-LAG3 Antibody

a. Compositions and Formulations

In some embodiments of the combination therapy methods, combinations, kits and uses provided herein, the combination therapy can be administered in one or more compositions, e.g., a pharmaceutical composition containing a LAG3 inhibitor (e.g. an anti-LAG3 antibody, such as relatlimab), and/or the T cell therapy, e.g., CAR T cell therapy.

In some embodiments, the composition, e.g., a pharmaceutical composition containing a LAG3 inhibitor, e.g., an anti-LAG3 antibody such as relatlimab, can include carriers such as a diluent, adjuvant, excipient, or vehicle with which the inhibitor(s), and/or the cells are administered. Examples of suitable pharmaceutical carriers are described in “Remington's Pharmaceutical Sciences” by E. W. Martin. Such compositions will contain a therapeutically effective amount of a LAG3 inhibitor, e.g., an anti-LAG3 antibody, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing a LAG3 inhibitor, e.g., an anti-LAG3 antibody such as relatlimab, can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, a LAG3 inhibitor, e.g., an anti-LAG3 antibody such as relatlimab, is typically formulated and administered in unit dosage forms or multiple dosage forms. Each unit dose contains a predetermined quantity of a therapeutically active LAG3 inhibitor, e.g., relatlimab, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of a LAG3 inhibitor, e.g., relatlimab. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

In some embodiments, the PD-1 inhibitor and the LAG3 inhibitor are formulated as a single composition. In some embodiments, simultaneous administration of the PD-1 inhibitor and the LAG3 inhibitor to the subject comprises administration of a single composition comprising the PD-1 inhibitor and the LAG3 inhibitor. In some embodiments, the PD-1 inhibitor and the LAG3 inhibitor are administered together as a single composition over the course of at or about 15 minutes, at or about 30 minutes, at or about 45 minutes, at or about 60 minutes, at or about 75 minutes, or at or about 90 minutes. In some embodiments, the PD-1 inhibitor and the LAG3 inhibitor are administered together as a single composition over the course of at or about 15 minutes. In some embodiments, the PD-1 inhibitor and the LAG3 inhibitor are administered as a single composition over the course of at or about 30 minutes. In some embodiments, the PD-1 inhibitor and the LAG3 inhibitor are administered as a single composition over the course of at or about 45 minutes. In some embodiments, the PD-1 inhibitor and the LAG3 inhibitor are administered as a single composition over the course of at or about 60 minutes. In some embodiments, the PD-1 inhibitor and the LAG3 inhibitor are administered as a single composition over the course of at or about 75 minutes. In some embodiments, the PD-1 inhibitor and the LAG3 inhibitor are administered as a single composition over the course of at or about 90 minutes.

In some embodiments, the PD-1 inhibitor and the LAG3 inhibitor are administered sequentially as separate compositions. In some embodiments, a first composition comprising one of the PD-1 inhibitor and the LAG3 inhibitor is formulated for administration, and a second composition comprising the other of the PD-1 inhibitor and the LAG3 inhibitor is formulated for administration. Thus, in some embodiments, administration of the PD-1 inhibitor and the LAG3 comprises administering a plurality of separate compositions. In some embodiments, the first composition comprises the PD-1 inhibitor.

In some embodiments, the composition comprising the PD-1 inhibitor is administered over the course of at or about 15 minutes, at or about 30 minutes, at or about 45 minutes, or at or about 60 minutes. In some embodiments, the composition comprising the PD-1 inhibitor is administered over the course of at or about 15 minutes. In some embodiments, the composition comprising the PD-1 inhibitor is administered over the course of at or about 30 minutes. In some embodiments, the composition comprising the PD-1 inhibitor is administered over the course of at or about 45 minutes.

In some embodiments, administration of the second composition is initiated at or about 5 minutes, at or about 10 minutes, at or about 15 minutes, at or about 20 minutes, at or about 25 minutes, at or about 30 minutes, at or about 35 minutes, at or about 40 minutes, or at or about 45 minutes after the administration of the first composition is complete. In some embodiments, administration of the second composition is initiated at or about 15 minutes after the administration of the first composition is complete. In some embodiments, administration of the second composition is initiated at or about 30 minutes after the administration of the first composition is complete. In some embodiments, the first composition comprises the PD-1 inhibitor. In some embodiments, the second composition comprises at or about 960 mg of the LAG3 inhibitor and is administered at or about 30 minutes after administration of the first composition comprising the PD-1 inhibitor.

In some embodiments, the composition comprising the LAG3 inhibitor is administered over the course of at or about 30 minutes, at or about 45 minutes, at or about 60 minutes, or at or about 75 minutes. In some embodiments, the composition comprising the LAG3 inhibitor is administered over the course of at or about 45 minutes, at or about 60 minutes, or at or about 75 minutes. In some embodiments, the composition comprising the LAG3 inhibitor is administered over the course of at or about 30 minutes. In some embodiments, the composition comprising the LAG3 inhibitor is administered over the course of at or about 45 minutes. In some embodiments, the composition comprising the LAG3 inhibitor is administered over the course of at or about 60 minutes. In some embodiments, the composition comprising the LAG3 inhibitor is administered over the course of at or about 75 minutes.

In some embodiments, the composition comprising the PD-1 inhibitor is administered first. In some embodiments, the composition comprising the PD-1 inhibitor is administered over the course of at or about 30 minutes. In some embodiments, between about 15 minutes and about 30 minutes after the administration of the PD-1 inhibitor ends, administration of the composition comprising the LAG3 inhibitor begins. In some embodiments, the composition comprising the LAG3 inhibitor is administered over the course of at or about 30 minutes. Thus, in some embodiments, the composition comprising the PD-1 inhibitor is administered over the course of about 30 minutes, and between about 15 minutes and 30 minutes after the administration of the PD-1 inhibitor ends, the composition comprising the LAG3 inhibitor is administered over the course of the subsequent 30 minutes.

b. Dosing

In some embodiments, the provided combination therapy methods involve administering to the subject a checkpoint inhibitor therapy (e.g. a PD-1 inhibitor and a LAG3 inhibitor) and a T cell therapy (e.g. CAR T cells). In some embodiments, the provided combination therapy methods involve initiation administration of the checkpoint inhibitor therapy (e.g. a PD-1 inhibitor and a LAG3 inhibitor) subsequent to initiation of the T cell therapy (e.g. CAR T cells). In some embodiments, the provided combination therapy methods involve initiating administration of a checkpoint inhibitor therapy (e.g. a PD-1 inhibitor and a LAG3 inhibitor) between about 1 day and about 3 weeks after initiation of administration of the T cell therapy (CAR T cells). In some embodiments, initiation of administration of the checkpoint inhibitor therapy (e.g. a PD-1 inhibitor and a LAG3 inhibitor) is between about one week and about two weeks after initiation of administration of the T cell therapy (e.g. CAR T cells).

In some embodiments, the provided combination therapy methods involve initiating administration of the T cell therapy (e.g. CAR T cells) on Day 1. In some embodiments, the provided combination therapy methods involve initiating administration of a checkpoint inhibitor therapy (e.g. a PD-1 inhibitor and a LAG3 inhibitor) between about Day 2 and Day 20. In some embodiments, the provided combination therapy methods involve initiating administration of a checkpoint inhibitor therapy (e.g. a PD-1 inhibitor and a LAG3 inhibitor) on about Day 8 or Day 15. In some embodiments, the provided combination therapy methods involve initiating administration of a checkpoint inhibitor therapy (e.g. a PD-1 inhibitor and a LAG3 inhibitor) on about Day 8. In some embodiments, the provided combination therapy methods involve initiating administration of a checkpoint inhibitor therapy (e.g. a PD-1 inhibitor and a LAG3 inhibitor) on about Day 15.

In some embodiments, the method involves initiating administration of the checkpoint inhibitor therapy (e.g. a PD-1 inhibitor and a LAG3 inhibitor) after activation-induced cell death (AICD) of the cells of the T cell therapy (e.g. CAR T cells) has peaked.

In some embodiments, the provided combination therapy comprises: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a PD-1 inhibitor and a LAG3 inhibitor to the subject in a dosing regimen comprising administration of at least two doses of the PD-1 inhibitor and at least two doses of the LAG3 inhibitor, wherein: (i) administration of the first dose of the PD-1 inhibitor and the first dose of the LAG3 inhibitor is between Day 2 and Day 20, inclusive; and (ii) a dose of the PD-1 inhibitor and a dose of the LAG3 inhibitor is administered about every two weeks (Q2W) or about every four weeks (Q4W) in an amount of between at or about 140 mg and at or about 580 mg, inclusive.

In some embodiments, the first dose of the LAG3 inhibitor is administered between Day 8 and Day 15, inclusive. In some embodiments, the first dose of the LAG3 inhibitor is administered on Day 8. In some embodiments, the first dose of the LAG3 inhibitor is administered on Day 15. In some embodiments, the amount of the PD-1 inhibitor is between at or about 160 mg and 560 mg.

In some embodiments, the methods comprise administering a dose of a LAG3 inhibitor about every two weeks (Q2W) or about every four weeks (Q4W). In some embodiments, the methods comprise administering a dose of a LAG3 inhibitor about every two weeks (Q2W). In some embodiments, the methods comprise administering a dose of a LAG3 inhibitor about every four weeks (Q4W). In some embodiments, the methods comprising administering a LAG3 inhibitor to the subject in a dosing regimen comprising administering a dose of the LAG3 inhibitor on each of the same days on which a dose of the PD-1 inhibitor is administered.

In some embodiments, the provided combination therapy comprises: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering a PD-1 inhibitor to the subject; and (3) administering a LAG3 inhibitor to the subject.

In some embodiments, a first dose of the LAG3 inhibitor is administered between Day 2 and Day 20, inclusive. In some embodiments, a first dose of the LAG3 inhibitor is administered between Day 8 and Day 15, inclusive. In some embodiments, a first dose of the LAG3 inhibitor is administered on Day 8. In some embodiments, a first dose of the LAG3 inhibitor is administered on Day 15. In some embodiments, a first dose of the PD-1 inhibitor and a first dose of the LAG3 inhibitor are independently administered, each between Day 2 and Day 20, inclusive. In some embodiments, the first dose of the PD-1 inhibitor and the first dose of the LAG3 inhibitor are administered on the same day. In some embodiments, each subsequent dose of the PD-1 inhibitor is administered about two weeks, about three weeks, or about four weeks after the previous dose of the PD-1 inhibitor.

In some embodiments, each dose of the LAG3 inhibitor is between about 60 mg and about 540 mg, inclusive. In some embodiments, each dose of the LAG3 inhibitor is between about 120 mg and about 480 mg. In some embodiments, each dose of the LAG3 inhibitor is about 120 mg. In some embodiments, each dose of the LAG3 inhibitor is about 240 mg. In some embodiments, each dose of the LAG3 inhibitor is about 480 mg.

In some embodiments, at least three doses of the LAG3 inhibitor are administered. In some embodiments, three doses, four doses, or six doses of the LAG3 inhibitor are administered.

In some embodiments, three doses of the LAG3 inhibitor are administered. In some embodiments, the second dose of the LAG3 inhibitor is administered about four weeks after the first dose of the LAG3 inhibitor. In some embodiments, each dose of the LAG3 inhibitor is about 240 mg. In some embodiments, the third dose of the LAG3 inhibitor is administered about five weeks after the second dose of the LAG3 inhibitor. In some embodiments, about 240 mg of the LAG3 inhibitor is administered on each of Days 8, 36, and 71. In some embodiments, the third dose of the LAG3 inhibitor is administered about four weeks after the second dose of the LAG3 inhibitor. In some embodiments, about 240 mg of the LAG3 inhibitor is administered on each of Days 15, 43, and 71.

In some embodiments, four doses of the LAG3 inhibitor are administered. In some embodiments, each dose of the LAG3 inhibitor is about 240 mg. In some embodiments, about 240 mg of the LAG3 inhibitor are administered on each of Days 8, 36, 64, and 85. In some embodiments, about 240 mg of the LAG3 inhibitor are administered on each of Days 15, 43, 64, and 85. In some embodiments, each dose of the LAG3 inhibitor is about 480 mg. In some embodiments, about 480 mg of the LAG3 inhibitor are administered on each of Days 8, 36, 64, and 85. In some embodiments, about 480 mg of the LAG3 inhibitor are administered on each of Days 15, 43, 64, and 85.

In some embodiments, six doses of the LAG3 inhibitor are administered. In some embodiments, each of the second and third doses of the LAG3 inhibitor are administered about two weeks after the previous dose of the LAG3 inhibitor. In some embodiments, each of the second, third, fifth, and six doses of the LAG3 inhibitor are administered about two weeks after the previous dose of the LAG3 inhibitor. In some embodiments, the fourth dose of the LAG3 inhibitor is administered about three weeks after the previous dose of the LAG3 inhibitor. In some embodiments, each dose of the LAG3 inhibitor is about 120 mg. In some embodiments, about 120 mg of the LAG3 inhibitor are administered on each of Days 8, 22, 36, 57, 71, and 85. In some embodiments, each dose of the LAG3 inhibitor is about 240 mg. In some embodiments, about 240 mg of the LAG3 inhibitor are administered on each of Days 8, 22, 36, 57, 71, and 85. In some embodiments, each dose of the LAG3 inhibitor is administered every two weeks (Q2W). In some embodiments, each dose of the LAG3 inhibitor is about 120 mg. In some embodiments, about 120 mg of the LAG3 inhibitor are administered on each of Days 15, 29, 43, 57, 71, and 85. In some embodiments, each dose of the LAG3 inhibitor is about 240 mg. In some embodiments, about 240 mg of the LAG3 inhibitor are administered on each of Days 15, 29, 43, 57, 71, and 85.

In some embodiments, doses of the PD-1 inhibitor and doses of the LAG3 inhibitor are administered with the same frequency. In some embodiments, each dose of the PD-1 inhibitor is administered on the same day as a dose of the LAG3 inhibitor or each dose of the LAG3 inhibitor is administered on the same day as a dose of the PD-1 inhibitor. In some embodiments, each dose of the PD-1 inhibitor is administered on the same day as a dose of the LAG3 inhibitor. In some embodiments, each dose of the LAG3 inhibitor is administered on the same day as a dose of the PD-1 inhibitor. In some embodiments, each dose of the PD-1 inhibitor is administered on the same day as a dose of the LAG3 inhibitor, and each dose of the LAG3 inhibitor is administered on the same day as a dose of the PD-1 inhibitor.

In some embodiments, doses of the LAG3 inhibitor are administered half as frequently as doses for the PD-1 inhibitor. In some embodiments, each dose of the PD-1 inhibitor is double the dose of the LAG3 inhibitor. In some embodiments, each dose of the PD-1 inhibitor is the same as the dose of the LAG3 inhibitor. In some embodiments, the PD-1 inhibitor and the LAG3 inhibitor are formulated in a single composition, such as described in Section I.A.4.

In some embodiments, each dose of the LAG3 inhibitor is administered in an amount between at or about 160 mg and at or about 1040 mg. In some embodiments, each dose of the LAG3 inhibitor is administered in an amount between at or about 160 mg and at or about 320 mg. In some embodiments, each dose of the LAG3 inhibitor is administered in an amount at or about 240 mg. In some embodiments, each dose of the LAG3 inhibitor is administered in an amount between at or about 400 mg and at or about 560 mg. In some embodiments, each dose of the LAG3 inhibitor is administered in an amount at or about 480 mg. In some embodiments, each dose of the LAG3 inhibitor is administered in an amount between at or about 880 mg and at or about 1040 mg. In some embodiments, each dose of the LAG3 inhibitor is administered in an amount at or about 960 mg.

In some embodiments, an amount of about 240 mg or about 480 mg of the LAG3 inhibitor s administered Q2W. In some embodiments, an amount of about 240 mg of the LAG3 inhibitor is administered Q2W. In some embodiments, an amount of about 480 mg of the LAG3 inhibitor is administered Q2W. In some embodiments, an amount of about 960 mg of the LAG3 inhibitor is administered Q4W.

In some embodiments, the LAG3 inhibitor is administered in a first cycle and a second cycle, as described further below.

### 1) First Cycle

In some embodiments, the provided combination therapy methods involve administering to the subject a checkpoint inhibitor therapy (e.g. a PD-1 inhibitor and a LAG3 inhibitor) and a T cell therapy (e.g. CAR T cells), wherein the checkpoint inhibitory therapy is administered in a first cycle and a second cycle.

In some embodiments, at least one dose of the LAG3 inhibitor is administered in the first cycle. In some embodiments, at least two doses of the LAG3 inhibitor are administered in the first cycle. In some embodiments, at least three doses of the LAG3 inhibitor are administered in the first cycle. In some embodiments, two doses of the LAG3 inhibitor are administered in the first cycle. In some embodiments, three doses of the LAG3 inhibitor are administered in the first cycle.

In some embodiments, the first dose of the LAG3 inhibitor of the first cycle is administered subsequent to administration of the T cell therapy. In some embodiments, the first dose of the LAG3 inhibitor of the first cycle is administered between about 1 day and three weeks after initiation of administration of the T cell therapy. In some embodiments, the first dose of the LAG3 inhibitor of the first cycle is administered about one week after initiation of administration of the T cell therapy. In some embodiments, the first dose of the LAG3 inhibitor of the first cycle is administered about two weeks after initiation of administration of the T cell therapy.

In some embodiments, the T cell therapy is administered on Day 1. In some embodiments, the first dose of the LAG3 inhibitor of the first cycle is administered between about Day 2 and about Day 20. In some embodiments, the first dose of the LAG3 inhibitor of the first cycle is administered between on about Day 8 or on about Day 15. In some embodiments, the first dose of the LAG3 inhibitor of the first cycle is administered between on about Day 8. In some embodiments, the first dose of the LAG3 inhibitor of the first cycle is administered between on about Day 15.

In some embodiments, a dose of the LAG3 inhibitor is administered on Days 8, 15, 22, 29, 36, and/or 43. In some embodiments, a dose of the LAG3 inhibitor is administered on Day 15. In some embodiments, a dose of the LAG3 inhibitor is administered on Day 22. In some embodiments, a dose of the LAG3 inhibitor is administered on Day 29. In some embodiments, a dose of the LAG3 inhibitor is administered on Day 36. In some embodiments, a dose of the LAG3 inhibitor is administered on Day 43. In some embodiments, doses of the LAG3 inhibitor are administered on Days 8 and 36. In some embodiments, doses of the LAG3 inhibitor are administered on Days 8, 22, and 36. In some embodiments, a dose of the LAG3 is administered on Days 15, 29, and 43.

In some embodiments, a first amount of the LAG3 inhibitor is administered for each dose of the first cycle. In some embodiments, the first amount of the LAG3 inhibitor is between about 100 mg and about 1050 mg, between about 100 mg and about 1000 mg, between about 100 mg and about 900 mg, between about 100 mg and about 850 mg, between about 100 mg and about 800 mg, between about 100 mg and about 750 mg, between about 100 mg and about 700 mg, between about 100 mg and about 650 mg, between about 100 mg and 600 mg, between about 100 mg and about 550 mg, between about 100 mg and about 500 mg, between about 100 mg and about 450 mg, between about 100 mg and about 400 mg, between about 100 mg and about 350 mg, between about 100 mg and about 300 mg, between about 100 mg and about 250 mg, between about 100 mg and about 200 mg, between about 100 mg and about 150 mg, between about 150 mg and about 1050 mg, between about 150 mg and about 1000 mg, between about 150 mg and about 900 mg, between about 150 mg and about 850 mg, between about 150 mg and about 800 mg, between about 150 mg and about 750 mg, between about 150 mg and about 700 mg, between about 150 mg and about 650 mg, between about 150 mg and 600 mg, between about 150 mg and about 550 mg, between about 150 mg and about 500 mg, between about 150 mg and about 450 mg, between about 150 mg and about 400 mg, between about 150 mg and about 350 mg, between about 150 mg and about 300 mg, between about 150 mg and about 250 mg, between about 150 mg and about 200 mg, between about 200 mg and about 1050 mg, between about 200 mg and about 1000 mg, between about 200 mg and about 900 mg, between about 200 mg and about 850 mg, between about 200 mg and about 800 mg, between about 200 mg and about 750 mg, between about 200 mg and about 700 mg, between about 200 mg and about 650 mg, between about 200 mg and 600 mg, between about 200 mg and about 550 mg, between about 200 mg and about 500 mg, between about 200 mg and about 450 mg, between about 200 mg and about 400 mg, between about 200 mg and about 350 mg, between about 200 mg and about 300 mg, between about 200 mg and about 250 mg, between about 250 mg and about 1050 mg, between about 250 mg and about 1000 mg, between about 250 mg and about 900 mg, between about 250 mg and about 850 mg, between about 250 mg and about 800 mg, between about 250 mg and about 750 mg, between about 250 mg and about 700 mg, between about 250 mg and about 650 mg, between about 250 mg and 600 mg, between about 250 mg and about 550 mg, between about 250 mg and about 500 mg, between about 250 mg and about 450 mg, between about 250 mg and about 400 mg, between about 250 mg and about 350 mg, between about 250 mg and about 300 mg, between about 300 mg and about 1050 mg, between about 300 mg and about 1000 mg, between about 300 mg and about 900 mg, between about 300 mg and about 850 mg, between about 300 mg and about 800 mg, between about 300 mg and about 750 mg, between about 300 mg and about 700 mg, between about 300 mg and about 650 mg, between about 300 mg and 600 mg, between about 300 mg and about 550 mg, between about 300 mg and about 500 mg, between about 300 mg and about 450 mg, between about 300 mg and about 400 mg, between about 300 mg and about 350 mg, between about 350 mg and about 1050 mg, between about 350 mg and about 1000 mg, between about 350 mg and about 900 mg, between about 350 mg and about 850 mg, between about 350 mg and about 800 mg, between about 350 mg and about 750 mg, between about 350 mg and about 700 mg, between about 350 mg and about 650 mg, between about 350 mg and 600 mg, between about 350 mg and about 550 mg, between about 350 mg and about 500 mg, between about 350 mg and about 450 mg, between about 350 mg and about 400 mg, between about 400 mg and about 1050 mg, between about 400 mg and about 1000 mg, between about 400 mg and about 900 mg, between about 400 mg and about 850 mg, between about 400 mg and about 800 mg, between about 400 mg and about 750 mg, between about 400 mg and about 700 mg, between about 400 mg and about 650 mg, between about 400 mg and 600 mg, between about 400 mg and about 550 mg, between about 400 mg and about 500 mg, between about 400 mg and about 450 mg, between about 450 mg and about 1050 mg, between about 450 mg and about 1000 mg, between about 450 mg and about 900 mg, between about 450 mg and about 850 mg, between about 450 mg and about 800 mg, between about 450 mg and about 750 mg, between about 450 mg and about 700 mg, between about 450 mg and about 650 mg, between about 450 mg and 600 mg, between about 450 mg and about 550 mg, between about 450 mg and about 500 mg, between about 500 mg and about 600 mg, between about 500 mg and about 550 mg, or between about 550 mg and about 600 mg.

In some embodiments, the first amount of the LAG3 inhibitor is between about 140 mg and about 1040 mg. In some embodiments, the first amount of the LAG3 inhibitor is between about 180 mg and about 1000 mg. In some embodiments, the first amount of the LAG3 inhibitor is between about 180 mg and about 300 mg. In some embodiments, the first amount of the LAG3 inhibitor is about 240 mg. In some embodiments, the first amount of the LAG3 inhibitor is between about 380 mg and 560 mg. In some embodiments, the first amount of the LAG3 inhibitor is about 480 mg. In some embodiments, the first amount of the LAG3 inhibitor is between about 840 mg and 1060 mg. In some embodiments, the first amount of the LAG3 inhibitor is about 960 mg.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and (3) administering a dose of a LAG3 inhibitor to the subject about every two weeks (Q2W) or about every four weeks (Q4W).

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and (3) administering a dose of a LAG3 inhibitor to the subject in a dosing regimen comprising administration of a dose of the LAG3 inhibitor on each of the same days on which a dose of the PD-1 inhibitor is administered.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and (3) administering a dose of a LAG3 inhibitor to the subject about every two weeks (Q2W) or about every four weeks (Q4W).

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and (3) administering a dose of a LAG3 inhibitor to the subject in a dosing regimen comprising administration of a dose of the LAG3 inhibitor on each of the same days on which a dose of the PD-1 inhibitor is administered.

In some embodiments, each dose of the LAG3 inhibitor is administered in a first amount during the first cycle and in a second amount during the second cycle.

In some embodiments, the first amount of the LAG3 inhibitor is between at or about 160 mg and at or about 320 mg. In some embodiments, the first amount of the LAG3 inhibitor is between at or about 200 mg and at or about 280 mg. In some embodiments, the first amount of the LAG3 inhibitor is at or about 240 mg. In some embodiments, the first amount of the LAG3 inhibitor is between at or about 400 mg and at or about 560 mg. In some embodiments, the first amount of the LAG3 inhibitor is between at or about 440 mg and at or about 520 mg. In some embodiments, the first amount of the LAG3 inhibitor is at or about 480 mg. In some embodiments, the first amount of the LAG3 inhibitor is between at or about 880 mg and at or about 1040 mg. In some embodiments, the first amount of the LAG3 inhibitor is between at or about 920 mg and at or about 1000 mg. In some embodiments, the first amount of the LAG3 inhibitor is at or about 960 mg.

In some embodiments, at or about 240 mg of the LAG3 inhibitor is administered on Days 8, 22, and 36. In some embodiments, at or about 480 mg of the LAG3 inhibitor is administered on Days 8, 22, and 36. In some embodiments, at or about 480 mg of the LAG3 inhibitor is administered on Days 8 and 36. In some embodiments, at or about 960 mg of the LAG3 inhibitor is administered on Days 8 and 36. In some embodiments, at or about 240 mg of the LAG3 inhibitor is administered on Days 15, 29, and 43. In some embodiments, at or about 480 mg of the LAG3 inhibitor is administered on Days 15, 29, and 43. In some embodiments, at or about 480 mg of the LAG3 inhibitor is administered on Day 15. In some embodiments, at or about 960 mg of the LAG3 inhibitor is administered on Day 15.

In some embodiments, the LAG3 inhibitor is an anti-LAG3 antibody. In some embodiments, the anti-LAG3 antibody is selected from the group consisting of: relatlimab, MK-4280, and ieramilimab, or a combination thereof. In some embodiments, the anti-LAG3 antibody is relatlimab.

### 2) Second Cycle

In some embodiments, the provided combination therapy methods involve administering to the subject a checkpoint inhibitor therapy (e.g. a PD-1 inhibitor and a LAG3 inhibitor) and a T cell therapy (e.g. CAR T cells), wherein the checkpoint inhibitory therapy is administered in a first cycle and a second cycle.

In some embodiments, at least one dose of the LAG3 inhibitor is administered in the second cycle. In some embodiments, at least two doses of the LAG3 inhibitor are administered in the second cycle. In some embodiments, two doses of the LAG3 inhibitor are administered in the second cycle.

In some embodiments, the first dose of the LAG3 inhibitor of the second cycle is administered between about six weeks and ten weeks after initiation of administration of the T cell therapy. In some embodiments, the first dose of the LAG3 inhibitor of the second cycle is administered about eight weeks after initiation of administration of the T cell therapy. In some embodiments, the first dose of the LAG3 inhibitor of the second cycle is administered about nine weeks after initiation of administration of the T cell therapy.

In some embodiments, the T cell therapy is administered on Day 1. In some embodiments, the first dose of the LAG3 inhibitor of the second cycle is administered between about Day 50 and about Day 65. In some embodiments, the first dose of the LAG3 inhibitor of the second cycle is administered between on about Day 50. In some embodiments, the second dose of the LAG3 inhibitor of the second cycle is administered between about Day 78 and Day 92. In some embodiments, the second dose of the LAG3 inhibitor of the second cycle is administered on about Day 85.

In some embodiments, a dose of the LAG3 inhibitor is administered on Day 57. In some embodiments, a dose of the LAG3 inhibitor is administered on Day 85. In some embodiments, the LAG3 inhibitor is administered on Days 57 and 85.

In some embodiments, a second amount of the LAG3 inhibitor is administered for each dose of the second cycle. In some embodiments, the second amount of the LAG3 inhibitor is between about 300 mg and 1100 mg, between about 300 mg and 1000 mg, between about 300 mg and 900 mg, between about 300 mg and 800 mg, between about 300 mg and 700 mg, between about 300 mg and 600 mg, between about 300 mg and about 550 mg, between about 300 mg and about 500 mg, between about 300 mg and about 450 mg, between about 400 mg and 1100 mg, between about 400 mg and 1000 mg, between about 400 mg and 900 mg, between about 400 mg and 800 mg, between about 400 mg and 700 mg, between about 400 mg and 600 mg, between about 400 mg and about 550 mg, between about 400 mg and about 500 mg, between about 400 mg and about 450 mg, between about 500 mg and 1100 mg, between about 500 mg and 1000 mg, between about 500 mg and 900 mg, between about 500 mg and 800 mg, between about 500 mg and 700 mg, between about 500 mg and about 600 mg, between about 500 mg and about 550 mg, between about 600 mg and 1100 mg, between about 600 mg and 1000 mg, between about 600 mg and 900 mg, between about 600 mg and 800 mg, between about 600 mg and 700 mg, between about 700 mg and 1100 mg, between about 700 mg and 1000 mg, between about 700 mg and 900 mg, between about 700 mg and 800 mg, between about 800 mg and 1100 mg, between about 800 mg and 1000 mg, between about 800 mg and 900 mg, between about 900 mg and 1100 mg, between about 1000 mg and 1000 mg, or between about 1000 mg and 1100 mg.

In some embodiments, the second amount of the LAG3 inhibitor is between about 360 mg and about 1080 mg. In some embodiments, the second amount of the LAG3 inhibitor is between about 880 mg and about 1040 mg. In some embodiments, the second amount of the LAG3 inhibitor is about 960 mg. In some embodiments, the second amount of the LAG3 inhibitor is between about 360 mg and about 540 mg. In some embodiments, the second amount of the LAG3 inhibitor is about 480 mg.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and (ii) administration of a second amount of the PD-1 inhibitor of between at or about 140 mg and at or about 580 mg, inclusive, about once every four weeks (Q4W) for a second cycle, wherein at least two doses of the second amount are administered in the second cycle, and the first dose of the second cycle is administered between Day 50 and Day 65; and (3) administering a dose of a LAG3 inhibitor to the subject about every two weeks (Q2W) or about every four weeks (Q4W).

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and (ii) administration of a second amount of the PD-1 inhibitor of between at or about 140 mg and at or about 580 mg, inclusive, about once every four weeks (Q4W) for a second cycle, wherein at least two doses of the second amount are administered in the second cycle, and the first dose of the second cycle is administered between Day 50 and Day 65; and (3) administering a dose of a LAG3 inhibitor to the subject in a dosing regimen comprising administration of a dose of the LAG3 inhibitor on each of the same days on which a dose of the PD-1 inhibitor is administered.

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and (ii) administration of a second amount of the PD-1 inhibitor of between at or about 380 mg and at or about 580 mg, inclusive, about once every four weeks (Q4W) for a second cycle, wherein at least two doses of the second amount are administered in the second cycle, and the first dose of the second cycle is administered between Day 50 and Day 65; and (3) administering a dose of a LAG3 inhibitor to the subject about every two weeks (Q2W) or about every four weeks (Q4W).

In some embodiments, the provided combination therapy methods include: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and (ii) administration of a second amount of the PD-1 inhibitor of between at or about 380 mg and at or about 580 mg, inclusive, about once every four weeks (Q4W) for a second cycle, wherein at least two doses of the second amount are administered in the second cycle, and the first dose of the second cycle is administered between Day 50 and Day 65; and (3) administering a dose of a LAG3 inhibitor to the subject in a dosing regimen comprising administration of a dose of the LAG3 inhibitor on each of the same days on which a dose of the PD-1 inhibitor is administered.

In some embodiments, at or about 480 mg of the LAG3 inhibitor is administered on Day 57. In some embodiments, at or about 960 mg of the LAG3 inhibitor is administered on Day 57. In some embodiments, at or about 480 mg of the LAG3 inhibitor is administered on Day 85. In some embodiments, at or about 960 mg of the LAG3 inhibitor is administered on Day 85. In some embodiments, at or about 480 mg of the LAG3 inhibitor is administered on Days 57 and 85. In some embodiments, at or about 960 mg of the LAG3 inhibitor is administered on Days 57 and 85.

In some embodiments, at or about 240 mg of the LAG3 inhibitor is administered on Days 8, 22, and 36, and at or about 480 mg of the LAG3 inhibitor is administered on Days 57 and 85. In some embodiments, at or about 480 mg of the LAG3 inhibitor is administered on Days 8, 22, and 36, and at or about 960 mg of the LAG3 inhibitor is administered on Days 57 and 85. In some embodiments, at or about 480 mg of the LAG3 inhibitor is administered on Days 8 and 36, and at or about 480 mg of the LAG3 inhibitor is administered on Days 57 and 85. In some embodiments, at or about 960 mg of the LAG3 inhibitor is administered on Days 8 and 36, and at or about 960 mg of the LAG3 inhibitor is administered on Days 57 and 85. In some embodiments, at or about 240 mg of the LAG3 inhibitor is administered on Days 15, 29, and 43, and at or about 480 mg of the LAG3 inhibitor is administered on Days 57 and 85. In some embodiments, at or about 480 mg of the LAG3 inhibitor is administered on Days 15, 29, and 43, and at or about 960 mg of the LAG3 inhibitor is administered on Days 57 and 85. In some embodiments, at or about 480 mg of the LAG3 inhibitor is administered on Day 15, and at or about 480 mg of the LAG3 inhibitor is administered on Days 57 and 85. In some embodiments, at or about 960 mg of the LAG3 inhibitor is administered on Day 15, and at or about 960 mg of the LAG3 inhibitor is administered on Days 57 and 85.

In some embodiments, the LAG3 inhibitor is an anti-LAG3 antibody. In some embodiments, the anti-LAG3 antibody is selected from the group consisting of: relatlimab, MK-4280, and ieramilimab, or a combination thereof. In some embodiments, the anti-LAG3 antibody is relatlimab.

### 3. Dosing

In some embodiments, a subject is administered a dose of engineered cells comprising T cells expressing a chimeric antigen receptor (CAR) that binds CD19 inhibitor and a checkpoint inhibitor therapy in a dosing regimen comprising (i) administration of the dose of engineered cells on Day 1; and (ii) administration of a PD-1 inhibitor (e.g. an anti-PD-1 antibody) in a first and second cycle, wherein the first dose of the first cycle is administered between about Day 2 and Day 20 and the first dose of the second cycle is administered between about Day 50 and Day 65.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising administration of 480 mg of the PD-1 inhibitor on Days 8, 36, 57, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising administration of 480 mg of the PD-1 inhibitor on Days 15, 57, and 85.

In some embodiments, a subject is administered a dose of engineered cells comprising T cells expressing a chimeric antigen receptor (CAR) that binds CD19 inhibitor and a checkpoint inhibitor therapy in a dosing regimen comprising (i) administration of the dose of engineered cells on Day 1; and (ii) administration of a PD-1 inhibitor (e.g. an anti-PD-1 antibody) and an LAG3 inhibitor (e.g. an anti-LAG3 antibody) in a first and second cycle, wherein the first dose of the first cycle is administered between about Day 2 and Day 20 and the first dose of the second cycle is administered between about Day 50 and Day 65.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85; and (3) administering to the subject a LAG3 inhibitor in a dosing regimen comprising: (i) administration of a first amount of the LAG3 inhibitor, wherein the first amount is 240 mg and is administered on Days 8, 22, and 36; and (ii) administration of a second amount of the LAG3 inhibitor, wherein the second amount is 480 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85; and (3) administering to the subject a LAG3 inhibitor in a dosing regimen comprising: (i) administration of a first amount of the LAG3 inhibitor, wherein the first amount is 480 mg and is administered on Days 8, 22, and 36; and (ii) administration of a second amount of the LAG3 inhibitor, wherein the second amount is 960 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 8, 36, 57, and 85; and (3) administering to the subject a LAG3 inhibitor in a dosing regimen comprising administration of 480 mg of the LAG3 inhibitor on Days 8, 36, 57, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 8, 36, 57, and 85; and (3) administering to the subject a LAG3 inhibitor in a dosing regimen comprising administration of 960 mg of the LAG3 inhibitor on Days 8, 36, 57, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85; and (3) administering to the subject a LAG3 inhibitor in a dosing regimen comprising: (i) administration of a first amount of the LAG3 inhibitor, wherein the first amount is 240 mg and is administered on Days 15, 29, and 43; and (ii) administration of a second amount of the LAG3 inhibitor, wherein the second amount is 480 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85; and (3) administering to the subject a LAG3 inhibitor in a dosing regimen comprising: (i) administration of a first amount of the LAG3 inhibitor, wherein the first amount is 480 mg and is administered on Days 15, 29, and 43; and (ii) administration of a second amount of the LAG3 inhibitor, wherein the second amount is 960 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 15, 57, and 85; and (3) administering to the subject a LAG3 inhibitor in a dosing regimen comprising administration of 480 mg of the LAG3 inhibitor on Days 15, 57, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 15, 57, and 85; and (3) administering to the subject a LAG3 inhibitor in a dosing regimen comprising administration of 960 mg of the LAG3 inhibitor on Days 15, 57, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 8, 22, 36, 57, 71, and 85; and (3) administering to the subject a LAG3 inhibitor in a dosing regimen comprising administration of 240 mg of the LAG3 inhibitor on Days 8, 36, and 71.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 15, 29, 43, 57, 71, and 85; and (3) administering to the subject a LAG3 inhibitor in a dosing regimen comprising administration of 240 mg on Days 15, 43, and 71.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 8, 22, 36, 57, 71, and 85; and (3) administering to the subject a LAG3 inhibitor in a dosing regimen comprising administration of 240 mg of the LAG3 inhibitor on Days 8, 22, 36, 57, 71, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 15, 29, 43, 57, 71, and 85; and (3) administering to the subject a LAG3 inhibitor in a dosing regimen comprising administration of 240 mg of the LAG3 inhibitor on Days 815, 29, 43, 57, 71, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor, wherein the first amount is 240 mg and is administered on Days 8, 22, and 36; and (ii) administration of a second amount of the PD-1 inhibitor, wherein the second amount is 480 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering to the subject a PD-1 inhibitor in a dosing regimen comprising: (i) administration of a first amount of the PD-1 inhibitor, wherein the first amount is 240 mg and is administered on Days 15, 29, and 43; and (ii) administration of a second amount of the PD-1 inhibitor, wherein the second amount is 480 mg and is administered on Days 57 and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 8, 22, 36, 57, 71, and 85; and (3) administering to the subject a LAG3 inhibitor in a dosing regimen comprising administration of 120 mg of the LAG3 inhibitor on Days 8, 22, 36, 57, 71, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 240 mg of the PD-1 inhibitor on Days 15, 29, 43, 57, 71, and 85; and (3) administering to the subject a LAG3 inhibitor in a dosing regimen comprising administration of 120 mg of the LAG3 inhibitor on Days 15, 29, 43, 57, 71, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 8, 36, 64, and 85; and (3) administering to the subject a LAG3 inhibitor in a dosing regimen comprising administration of 240 mg of the LAG3 inhibitor on Days 8, 36, 64, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 15, 43, 64, and 85; and (3) administering to the subject a LAG3 inhibitor in a dosing regimen comprising administration of 240 mg of the LAG3 inhibitor on Days 15, 43, 64, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 8, 36, 64, and 85; and 3) administering to the subject a LAG3 inhibitor in a dosing regimen comprising administration of 480 mg of the LAG3 inhibitor on Days 8, 36, 64, and 85.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; (2) administering to the subject a PD-1 inhibitor in a dosing regimen comprising administration of 480 mg of the PD-1 inhibitor on Days 15, 43, 64, and 85; and (3) administering to the subject a LAG3 inhibitor in a dosing regimen comprising administration of 480 mg of the LAG3 inhibitor on Days 15, 43, 64, and 85.

### 4. Anti-PD-1 Antibody and Anti-LAG3 Antibody Co-Formulation

a. Compositions and Formulations

In some embodiments of the combination therapy methods, combinations, kits and uses provided herein, the combination therapy can be administered in one composition, e.g., a pharmaceutical composition containing a PD-1 inhibitor (e.g. an anti-PD-1 antibody, such as nivolumab), and and LAG3 inhibitor (e.g., an anti-LAG3 antibody, such as relatlimab). In some embodiments, the pharmaceutical composition contains nivolumab and relatlimab.

In some embodiments, the composition, e.g., a pharmaceutical composition containing a PD-1 inhibitor and a LAG3 inhibitor, e.g., an anti-PD-1 antibody such as nivolumab and an anti-LAG3 antibody such as relatlimab, can include carriers such as a diluent, adjuvant, excipient, or vehicle with which the inhibitors are administered. Examples of suitable pharmaceutical carriers are described in “Remington's Pharmaceutical Sciences” by E. W. Martin. Such compositions will contain a therapeutically effective amount of a PD-1 inhibitor and a LAG3 inhibitor, e.g., an anti-PD-1 antibody and an anti-LAG3 antibody, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing a PD-1 inhibitor and a LAG3 inhibitor, e.g., an anti-PD-1 antibody such as nivolumab and an anti-LAG3 antibody such as relatlimab, can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, the pharmaceutical compositions can be formulated for intravenous (IV) administration. In some embodiments, the pharmaceutical composition contains nivolumab and relatlimab and is formulated for IV administration,

In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, a PD-1 inhibitor and a LAG3 inhibitor, e.g., an anti-PD-1 antibody such as nivolumab and an anti-LAG3 antibody such as relatlimab, are typically formulated and administered in unit dosage forms or multiple dosage forms. Each unit dose contains a predetermined quantity of a therapeutically active PD-1 and LAG3 inhibitor, e.g., nivolumab and relatlimab, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of a PD-1 and a LAG3 inhibitor, e.g., nivolumab and relatlimab. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons

b. Dosing

In some embodiments, the provided combination therapy methods involve administering to the subject (1) a checkpoint inhibitor therapy (e.g., a single composition comprising a PD-1 inhibitor and a LAG3 inhibitor); and (2) a T cell therapy (e.g. CAR T cells). In some embodiments, the provided combination therapy methods involve initiation of administration of the checkpoint inhibitor therapy (e.g. the composition comprising the PD-1 inhibitor and the LAG3 inhibitor) subsequent to initiation of the T cell therapy (e.g. CAR T cells). In some embodiments, the provided combination therapy methods involve initiating administration of the checkpoint inhibitor therapy (e.g. the composition comprising the PD-1 inhibitor and the LAG3 inhibitor) between about 1 day and about 3 weeks after initiation of administration of the T cell therapy (CAR T cells). In some embodiments, initiation of administration of the checkpoint inhibitor therapy (e.g. the composition comprising the PD-1 inhibitor and the LAG3 inhibitor) is between about one week and about two weeks after initiation of administration of the T cell therapy (e.g. CAR T cells).

In some embodiments, the provided combination therapy methods involve initiating administration of the T cell therapy (e.g. CAR T cells) on Day 1. In some embodiments, the provided combination therapy methods involve initiating administration of the checkpoint inhibitor therapy (e.g. the composition comprising the PD-1 inhibitor and the LAG3 inhibitor) between about Day 2 and Day 20. In some embodiments, the provided combination therapy methods involve initiating administration of a checkpoint inhibitor therapy (e.g. the composition comprising the PD-1 inhibitor and the LAG3 inhibitor) on about Day 8 or Day 15. In some embodiments, the provided combination therapy methods involve initiating administration of a checkpoint inhibitor therapy (e.g. the composition comprising the PD-1 inhibitor and the LAG3 inhibitor) on about Day 8. In some embodiments, the provided combination therapy methods involve initiating administration of a checkpoint inhibitor therapy (e.g. the composition comprising the PD-1 inhibitor and the LAG3 inhibitor) on about Day 15.

In some embodiments, the method involves initiating administration of the checkpoint inhibitor therapy (e.g. the composition comprising the PD-1 inhibitor and the LAG3 inhibitor) after activation-induced cell death (AICD) of the cells of the T cell therapy (e.g. CAR T cells) has peaked.

In some embodiments, the provided combination therapy comprises: (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) administering a composition comprising a PD-1 inhibitor and a LAG3 inhibitor to the subject. In some embodiments, the composition comprises nivolumab and relatlimab. In some embodiments, the composition comprising nivolumab and relatlimab is administered to the subject about every four weeks (Q4W). In some embodiments, the composition comprises 480 mg nivolumab and 160 mg relatlimab. In some embodiments, the composition comprising 480 mg nivolumab and 160 mg relatlimab is administered to the subject about every four weeks (Q4W). In some embodiments, the composition is administered Q4W until disease progression or unacceptable toxicity occurs. In some embodiments, the composition is administered Q4W until disease progression occurs. In some embodiments, the composition is administered Q4W until unacceptable toxicity occurs.

In some embodiments, the method of treating a subject comprises (1) administering to a subject having a CD19-expressing cancer a cell therapy comprising a dose of engineered cells comprising T cells expressing a chimeric antigen receptor (CAR) that binds cluster of differentiation 19 (CD19) on Day 1; and (2) every four weeks (Q4W), administering to the subject 480 mg of a PD-1 inhibitor and 160 mg of a LAG3 inhibitor. In some embodiments, administration of the PD-1 inhibitor and the LAG3 inhibitor is by intravenous (i.v.) administration of a single composition comprising the PD-1 inhibitor and the LAG3 inhibitor.

### B. Administration of a Cell Therapy

### 1. T Cell-Engaging Therapy

In some embodiments, the T cell therapy is or comprises a T cell-engaging therapy that is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the T cell-engaging therapy is or comprises an antibody or antigen-binding fragment.

In some embodiments, the T cell-engaging therapy is a bispecific antibody containing at least one antigen-binding domain binding to an activating component of the T cell (e.g. a T cell surface molecule, e.g. CD3 or CD2) and at least one antigen-binding domain binding to a surface antigen on a target cell, such as a surface antigen on a tumor or cancer cell, for example any of the listed antigens as described herein, e.g. CD19. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

In some embodiments, bi-specific T cell engagers (BiTE) are used in connection with the provided methods, uses, articles of manufacture. In some embodiments, bi-specific T cell engagers have specificity toward two particular antigens (or markers or ligands). In some embodiments, the antigens are expressed on the surface of a particular type of cell. In particular embodiments, the first antigen is associated with an immune cell or an engineered immune cell, and the second antigen is associated with a target cell of the particular disease or condition, such as a cancer.

Numerous methods of producing bi-specific T cell engagers are known, including fusion of two different hybridomas (Milstein and Cuello, Nature 1983; 305:537-540), and chemical tethering though heterobifunctional cross linkers (Staerz et al. Nature 1985; 314:628-631). Among exemplary bi-specific antibody T cell-engaging molecules are those which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010).

In certain embodiments, the bi-specific T cell engager is a molecule encoded by a polypeptide construct. In certain embodiments, the polypeptide construct contains a first component comprising an antigen-binding domain binding to an activating portion of an immune cell or engineered immune cell, and a second component comprising an antigen-binding domain binding to a surface antigen (e.g. target or tumor associated antigen (TAA)) associated with a particular disease or condition (e.g. cancer). In some embodiments, the first and second components are coupled by a linker. In some embodiments, the first component is coupled to a leader sequence encoding a CD33 signal peptide.

In some embodiments, the polypeptide is a construct containing from N-terminus to C-terminus: a first component comprising an antigen-binding domain binding to an activating portion of the T cell, a peptide linker, and a second component comprising an antigen-binding domain binding to a surface antigen (e.g. target or tumor associated antigen (TAA)) associated with a disease or condition (e.g. cancer).

In some aspects, an activating component of the T cell I a T cell surface molecule, such as CD3 or CD2. In some embodiments, the surface antigen of the target cell is a tumor associated antigen (TAA). In some aspects, the TAA contains one or more epitopes. In some embodiments, the peptide linker is or comprises a cleavable peptide linker.

In some embodiments, the antigen binding domain of the first component of the bi-specific T cell engager engages a receptor on an endogenous immune cell in the periphery of the tumor. In some embodiments, the endogenous immune cell is a T cell. In some aspects, the engagement of the endogenous T cell receptor redirects the endogenous T cells to the tumor. In some aspects, the engagement of the endogenous T cell receptor recruits tumor infiltrating lymphocytes (TILs) to the tumor. In some aspects, the engagement of the endogenous T cell receptor activates the endogenous immune repertoire.

In some embodiments, the simultaneous or near simultaneous binding of the bi-specific T cell engager to both of its targets (e.g. the immune cell and the TAA) can result in a temporary interaction between the target cell and T cell, thereby resulting in activation (e.g. cytotoxic activity, cytokine release), of the T cell and subsequent lysis of the target cell.

In some embodiments, the first component of the bi-specific T cell engager is or comprises an antigen binding domain that binds to an activating component of a T cell. In some embodiments, the activating component of the T cell is a surface molecule. In some embodiments, the surface molecule is or comprises a T-cell antigen. Exemplary T-cell antigens include but are not limited to CD2, CD3, CD4, CD5, CD6, CD8, CD25, CD28, CD30, CD40, CD44, CD45, CD69 and CD90. In some aspects, the binding of the bispecific T cell engaging molecule with the T cell antigen stimulates and/or activates the T cell.

In some embodiments, the anti-T cell binding domain includes an antibody or an antigen-binding fragment thereof selected from the group consisting of a Fab fragment, a F(ab′)₂ fragment, an Fv fragment, an scFv, a scAb, a dAb, a single domain heavy chain antibody, and a single domain light chain antibody.

In some embodiments, the T cell binding domain on the bi-specific T cell engager is an anti-CD3 domain. In some aspects, the anti-CD3 domain is an scFv. In some embodiments, the anti-CD3 domain of the bi-specific T cell engager binds to a subunit of the CD3 complex on a receptor on a T cell. In some aspects, the receptor is on an endogenous T cell. In some embodiments, the receptor is on an engineered immune cell further expressing a recombinant receptor. The effects of CD3 engagement of T cells is well known in the art, and include but are not limited to T cell activation and other downstream cell signaling. Any of such bi-specific T cell engagers can be used in the provided disclosure herein.

In some embodiments, the second component of the bi-specific T cell engager comprising an antigen-binding domain binding to a surface antigen associated with a disease or condition is a tumor or cancer antigen. In some embodiments, among the antigens targeted by the bi-specific T cell engager are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen includes avP6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is CD19.

In some embodiments, both antigen binding domains, including the first antigen binding domain and the second antigen binding domain, comprise an antibody or an antigen-binding fragment.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single VH or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the bi-specific T cell engager comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In certain embodiments, the antigen binding domains are single chain variable fragments (scFv). In some embodiments, the scFv is a tandem scFv containing a heavy and a light chain. In some embodiments, the heavy and light chains are connected by peptide linkers. In some embodiments, the linker is composed primarily of serines and glycines. In some aspects, the linkage of the heavy chain and the light chain forms a single polypeptide antigen binding domain.

In certain embodiments, the first antigen binding domain of the bi-specific T cell engager is an anti-CD3 scFv. In certain embodiments, the second antigen binding domain of the bi-specific T cell engager is an anti-CD19 scFv.

In some aspects, the bi-specific T cell engager polypeptide constructs contain a linker that joins the first component comprising the antigen-binding domain that binds to an activating portion of the T cell, to the second component comprising an antigen-binding domain binding to a surface antigen (e.g. target or tumor associated antigen (TAA)) associated with a particular disease or condition. In some aspects, the linker is a short, medium or long linker.

In some embodiments, the linker is a peptide linker which is cleavable. In some aspects, the cleavable linker includes a sequence that is a substrate for a protease. In some embodiments, the sequence comprises a bond that can be broken under in vivo conditions. In some cases, the linker sequence is selectively cleaved by a protease present in a physiological environment. In some aspects, the environment is separate from the tumor microenvironment. In some embodiments, the protease is found in the periphery of the tumor.

In some embodiments, the selectively cleavable linker is cleaved by a protease produced by cells that do not co-localize with the tumor. In some embodiments, the selectively cleavable linker is not cleaved by proteases that are in the proximity of the tumor microenvironment. In some embodiments, the cleavage of the linker by the protease renders the bi-specific T cell engaging molecule inactive. In some embodiments, the protease is found in the circulating blood of a subject. In some embodiments, the protease is a part of the intrinsic or extrinsic coagulation pathway. In some aspects, the protease is a serine protease. In some aspects, the protease comprises but is not limited to a thrombin, factor X, factor XI, factor XII, and plasmin.

Among such exemplary bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). In some embodiments, the T-cell engaging therapy is blinatumomab or AMG 330. Any of such T cell-engagers can be used in used in the provided methods.

The immune system stimulator and/or the T cell engaging therapy can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the immunotherapy is administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In certain embodiments, one or more doses of a T cell engaging therapy are administered. In particular embodiments, between or between about 0.001 μg and about 5,000 μg, inclusive, of the T cell engaging therapy is administered. In particular embodiments, between or between about 0.001 μg and 1,000 μg, 0.001 μg to 1 μg, 0.01 μg to 1 μg, 0.1 μg to 10 μg, 0.01 μg to 1 μg, 0.1 μg and 5 μg, 0.1 μg and 50 μg, 1 μg and 100 μg, 10 μg and 100 μg, 50 μg and 500 μg, 100 μg and 1,000 μg, 1,000 μg and 2,000 μg, or 2,000 μg and 5,000 μg of the T cell engaging therapy is administered. In some embodiments, the dose of the T cell engaging therapy is or includes between or between about 0.01 μg/kg and 100 mg/kg, 0.1 μg/kg and 10 μg/kg, 10 μg/kg and 50 μg/kg, 50 μg/kg and 100 μg/kg, 0.1 mg/kg and 1 mg/kg, 1 mg/kg and 10 mg/kg, 10 mg/kg and 100 mg/kg, 100 mg/kg and 500 mg/kg, 200 mg/kg and 300 mg/kg, 100 mg/kg and 250 mg/kg, 200 mg/kg and 400 mg/kg, 250 mg/kg and 500 mg/kg, 250 mg/kg and 750 mg/kg, 50 mg/kg and 750 mg/kg, 1 mg/kg and 10 mg/kg, or 100 mg/kg and 1,000 mg/kg, each inclusive. In some embodiments, the dose of the T cell engaging therapy is at least or at least about or is or is about 0.1 μg/kg, 0.5 μg/kg, 1 μg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1,000 mg/kg. In particular embodiments, the T cell engaging therapy is administered orally, intravenously, intraperitoneally, transdermally, intrathecally, intramuscularly, intranasally, transmucosally, subcutaneously, or rectally.

### 2 Cell Therapy

In some embodiments, the T cell therapy, is a cell-based therapy that is or comprises administration of cells, such as immune cells, for example T cells, that target a molecule expressed on the surface of a lesion, such as a tumor or a cancer. In some aspects, the cell therapy is a tumor infiltrating lymphocytic (TIL) therapy, a transgenic TCR therapy, or a recombinant-receptor expressing cell therapy, which is a T cell therapy, which optionally is a chimeric antigen receptor (CAR)-expressing cell therapy. In some embodiments, the T cell therapy includes administering T cells engineered to express a chimeric antigen receptor (CAR). In some aspects, the T cell therapy is an adoptive T cell therapy comprising T cells that specifically recognize and/or target an antigen associated with the cancer, such as an antigen associated with a B cell malignancy, e.g. a lymphoma, such as a non-Hodgkin lymphoma (NHL). In some aspects, the T cell therapy comprises T cells engineered with a chimeric antigen receptor (CAR) comprising an antigen binding domain that binds, such as specifically binds, to the antigen. In some cases, the antigen targeted by the T cell therapy is CD19.

In some embodiments, the immune cells express a T cell receptor (TCR) or other antigen-binding receptor. In some embodiments, the immune cells express a recombinant receptor, such as a transgenic TCR or a chimeric antigen receptor (CAR). In some embodiments, the cells are autologous to the subject. In some embodiments, the cells are allogeneic to the subject. Exemplary of such cell therapies, e.g. T cell therapies, for use in the provided methods are described below.

In some embodiments, the provided cells express and/or are engineered to express receptors, such as recombinant receptors, including those containing ligand-binding domains or binding fragments thereof, and T cell receptors (TCRs) and components thereof, and/or functional non-TCR antigen receptors, such as chimeric antigen receptors (CARs). In some embodiments, the recombinant receptor contains an extracellular ligand-binding domain that specifically binds to an antigen. In some embodiments, the recombinant receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

Among the engineered cells, including engineered cells containing recombinant receptors, are described in Section II below. Exemplary recombinant receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416,and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the genetically engineered antigen receptors include a CAR as described in U.S. Pat. No. 7,446,190, and those described in International Patent Application Publication No.: WO/2014055668 A1.

Exemplary CAR T cell therapies that target CD19 include those investigated or being investigated in clinical trials NCT02644655, NCT03744676, NCT01087294, NCT03366350, NCT03790891, NCT03497533, NCT04007029, NCT03960840, NCT04049383, NCT04094766, NCT03366324, NCT02546739, NCT03448393, NCT03467256, NCT03488160, NCT04012879, NCT03016377, NCT03468153, NCT03483688, NCT03398967, NCT03229876, NCT03455972, NCT03423706, NCT03497533, and NCT04002401, including FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel). Exemplary engineered cells include, but are not limited to, BREYANZI®, TECARTUS™, KYMRIAH™, YESCARTA™, UCART19, and ALLO-501. In some aspects, the engineered cells include any of those described in Marofi et al., Front. Immunol. (2021) 12:681984, which is incorporated by reference herein in its entirety.

Methods for administration of cells for adoptive cell therapy are known and may be used in connection with the provided methods, compositions and articles of manufacture and kits. For example, adoptive T cell therapy methods are described, e.g., in US Patent Application Publication No.

2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs.CD8 T cells.

The cells can be administered by any suitable means. The cells are administered in a dosing regimen to achieve a therapeutic effect, such as a reduction in tumor burden. Dosing and administration may depend in part on the schedule of administration of the checkpoint inhibitor therapy, which can be administered subsequent to initiation of administration of the cell therapy, such as T cell therapy, e.g. CAR T cell therapy. Various dosing schedules of the cell therapy include but are not limited to single or multiple administrations over various time-points, bolus administration, and pulse infusion.

a. Compositions and Formulations

In some embodiments, the dose of cells of the T cell therapy comprising cells engineered with a recombinant antigen receptor, e.g. CAR or TCR, is provided as a composition or formulation, such as a pharmaceutical composition or formulation. Such compositions can be used in accord with the provided methods and/or with the provided articles of manufacture or compositions, such as in the treatment of a B cell malignancy (e.g. a lymphoma, such as non-Hodgkin lymphoma; NHL).

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the T cell therapy, such as engineered T cells (e.g. CAR T cells), is formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat the disease or condition, such as a therapeutically effective or prophylactically effective amount. Therapeutic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

b. Dosing

The cells of the T cell therapy can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In some embodiments, a dose of cells is administered to subjects in accord with the provided methods, and/or with the provided articles of manufacture or compositions. In some embodiments, the size or timing of the doses is determined as a function of the particular disease or condition (e.g., cancer, e.g., B cell malignancy) in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about one million to at or about 100 billion cells and/or that amount of cells per kilogram of body weight, such as, e.g., 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight. Dosages may vary depending on attributes particular to the disease or disorder and/or patient and/or other treatments.

In some embodiments, the dose of cells comprises from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1.1×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells.

In some embodiments, the dose of cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 1.1×10⁸ CAR-expressing cells, at least or at least about 2.5×10⁸ CAR-expressing cells, or at least or at least about 5×10⁸ CAR-expressing cells.

In some embodiments, the dose of cells is a flat dose of cells or fixed dose of cells such that the dose of cells is not tied to or based on the body surface area or weight of a subject.

In some embodiments, for example, where the subject is a human, the dose includes fewer than at or about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 6×10⁷, 7×10⁷, 8×10⁷, 9×10⁷, 1×10⁸, 1.1×10⁸, 1.2×10⁸, 1.3×10⁸, 1.4×10⁸, 1.5×10⁸, 2×10⁸, 3×10⁸, 4×10⁸ or 5×10⁸ total such cells, or the range between any two of the foregoing values. In some embodiments, where the subject is a human, the dose includes between at or about 1×10⁶ and at or 3×10⁸ total recombinant receptor (e.g., CAR)-expressing cells, e.g., in the range of at or about 1×10⁷ to at or about 2×10⁸ such cells, such as at or about 1×10⁷, 5×10⁷, 1×10⁸, 1.1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁵ to at or about 5×10⁸ total recombinant receptor (e.g. CAR)-expressing T cells or total T cells, from at or about 1×10⁵ to at or about 1×10⁸ total recombinant receptor (e.g. CAR)-expressing T cells or total T cells, from at or about 5×10⁵ to at or about 1×10⁷ total recombinant receptor (e.g. CAR)-expressing T cells or total T cells, or from at or about 1×10⁶ to at or about 1×10⁷ total recombinant receptor (e.g. CAR)-expressing T cells or total T cells, each inclusive. In some embodiments, the dose of cells comprises the administration of from at or about 2.5×10⁷ total recombinant receptor (e.g. CAR)-expressing T cells. In some embodiments, the dose of cells comprises the administration of from at or about 5×10⁷ total recombinant receptor (e.g. CAR)-expressing T cells. In some embodiments, the dose of cells comprises the administration of from at or about 6×10⁷ total recombinant receptor (e.g. CAR)-expressing T cells. In some embodiments, the dose of cells comprises the administration of from at or about 7×10⁷ total recombinant receptor (e.g. CAR)-expressing T cells. In some embodiments, the dose of cells comprises the administration of from at or about 8×10⁷ total recombinant receptor (e.g. CAR)-expressing T cells. In some embodiments, the dose of cells comprises the administration of from at or about 9×10⁷ total recombinant receptor (e.g. CAR)-expressing T cells. In some embodiments, the dose of cells comprises the administration of from at or about 1×10⁸ total recombinant receptor (e.g. CAR)-expressing T cells. In some embodiments, the dose of cells comprises the administration of from at or about 1.1×10⁸ total recombinant receptor (e.g. CAR)-expressing T cells. In some embodiments, the dose of cells comprises the administration of from at or about 1.2×10⁸ total recombinant receptor (e.g. CAR)-expressing T cells. In some embodiments, the dose of cells comprises the administration of from at or about 1.3×10⁸ total recombinant receptor (e.g. CAR)-expressing T cells. In some embodiments, the dose of cells comprises the administration of from at or about 1.4×10⁸ total recombinant receptor (e.g. CAR)-expressing T cells. In some embodiments, the dose of cells comprises the administration of from at or about 1.5×10⁸ total recombinant receptor (e.g. CAR)-expressing T cells.

In some embodiments, the T cells of the dose include CD4+ T cells, CD8+ T cells, or CD4+ and CD8+ T cells.

In some embodiments, for example, where the subject is human, the CD8+ T cells of the dose, including in a dose including CD4+ and CD8+ T cells, includes between at or about 1×10⁶ and at or about 1×10⁸ total recombinant receptor (e.g., CAR)-expressing CD8+ cells, e.g., in the range of at or about 5×10⁶ to at or about 1×10⁸ such cells, such cells at or about 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁷ to at or about 0.75×10⁸ total recombinant receptor-expressing CD8+ T cells, from at or about 1×10⁷ to at or about 2.5×10⁷ total recombinant receptor-expressing CD8+ T cells, from at or about 1×10⁷ to at or about 0.75×10⁵ total recombinant receptor-expressing CD8+ T cells, each inclusive. In some embodiments, the dose of cells comprises the administration of at or about 1×10⁷, 2.5×10⁷, 5×10⁷, 6×10⁷, 7×10⁷, 7.5×10⁷, 8×10⁷, 9×10⁷, 1×10⁸, 1.1×10⁸, 1.2×10⁸, 1.3×10⁸, 1.4×10⁸, 1.5×10⁸, or 5×10⁸ total recombinant receptor-expressing CD8+ T cells.

In some embodiments, for example, where the subject is human, the CD4+ T cells of the dose, including in a dose including CD4+ and CD8+ T cells, includes between at or about 1×10⁶ and at or about 1×10⁸ total recombinant receptor (e.g., CAR)-expressing CD4+ cells, e.g., in the range of at or about 5×10⁶ to 1×10⁸ such cells, such at or about 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from at or about 1×10⁷ to at or about 0.75×10⁸ total recombinant receptor-expressing CD4+ T cells, from at or about 1×10⁷ to at or about 2.5×10⁷ total recombinant receptor-expressing CD4+ T cells, from at or about 1×10⁷ to at or about 0.75×10⁸ total recombinant receptor-expressing CD4+ T cells, each inclusive. In some embodiments, the dose of cells comprises the administration of at or about 1×10⁷, 2.5×10⁷, 5×10⁷ 7.5×10⁷, 1×10⁸, 1.5×10⁸, or 5×10⁸ total recombinant receptor-expressing CD4+ T cells. In some embodiments, the dose of cells comprises the administration of between about 5×10⁷ and about 1.1×10⁸ total recombinant receptor-expressing CD4+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+ and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some embodiments, the first composition and the second composition are mixed prior to the administration into the subject. In some embodiments, the first composition and the second composition are mixed shortly (e.g., within at or about 6 hours, 5 hours, 4 hours, 3 hours, 2 hours, 1.5 hours, 1 hour, or 0.5 hour) before the administration, In some embodiments, the first composition and the second composition are mixed immediately before the administration.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition. In some composition, the first composition comprising CD8+ CAR-expressing T cells is administered prior to the second composition comprising CD4+ CAR-expressing T cells.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

### 3. Lymphodepleting Therapy

In some aspects, the provided methods can further include administering one or more lymphodepleting therapies, such as prior to or simultaneous with initiation of administration of the T cell therapy (e.g. CAR-expressing T cells). In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the initiation of the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the initiation of the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the initiation of administration of the dose of cells. In some embodiments, the subject is administered a preconditioning agent between 2 and 7, inclusive, such as at 2, 3, 4, 5, 6, or 7, days prior to the initiation of the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m²″24 mg/m² and 35 mg/m², 20 mg/m² and 30 mg/m², or 24 mg/m² and 26 mg/m². In some instances, the subject is administered 25 mg/m² of fludarabine. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells. In some embodiments, the subject is administered about 300 mg/m² cyclophosphamide and about 30 mg/m² fludarabine each daily for 3 days. In some embodiments, the preconditioning administration schedule ends between 2 and 7, inclusive, such as at 2, 3, 4, 5, 6, or 7, days prior to the initiation of the administration of the dose of cells.

In one exemplary dosage regimen, prior to receiving the first dose of CAR-expressing cells, subjects receive a lymphodepleting preconditioning chemotherapy of cyclophosphamide and fludarabine (CY/FLU), which is administered at least two days before the first dose of CAR-expressing cells and generally no more than 7 days before administration of cells. After preconditioning treatment, subjects are administered the dose of CAR-expressing T cells as described above.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells (e.g. CAR T cells) are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject. Therefore, in some embodiments, the dose of preconditioning agent given in the method which is a combination of a checkpoint inhibitor therapy and a T cell therapy is higher or lower than the dose given in the method without the checkpoint inhibitor therapy. In some embodiments, the dose of preconditioning agent given in the method which is a combination of a checkpoint inhibitor therapy and a cell therapy is higher than the dose given in the method without the checkpoint inhibitor therapy. In some embodiments, the dose of preconditioning agent given in the method which is a combination of a checkpoint inhibitor therapy and a cell therapy is lower than the dose given in the method without the checkpoint inhibitor therapy.

### C. Subjects

Provided herein are cells, populations, and compositions for administration to a subject having a CD19-expressing cancer to be treated, e.g., via adoptive cell therapy, such as adoptive T cell therapy. In some embodiments, the CD19-expressing cancer is a B cell malignancy. In some embodiments, the CD19-expressing cancer is a leukemia or a lymphoma. In some embodiments, the CD19-expressing cancer is chronic lymphocytic leukemia (CLL), small lymphocytic lymphoma (SLL), acute lymphoblastic leukemia (ALL), acute myelogenous leukemia (AML), chronic myelogenous leukemia (CML), myelodysplastic syndrome (MDS), non-Hodgkin lymphoma (NHL), or a subtype of NHL, such as diffuse large B-cell lymphoma (DLBCL). In some embodiments, the cancer is a leukemia. In particular, among provided embodiments are methods of treating subjects with a CD19-expressing cancer such as a non-Hodgkin lymphoma. Thus, in some embodiments, the cells, populations, and compositions provided herein are for administration to a subject having a non-Hodgkin lymphoma (NHL).

In some embodiments, the subject has a high risk NHL. In some embodiments, the subjects are a heavily pretreated population of subjects with NHL. In some embodiments, the subjects have relapsed or are refractory to at least two prior lines of therapy for the NHL. In some embodiments, the subjects have relapsed or are refractory to two prior lines of therapy for the NHL. In some embodiments, at least one of the two prior lines of therapy is a CD20-targeting agent and an anthracycline. In some embodiments, the subject has not been treated with an anti-PD-1 agent. In some embodiments, the subject has not been treated with an anti-PD-L1 agent. In some embodiments, the subject has not been treated with an anti-LAG3 agent.

In some embodiments, the subject has not been previously treated with a gene therapy product or an adoptive T cell therapy. In some embodiments, the subject has previously received a hematopoietic stem cell transplant (HSCT), e.g. allogeneic HSCT or autologous HSCT. In some embodiments, the subject has not received an allogeneic HSCT within 90 days of leukapheresis.

In some embodiments, at or prior to administration of the dose of cells, the subject does not have a prior history of malignancies, other than R/R aggressive NHL, unless the subject has been free of the disease for greater than or equal to 2 years.

In some aspects, NHL can be staged based on the Lugano classification (see, e.g., Cheson et al., (2014) JCO 32(27):3059-3067; Cheson, B. D. (2015) Chin Clin Oncol 4(1):5). In some cases, the stages are described by Roman numerals I through IV (1-4), and limited stage (I or II) lymphomas that affect an organ outside the lymph system (an extranodal organ) are indicated by an E. Stage I represents involvement in one node or a group of adjacent nodes, or a single extranodal lesions without nodal involvement (IE). Stage 2 represents involvement in two or more nodal groups on the same side of the diaphragm or stage I or II by nodal extent with limited contiguous extranodal involvement (IIE). Stage III represents involvement in nodes on both sides of the diaphragm or nodes above the diaphragm with spleen involvement. Stage IV represents involvement in additional non-contiguous extra-lymphatic involvement. In addition, “bulky disease” can be used to describe large tumors in the chest, in particular for stage II. The extent of disease is determined by positron emission tomography (PET)-computed tomography (CT) for avid lymphomas, and CT for non-avid histologies. In some of any embodiments, at or prior to the administration of the dose of cells, the subject to be treated according to the provided embodiments has a positron emission tomography (PET)-positive disease (e.g. Deauville score of 4 or 5; Barrington et al., J Clin Oncol. (2014) 32(27):3048-58). In some of any embodiments, at or prior to the administration of the dose of cells, the subject to be treated according to the provided embodiments has a computed tomography (CT) measurable disease (e.g. per Lugano classification). In some of any embodiments, at or prior to the administration of the dose of cells, the subject to be treated according to the provided embodiments has a PET-positive and CT measurable disease. In some of any embodiments, at or prior to the administration of the dose of cells, the subject to be treated according to the provided embodiments has a sum of product of perpendicular diameters (SPD) of up to 6 index lesions of greater than or equal to 25 cm² by CT scan. In some embodiments, the subject has adequate organ function at or prior to administration of the dose of cells.

In some embodiments, the subject has an Eastern Cooperative Oncology Group (ECOG) performance status of 0 or 1. In some embodiments, the Eastern Cooperative Oncology Group (ECOG) performance status indicator can be used to assess or select subjects for treatment, e.g., subjects who have had poor performance from prior therapies (see, e.g., Oken et al., (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sørensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease performance without |
| | restriction |
| 1 | Restricted in physically strenuous activity but ambulatory and |
| | able to carry out work of a light or sedentary nature, e.g., |
| | light house work, office work |
| 2 | Ambulatory and capable of all self-care but unable to carry out |
| | any work activities; up and about more than 50% of waking |
| | hours |
| 3 | Capable of only limited self-care; confined to bed or chair |
| | more than 50% of waking hours |
| 4 | Completely disabled; cannot carry on any self-care; totally |
| | confined to bed or chair |
| 5 | Dead |

In some embodiments, the subject is or has been identified as having an ECOG status of 0 or 1; and/or the subject does not have an ECOG status of >1. In some embodiments, the subject has an ECOG status of 0. In some embodiments, the subject has an ECOG status of 1.

In some embodiments, the subject is at least 18 years of age. In particular embodiments, the provided methods can result in favorable outcomes and low toxicity rates in a group of subjects that are older, including in subjects greater than 60 years of age or older.

In some embodiments, the disease or condition is a tumor or a cancer. In some embodiments, the cancer is a CD19-expressing cancer. In some aspects, the disease or condition is a B cell malignancy, such as a lymphoma. In some embodiments, the subjects have or are suspect of having a lymphoma, such as a non-Hodgkin lymphoma (NHL).

Non-Hodgkin lymphomas (NHLs) comprise a heterogeneous group of malignancies. Some subjects with NHL may survive without treatment while others may require immediate intervention. In some cases, subjects with NHL may be classified into groups that may inform disease prognosis and/or recommended treatment strategy. In some cases, these groups may be “low risk,” “intermediate risk,” “high risk,” and/or “very high risk” and patients may be classified as such depending on a number of factors including, but not limited to, genetic abnormalities and/or morphological or physical characteristics. In some embodiments, subjects treated in accord with the methods, and/or with the articles of manufacture or compositions, are classified or identified based on the risk of NHL. In some embodiments, the subject is one that has high risk NHL.

DLBCL is the most frequent lymphoma subtype, representing approximately 30% of all NHL cases. Diffuse large B-cell lymphoma is a heterogeneous disease with several histological and molecular subtypes. The largest subgroup is DLBCL not otherwise specified (NOS). Molecular profiling by gene expression profiling based on biologic similarity to normal stages of B-cell development (cell of origin; COO) helped to further divide DLBCL into germinal center-like (GCB), activated B-cell-like (ABC) tumors, and primary mediastinal large B-cell lymphoma (PMBCL), a distinct clinical entity (Lenz, N Engl J Med. 2008 Nov. 27; 359(22):2313-23).

Within the GCB group a specific high-risk group is defined by concurrent chromosomal rearrangements of c-MYC and the anti-apoptotic oncogene BCL2 or BCL6, referred to as double-hit lymphomas (DHL). In addition, in some cases there is a concurrent rearrangement of c-MYC and both antiapoptotic oncogenes BCL2 and BCL6, which are referred to as triple-hit lymphoma. DHL represents approximately 5% of de novo cases of DLBCL with very poor OS of <12 months when treated with R-CHOP (Camicia, Mol Cancer. 2015 Dec. 11; 14(1):207). Newer data suggest negative prognostic impact of P53 mutations or deletions in DLBCL (Schiefer, Medicine (Baltimore) 2015 December; 94(52):e2388). In some embodiments, the subject has or has been identified as having a double/triple hit lymphoma or a lymphoma of the double/triple hit molecular subtypes. In some embodiments, the lymphoma is a double hit lymphoma characterized by the presence of MYC (myelocytomatosis oncogene), BCL2 (B-cell lymphoma 2), and/or BCL6 (B-cell lymphoma 6) gene rearrangements (e.g., translocations). In some embodiments, the lymphoma is a triple hit lymphoma characterized by the presence of MYC, BCL2, and BCL6 gene rearrangements; see, e.g., Aukema et al., (2011) Blood 117:2319-2331. In aspects, the therapy is indicated for such subjects and/or the instructions indicate administration to a subject within such population. In some embodiments, based on the 2016 WHO criteria (Swerdlow et al., (2016) Blood 127(20):2375-2390), double/triple hit lymphoma can be considered high-grade B-cell lymphoma, with MYC and BCL2 and/or BCL6 rearrangements with DLBCL histology (double/triple hit).

Despite follicular lymphoma (FL) being an indolent lymphoma type, Grade 3B FL is regarded as aggressive lymphoma. Clinical behavior is very similar to DLBCL and FL frequently undergoes histological transformation into DLBCL. Consequently, current guidelines recommend to treat FL Grade 3B according to the DLBCL treatment algorithm (National Comprehensive Cancer Network (NCCN), 2016; Dreyling, Clin Cancer Res. 2014 Oct. 15; 20(20):5194-206). These subjects are generally treated with an anthracycline-based chemotherapy combined with rituximab (eg, R-CHOP) and have a similar prognosis to that of de novo DLBCL.

Despite overall improvement in outcomes of DLBCL, approximately one-third of subjects will develop relapsed/refractory (R/R) disease that remains a major cause of mortality. Refractory disease is defined as a <50% decrease in lesion size or the appearance of new lesions. Relapsed disease reflects the (re)appearance of lesions after attainment of a partial or complete response (PR or CR) (Cheson, J Clin Oncol. 2007 Feb. 10; 25(5):579-86). Relapsed/refractory subjects have a poor prognosis, particularly those who do not respond to second line chemotherapy with a median OS of 4.4 months (Van Den Neste, Bone Marrow Transplant. 2016 January; 51(1):51-7).

In some embodiments, the NHL is a histologically confirmed aggressive B-cell NHL. In some embodiments, the NHL is diffuse large B-cell lymphoma (DLBCL) not otherwise specified (NOS), including transformed indolent NHL; follicular lymphoma Grade 3B, T cell/histiocyte-rich large B-cell lymphoma, Epstein-Barr virus (EBV) positive DLBCL NOS, primary mediastinal (thymic) large B-cell lymphoma, or high grade B-cell lymphoma with MYC and BCL2 and/or BCL6 rearrangements with DLBCL histology (double/triple-hit lymphoma (DHL/THL)). In some embodiments, the subject has elevated disease burden at the time of screening (e.g. prior to administration of the combination therapy). In some embodiments, screening takes place between about one week and about two weeks prior to leukapheresis. In some embodiments, a subject has elevated disease burden if the sum of product of perpendicular diameters (SPD) of index lesions (e.g. up to 6 index lesions) is greater than or equal to 25 cm² by CT scan. In some embodiments, a subject have Richter's transformation chronic lymphocytic leukemia (tCLL). In some embodiments, a subject has tCLL and does not have elevated disease burden.

The provided methods are for treatment of subjects that have relapsed or are refractory to (R/R) a prior therapy. In some embodiments, prior to the administration of the combination therapy of a T cell therapy and a checkpoint inhibitor therapy, the subject has been treated with one or more prior therapies for the CD19-expressing cancer. In any of the provided embodiments, the NHL is a relapsed and/or refractory NHL. In some embodiments, the NHL is a relapsed NHL. In some embodiments, the NHL is a refractory NHL. In any of the provided embodiments, the subject has relapsed or is refractory to at least two prior lines of systemic therapy for the NHL. In some embodiments, at least one of the at least two prior lines of therapy includes an anti-CD20 therapy (e.g. an anti-CD20 antibody) and an anthracycline. In some embodiments, one of the at least two prior lines of therapy is a hematopoietic stem cell transplant (HSCT). In some embodiments, the HSCT is not allogeneic HSCT. In some embodiments, one of the at least two prior lines of therapy is not an anti-PD-1 or an anti-PD-L1 therapy (e.g. an anti-PD-1 antibody or an anti-PD-L1 antibody). In some embodiments, one of the at least two prior lines of therapy is not an anti-LAG3 therapy (e.g. an anti-LAG3 antibody). In some embodiments, a subject with transformed disease has had at least two prior lines of systemic therapy for the transformed disease (e.g. the DLBCL). In some embodiments, a prior line of therapy is not a line of therapy provided for a previous indolent condition (e.g. follicular lymphoma (FL), CLL). In some embodiments, if a subject previously received an anthracycline for indolent disease, the subject is not required to have received anthracycline for DLBCL.

In some embodiments, the subject has been subject to more than one, two three, four, five, or six prior therapies. In some embodiments, the subject has been subject to one prior therapy. In some embodiments, the subject has been subject to about two to four prior therapies. In some embodiments, the subject has been subject to about five to six prior therapies. In some embodiments, the subject has been subject to more than six prior therapies.

In some embodiments, the subject has had poor prognosis after treatment with standard therapy and/or has failed one or more lines of previous therapy. In some embodiments, the subject has been treated or has previously received at least or about at least or about 1, 2, 3, 4, 5, 6, or 7 other therapies for treating the NHL other than a lymphodepleting therapy. In some embodiments, the subject has been previously treated with chemotherapy or radiation therapy. In some aspects, the subject is refractory or non-responsive to the other therapy or therapeutic agent. In some embodiments, the subject has persistent or relapsed disease, e.g., following treatment with another therapy or therapeutic intervention, including chemotherapy or radiation. In some embodiments, the combination therapy is administered to subjects that have progressed on a prior treatment. In some embodiments, the combination therapy is administered to subjects that have stopped responding to a prior therapy. In some embodiments, the combination therapy is administered to subjects that have relapsed following a remission after a prior treatment. In some embodiments, the combination therapy is administered to subjects that are refractory to a prior treatment. In some embodiments, the combination therapy is administered to subjects that have less than an optimal response (e.g., a complete response, a partial response or a stable disease) to a prior therapy.

In some embodiments, the provided methods implement flat dosing, e.g. total number of CAR+ cells, total number of CAR+CD8+ T cells and/or CAR+CD4+ T cells, such as to administer a precise or fixed dose of such cell type(s) to each of a group of subjects treated, including subjects of variable weight. Thus, the provided methods include methods in which the dose of cells is a flat dose of cells or fixed dose of cells such that the dose of cells is not tied to or based on the body surface area or weight of a subject. In some embodiments, such methods minimize or reduce the chance of administering too many cells to the subject, which may increase the risk of a toxic outcome associated with administration of the CAR-T cells.

In some embodiments, the dose of T cells comprises between about 5×10⁷ recombinant receptor (e.g. CAR)-expressing T cells and about 1.1×10⁸ recombinant receptor (e.g. CAR)-expressing T cells. In some embodiments, the dose of T cells comprises at or about 5×10⁷ recombinant receptor (e.g. CAR)-expressing T cells. In some embodiments, the dose of T cells comprises at or about 6×10⁷ recombinant receptor (e.g. CAR)-expressing T cells. In some embodiments, the dose of T cells comprises at or about 7×10⁷ recombinant receptor (e.g. CAR)-expressing T cells. In some embodiments, the dose of T cells comprises at or about 0.75×10⁸ recombinant receptor (e.g. CAR)-expressing CD8⁺ T cells. In some embodiments, the dose of T cells comprises at or about 8×10⁷ recombinant receptor (e.g. CAR)-expressing T cells. In some embodiments, the dose of T cells comprises at or about 9×10⁷ recombinant receptor (e.g. CAR)-expressing T cells. In some embodiments, the dose of T cells comprises at or about 1×10⁸ recombinant receptor (e.g. CAR)-expressing T cells. In some embodiments, the dose of T cells comprises at or about 1.1×10⁸ recombinant receptor (e.g. CAR)-expressing T cells. In some embodiments, the dose of T cells comprises at or about 1.5×10⁸ recombinant receptor (e.g. CAR)-expressing T cells. In some embodiments, the T cells of the dose include CD4⁺ and CD8⁺ T cells. In some embodiments, the number of cells is the number of such cells that are viable cells.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8⁺- and CD4⁺-enriched populations, respectively, e.g., CD4⁺ and/or CD8⁺ T cells each individually including cells genetically engineered to express the recombinant receptor. In some embodiments, the administration of the dose comprises administration of a first composition comprising a dose of CD8⁺ T cells or a dose of CD4⁺ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In particular embodiments, the separate administrations are carried out sequentially by administering, in any order, a first composition comprising a dose of CD8⁺ T cells or a dose of CD4⁺ T cells and a second composition comprising the other of the dose of CD4⁺ T cells and the CD8⁺ T cells. In some embodiments, the dose comprises a first composition and a second composition, and the first composition and second composition are administered within 48 hours of each other, such as no more than 36 hours of each other or not more than 24 hours of each other. In some embodiments, the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart. In some embodiments, the first composition and the second composition are administered less than 2 hours apart.

In some composition, the first composition, e.g., first composition of the dose, comprises CD4⁺ T cells. In some composition, the first composition, e.g., first composition of the dose, comprises CD8⁺ T cells. In some embodiments, the first composition is administered prior to the second composition. In particular embodiments, the CD8+ T cells are administered prior to the CD4+ T cells.

In some embodiments, the dose or composition of cells includes a defined or target ratio of CD4⁺ cells expressing a recombinant receptor (e.g. CAR) to CD8⁺ cells expressing a recombinant receptor (e.g. CAR) and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the provided methods and uses provide for or achieve improved or more durable responses or efficacy as compared to certain alternative methods, such as in particular groups of subjects treated, such as in patients with a CD19-expressing cancer, such as NHL, including those with high-risk disease. In some embodiments, the methods are advantageous by virtue of administering a T cell therapy, such as a composition including cells for adoptive cell therapy, e.g., such as a CAR-expressing T cells, e.g. anti-CD19 CAR+ T cells, and a checkpoint therapy (e.g. an anti-PD-1 antibody, and optionally an anti-LAG3 antibody). In some embodiments, the methods also include, prior to administration of the T cell therapy, administration of a lymphodepleting therapy to the subject, e.g. such as cyclophosphamide, fludarabine, or combinations thereof.

### D. Response, Efficacy, and Survival

In some embodiments, the administration in accord with the provided methods effectively treats the subject despite the subject having become resistant to another therapy. In some embodiments, at least 30%, at least 35%, at least 40% at least 50%, at least 60%, at least 70%, or at least 80%, of subjects treated according to the method achieve complete remission (CR). In some embodiments, at least about 40%, at least about 50%, at least about 60%, at least about 70%, at least 80%, or at least 90% of the subjects treated according to the method achieve an objective response (OR). In some embodiments, at least or at least about 50% of subjects, at least or at least about 60% of the subjects, at least or at least about 70% of the subjects, at least or at least about 80% of the subjects or at least or at least about 90% of the subjects treated according to the method achieve CR and/or achieve an objective response (OR). In some embodiments, criteria assessed for effective treatment includes overall response rate (ORR; also known in some cases as objective response rate), complete response (CR; also known in some cases as complete remission), complete response rate (CRR); duration of response (DOR), progression-free survival (PFS), and/or overall survival (OS).

In some embodiments, at least 40%, at least 50%, at least 60%, or at least 70% of subjects treated according to the methods provided herein achieve complete remission (CR; also known in some cases as complete response), exhibit progression-free survival (PFS) and/or overall survival (OS) for greater than at or about 3 months, 6 months or 12 months or greater than 13 months or approximately 14 months. In some embodiments, on average, subjects treated according to the method exhibit a median PFS or OS of greater than at or about 6 months, 12 months, or 18 months. In some embodiments, the subject exhibits PFS or OS following therapy for at least at or about 6, 12, 18 or more months or longer.

In some embodiments, the subjects treated according to the provided methods exhibits a CRR of at least 50%, at least 60%, at least 70%, at least 80%, or at least 90%. In some embodiments, the complete response rate (CRR) is calculated as the percentage of subjects with the best overall response (BOR) up to 12 months, up to 18 months, up to 24 months, up to 36 months or longer.

In some aspects, response rates in subjects, such as subjects with NHL, are based on the Lugano criteria. (Cheson et al., Blood. 2016; 128(21):2489-96.). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake ≤mediastinum; 3, uptake >mediastinum but <liver; 4, uptake moderately >liver; 5, uptake markedly higher than liver and/or new lesions; X, new areas of uptake unlikely to be related to lymphoma. In some aspects, response assessed using the Lugano criteria involves the use of magnetic resonance imaging (MRI) as appropriate. In some aspects response assessment may be performed at baseline (e.g. prior to any of the methods provided herein), 1 month, 3 months, 6 months, 9 months, 12 months, 18 months, and/or 24 months following administration of the T cell therapy. In some aspects, response assessment is be performed at baseline, 1 month, 3 months, 6 months, 9 months, 12 months, 18 months, and 24 months following administration of the T cell therapy.

In some aspects, a complete response (CR) as described using the Lugano criteria involves a complete metabolic response and a complete radiologic response at various measurable sites. In some aspects, these sites include lymph nodes and extralymphatic sites, wherein a CR is described as a score of 1, 2, or 3 with or without a residual mass on the 5-point scale, when PET-CT is used. In some aspects, extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake.

In some aspects, response is assessed in the lymph nodes using CT, wherein a CR is described as no extralymphatic sites of disease and target nodes/nodal masses must regress to <1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology. Further sites may include assessment of organ enlargement, which should regress to normal. In some aspects, non-measured lesions and new lesions are assessed, which in the case of CR should be absent (Chessen et al., Blood. 2016 Nov. 24; 128(21):2489-96).

In some aspects, a partial response (PR; also known in some cases as partial remission) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease.

In some aspects, response is assessed in the lymph nodes using CT, wherein a PR is described as ≥50% decrease in SPD of up to 6 target measurable nodes and extranodal sites. If a lesion is too small to measure on CT, 5 mm×5 mm is assigned as the default value; if the lesion is no longer visible, the value is 0 mm×0 mm; for a node>5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, non-measured lesions and new lesions are assessed, which in the case of PR should be absent/normal, regressed, but no increase. No response/stable disease (SD) or progressive disease (PD) can also be measured using PET-CT and/or CT based assessments. (Chessen et al., Blood. 2016 Nov. 24; 128(21):2489-96).

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative dosing regimen, such as one in which the subject receives one or more alternative therapeutic agents, one in which the subject does not receive a dose of cells and/or a lymphodepleting agent in accord with the provided methods, one in which the subject receives a dose of cells without a checkpoint inhibitor therapy, and/or with the provided articles of manufacture or compositions. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified.

In some embodiments, the event-free survival rate or overall survival rate of the subject is improved by the methods, as compared with other methods, for example, methods in which the subject receives one or more alternative therapeutic agents, one in which the subject does not receive a dose of cells and/or a lymphodepleting agent in accord with the provided methods, one in which the subject receives a dose of cells but does not receive a checkpoint inhibitor therapy, and/or with the provided articles of manufacture or compositions. For example, in some embodiments, event-free survival rate or probability for subjects treated by the methods at 6 months following the dose is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods, for example, methods in which the subject receives one or more alternative therapeutic agents and/or one in which the subject does not receive a dose of cells and/or a lymphodepleting agent in accord with the provided methods, and/or with the provided articles of manufacture or compositions. For example, in some embodiments, the probability of relapse at 6 months following the first dose is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (a CAR or TCR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR⁺ T cells in a biological sample obtained from the patient, e.g., blood, can be determined at a period of time after administration of the cell therapy, e.g., to determine the pharmacokinetics of the cells. In some embodiments, number of CAR⁺ T cells, optionally CAR⁺ CD8⁺ T cells and/or CAR⁺ CD4⁺ T cells, detectable in the blood of the subject, or in a majority of subjects so treated by the method, is greater than 1 cells per μL, greater than 5 cells per μL or greater than per 10 cells per μL. In some embodiments, the number of CAR⁺ T cells in a biological sample obtained from the patient, e.g., blood, can be determined via PCR for the CAR transgene.

### E. Toxicity

In some embodiments, subjects treated according to any of the provided methods are assessed for one or more signs or symptoms of toxicity that may be associated with the administered cells. Administration of adoptive T cell therapy, such as treatment with T cells expressing chimeric antigen receptors, can induce toxic effects or outcomes such as cytokine release syndrome and neurotoxicity. In some examples, such effects or outcomes parallel high levels of circulating cytokines, which may underlie the observed toxicity.

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013).

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. In some aspects, fever, especially high fever (≥38.5° C. or ≥101.3° F.), is associated with CRS or risk thereof. In some cases, features or symptoms of CRS mimic infection. In some embodiments, infection is also considered in subjects presenting with CRS symptoms, and monitoring by cultures and empiric antibiotic therapy can be administered. Other symptoms associated with CRS can include cardiac dysfunction, adult respiratory distress syndrome, renal and/or hepatic failure, coagulopathies, disseminated intravascular coagulation, and capillary leak syndrome.

CRS may be treated using anti-inflammatory therapy such as an anti-IL-6 therapy, e.g., anti-IL-6 antibody, e.g., tocilizumab, or antibiotics or other agents as described. Outcomes, signs and symptoms of CRS are known and include those described herein. In some embodiments, where a particular administration effects or does not effect a given CRS-associated outcome, sign, or symptom, particular outcomes, signs, and symptoms and/or quantities or degrees thereof may be specified.

In the context of administering CAR-expressing cells, CRS typically occurs within two weeks after infusion of cells that express a CAR. See Abramson et al., J Clin Onc.

2018; 36(15_suppl):7505. In some cases, CRS occurs less than 3 days or more than 21 days after CAR T cell infusion. In non-Hodgkin lymphoma (NHL) subjects treated with JCAR017, CRS usually occurs within two weeks after infusion. See Abramson et al., Blood 2017; 130:581. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary symptoms associated with CRS include fever, fatigue, nausea, headache, rigors, chills, hypotension, dyspnea, acute respiratory distress syndrome (ARDS), encephalopathy, ALT/AST elevation, renal failure, cardiac disorders, hypotension, hypoxia, myalagia/arthraligia, anorexia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly. Other exemplary signs or symptoms associated with CRS include hemodynamic instability, febrile neutropenia, increase in serum C-reactive protein (CRP), changes in coagulation parameters (for example, international normalized ratio (INR), prothrombin time (PTI) and/or fibrinogen), changes in cardiac and other organ function, and/or absolute neutrophil count (ANC).

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, neurotoxicity (NT) can be observed concurrently with CRS.

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

CRS criteria that appear to correlate with the onset of CRS to predict which patients are more likely to be at risk for developing sCRS have been developed (see Davilla et al. Science translational medicine. 2014; 6(224):224ra25; Abramson et al., J Clin Onc. 2018; 36(15_suppl):7505). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. In some embodiments, the criteria reflective of CRS grade are those detailed in Table 2 below.

TABLE 2
Grading Criteria for Cytokine Release Syndrome

| | | Cytokine | | | |
|---|---|---|---|---|---|
| | | Release | | | CRS Grade 4 |
| | | Syndrome | CRS Grade 2 | CRS Grade 3 | (life- |
| | | (CRS) | (moderate) | (severe) | threatening) |
| | | Grade 1 | CRS grade is defined by the most severe symptom | | |
| | Symptoms/Signs | (mild) | (excluding fever) | | |
| Vital | Temperature ≥38.5° | Yes | Any | Any | Any |
| Signs | C./101.3° F. | | | | |
| | Systolic blood | N/A | Responds to | Needs high- | Life- |
| | pressure (SBP) ≤90 | | intravenous (IV) | dose or | threatening |
| | mmHg | | fluids or single | multiple | |
| | | | low-dose | vasopressors | |
| | | | vasopressor | | |
| | Need for oxygen to | N/A | Fraction of inspired | FiO₂ ≥40% | Needs |
| | reach oxygen | | oxygen | | ventilator |
| | saturation | | (FiO₂) <40% | | support |
| | (SaO₂) >90% | | | | |
| Organ | | N/A | Grade 2 | Grade 3 or | Grade 4 |
| Toxicity | | | | transaminitis | (excluding |
| | | | | Grade 4 | transaminitis) |

In some embodiments, high-dose vasopressor therapy include those described in Table 3 below.

TABLE 3
High dose vasopressors (all doses required for ≥3 hours)

| Vasopressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent |
| | (NE) of ≥10 μg/min^{a} |
| If on combination vasopressors | Norepinephrine equivalent of ≥20 |
| (not vasopressin) | μg/min^{a} |

^{a}VASST Trial Vasopressor Equivalent Equation: Norepinephrine equivalent dose = [norepinephrine (μg/min)] + [dopamine (μg/kg/min) ÷ 2] + [epinephrine (μg/min)] + [phenylephrine (μg/min) ÷ 10]

In some embodiments, the toxic outcome is a severe CRS. In some embodiments, the toxic outcome is the absence of severe CRS (e.g. moderate or mild CRS).

In some embodiments, fever and/or levels of C-reactive protein (CRP) can be measured. In some embodiments, the CRS-associated serum factors or CRS-related outcomes include an increase in the level and/or concentration of inflammatory cytokines and/or chemokines, including Flt-3L, fracktalkine, granulocyte macrophage colony stimulating factor (GM-CSF), interleukin-1 beta (IL-103), IL-2, IL-5, IL-6, IL-7, IL-8, IL-10, IL-12, interferon gamma (IFN-γ), macrophage inflammatory protein (MIP)-1, MIP-1, sIL-2Rα, or tumor necrosis factor alpha (TNFα). In some embodiments, the factor or outcome includes C reactive protein (CRP). In some embodiments, subjects that are measured to have high levels of CRP do not have CRS. In some embodiments, a measure of CRS includes a measure of CRP and another factor indicative of CRS.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor. In some embodiments, severe CRS is treated with additional T cell depleting therapies such as cyclophosphamide (Brudno et al., Blood. 2016; 127(26):3321-30).

The method of measuring or detecting the various outcomes may be specified.

In some aspects, the toxic outcome is or is associated with neurotoxicity. In some embodiments, symptoms associated with a clinical risk of neurotoxicity include confusion, delirium, aphasia, expressive aphasia, obtundation, myoclonus, lethargy, altered mental status, convulsions, seizure-like activity, seizures (optionally as confirmed by electroencephalogram (EEG)), elevated levels of beta amyloid (AD), elevated levels of glutamate, and elevated levels of oxygen radicals. In some embodiments, neurotoxicity is graded based on severity (e.g., using a Grade 1-5 scale (see, e.g., National Cancer Institute-Common Toxicity Criteria version 5.00 (NCI CTCAE version 5.0)

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 23 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, severe neurotoxicity includes neurotoxicity with a grade of 3 or greater, such as set forth in Table 4.

TABLE 4
Exemplary Grading Criteria for Neurotoxicity

| Grade | Description of Symptoms |
|---|---|
| 1 | Transient or mild discomfort; no limitation in activity; no medical |
| Asymptomatic or Mild | intervention/therapy required |
| 2 | Presence of symptoms that limit instrumental activities of daily living |
| Moderate | (ADL), such as preparing meals, shopping for groceries or clothes, |
| | using the telephone, managing money |
| 3 | Presence of symptoms that limit self-care ADL, such as bathing, |
| Severe | dressing and undressing, feeding self, using the toilet, taking |
| | medications |
| 4 | Symptoms that are life-threatening, requiring urgent intervention |
| Life-threatening | |
| 5 | Death |
| Fatal | |

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

In some embodiments, the resulting response observed in subjects treated in accord with the provided methods, and/or with the provided articles of manufacture or compositions, is associated with or results in a low risk of any toxicity or a low risk of severe toxicity in a majority of the subjects treated. In some embodiments, greater than or greater than about 30%, 35%, 40%, 50%, 55%, 60%, 70%, 80%, or 90% or more of the subjects treated according to the provided methods and/or with the provided articles of manufacture or compositions do not exhibit any grade of CRS or any grade of neurotoxicity (NT). In some embodiments, greater than or greater than about 50%, 60%, 70%, 80%, 90%, 95% or more of the subjects treated according to the provided methods and/or with the provided articles of manufacture or compositions do not exhibit severe CRS or grade 3 or higher CRS. In some embodiments, greater than or greater than about 50%, 60%, 70%, 80%, 90% or 95% or more of the subjects treated according to the provided methods, and/or with the provided articles of manufacture or compositions, do not exhibit severe neurotoxicity or grade 3 or higher neurotoxicity, such as grade 4 or 5 neurotoxicity.

In some embodiments, at least at or about 45%, 50%, 60%, 65%, 70%, 75%, 80%, 85%, 90%, or 95% of subjects treated according to the method and/or with the provided articles of manufacture or compositions do not exhibit early onset CRS or neurotoxicity and/or do not exhibit onset of CRS earlier than 1 day, 2 days, 3 days or 4 days following initiation of the administration. In some embodiments, at least at or about 45%, 50%, 60%, 65%, 70%, 75%, 80%, 85%, 90%, or 95% of subjects treated according to the methods, and/or with the provided articles of manufacture or compositions, do not exhibit onset of neurotoxicity earlier than 3 days, 4 days, 5 days, six days or 7 days following initiation of the administration. In some aspects, the median onset of neurotoxicity among subjects treated according to the methods, and/or with the provided articles of manufacture or compositions, is at or after the median peak of, or median time to resolution of, CRS in subjects treated according to the method. In some cases, the median onset of neurotoxicity among subjects treated according to the method is greater than at or about 8, 9, 10, or 11 days.

### II. Cell Therapy and Cell Engineering

In some embodiments, the cells contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8+ or CD4+ cells are enriched or selected. Among the compositions are pharmaceutical compositions and formulations for administration, such as for adoptive cell therapy. Also provided are therapeutic methods for administering the cells and compositions to subjects, e.g., patients.

Thus, in some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

### A. Recombinant Receptors

In some embodiments, the cell therapy, e.g. T cell therapy, for use in accord with the provided combination therapy methods includes administering engineered cells expressing recombinant receptors designed to recognize and/or specifically bind to molecules associated with the disease or condition, such as a cancer (a non-Hodgkin lymphoma; NHL), and result in a response, such as an immune response against such molecules upon binding to such molecules. The receptors may include chimeric receptors, e.g., chimeric antigen receptors (CARs), and other transgenic antigen receptors including transgenic T cell receptors (TCRs).

### 1. Chimeric Antigen Receptors

In some embodiments of the provided methods and uses, the engineered cells, such as T cells, express a chimeric receptor, such as a chimeric antigen receptor (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the engineered cells, such as T cells, express a recombinant receptor such as a chimeric antigen receptor (CAR) with specificity for a particular antigen (or marker or ligand), such as an antigen expressed on the surface of a particular cell type. In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

The chimeric receptors, such as CARs, generally include an extracellular antigen binding domain that is an antigen-binding portion or portions of an antibody molecule. In some embodiments, the antigen-binding domain is a portion of an antibody molecule, generally a variable heavy (V_{H}) chain region and/or variable light (V_{L}) chain region of the antibody, e.g., an scFv antibody fragment. In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment. In some embodiments, the CAR contains an antibody or an antigen-binding fragment (e.g. scFv) that specifically recognizes an antigen, such as an intact antigen, expressed on the surface of a cell.

Among the antigen receptors are a CAR containing an extracellular antigen binding domain, such as antibody or antigen-binding fragment, that exhibits TCR-like specificity directed against peptide-MHC complexes, which also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, a and f, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen CJ. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 5, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 5
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat | | | | |
| Number- | | | | |
| ing¹) | | | | |
| CDR-H1 | H31--H35 | H26--H32 | H26--H35 | H30--H35 |
| (Chothia | | | | |
| Number- | | | | |
| ing²) | | | | |
| CDR-H2 | H50--H65 | H52--H56 | H50--H58 | H47--H58 |
| CDR-H3 | H95--H102 | H95--H102 | H95--H102 | H93--H101 |

¹- Kabat et al. (1991), “Sequences of Proteins of Immunological Interest,” 5th Ed. Public Health Service, National Institutes of Health, Bethesda, MD
²- Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the recombinant receptor, such as a chimeric receptor (e.g. CAR), includes an extracellular antigen binding domain, such as an antibody or antigen-binding fragment (e.g. scFv), that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen targeted by the receptor is or comprises selected from among avP6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen targeted by the receptor is or includes CD20, CD19, CD22, ROR1, CD45, CD21, CD5, CD33, Igkappa, Iglambda, CD79a, CD79b or CD30. In some embodiments, the antigen targeted by the receptor is or includes CD19. In some embodiments, the disease or condition is a B cell malignancy, and the antigen is CD19. In some embodiments, the disease or condition is a non-Hodgkin lymphoma (NHL), and the antigen is CD19.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19. In some embodiments, any of such antigens are antigens expressed on human B cells.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III 302). In some embodiments, the FMC63 antibody comprises the CDR-H1 and CDR-H2 set forth in SEQ ID NO: 38 and 39, respectively, the CDR-H3 set forth in SEQ ID NO: 40 or 54, the CDR-L1 set forth in SEQ ID NO: 35, the CDR-L2 set forth in SEQ ID NO: 36 or 55 and the CDR-L3 sequences set forth in SEQ ID NO: 37 or 56. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:41 and a variable light chain region of FMC63 set forth in SEQ ID NO:42, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:59. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:50 and a variable light chain region of SJ25C1 set forth in SEQ ID NO:51, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some aspects, the recombinant receptor, e.g., a chimeric antigen receptor, includes an extracellular portion containing one or more ligand- (e.g., antigen-) binding domains, such as an antibody or fragment thereof, and one or more intracellular signaling region or domain (also interchangeably called a cytoplasmic signaling domain or region). In some embodiments, the antibody or fragment includes an scFv. In some aspects, the chimeric antigen receptor includes an extracellular portion containing an antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s)

In some embodiments, the recombinant receptor such as the CAR, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687, U.S. Pat. No. 8,822,647 or published app. No. US2014/0271635.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains, such as set forth in SEQ ID NO:4. In some embodiments, the spacer the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity SEQ ID NO: 1. In some embodiments, the spacer comprises the sequence set forth in SEQ ID NO: 1.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular domain and transmembrane can be linked directly or indirectly. In some embodiments, the extracellular domain and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the recombinant receptor, e.g. CAR, includes at least one intracellular signaling component or components, such as an intracellular signaling region or domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8alpha, CD8beta, CD4, CD25, or CD16. For example, in some aspects, the CAR or other chimeric receptor includes a chimeric molecule between CD3-zeta (CD3-ζ) or Fc receptor γ and CD8alpha, CD8beta, CD4, CD25 or CD16.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3((Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15. In some embodiments, the intracellular signaling region comprises the sequence of amino acids set forth in SEQ ID NO: 13 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13. In some embodiments, the intracellular signaling region comprises the sequence of amino acids set forth in SEQ ID NO: 13.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12. In some embodiments, the intracellular region comprises an intracellular costimulatory signaling domain comprising the sequence of amino acids set forth in SEQ ID NO: 12.

In some aspects, the same CAR includes both the primary (or activating) cytoplasmic signaling regions and costimulatory signaling components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto. In some embodiments, the transmembrane domain comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8. In some embodiments, the transmembrane domain comprises the sequence of amino acids set forth in SEQ ID NO: 8. In some embodiments, the transmembrane domain comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto. In some embodiments, the transmembrane domain comprises the sequence of amino acids set forth in SEQ ID NO: 9.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12. In some embodiments, the intracellular domain comprises an intracellular costimulatory signaling domain comprising the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12. In some embodiments, the intracellular domain comprises an intracellular costimulatory signaling domain comprising the sequence of amino acids set forth in SEQ ID NO: 12.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3((Accession No.: P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15. In some embodiments, the intracellular signaling domain comprises the sequence of amino acids as set forth in SEQ ID NO: 13 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13. In some embodiments, the intracellular signaling domain comprises the sequence of amino acids as set forth in SEQ ID NO: 13.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 17, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 16, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No.

20070116690.

In some of any of the embodiments, the CAR comprises, in order, an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further includes a spacer between the transmembrane domain and the scFv;

In some of any of the embodiments, the CAR includes, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain.

In some of any of the embodiments, the CAR comprises or consists of, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain. In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain comprises SEQ ID NO: 13 or 14 or 15 or a variant thereof having at least 85%, 86%, 87%, 88%, 89%, 90%, 91%, 92%, 93%, 94%, 95%, 96%, 97%, 98%, 99% or more sequence identity thereto; and/or the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a V_{H}, a linker, optionally comprising SEQ ID NO: 59, and a V_{L}, and/or the scFv comprises a flexible linker and/or comprises the amino acid sequence set forth as SEQ ID NO: 59.

In some embodiments, the spacer comprises or consists of SEQ ID NO: 1, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 30, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 31, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 33, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

In some embodiments, the spacer comprises or consists of SEQ ID NO: 34, the costimulatory domain comprises SEQ ID NO: 12 or variant thereof having at least 85%, 86%, 87%, 88%, 89%, 90%, 91%, 92%, 93%, 94%, 95%, 96%, 97%, 98%, 99% or more sequence identity thereto, the transmembrane domain is of CD28 or comprises SEQ ID NO: 9 or a variant thereof having at least 85%, 86%, 87%, 88%, 89%, 90%, 91%, 92%, 93%, 94%, 95%, 96%, 97%, 98%, 99% or more sequence identity thereto, the scFv contains the binding domain of or CDRs of or V_{H} and V_{L} of FMC63, the primary signaling domain contains SEQ ID NO: 13, 14, or 15, and/or a variant thereof having at least 85%, 86%, 87%, 88%, 89%, 90%, 91%, 92%, 93%, 94%, 95%, 96%, 97%, 98%, 99% or more sequence identity thereto.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

### 2 T Cell Receptors

In some embodiments, engineered cells, such as T cells, used in connection with the provided methods, uses, articles of manufacture or compositions are cells that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the a form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the a and 3 chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4⁺ or CD8⁺ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR a chain variable region sequence is fused to the N terminus of a sequence corresponding to a TCR a chain constant region extracellular sequence, and a second polypeptide wherein a sequence corresponding to a TCR 3 chain variable region sequence is fused to the N terminus a sequence corresponding to a TCR 3 chain constant region extracellular sequence, the first and second polypeptides being linked by a disulfide bond. In some embodiments, the bond can correspond to the native inter-chain disulfide bond present in native dimeric a TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable a domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wulfing, C. and Pluckthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an a chain variable region sequence fused to the N terminus of an a chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an a chain variable region sequence fused to the N terminus of a sequence a chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO:22). In some embodiments, the linker has the sequence GSADDAKKDAAKKDGKS (SEQ ID NO:23)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁵ and 10⁻¹² M and all individual values and ranges therein. In some embodiments, the target antigen is an MHC-peptide complex or ligand.

In some embodiments, nucleic acid or nucleic acids encoding a TCR, such as a and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBIl21 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

### B. Methods of Engineering Cells

In some embodiments, the provided methods involve administering to a subject having a disease or condition (e.g. a cancer such as NHL) cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In particular embodiments, the engineered cells are produced by a process that generates an output composition of enriched T cells from one or more input compositions and/or from a single biological sample. In certain embodiments, the output composition contains cells that express a recombinant receptor, e.g., a CAR, such as an anti-CD19 CAR. In particular embodiments, the cells of the output compositions are suitable for administration to a subject as a therapy, e.g., an autologous cell therapy. In some embodiments, the output composition is a composition of enriched CD4+ or CD8+ T cells.

In some embodiments, the process for generating or producing engineered cells is by a process that includes some or all of the steps of: collecting or obtaining a biological sample; isolating, selecting, or enriching input cells from the biological sample; cryopreserving and storing the input cells; thawing and/or incubating the input cells under stimulating conditions; engineering the stimulated cells to express or contain a recombinant polynucleotide, e.g., a polynucleotide encoding a recombinant receptor such as a CAR; cultivating the engineered cells, e.g. to a threshold amount, density, or expansion; formulating the cultivated cells in an output composition; and/or cryopreserving and storing the formulated output cells until the cells are released for infusion and/or are suitable to be administered to a subject. In certain embodiments, the process is performed with two or more input compositions of enriched T cells, such as a separate CD4+ composition and a separate CD8+ composition, that are separately processed and engineered from the same starting or initial biological sample and re-infused back into the subject at a defined ratio, e.g. 1:1 ratio of CD4+ to CD8+ T cells. In some embodiments, the enriched T cells are or include engineered T cells, e.g., T cells transduced to express a recombinant receptor.

In particular embodiments, an output composition of engineered cells expressing a recombinant receptor (e.g. anti-CD19 CAR) is produced from an initial and/or input composition of cells. In some embodiments, the input composition is a composition of enriched CD3+ T cells, enriched CD4+ T cells, and/or enriched CD8+ T cells (herein after also referred to as compositions of enriched T cells, compositions of enriched CD4+ T cells, and compositions of enriched CD8+ T cells, respectively). In some embodiments, a composition enriched in CD4+ T cells contains at least 60%, 65%, 70%, 75%, 80%, 85%, 90%, 95%, 98%, 99%, or 99.9% CD4+ T cells. In particular embodiments, the composition of enriched CD4+ T cells contains about 100% CD4+ T cells. In certain embodiments, the composition of enriched CD4+ T cells includes or contains less than 20%, less than 10%, less than 5%, less than 1%, less than 0.1%, or less than 0.01% CD8+ T cells, and/or contains no CD8+ T cells, and/or is free or substantially free of CD8+ T cells. In some embodiments, the populations of enriched CD4+ T cells consist essentially of CD4+ T cells. In some embodiments, a composition enriched in CD8+ T cells contains at least 75%, 80%, 85%, 90%, 95%, 98%, 99%, or 99.9% CD8+ T cells, or contains or contains about 100% CD8+ T cells. In certain embodiments, the composition of enriched CD8+ T cells includes or contains less than 20%, less than 10%, less than 5%, less than 1%, less than 0.1%, or less than 0.01% CD4+ T cells, and/or contains no CD4+ T cells, and/or is free or substantially free of CD4+ T cells. In some embodiments, the populations of enriched CD8+ T cells consist essentially of CD8+ T cells.

In some embodiments, a composition enriched in CD3+ T cells contains at least 60%, 65%, 70%, 75%, 80%, 85%, 90%, 95%, 98%, 99%, or 99.9% CD3+ T cells. In particular embodiments, the composition of enriched CD3+ T cells contains about 100% CD3+ T cells. In certain embodiments, the composition of enriched CD3+ T cells includes CD4+ and CD8+ T cells that are at a ratio of CD4+ T cells to CD8+ T cells of between approximately 1:3 and approximately 3:1, such as approximately 1:1.

In certain embodiments, the process for producing engineered cells further can include one or more of: activating and/or stimulating a cells, e.g., cells of an input composition; genetically engineering the activated and/or stimulated cells, e.g., to introduce a polynucleotide encoding a recombinant protein by transduction or transfection; and/or cultivating the engineered cells, e.g., under conditions that promote proliferation and/or expansion. In particular embodiments, the provided methods may be used in connection with harvesting, collecting, and/or formulating output compositions produced after the cells have been incubated, activated, stimulated, engineered, transduced, transfected, and/or cultivated.

In some embodiments, engineered cells, such as those that express an anti-CD19 CAR, used in accord with the provided methods are produced or generated by a process for selecting, isolating, activating, stimulating, expanding, cultivating, and/or formulating cells. In some embodiments, such methods include any as described.

In some embodiments, at least one separate composition of enriched CD4+ T cells and at least one separate composition of enriched CD8+ T cells are isolated, selected, enriched, or obtained from a single biological sample, e.g., a sample of PBMCs or other white blood cells from the same donor such as a patient or healthy individual. In some embodiments, a separate composition of enriched CD4+ T cells and a separate composition of enriched CD8+ T cells originated, e.g., are initially isolated, selected, and/or enriched, from the same biological sample, such as a single biological sample obtained, collected, and/or taken from a single subject. In some embodiments, a biological sample is first subjected to selection of CD4+ T cells, where both the negative and positive fractions are retained, and the negative fraction is further subjected to selection of CD8+ T cells. In other embodiments, a biological sample is first subjected to selection of CD8+ T cells, where both the negative and positive fractions are retained, and the negative fraction is further subjected to selection of CD4+ T cells. In some embodiments, methods of selection are carried out as described in International PCT publication No. WO2015/164675. In some aspects, a biological sample is first positively selected for CD8+ T cells to generate at least one composition of enriched CD8+ T cells, and the negative fraction is then positively selected for CD4+ T cells to generate at least one composition of enriched CD4+ T cells, such that the at least one composition of enriched CD8+ T cells and the at least one composition of enriched CD4+ T cells are separate compositions from the same biological sample, e.g., from the same donor patient or healthy individual. In some aspects, two or more separate compositions of enriched T cells, e.g., at least one being a composition of enriched CD4+ T cells and at least one being a separate composition of enriched CD8+ T cells from the same donor, are separately frozen, e.g., cryoprotected or cryopreserved in a cryopreservation media.

In some aspects, two or more separate compositions of enriched T cells, e.g., at least one being a composition of enriched CD4+ T cells and at least one being a separate composition of enriched CD8+ T cells from the same biological sample, are activated and/or stimulated by contacting with a stimulatory reagent (e.g., by incubation with CD3/CD28 conjugated magnetic beads for T cell activation). In some aspects, each of the activated/stimulated cell composition is engineered, transduced, and/or transfected, e.g., using a viral vector encoding a recombinant protein (e.g. CAR), to express the same recombinant protein in the CD4+ T cells and CD8+ T cells of each cell composition. In some aspects, the method comprises removing the stimulatory reagent, e.g., magnetic beads, from the cell composition. In some aspects, a cell composition containing engineered CD4+ T cells and a cell composition containing engineered CD8+ T cells are separately cultivated, e.g., for separate expansion of the CD4+ T cell and CD8+ T cell populations therein. In certain embodiments, a cell composition from the cultivation is harvested and/or collected and/or formulated, e.g., by washing the cell composition in a formulation buffer. In certain embodiments, a formulated cell composition comprising CD4+ T cells and a formulated cell composition comprising CD8+ T cells is frozen, e.g., cryoprotected or cryopreserved in a cryopreservation media. In some aspects, engineered CD4+ T cells and CD8+ T cells in each formulation originate from the same donor or biological sample and express the same recombination protein (e.g., CAR, such as anti-CD19 CAR). In some aspects, a separate engineered CD4+ formulation and a separate engineered CD8+ formulation are administered at a defined ratio, e.g. 1:1, to a subject in need thereof such as the same donor.

In some aspects, two or more separate compositions of enriched T cells, e.g., at least one being a composition of enriched CD4+ T cells and at least one being a separate composition of enriched CD8+ T cells from the same biological sample, selected from a sample from a subject and then are combined at a defined ratio, e.g. 1:1. In some embodiments, the combined composition enriched in CD4+ and CD8+ T cells are activated and/or stimulated by contacting with a stimulatory reagent (e.g., by incubation with CD3/CD28 conjugated magnetic beads for T cell activation). In some aspects, the activated/stimulated cell composition is engineered, transduced, and/or transfected, e.g., using a viral vector encoding a recombinant protein (e.g. CAR), to express the recombinant protein in the CD4+ T cells and CD8+ T cells of the cell composition. In some aspects, the method comprises removing the stimulatory reagent, e.g., magnetic beads, from the cell composition. In some aspects, the cell composition containing engineered CD4+ T cells and engineered CD8+ T cells are cultivated, e.g., for expansion of the CD4+ T cell and CD8+ T cell populations therein. In certain embodiments, a cell composition from the cultivation is harvested and/or collected and/or formulated, e.g., by washing the cell composition in a formulation buffer. In certain embodiments, a formulated cell composition comprising recombinant receptor (e.g. CAR) engineered CD4+ T cells and CD8+ T cells is frozen, e.g., cryoprotected or cryopreserved in a cryopreservation media. In some aspects, engineered CD4+ T cells and CD8+ T cells in the formulation originate from the same donor or biological sample and express the same recombinant protein (e.g., CAR, such as anti-CD19 CAR).

In some aspects, a composition of enriched CD3+ T cells is selected from a sample from a subject. In some embodiments, the composition enriched in CD3+ T cells is activated and/or stimulated by contacting with a stimulatory reagent (e.g., by incubation with CD3/CD28 conjugated magnetic beads for T cell activation). In some aspects, the activated/stimulated cell composition is engineered, transduced, and/or transfected, e.g., using a viral vector encoding a recombinant protein (e.g. CAR), to express the recombinant protein in the T cells of the cell composition. In some aspects, the method comprises removing the stimulatory reagent, e.g., magnetic beads, from the cell composition. In some aspects, the cell composition containing engineered CD3+ T cells are cultivated, e.g., for expansion of the T cells populations therein. In certain embodiments, a cell composition from the cultivation is harvested and/or collected and/or formulated, e.g., by washing the cell composition in a formulation buffer. In certain embodiments, a formulated cell composition comprising recombinant receptor (e.g. CAR) engineered CD3+ T cells is frozen, e.g., cryoprotected or cryopreserved in a cryopreservation media. In some aspects, engineered CD3+ T cells in the formulation express a CAR, such as anti-CD19 CAR.

### 1. Cells and Preparation of Cells for Genetic Engineering

In some embodiments, cells, such as T cells, used in connection with the provided methods, uses, articles of manufacture or compositions are cells have been genetically engineered to express a recombinant receptor, e.g., a CAR or a TCR described herein. In some embodiments, the engineered cells are used in the context of cell therapy, e.g., adoptive cell therapy. In some embodiments, the engineered cells are immune cells. In some embodiments, the engineered cells are T cells, such as CD4+ and CD8+ T cells, CD4+ T cells, or CD8+ T cells.

In some embodiments, the nucleic acids, such as nucleic acids encoding a recombinant receptor, are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. In some cases, an apheresis or leukapheresis sample is obtained from the subject about 2 weeks and about 6 weeks, such as about 4 weeks, prior to administration of the cell therapy (e.g. CAR T cells). In some cases, the apheresis or leukapheresis sample is obtained from the subject about 4 weeks prior to administration of the cell therapy (e.g. CAR T cells). The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD3, CD4 and/or CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or at least about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or at least about 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° Celsius at a rate of 1° per minute and stored in the vapor phase of a liquid nitrogen storage tank.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

### 2 Activation and Stimulation

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

For example, the stimulating conditions can include incubation using anti-CD3/anti-CD28 conjugated magnetic beads (e.g., DYNABEADS® M-450 CD3/CD28 T Cell Expander).

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or at least about or about or 10 mL, 20 mL, 30 mL, 40 mL, 50 mL, 60 mL, 70 mL, 80 mL, 90 mL, 100 mL, 150 mL or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or at least about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours, or 12 hours and 24 hours, inclusive.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF).

In some embodiments, the stimulation results in activation and/or proliferation of the cells, for example, prior to transduction.

### 3. Vectors and Methods for Genetic Engineering

In some embodiments, engineered cells, such as T cells, used in connection with the provided methods, uses, articles of manufacture or compositions are cells have been genetically engineered to express a recombinant receptor, e.g., a CAR or a TCR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g. anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are genetically engineered separately.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described.

In some embodiments, methods for genetic engineering are carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the contacting can be effected with centrifugation, such as spinoculation (e.g., centrifugal inoculation). In some embodiments, the composition containing cells, the vector, e.g., viral particles and reagent can be rotated, generally at relatively low force or speed, such as speed lower than that used to pellet the cells, such as from or from about 600 rpm to 1700 rpm (e.g., at or about or at least 600 rpm, 1000 rpm, or 1500 rpm or 1700 rpm). In some embodiments, the rotation is carried at a force, e.g., a relative centrifugal force, of from or from about 100 g to 3200 g (e.g., at or about or at least at or about 100 g, 200 g, 300 g, 400 g, 500 g, 1000 g, 1500 g, 2000 g, 2500 g, 3000 g, or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, during at least a part of the genetic engineering, e.g. transduction, and/or subsequent to the genetic engineering the cells are transferred to a bioreactor bag assembly for culture of the genetically engineered cells, such as for cultivation or expansion of the cells.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle. In some embodiments, the cells to be transfected or transduced are or comprise primary cells obtained from a subject, such as cells enriched and/or selected from a subject.

In some embodiments, the concentration of cells to be transduced of the composition is from or from about 1.0×10⁵ cells/mL to 1.0×10⁸ cells/mL, such as at least or at least about or about 1.0×10⁵ cells/mL, 5×10⁵ cells/mL, 1×10⁶ cells/mL, 5×10⁶ cells/mL, 1×10⁷ cells/mL, 5×10⁷ cells/mL, or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5, or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or at least about 30 minutes, 1 hour, 2 hours, 6 hours, 12 hours, 24 hours, 36 hours, or more.

In some embodiments, contacting is performed in solution. In some embodiments, the cells and viral particles are contacted in a volume of from or from about 0.5 mL to 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL, or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol.

2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### 4. Cultivation, Expansion and Formulation of Engineered Cells

In some embodiments, the methods for generating the engineered cells, e.g., for cell therapy in accord with any of provided methods, uses, articles of manufacture or compositions, include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. Thus, in some embodiments, a composition of CAR-positive T cells that has been engineered by transduction or transfection with a recombinant polynucleotide encoding the CAR, is cultivated under conditions that promote proliferation and/or expansion.

In certain embodiments, the one or more compositions of engineered T cells are or include two separate compositions of enriched T cells, such as two separate compositions of enriched T cells that have been engineered with a polynucleotide encoding a recombinant receptor, e.g. a CAR. In particular embodiments, two separate compositions of enriched T cells, e.g., two separate compositions of enriched T cells selected, isolated, and/or enriched from the same biological sample, are separately cultivated under stimulating conditions, such as subsequent to a step of genetically engineering, e.g., introducing a recombinant polypeptide to the cells by transduction or transfection. In certain embodiments, the two separate compositions include a composition of enriched CD4+ T cells, such as a composition of enriched CD4+ T cells that have been engineered with a polynucleotide encoding a recombinant receptor, e.g. a CAR. In particular embodiments, the two separate compositions include a composition of enriched CD8+ T cells, such as a composition of enriched CD4+ T cells that have been engineered with a polynucleotide encoding a recombinant receptor, e.g. a CAR. In some embodiments, two separate compositions of enriched CD4+ T cells and enriched CD8+ T cells, such as a composition of enriched CD4+ T cells and a composition of enriched CD8+ T cells that have each been separately engineered with a polynucleotide encoding a recombinant receptor, e.g. a CAR, are separately cultivated, e.g., under conditions that promote proliferation and/or expansion.

In some embodiments, cultivation is carried out under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In particular embodiments, the cells are cultivated in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines, e.g. a recombinant cytokine, is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more recombinant cytokine includes IL-2, IL-7 and/or IL-15. In some embodiments, the cells, e.g., engineered cells, are cultivated in the presence of a cytokine, e.g., a recombinant human cytokine, at a concentration of between 1 IU/mL and 2,000 IU/mL, between 10 IU/mL and 100 IU/mL, between 50 IU/mL and 200 IU/mL, between 100 IU/mL and 500 IU/mL, between 100 IU/mL and 1,000 IU/mL, between 500 IU/mL and 2,000 IU/mL, or between 100 IU/mL and 1,500 IU/mL.

In some embodiments, the cultivation is performed under conditions that generally include a temperature suitable for the growth of primary immune cells, such as human T lymphocytes, for example, at least about 25 degrees Celsius, generally at least about 30 degrees Celsius, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, 9 days or more.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7° 6°, 5°, 4°, 3°, 2°, or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RPM or 10 RPM.

In some embodiments, the methods for manufacturing, generating or producing a cell therapy and/or engineered cells, in accord with the provided methods, uses or articles of manufacture, may include formulation of cells, such as formulation of genetically engineered cells resulting from the processing steps prior to or after the incubating, engineering, and cultivating, and/or one or more other processing steps as described. In some embodiments, one or more of the processing steps, including formulation of cells, can be carried out in a closed system. In some cases, the cells are processed in one or more steps (e.g. carried out in the centrifugal chamber and/or closed system) for manufacturing, generating or producing a cell therapy and/or engineered cells may include formulation of cells, such as formulation of genetically engineered cells resulting from the transduction processing steps prior to or after the culturing, e.g. cultivation and expansion, and/or one or more other processing steps as described. In some embodiments, the genetically engineered cells are formulated as unit dose form compositions including the number of cells for administration in a given dose or fraction thereof.

In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, such as in the treatment of diseases, conditions, and disorders, or in detection, diagnostic, and prognostic methods, and uses and articles of manufacture. In some cases, the cells can be formulated in an amount for dosage administration, such as for a single unit dosage administration or multiple dosage administration.

In some embodiments, the cells can be formulated into a container, such as a bag or vial. In some embodiments, the vial may be an infusion vial. In some embodiments, the vial is formulated with a single unit dose of the engineered cells, such as including the number of cells for administration in a given dose or fraction thereof.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

In some embodiments, the formulation buffer contains a cryopreservative. In some embodiments, the cell are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and 5%, between 0.25% and 4%, between 0.5% and 2%, or between 1% and 2% HSA.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from or from about 10 mL to 1000 mL, such as at least or at least about or about or 50 mL, 100 mL, 200 mL, 300 mL, 400 mL, 500 mL, 600 mL, 700 mL, 800 mL, 900 mL, or 1000 mL.

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as the vials of the biomedical material vessels described herein, that is operably linked as part of a closed system with the centrifugal chamber. In some embodiments, the biomedical material vessels are configured for integration and or operable connection and/or is integrated or operably connected, to a closed system or device that carries out one or more processing steps. In some embodiments, the biomedical material vessel is connected to a system at an output line or output position. In some cases, the closed system is connected to the vial of the biomedical material vessel at the inlet tube. Exemplary close systems for use with the biomedical material vessels described herein include the Sepax® and Sepax® 2 system.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of vials, can be sterilely connected to one or more, generally two or more, such as at least 3, 4, 5, 6, 7, 8, or more of the ports of the multi-port output. For example, in some embodiments, one or more containers, e.g., biomedical material vessels, can be attached to the ports, or to fewer than all of the ports. Thus, in some embodiments, the system can effect expression of the output composition into a plurality of vials of the biomedical material vessels.

In some aspects, cells can be expressed to the one or more of the plurality of output containers, e.g., vials, in an amount for dosage administration, such as for a single unit dosage administration or multiple dosage administration. For example, in some embodiments, the vials, may each contain the number of cells for administration in a given dose or fraction thereof. Thus, each vial, in some aspects, may contain a single unit dose for administration or may contain a fraction of a desired dose such that more than one of the plurality of vials, such as two of the vials, or 3 of the vials, together constitute a dose for administration. In some embodiments, 4 vials together constitute a dose for administration.

Thus, the containers, e.g. bags or vials, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject. In some aspects, the provided articles of manufacture includes one or more of the plurality of output containers.

In some embodiments, each of the containers, e.g. bags or vials, individually comprises a unit dose of the cells. Thus in some embodiments, each of the containers comprises the same or approximately or substantially the same number of cells. In some embodiments, each unit dose contains at or about or at least or at least about 1×10⁶, 2×10⁶, 5×10⁶, 1×10⁷, 5×10⁷, or 1×10⁸ engineered cells, total cells, T cells, or PBMCs. In some embodiments, each unit dose contains at or about or at least or at least about 1×10⁶, 2×10⁶, 5×10⁶, 1×10⁷, 5×10⁷, or 1×10⁸ CAR+ T cells that are CD3+, such as CD4+ or CD8+, or a viable subset thereof. In some embodiments, the volume of the formulated cell composition in each container, e.g. bag or vial, is between at or about 10 mL and at or about 100 mL, such as at or about or at least or at least about 20 mL, 30 mL, 40 mL, 50 mL, 60 mL, 70 mL, 80 mL, 90 mL, or 100 mL. In some embodiments, the volume of the formulated cell composition in each container, e.g. bag or vial, is between at or about 1 mL and at or about 10 mL, such as between at or about 1 mL and at or about 5 mL. In some embodiments, the volume of the formulated cell composition in each container, e.g. bag or vial, is between at or about 4 mL and at or about 5 mL. In some embodiments, the volume of the formulated cell composition in each container, e.g. bag or vial, is or is about 4.4 mL. In some embodiments, the volume of the formulated cell composition in each container, e.g. bag or vial, is or is about 4.5 mL. In some embodiments, the volume of the formulated cell composition in each container, e.g. bag or vial, is or is about 4.6 mL. In some embodiments, the volume of the formulated cell composition in each container, e.g. bag or vial, is or is about 4.7 mL. In some embodiments, the volume of the formulated cell composition in each container, e.g. bag or vial, is or is about 4.8 mL. In some embodiments, the volume of the formulated cell composition in each container, e.g. bag or vial, is or is about 4.9 mL. In some embodiments, the volume of the formulated cell composition in each container, e.g. bag or vial, is or is about 5.0 mL.

In some embodiments, the formulated cell composition has a concentration of greater than at or about 0.5×10⁶ recombinant receptor-expressing (e.g. CAR⁺)/CD3+ cells or such viable cells per mL, greater than at or about 1.0×10⁶ recombinant receptor-expressing (e.g. CAR⁺)/CD3+ cells or such viable cells per mL, greater than at or about 1.5×10⁶ recombinant receptor-expressing (e.g. CAR⁺)/CD3+ cells or such viable cells per mL, greater than at or about 2.0×10⁶ recombinant receptor-expressing (e.g. CAR⁺)/CD3+ cells or such viable cells per mL. greater than at or about 2.5×10⁶ recombinant receptor-expressing (e.g. CAR⁺)/CD3+ cells or such viable cells per mL, greater than at or about 2.6×10⁶ recombinant receptor-expressing (e.g. CAR⁺)/CD3+ cells or such viable cells per mL, greater than at or about 2.7×10⁶ recombinant receptor-expressing (e.g. CAR⁺)/CD3+ cells or such viable cells per mL, greater than at or about 2.8×10⁶ recombinant receptor-expressing (e.g. CAR⁺)/CD3+ cells or such viable cells per mL, greater than at or about 2.9×10⁶ recombinant receptor-expressing (e.g. CAR⁺)/CD3+ cells or such viable cells per mL greater than at or about 3.0×10⁶ recombinant receptor-expressing (e.g. CAR⁺)/CD3+ cells or such viable cells per mL, greater than at or about 3.5×10⁶ recombinant receptor-expressing (e.g. CAR⁺)/CD3+ cells or such viable cells per mL, greater than at or about 4.0×10⁶ recombinant receptor-expressing (e.g. CAR⁺)/CD3+ cells or such viable cells per mL, greater than at or about 4.5×10⁶ recombinant receptor-expressing (e.g. CAR⁺)/CD3+ cells or such viable cells per mL or greater than at or about 5×10⁶ recombinant receptor-expressing (e.g. CAR⁺)/CD3+ cells or such viable cells per mL. In some embodiments, the CD3+ cells are CD4+ T cells. In some embodiments, the CD3+ cells are CD8+ T cells. In some embodiments, the CD3+ T cells are CD4+ and CD8+ T cells.

In some embodiments, the cells in the container, e.g. bag or vials, can be cryopreserved. In some embodiments, the container, e.g. vials, can be stored in liquid nitrogen until further use.

In some embodiments, such cells produced by the method, or a composition comprising such cells, are administered to a subject for treating a disease or condition, for example, in accord with the methods, uses and articles of manufacture described herein.

### III. Articles of Manufacture and Kits

Also provided are articles of manufacture containing a checkpoint inhibitor therapy, e.g., an anti-PD-1 antibody and optionally an anti-LAG3 antibody, and components for the cell therapy, e.g., a T cell therapy, e.g. CAR T cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the engineered cells used for the cell therapy, e.g. an engineered T cell therapy; and (b) a second container with a composition contained therein, wherein the composition includes the checkpoint inhibitor therapy, e.g., an anti-PD-1 antibody and optionally an anti-LAG3 antibody.

In some embodiments, the first container comprises a first composition and a second composition, wherein the first composition comprises a first population of the engineered cells used for the immunotherapy, e.g., CD4+ T cell therapy, and the second composition comprises a second population of the engineered cells, wherein the second population may be engineered separately from the first population, e.g., CD8+ T cell therapy. In some embodiments, the first and second cell compositions contain a defined ratio of the engineered cells, e.g., CD4+ and CD8+ cells (e.g., 1:1 ratio of CD4+:CD8+ CAR+ T cells).

In some embodiments, the first container comprises a first composition, a second composition, and a third composition, wherein the first composition comprises a first population of the engineered cells used for the immunotherapy, e.g., CD4+ T cell therapy, the second composition comprises a second population of the engineered cells, wherein the second population may be engineered separately from the first population, e.g., CD8+ T cell therapy, and the third composition comprises a checkpoint inhibitory therapy (e.g. an anti-PD-1 antibody and optionally an anti-LAG3 antibody). In some embodiments, the first and second cell compositions contain a defined ratio of the engineered cells, e.g., CD4+ and CD8+ cells (e.g., 1:1 ratio of CD4+:CD8+ CAR+ T cells).

The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### V. Exemplary Embodiments

Among the provided embodiments are:
1. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
   (i) administration of the first dose is between Day 2 and Day 20, inclusive; and
   (ii) a dose is administered about every two weeks (Q2W) or about every four weeks (Q4W) in an amount of between at or about 140 mg and at or about 580 mg, inclusive.
2. The method of embodiment 1, wherein the first dose is administered between Day 8 and Day 15, inclusive.
3. The method of embodiment 1 or embodiment 2, wherein the first dose is administered on Day 8.
4. The method of embodiment 1 or embodiment 2, wherein the first dose is administered on Day 15.
5. The method of any of embodiments 1-4, wherein the amount of the PD-1 inhibitor is between at or about 160 mg and 560 mg.
6. The method of any of embodiments 1-5, wherein the amount of the PD-1 inhibitor is at or about 240 mg or at or about 480 mg.
7. The method of any of embodiments 1-6, wherein the amount of the PD-1 inhibitor is 240 mg.
8. The method of any of embodiments 1-6, wherein the amount of the PD-1 inhibitor is 480 mg.
9. The method of any of embodiments 1-8, wherein a dose is administered about every two weeks (Q2W).
10. The method of any of embodiments 1-8, wherein a dose is administered about every four weeks (Q4W).
11. The method of any of embodiments 1-10, wherein the amount of the PD-1 inhibitor is 240 mg and a dose is administered about Q2W or the amount of the PD-1 inhibitor is 480 mg and a dose is administered about Q4W.
12. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising:
   (i) administration of a first amount of the PD-1 inhibitor of between at or about 140 mg and at or about 580 mg, inclusive, for a first cycle, wherein at least one dose of the first amount of the PD-1 inhibitor is administered in the first cycle and the first dose of the first cycle is administered between Day 2 and Day 20, inclusive; and
   (ii) administration of a second amount of the PD-1 inhibitor of between at or about 380 mg and at or about 580 mg, inclusive, about once every four weeks (Q4W) for a second cycle, wherein at least two doses of the second amount of the PD-1 inhibitor are administered in the second cycle and the first dose of the second cycle is administered between Day 50 and Day 65.
13. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
   (i) administration of a first amount of the PD-1 inhibitor of between at or about 140 and at or about 340 mg, inclusive, once every two weeks (Q2W) or once every four weeks (Q4W) for a first cycle, wherein at least two doses of the first amount of the PD-1 inhibitor are administered in the first cycle and the first dose of the first cycle is administered between Day 2 and Day 20, inclusive; and
   (ii) administration of a second amount of the PD-1 inhibitor of between at or about 140 mg and at or about 580 mg, inclusive, about once every four weeks (Q4W) for a second cycle, wherein at least two doses of the second amount are administered in the second cycle, and the first dose of the second cycle is administered between Day 50 and Day 65.
14. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
(i) administration of a first amount of a PD-1 inhibitor of between at or about 380 mg and at or about 580 mg, inclusive, for a first cycle, wherein at least one dose of the first amount of the PD-1 inhibitor is administered in the first cycle and the first dose of the first cycle is administered between Day 2 and Day 20; and
(ii) administration of a second amount of the PD-1 inhibitor of between at or about 380 mg and at or about 580 mg, inclusive, about once every four weeks (Q4W) for a second cycle, wherein at least two doses of the second amount are administered in the second cycle, and the first dose of the second cycle is administered between Day 50 and Day 65.
15. The method of any of embodiments 12-14, wherein the first dose of the first cycle is administered between Day 8 and Day 15, inclusive.
16. The method of any of embodiments 12-15, wherein the first dose of the first cycle is administered on Day 8.
17. The method of any of embodiments 12-15, wherein the dose of the first cycle is administered on Day 15.
18. The method of any of embodiments 12, 13, and 15-17, wherein the first amount of the PD-1 inhibitor is between at or about 160 mg and at or about 320 mg, inclusive.
19. The method of any of embodiments 12, 13, and 15-18, wherein the first amount of the PD-1 inhibitor is between at or about 200 mg and at or about 280 mg, inclusive.
20. The method of any of embodiments 12, 13, and 15-19, wherein the first amount of the PD-1 inhibitor is at or about 240 mg.
21. The method of embodiment 14, wherein the first amount of the PD-1 inhibitor is between at or about 400 mg and at or about 560 mg.
22. The method of embodiment 14 or embodiment 21, wherein the first amount of the PD-1 inhibitor is between at or about 440 mg and at or about 520 mg.
23. The method of any of embodiments 14, 21, or 22, wherein the first amount of the PD-1 inhibitor is at or about 480 mg.
24. The method of any of embodiments 12-23, wherein the second amount of the PD-1 inhibitor is between at or about 400 mg and at or about 560 mg, inclusive.
25. The method of any of embodiments 12-24, wherein the second amount of the PD-1 inhibitor is between at or about 440 mg and at or about 520 mg, inclusive.
26. The method of any of embodiments 12-25, wherein the second amount of the PD-1 inhibitor is at or about 480 mg.
27. The method of any of embodiments 12-26, wherein the first cycle is for at least about four weeks after administration of the first dose of the first cycle.
28. The method of any of embodiments 12-27, wherein the first cycle is for up to about five weeks after administration of the first dose of the first cycle.
29. The method of any of embodiments 12-27, wherein the first cycle is for up to about six weeks after administration of the first dose of the first cycle.
30. The method of any of embodiments 12-27, wherein the first cycle is for up to about seven weeks after administration of the first dose of the first cycle.
31. The method of any of embodiments 12-30, wherein the first cycle is for up to about eight weeks after administration of the dose of engineered T cells.
32. The method of any of embodiments 12, 13, 15, 16, 18-20, 24-27, 30, and 31, wherein doses of the first amount of the PD-1 inhibitor are administered on Days 8, 22 and 36.
33. The method of any of embodiments 12, 13, 15, 16, 21-27, 30, and 31, wherein doses of the first amount of the PD-1 inhibitor are administered on Days 8 and 36.
33. The method of any of embodiments 12, 13, 15, 17-20, 24-27, 29, and 31, wherein doses of the first amount of the PD-1 inhibitor are administered on Days 15, 29 and 43.
34. The method of any embodiments 14, 15, 17, 21-27, 29, and 31, wherein the at least one dose of the first amount of the PD-1 inhibitor is one dose that is administered on Day 15.
35. The method of any of embodiments 12-34, wherein the second cycle is for up to at least about three months after the administration of the dose of engineered T cells.
36. The method of any of embodiments 12-35, wherein the second cycle is for up to about three months after the administration of the dose of engineered T cells.
37. The method of any of embodiments 12-36, wherein the first and second dose of the second amount of the PD-1 inhibitor are administered on Days 57 and 85, respectively.
38. The method of any of embodiments 12, 13, 15, 16, 18-30, 32, 33, and 35-37, wherein doses of the first amount of the PD-1 inhibitor are administered on Days 8, 22, and 36; and the first and second dose of the second amount of the PD-1 inhibitor are administered on Days 57 and 85, respectively.
39. The method of any of embodiments 12, 13, 15, 16, 18-30, 33, and 35-37, wherein doses of the first amount of the PD-1 inhibitor are administered on Days 8 and 36; and the first and second dose of the second amount of the PD-1 inhibitor are administered on Days 57 and 85, respectively.
40. The method of any of embodiments 12, 13, 15, 17-29, 33, and 35-37, wherein doses of the first amount of the PD-1 inhibitor are administered on Days 15, 29, and 43; and the first and second dose of the second amount of the PD-1 inhibitor are administered on Days 57 and 85, respectively.
41. The method of any of embodiments 12, 14, 15, 17-29, and 34-37, wherein the at least one dose of the first amount of the PD-1 inhibitor is one dose that is administered on Day 15; and the first and second dose of the second amount of the PD-1 inhibitor are administered on Days 57 and 85, respectively.
42. The method of any of embodiments 1-41, further comprising administering a dose of a LAG3 inhibitor to the subject about every two weeks (Q2W).
43. The method of any of embodiments 1-41, further comprising administering a dose of a LAG3 inhibitor to the subject about every four weeks (Q4W).
44. The method of any of embodiments 1-43, further comprising administering a LAG3 inhibitor to the subject in a dosing regimen comprising administration of a dose of the LAG3 inhibitor on each of the same days on which a dose of the PD-1 inhibitor is administered.
45. The method of any of embodiments 42-44, wherein each dose of the LAG3 inhibitor is administered in an amount between at or about 160 mg and at or about 1040 mg.
46. The method of any of embodiments 42-45, wherein each dose of the LAG3 inhibitor is administered in an amount between at or about 160 mg and at or about 320 mg.
47. The method of any of embodiments 42-46, wherein each dose of the LAG3 inhibitor is administered in an amount at or about 240 mg.
48. The method of any of embodiments 42-45, wherein each dose of the LAG3 inhibitor is administered in an amount between at or about 400 mg and at or about 560 mg.
49. The method of any of embodiments 42-45 and 48, wherein each dose of the LAG3 inhibitor is administered in an amount at or about 480 mg.
50. The method of any of embodiments 42-45, wherein each dose of the LAG3 inhibitor is administered in an amount between at or about 880 mg and at or about 1040 mg.
51. The method of any of embodiments 42-45 and 50, wherein each dose of the LAG3 inhibitor is administered in an amount at or about 960 mg.
52. The method of any of embodiments 42-45, wherein each dose of the LAG3 inhibitor is administered in a first amount during the first cycle and in a second amount during the second cycle.
53. The method of embodiment 52, wherein the first amount of the LAG3 inhibitor is between at or about 160 mg and at or about 320 mg.
54. The method of embodiment 52 or embodiment 53, wherein the first amount of the LAG3 inhibitor is between at or about 200 mg and at or about 280 mg.
55. The method of any of embodiments 52-54, wherein the first amount of the LAG3 inhibitor is at or about 240 mg.
56. The method of embodiment 52, wherein the first amount of the LAG3 inhibitor is between at or about 400 mg and at or about 560 mg.
57. The method of embodiment 52 or embodiment 56, wherein the first amount of the LAG3 inhibitor is between at or about 440 mg and at or about 520 mg.
58. The method of any of embodiments 52, 56, and 57, wherein the first amount of the LAG3 inhibitor is at or about 480 mg.
59. The method of embodiment 52, wherein the first amount of the LAG3 inhibitor is between at or about 880 mg and at or about 1040 mg.
60. The method of embodiment 52 or embodiment 59, wherein the first amount of the LAG3 inhibitor is between at or about 920 mg and at or about 1000 mg.
61. The method of any of embodiments 52, 59, and 60, wherein the first amount of the LAG3 inhibitor is at or about 960 mg.
62. The method of any of embodiments 52-61, wherein the second amount of the LAG3 inhibitor is between at or about 400 mg and at or about 560 mg.
63. The method of any of embodiments 52-62, wherein the second amount of the LAG3 inhibitor is between at or about 440 mg and at or about 520 mg.
64. The method of any of embodiments 52-63, wherein the second amount of the LAG3 inhibitor is at or about 480 mg.
65. The method of any of embodiments 52-61, wherein the second amount of the LAG3 inhibitor is between at or about 880 mg and at or about 1040 mg.
66. The method of any of embodiments 52-61 and 65, wherein the second amount of the LAG3 inhibitor is between at or about 920 mg and at or about 1000 mg.
67. The method of any of embodiments 51-61, 65, and 66, wherein the second amount of the LAG3 inhibitor is at or about 960 mg.
68. The method of any of embodiments 52-55 and 62-64, wherein the first amount of the LAG3 inhibitor is at or about 240 mg, and the second amount of the LAG3 inhibitor is at or about 480 mg.
69. The method of any of embodiments 52, 56-58, and 62-64, wherein the first amount of the LAG3 inhibitor is at or about 480 mg, and the second amount of the LAG3 inhibitor is at or about 480 mg.
70. The method of any of embodiments 52, 56-58, and 65-67, wherein the first amount of the LAG3 inhibitor is at or about 480 mg, and the second amount of the LAG3 inhibitor is at or about 960 mg.
71. The method of any of embodiments 52, 59-61, and 65-67 wherein the first amount of the LAG3 inhibitor is at or about 960 mg, and the second amount of the LAG3 inhibitor is at or about 960 mg.
72. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising:
   (i) administration of a first amount of the PD-1 inhibitor, wherein the first amount is 240 mg and is administered on Days 8, 22, and 36; and
   (ii) administration of a second amount of the PD-1 inhibitor, wherein the second amount is 480 mg and is administered on Days 57 and 85; and
(3) administering to the subject a LAG3 inhibitor in a dosing regimen comprising:
   (i) administration of a first amount of the LAG3 inhibitor, wherein the first amount is 240 mg and is administered on Days 8, 22, and 36; and
   (ii) administration of a second amount of the LAG3 inhibitor, wherein the second amount is 480 mg and is administered on Days 57 and 85.
73. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising:
   (i) administration of a first amount of the PD-1 inhibitor, wherein the first amount is 240 mg and is administered on Days 8, 22, and 36; and
   (ii) administration of a second amount of the PD-1 inhibitor, wherein the second amount is 480 mg and is administered on Days 57 and 85; and
(3) administering to the subject a LAG3 inhibitor in a dosing regimen comprising:
   (i) administration of a first amount of the LAG3 inhibitor, wherein the first amount is 480 mg and is administered on Days 8, 22, and 36; and
   (ii) administration of a second amount of the LAG3 inhibitor, wherein the second amount is 960 mg and is administered on Days 57 and 85.
74. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
   (i) administration of a first amount of the PD-1 inhibitor, wherein the first amount is 240 mg and is administered on Days 8, 22, and 36; and
   (ii) administration of a second amount of the PD-1 inhibitor, wherein the second amount is 480 mg and is administered on Days 57 and 85.
75. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 8, 36, 57, and 85; and
(3) administering to the subject a LAG3 inhibitor in a dosing regimen comprising administration of 480 mg of the LAG3 inhibitor on Days 8, 36, 57, and 85.
76. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 8, 36, 57, and 85; and
(3) administering to the subject a LAG3 inhibitor in a dosing regimen comprising administration of 960 mg of the LAG3 inhibitor on Days 8, 36, 57, and 85.
77. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising administration of 480 mg of the PD-1 inhibitor on Days 8, 36, 57, and 85.
78. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising:
   (i) administration of a first amount of the PD-1 inhibitor, wherein the first amount is 240 mg and is administered on Days 15, 29, and 43; and
   (ii) administration of a second amount of the PD-1 inhibitor, wherein the second amount is 480 mg and is administered on Days 57 and 85; and
(3) administering to the subject a LAG3 inhibitor in a dosing regimen comprising:
   (i) administration of a first amount of the LAG3 inhibitor, wherein the first amount is 240 mg and is administered on Days 15, 29, and 43; and
   (ii) administration of a second amount of the LAG3 inhibitor, wherein the second amount is 480 mg and is administered on Days 57 and 85.
79. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising:
   (i) administration of a first amount of the PD-1 inhibitor, wherein the first amount is 240 mg and is administered on Days 15, 29, and 43; and
   (ii) administration of a second amount of the PD-1 inhibitor, wherein the second amount is 480 mg and is administered on Days 57 and 85; and
(3) administering to the subject a LAG3 inhibitor in a dosing regimen comprising:
   (i) administration of a first amount of the LAG3 inhibitor, wherein the first amount is 480 mg and is administered on Days 15, 29, and 43; and
   (ii) administration of a second amount of the LAG3 inhibitor, wherein the second amount is 960 mg and is administered on Days 57 and 85.
80. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
   (i) administration of a first amount of the PD-1 inhibitor, wherein the first amount is 240 mg and is administered on Days 15, 29, and 43; and
   (ii) administration of a second amount of the PD-1 inhibitor, wherein the second amount is 480 mg and is administered on Days 57 and 85.
81. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 15, 57, and 85; and
(3) administering to the subject a LAG3 inhibitor in a dosing regimen comprising administration of 480 mg of the LAG3 inhibitor on Days 15, 57, and 85.
82. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 15, 57, and 85; and
(3) administering to the subject a LAG3 inhibitor in a dosing regimen comprising administration of 960 mg of the LAG3 inhibitor on Days 15, 57, and 85.
83. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising administration of 480 mg of the PD-1 inhibitor on Days 15, 57, and 85.
84. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering a PD-1 inhibitor to the subject; and
(3) administering a LAG3 inhibitor to the subject.
85. A method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1;
(2) administering a PD-1 inhibitor to the subject; and
(3) administering a LAG3 inhibitor to the subject.
86. The method of embodiment 84 or embodiment 85, wherein a first dose of the PD-1 inhibitor and a first dose of the LAG3 inhibitor are independently administered, each between Day 2 and Day 20, inclusive.
87. The method of embodiment 86, wherein the first dose of the PD-1 inhibitor and the first dose of the LAG3 inhibitor are administered on the same day.
88. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
   (i) administration of a first dose of the PD-1 inhibitor is between Day 2 and Day 20, inclusive; and
   (ii) each dose of the PD-1 inhibitor is between at or about 140 mg and at or about 580 mg, inclusive.
89. A method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1; and
(2) administering a PD-1 inhibitor to the subject in a dosing regimen comprising administration of at least two doses, wherein:
   (i) administration of a first dose of the PD-1 inhibitor is between Day 2 and Day 20, inclusive; and
   (ii) each dose of the PD-1 inhibitor is between at or about 140 mg and at or about 580 mg, inclusive.
90. The method of any of embodiments 84-89, wherein each subsequent dose of the PD-1 inhibitor is administered about two weeks, about three weeks, or about four weeks after the previous dose of the PD-1 inhibitor.
91. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
   (i) administration of a first dose of the PD-1 inhibitor is between Day 2 and Day 20, inclusive; and
   (ii) each subsequent dose of the PD-1 inhibitor is administered about two weeks, about three weeks, or about four weeks after the previous dose of the PD-1 inhibitor.
92. A method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1; and
(2) administering a PD-1 inhibitor to the subject in a dosing regimen comprising administration of at least two doses, wherein:
   (i) administration of a first dose of the PD-1 inhibitor is between Day 2 and Day 20, inclusive; and
   (ii) each subsequent dose of the PD-1 inhibitor is administered about two weeks, about three weeks, or about four weeks after the previous dose of the PD-1 inhibitor.
93. The method of any of embodiments 84-87 and 90-92, wherein each dose of the PD-1 inhibitor is between at or about 140 mg and at or about 580 mg, inclusive.
94. The method of any of embodiments 1-93, wherein a first dose of the PD-1 inhibitor is administered between Day 8 and Day 15, inclusive.
95. The method of any of embodiments 84-94, wherein a first dose of the PD-1 inhibitor is administered on Day 8.
96. The method of any of embodiments 84-94, wherein a first dose of the PD-1 inhibitor is administered on Day 15.
97. The method of any of embodiments 84-96, wherein the PD-1 inhibitor is administered for no longer than about three months.
98. The method of any of embodiments 84-97, wherein a final dose of the PD-1 inhibitor is administered between about Day 80 and about Day 90, optionally wherein the final dose of the PD-1 inhibitor is administered at about Day 85.
99. The method of any of embodiments 84-98, wherein each dose of the PD-1 inhibitor is between at or about 160 mg and 560 mg.
100. The method of any of embodiments 84-99, wherein each dose of the PD-1 inhibitor is at or about 240 mg, or at or about 480 mg.
101. The method of any of embodiments 84-100, wherein at least one dose of the PD-1 inhibitor is 240 mg, and at least one dose of the PD-1 inhibitor is 480 mg.
102. The method of any of embodiments 84-101, wherein at least four doses of the PD-1 inhibitor are administered.
103. The method of any of embodiments 84-102, wherein four doses, five doses, or six doses of the PD-1 inhibitor are administered.
104. The method of embodiment 102 or embodiment 103, wherein the first three doses of the PD-1 inhibitor are administered every two weeks (Q2W).
105. The method of any of embodiments 104, wherein each dose of the PD-1 inhibitor is administered every two weeks (Q2W).
106. The method of any of embodiments 102-104, wherein the fourth dose of the PD-1 inhibitor is administered about three weeks or about four weeks after the previous dose of the PD-1 inhibitor.
107. The method of any of embodiments 102-104 and 106, wherein five doses of the PD-1 inhibitor are administered, and the fifth dose of the PD-1 inhibitor is administered about four weeks after the fourth dose of the PD-1 inhibitor.
108. The method of any of embodiments 84-107, wherein about 240 mg of the PD-1 inhibitor is administered on each of Days 8, 22, and 36.
109. The method of any of embodiments 84-107, wherein about 240 mg of the PD-1 inhibitor is administered on each of Days 15, 29, and 43.
110. The method of any of embodiments 84-107, wherein about 480 mg of the PD-1 inhibitor is administered on each of Days 8, 36, 64, and 85.
111. The method of any of embodiments 84-107, wherein about 480 mg of the PD-1 inhibitor is administered on each of Days 15, 43, 64, and 85.
112. The method of any of embodiments 87-111, further comprising administering a LAG3 inhibitor to the subject.
113. The method of embodiment 112, wherein a first dose of the LAG3 inhibitor is administered between Day 2 and Day 20, inclusive.
114. The method of any of embodiments 84-86 and 113, wherein a first dose of the LAG3 inhibitor is administered between Day 8 and Day 15, inclusive.
115. The method of any of embodiments 84-86, 113, and 114, wherein a first dose of the LAG3 inhibitor is administered on Day 8.
116. The method of embodiment 84-86, 113, and 114, wherein a first dose of the LAG3 inhibitor is administered on Day 15.
117. The method of any of embodiments 84-86 and 112-116, wherein each dose of the LAG3 inhibitor is between about 60 mg and about 540 mg, inclusive.
118. The method of any of embodiments 84-86 and 112-117, wherein each dose of the LAG3 inhibitor is between about 120 mg and about 480 mg.
119. The method of any of embodiments 84-86 and 112-118, wherein each dose of the LAG3 inhibitor is about 120 mg.
120. The method of any of embodiments 84-86 and 112-118, wherein each dose of the LAG3 inhibitor is about 240 mg.
121. The method of any of embodiments 84-86 and 112-118, wherein each dose of the LAG3 inhibitor is about 480 mg.
122. The method of any of embodiments 84-86 and 112-121, wherein at least three doses of the LAG3 inhibitor are administered.
123. The method of any of embodiments 84-86 and 112-122, where three doses, four doses, or six doses of the LAG3 inhibitor are administered.
124. The method of embodiment 122 or embodiment 123, wherein the first three doses of the LAG3 inhibitor are administered every two weeks (Q2W).
125. The method of any of embodiments 84-86 and 112-124, wherein each dose of the LAG3 inhibitor is administered every two weeks (Q2W).
126. The method of any of embodiments 122-124, wherein the second dose of the LAG3 inhibitor is administered about four weeks after the first dose of the LAG3 inhibitor.
127. The method of any of embodiments 84-86 and 112-126, wherein doses of the PD-1 inhibitor and doses of the LAG3 inhibitor are administered with the same frequency.
128. The method of any of embodiments 84-86 and 112-127, wherein (i) each dose of the PD-1 inhibitor is administered on the same day as a dose of the LAG3 inhibitor; and/or (ii) each dose of the LAG3 inhibitor is administered on the same day as a dose of the PD-1 inhibitor.
129. The method of any of embodiments 84-86 and 112-126, wherein doses of the LAG3 inhibitor are administered half as frequently as doses of the PD-1 inhibitor.
130. The method of any of embodiments 84-86 and 112-129, wherein each dose of the PD-1 inhibitor is double the dose of the LAG3 inhibitor.
131. The method of any of embodiments 84-86 and 112-129, wherein each dose of the PD-1 inhibitor is the same as the dose of the LAG3 inhibitor.
132. The method of any of embodiments 84-86, 112-128, 130, and 131, wherein the PD-1 inhibitor and the LAG3 inhibitor are formulated in a single composition, optionally for intravenous administration.
133. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 8, 22, 36, 57, 71, and 85; and
(3) administering to the subject a LAG3 inhibitor in a dosing regimen comprising administration of 240 mg of the LAG3 inhibitor on Days 8, 36, and 71.
134. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 15, 29, 43, 57, 71, and 85; and
(3) administering to the subject a LAG3 inhibitor in a dosing regimen comprising administration of 240 mg on Days 15, 43, and 71.
135. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 8, 22, 36, 57, 71, and 85; and
(3) administering to the subject a LAG3 inhibitor in a dosing regimen comprising administration of 240 mg of the LAG3 inhibitor on Days 8, 22, 36, 57, 71, and 85.
136. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 15, 29, 43, 57, 71, and 85; and
(3) administering to the subject a LAG3 inhibitor in a dosing regimen comprising administration of 240 mg of the LAG3 inhibitor on Days 815, 29, 43, 57, 71, and 85.
137. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
   (i) administration of a first amount of the PD-1 inhibitor, wherein the first amount is 240 mg and is administered on Days 8, 22, and 36; and
   (ii) administration of a second amount of the PD-1 inhibitor, wherein the second amount is 480 mg and is administered on Days 57 and 85.
138. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
   (i) administration of a first amount of the PD-1 inhibitor, wherein the first amount is 240 mg and is administered on Days 15, 29, and 43; and
   (ii) administration of a second amount of the PD-1 inhibitor, wherein the second amount is 480 mg and is administered on Days 57 and 85.
139. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 8, 22, 36, 57, 71, and 85; and
(3) administering to the subject a LAG3 inhibitor in a dosing regimen comprising administration of 120 mg of the LAG3 inhibitor on Days 8, 22, 36, 57, 71, and 85.
140. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 15, 29, 43, 57, 71, and 85; and
(3) administering to the subject a LAG3 inhibitor in a dosing regimen comprising administration of 120 mg of the LAG3 inhibitor on Days 15, 29, 43, 57, 71, and 85.
141. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 8, 36, 64, and 85; and
(3) administering to the subject a LAG3 inhibitor in a dosing regimen comprising administration of 240 mg of the LAG3 inhibitor on Days 8, 36, 64, and 85.
142. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 15, 43, 64, and 85; and
(3) administering to the subject a LAG3 inhibitor in a dosing regimen comprising administration of 240 mg of the LAG3 inhibitor on Days 15, 43, 64, and 85.
143. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 8, 36, 64, and 85; and
(3) administering to the subject a LAG3 inhibitor in a dosing regimen comprising administration of 480 mg of the LAG3 inhibitor on Days 8, 36, 64, and 85.
144. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 15, 43, 64, and 85; and
(3) administering to the subject a LAG3 inhibitor in a dosing regimen comprising administration of 480 mg of the LAG3 inhibitor on Days 15, 43, 64, and 85.
145. The method of any of embodiments 1-144, wherein the PD-1 inhibitor is an anti-PD-1 antibody.
146. The method of embodiment 145, wherein the anti-PD-1 antibody comprises a heavy chain variable (VH) region comprising a CDR1, a CDR2, and a CDR3 comprising the amino acid sequences set forth in SEQ ID NOS: 60, 61, and 62 respectively, and a light chain variable (VL) region comprising a CDR1, a CDR2, and a CDR3 comprising the amino acid sequences set forth in SEQ ID NOS: 63, 64, and 65 respectively.
147. The method of embodiment 146, wherein the VH region comprises the amino acid sequence set forth in SEQ ID NO: 66, and the VL region comprises the amino acid sequence set forth in SEQ ID NO: 67.
148. The method of any of embodiments 145-147, wherein the anti-PD-1 antibody is nivolumab.
149. The method of any of embodiments 1-148, wherein the LAG3 inhibitor is an anti-LAG3 antibody.
150. The method of embodiment 149, wherein the anti-LAG3 antibody comprises a heavy chain variable (VH) region comprising a CDR1, a CDR2, and a CDR3 comprising the amino acid sequences set forth in SEQ ID NOS: 68, 69, and 70 respectively, and a light chain variable (VL) region comprising a CDR1, a CDR2, and a CDR3 comprising the amino acid sequences set forth in SEQ ID NOS: 71, 72, and 73 respectively.
151. The method of embodiment 150, wherein the VH region comprises the amino acid sequence set forth in SEQ ID NO: 74, and the VL region comprises the amino acid sequence set forth in SEQ ID NO: 75.
152. The method of any of embodiments 149-151, wherein the anti-LAG3 antibody is relatlimab.
153. The method of any of embodiments 1-152, further comprising administering a lymphodepleting therapy to the subject prior to administration of the dose of engineered T cells.
154. The method of embodiment 153, wherein the lymphodepleting therapy is completed within about 7 days prior to initiation of the administration of the dose of engineered T cells.
155. The method of embodiment 153 or embodiment 154, wherein the administration of the lymphodepleting therapy is completed within about 2 to 7 days prior to initiation of the administration of the dose of engineered T cells.
156. The method of any of embodiments 153-155, wherein the lymphodepleting therapy comprises the administration of fludarabine and/or cyclophosphamide.
157. The method of any of embodiments 153-156, wherein the lymphodepleting therapy comprises administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days.
158. The method of any one of embodiments 153-157, wherein the lymphodepleting therapy comprises administration of cyclophosphamide at or about 300 mg/m² and fludarabine at or about 30 mg/m² daily concurrently for 3 days.
159. The method of any of embodiments 1-158, wherein CD19 is human CD19.
160. The method of any of embodiments 1-159, wherein the chimeric antigen receptor (CAR) comprises an scFv comprising the variable heavy chain region and the variable light chain region of the antibody FMC63, a spacer that is 15 amino acids or less and contains an immunoglobulin hinge region or a modified version thereof, a transmembrane domain, and an intracellular signaling domain comprising a signaling domain of a CD3-zeta (CD3ζ) chain and a costimulatory signaling region that is a signaling domain of 4-1BB.
161. The method of embodiment 160, wherein the immunoglobulin hinge region or a modified version thereof comprises the formula X₁PPX₂P, wherein X₁ is glycine, cysteine or arginine and X₂ is cysteine or threonine (SEQ ID NO:58).
162. The method of embodiment 160 or embodiment 161, wherein the immunoglobulin hinge region or a modified version thereof is an IgG1 hinge or a modified version thereof.
163. The method of embodiment 160 or embodiment 161, wherein the immunoglobulin hinge region or a modified version thereof is an IgG4 hinge or a modified version thereof.
164. The method of any of embodiments 160-163, wherein the spacer comprises the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.
165. The method of any of embodiments 160-164, wherein the spacer consists of the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.
166. The method of any of embodiments 160-165, wherein the spacer is at or about 12 amino acids in length.
167. The method of any of embodiments 160-166, wherein the spacer comprises the sequence set forth in SEQ ID NO: 1.
168. The method of any of embodiments 160-167, wherein the spacer consists of the sequence set forth in SEQ ID NO: 1.
169. The method of any of embodiments 160-168, wherein the transmembrane domain is a transmembrane domain of CD28.
170. The method of any of embodiments 160-169, wherein the transmembrane domain comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8.
171. The method of any of embodiments 160-170, wherein the costimulatory domain comprises the sequence set forth in SEQ ID NO: 12 or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 12.
172. The method of any of embodiments 160-171, wherein the signaling domain of a CD3-zeta (CD3ζ) chain comprises the sequence set forth in SEQ ID NO: 13, 14, or 15, or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 13, 14, or 15.
173. The method of any of embodiments 160-172, wherein the scFv comprises a CDRL1 sequence of SEQ ID NO: 35, a CDRL2 sequence of SEQ ID NO: 55, and a CDRL3 sequence of SEQ ID NO: 56; and a CDRH1 sequence of SEQ ID NO: 38, a CDRH2 sequence of SEQ ID NO: 39, and a CDRH3 sequence of SEQ ID NO: 54.
174. The method of any of embodiments 160-172, wherein the scFv comprises a CDRL1 sequence of SEQ ID NO: 35, a CDRL2 sequence of SEQ ID NO: 36, and a CDRL3 sequence of SEQ ID NO: 37; and a CDRH1 sequence of SEQ ID NO: 38, a CDRH2 sequence of SEQ ID NO: 39, and a CDRH3 sequence of SEQ ID NO: 40.
175. The method of any of embodiments 160-174, wherein the scFv comprises, in order from N-terminus to C-terminus, a V_{L} comprising the sequence set forth in SEQ ID NO: 42, and a V_{H}, comprising the sequence set forth in SEQ ID NO: 41.
176. The method of any of embodiments 160-175, wherein the scFv comprises the sequence set forth in SEQ ID NO: 43.
177. The method of any of embodiments 160-176, wherein the CAR contains in order from N-terminus to C-terminus: an extracellular antigen-binding domain that is the scFv set forth in SEQ ID NO: 43, the spacer set forth in SEQ ID NO: 1, the transmembrane domain set forth in SEQ ID NO: 8, the 4-1BB costimulatory signaling domain set forth in SEQ ID NO: 12, and the signaling domain of a CD3-zeta (CD3ζ) chain set forth in SEQ ID NO: 13.
178. The method of any of embodiments 1-177, wherein the dose of the engineered T cells comprises CD4+ T CAR-expressing cells and CD8+ CAR-expressing T cells.
179. The method of any of embodiments 1-178, wherein the dose of engineered T cells comprises between about 5×10⁷ CAR-expressing T cells and about 1.1×10⁸ CAR-expressing T cells, inclusive of each.
180. The method of any of embodiments 1-179, wherein the dose of engineered T cells comprises about 5×10⁷ CAR-expressing T cells.
181. The method of any of embodiments 1-179, wherein the dose of engineered T cells comprises about 1×10⁸ CAR-expressing T cells.
182. The method of any of embodiments 178-181, wherein administration of the dose of engineered T cells comprises administering a plurality of separate compositions, wherein the plurality of separate compositions comprises a first composition comprising the CD8+ CAR-expressing T cells and a second composition comprising the CD4+ CAR-expressing T cells.
183. The method of embodiment 182, wherein:
the first composition and the second composition are administered 0 to 12 hours apart, 0 to 6 hours apart, or 0 to 2 hours apart, or wherein the administration of the first composition and the administration of the second composition are carried out on the same day, between about 0 and about 12 hours apart, between about 0 and about 6 hours apart, or between about 0 and 2 hours apart; and/or
the initiation of administration of the first composition and the initiation of administration of the second composition are carried out between about 1 minute and about 1 hour apart or between about 5 minutes and about 30 minutes apart.
184. The method of embodiment 182 or embodiment 183, wherein the first composition and the second composition are administered no more than 2 hours, no more than 1 hour, no more than 30 minutes, no more than 15 minutes, no more than 10 minutes, or no more than 5 minutes apart.
185. The method of any of embodiments 182-184, wherein the first composition and the second composition are administered less than 2 hours apart.
186. The method of any of embodiments 182-185, wherein the first composition comprising the CD8⁺ CAR-expressing T cells is administered prior to the second composition comprising the CD4⁺ CAR-expressing T cells.
187. The method of any of embodiments 1-186, wherein the cells of the dose of the engineered T cells are administered intravenously.
188. The method of any of embodiments 1-187, wherein the T cells are primary T cells obtained from a sample from the subject, optionally wherein the sample is a whole blood sample, an apheresis sample, or a leukapheresis sample.
189. The method of embodiment 188, wherein the sample is obtained from the subject prior to administration of the lymphodepleting therapy to the subject.
190. The method of any of embodiments 1-189, wherein the T cells are autologous to the subject.
191. The method of any of embodiments 1-190, wherein the subject is human.
192. The method of any of embodiments 1-191, wherein the CD19-expressing cancer is a B cell malignancy.
193. The method of any of embodiments 1-192, wherein the CD19-expressing cancer is a myeloma, a leukemia, or a lymphoma.
194. The method of any of embodiments 1-193, wherein the CD19-expressing cancer is an acute lymphoblastic leukemia (ALL), adult ALL, chronic lymphoblastic leukemia (CLL), a small lymphocytic lymphoma (SLL), non-Hodgkin lymphoma (NHL), or a large B cell lymphoma.
195. The method of any of embodiments 1-194, wherein the CD19-expressing cancer is a non-Hodgkin lymphoma (NHL).
196. The method of any embodiment 194 or embodiment 195, wherein the NHL is selected from the group consisting of: diffuse large B-cell lymphoma (DLBCL) not otherwise specified (NOS) including transformed indolent NHL, follicular lymphoma Grade 3B (FL3B), T cell/histiocyte-rich large B-cell lymphoma, Epstein-Barr virus (EBV) positive DLBCL NOS, primary mediastinal (thymic) large B-cell lymphoma, Richter's Transformation and high grade B-cell lymphoma with MYC and BCL2 and/or BCL6 rearrangements with DLBCL histology (double/triple-hit lymphoma).
197. The method of any of embodiments 194-196, wherein the NHL is a relapsed/refractory (R/R) NHL.
198. The method of any of embodiments 194-197, wherein the subject is relapsed or refractory to at least two prior lines of systemic therapy for the CD19-expressing cancer.
199. The method of embodiment 198, wherein at least one of the at least two prior lines of systemic therapy includes a CD20-targeted agent and an anthracycline.
200. The method of any of embodiments 1-199, wherein the subject has an ECOG performance status of 0 or 1.
201. The method of any of embodiments 1-200, wherein the subject has positron-emission tomography (PET)-positive disease.
202. The method of any of embodiments 1-201, wherein the subject has computed tomography (CT) measurable disease.
203. The method of any of embodiments 1-202, wherein the subject has a sum of product of perpendicular diameters (SPD) of up to 6 index lesions of greater than or equal to 25 cm², optionally by CT scan.
204. Use of a PD-1 inhibitor and a LAG3 inhibitor in the manufacture of a medicament for treating a subject having a CD19-expressing cancer, wherein the subject was previously administered a cell therapy comprising a dose of engineered cells comprising T cells expressing a chimeric antigen receptor (CAR) that binds cluster of differentiation 19 (CD19) on Day 1.
205. The use of embodiment 204, wherein the medicament is to be administered to the subject between Day 2 and Day 20.
206. Use of a PD-1 inhibitor in the manufacture of a medicament for treating a subject having a CD19-expressing cancer, wherein:
(1) at least two doses of the medicament are to be administered to the subject;
(2) a first dose of the medicament is to be administered to the subject between Day 2 and Day 20, inclusive;
(3) each dose of the medicament comprises between at or about 140 mg and at or about 580 mg of the PD-1 inhibitor, inclusive; and
(4) the subject was previously administered a cell therapy comprising a dose of engineered cells comprising T cells expressing a chimeric antigen receptor (CAR) that binds cluster of differentiation 19 (CD19) on Day 1.
207. The use of any of embodiments 204-206, wherein each subsequent dose of the medicament is to be administered about two weeks, about three weeks, or about four weeks after the previous dose of the medicament.
208. Use of a PD-1 inhibitor in the manufacture of a medicament for treating a CD19-expressing cancer, wherein:
(1) at least two doses of the medicament are to be administered to the subject;
(2) a first dose of the medicament is to be administered to the subject between Day 2 and Day 20, inclusive;
(3) each subsequent dose of the medicament is to be administered about two weeks, about three weeks, or about four weeks after the previous dose of the medicament; and
(4) the subject was previously administered a cell therapy comprising a dose of engineered cells comprising T cells expressing a chimeric antigen receptor (CAR) that binds cluster of differentiation 19 (CD19) on Day 1.
209. The use of any of embodiments 204, 205, 207, and 208, wherein each dose of the medicament comprises between at or about 140 mg and at or about 580 mg of the PD-1 inhibitor, inclusive.
210. The use of any of embodiments 206-209, wherein the subject is administered a LAG3 inhibitor following administration of the cell therapy.
211. A combination of a PD-1 inhibitor and a LAG3 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering a PD-1 inhibitor to the subject; and
(3) administering a LAG3 inhibitor to the subject.
212. The combination for use of embodiment 211, wherein a first dose of the PD-1 inhibitor and a first dose of the LAG3 inhibitor are independently administered, each between Day 2 and Day 20.
213. The combination for use of embodiment 212, wherein the first dose of the PD-1 inhibitor and the first dose of the LAG3 inhibitor are administered on the same day.
214. A PD-1 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
   (i) administration of a first dose of the PD-1 inhibitor is between Day 2 and Day 20, inclusive; and
   (ii) each dose of the PD-1 inhibitor is between at or about 140 mg and at or about 580 mg, inclusive.
215. The combination for use of any of embodiments 211-213 or the PD-1 inhibitor for use of embodiment 214, wherein each subsequent dose of the PD-1 inhibitor is administered about two weeks, about three weeks, or about four weeks after the previous dose of the PD-1 inhibitor.
216. A PD-1 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
   (i) a first dose of the PD-1 inhibitor is administered between Day 2 and Day 20, inclusive; and
   (ii) each subsequent dose of the PD-1 inhibitor is administered about two weeks, about three weeks, or about four weeks after the previous dose of the PD-1 inhibitor.
217. The combination for use of any of embodiments 211-213 or the PD-1 inhibitor for use of embodiment 216, wherein each dose of the PD-1 inhibitor is between at or about 140 mg and at or about 580 mg, inclusive.
218. The PD-1 inhibitor for use of any of embodiments 214-217, wherein the method further comprises administering a LAG3 inhibitor to the subject.

### VI. Examples

The following examples are included for illustrative purposes only and are not intended to limit the scope of the invention.

### Example 1: Administration of Anti-CD19 CAR-Expressing Cells to Subjects with Relapsed/Refractory Non-Hodgkin Lymphoma

Therapeutic CAR T cell compositions containing autologous T cells expressing a chimeric antigen-receptor (CAR) specific for CD19 were administered to subjects with B-cell Non-Hodgkin lymphoma.

Specifically, autologous anti-CD19 CAR-expressing therapeutic T cell compositions for administration were generated by a process including immunoaffinity-based (e.g., immunomagnetic selection) enrichment of CD4⁺ and CD8⁺ cells from leukapheresis samples from the individual subjects to be treated, including subjects with diffuse large B-cell lymphoma (DLBCL) not otherwise specified (NOS; de novo or transformed follicular lymphoma (tFL)), high-grade B-cell lymphoma with MYC and BCL2 and/or BCL6 rearrangements with DLBCL histology (HGBL), primary mediastinal B-cell lymphoma (PMBCL), mantle cell lymphoma (MCL), follicular lymphoma Grade 3B (FL3B), and primary central nervous system lymphoma (PCNSL). Isolated CD4⁺ and CD8⁺ T cells were separately activated and independently transduced with a viral vector (e.g., lentiviral vector) encoding an anti-CD19 CAR, followed by separate expansion and cryopreservation of the engineered cell populations in a low-volume. The CAR contained an anti-CD19 scFv derived from a murine antibody (variable region derived from FMC63, V_{L}-linker-V_{H} orientation), an immunoglobulin-derived spacer, a transmembrane domain derived from CD28, a costimulatory region derived from 4-1BB, and a CD3-zeta intracellular signaling domain. The viral vector further contained sequences encoding a truncated receptor, which served as a surrogate marker for CAR expression; separated from the CAR sequence by a T2A ribosome skip sequence.

The CD4+ and CD8+ cryopreserved cell compositions were thawed prior to intravenous administration. The therapeutic T cell dose was administered as a defined cell composition by administering a formulated CD4⁺ CAR⁺ cell population and a formulated CD8⁺ CAR⁺ population administered at a target ratio of approximately 1:1.

Prior to CAR+ T cell infusion, subjects received a lymphodepleting chemotherapy with fludarabine (flu, 30 mg/m²/day) and cyclophosphamide (Cy, 300 mg/m²/day) for three (3) days.

The subjects received CAR-expressing T cells 2-7 days after lymphodepletion. Subjects were administered a single or double dose of CAR-expressing T cells (each single dose via separate infusions at a 1:1 ratio of CD4+ CAR-expressing T cells and CD8+ CAR-expressing T cells, respectively) as follows, and monitored for safety and efficacy: a single dose of dose level 1 (DL-1) containing 5×10⁷ total CAR-expressing T cells, a double dose of DL1 in which each dose was administered approximately fourteen (14) days apart, or a single dose of dose level 2 (DL-2) containing 1×10⁸ total CAR-expressing T cells. The dose and numbers of T cell subsets for the administered compositions are set forth in Table E1.

TABLE E1
Target Dose Levels and Number of T cell subsets for
cell compositions containing anti-CD19 CAR T cells

| | Helper T cell | Cytotoxic T Cell | |
|---|---|---|---|
| Dose | (T_{H}) Dose | (T_{C}) Dose | Total T Cell Dose |
| Level | (CD4⁺CAR⁺) | (CD8⁺CAR⁺) | (CD3⁺ CAR⁺) |
| 1 | 25 × 10⁶ | 25 × 10⁶ | 50 × 10⁶ |
| 2 | 50 × 10⁶ | 50 × 10⁶ | 100 × 10⁶ |

The expression of both programmed cell death protein 1 (PD-1) and lymphocyte activation gene 3 protein (LAG3) in T cells were assessed among the treated subjects after administering the dose of CAR-expressing T cells.

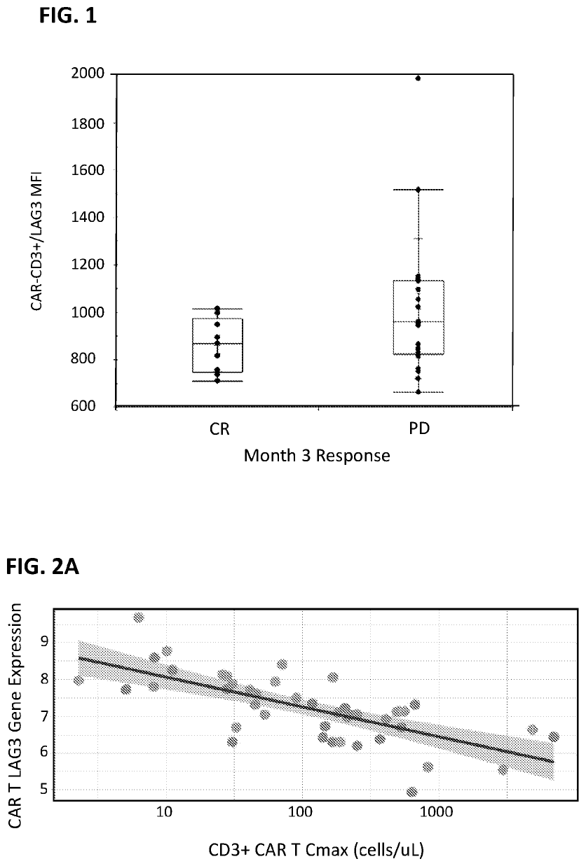

Expression of lymphocyte activation gene 3 (LAG3) protein by endogenous CD3+ T cells was analyzed in subjects administered the therapeutic compositions. Among subjects who went on to exhibit either complete response (CR) or progressive disease (PD) three months post-CAR T cell treatment, LAG3 protein expression by endogenous T cells (CD3+ T cells that are negative for the CAR, CAR-) at the time of peak expansion of the administered CAR T cells (Cₘₐₓ) tended to be higher in subjects who exhibited PD three months post-treatment, as assessed by flow cytometry of peripheral blood samples (FIG. 1).

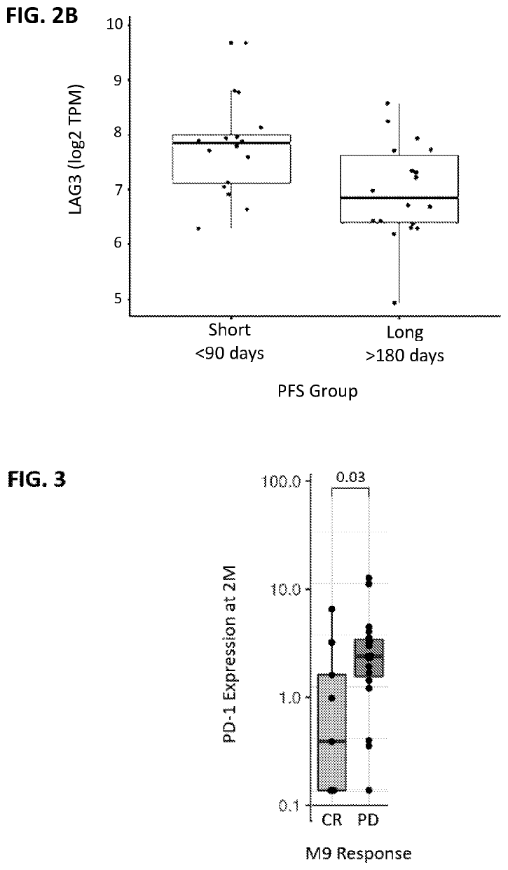

LAG3 gene expression by administered CAR T cells was also analyzed in subjects administered the therapeutic compositions. As shown in FIG. 2A, LAG3 gene expression by the CAR T cells was observed to inversely correlate with peak expansion of the CAR T cells (Cₘₐₓ). In addition, lower LAG3 gene expression by endogenous T cells and administered CAR T cells was observed in subjects with longer progression-free survival (PFS; >180 days), compared to subjects with shorter PFS (<90 days) (p=0.012; FIG. 2B).

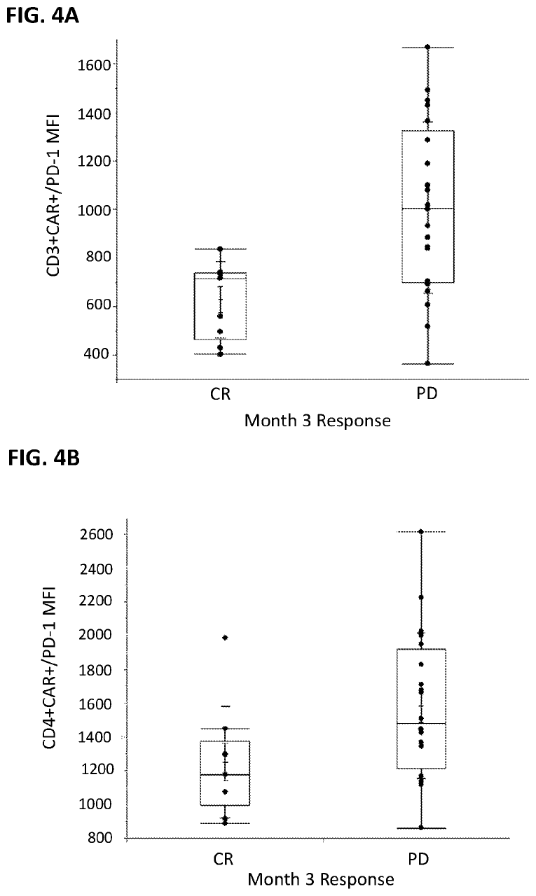

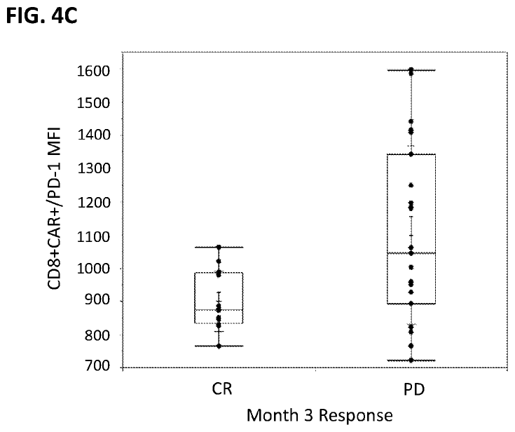

PBMC samples from patients were sorted by FACS into CAR+CD3+ T cells and CAR-CD3+ T cells, and PD-1 gene expression of the two cell populations was assessed by RNAseq. PD-1 expression by endogenous T cells (CD3+ CAR− T cells) was observed to be higher at two months post-CAR T cell treatment in patients who exhibited PD at nine months post-treatment, as compared to patents who exhibited CR at nine months post-treatment (FIG. 3). Among subjects exhibiting CR at three months post-CAR T cell treatment, PD-1 expression by the CAR T cells was found to be decreased at peak expansion of the cells (Cₘₐₓ) (p=0.004; FIG. 4A). Similar results were observed for both CD4+ CAR+ T cells (p=0.05; FIG. 4B) and CD8+ CAR+ T cells (p=0.03; FIG. 4C).

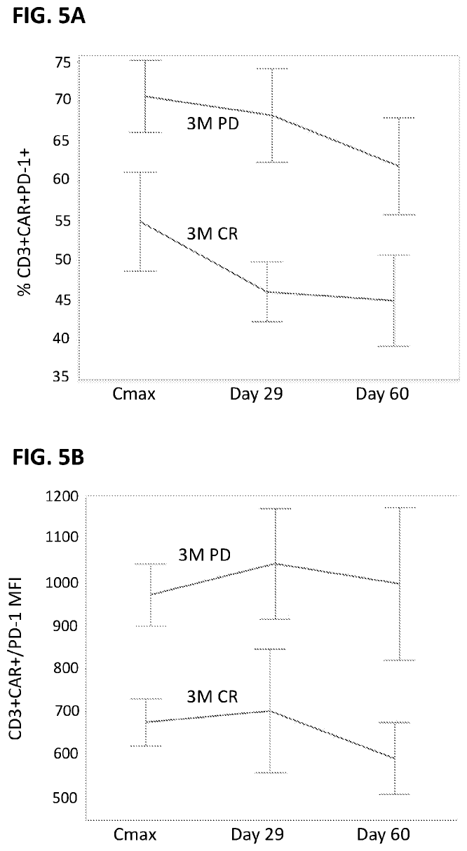

PD-1 protein expression was assessed by flow cytometry of peripheral blood samples. The association of CAR+PD-1+ cells to response to CAR T cell treatment was observed to persist over time, as assessed at Cₘₐₓ, 29 days post-treatment, and 60 days post-treatment, as assessed by either the percentage of CAR+PD-1+ cells (FIG. 5A) or the intensity of PD-1 expression on CAR+ cells (FIG. 5B).

Together, the results show that in subjects administered CAR T cells, exhaustion markers including PD-1 and LAG3 are upregulated on both the administered CAR T cells and on endogenous T cells in subjects that do not respond, or do not respond as well, to therapy. These results are consistent with an observation that higher LAG3 and PD-1 expression by endogenous T cells or administered CAR T cells may be associated with decreased CAR T expansion and a worse response to CAR T cell therapy. These data support that addition of one or both of LAG3 blockade and PD-1 blockade in combination with administration of CAR T cells may improve responses to CAR T cell therapy, such as by blocking inhibitory signals and preventing or restoring T cells from exhaustion.

### Example 2: Protocol for Treatment of Relapsed/Refractory Non-Hodgkin Lymphoma with CD19-Targeting CAR T Cells, Nivolumab, and Relatlimab

Therapeutic CAR T cell compositions containing autologous T cells expressing a chimeric antigen-receptor (CAR) specific for CD19 are administered as a combination therapy with nivolumab and/or relatlimab to subjects with relapsed/refractory (R/R) aggressive B-cell Non-Hodgkin lymphoma (NHL).

Prior to CAR⁺ T cell infusion, leukapheresis samples for the enrichment of the autologous CD4+ and CD8+ T cells are obtained from subjects. In some cases, the leukapheresis samples are obtained from subjects about 28 days prior to administration of the CAR T cells.

Specifically, autologous anti-CD19 directed therapeutic T cell compositions are generated by a process including immunoaffinity-based (e.g., immunomagnetic selection) enrichment of CD4⁺ and CD8⁺ cells from leukapheresis samples from adult human subjects (e.g. ≥18 years-old) with relapsed/refractory (R/R) aggressive NHL. Subjects have histologically confirmed aggressive B-cell NHL, including diffuse large B-cell lymphoma (DLBCL) not otherwise specified (NOS), including transformed indolent NHL; follicular lymphoma Grade 3B (FL 3B); T cell/histiocyte-rich large B-cell lymphoma; Epstein-Barr virus (EBV) positive DLBCL, NOS; primary mediastinal (thymic) large B-cell lymphoma; Richter's Transformation and high grade B-cell lymphoma with MYC and BCL2 and/or BCL6 rearrangements with DLBCL histology (double/triple-hit lymphoma; DHL/THL). All subjects exhibit an Eastern Cooperative Oncology Group (ECOG) performance status of 0 or 1; positron-emission tomography (PET)-positive (e.g. Deauville score of 4 or 5) and computed tomography (CT) measurable disease (e.g. per Lugano classification; Cheson et al., (2014) J. Clin. Oncol. 32(27); 3059-68); and a sum of product of perpendicular diameters (SPD) of up to 6 index lesions of greater than or equal to 25 cm² (e.g. by CT scan). Subjects with Richter's Transformation are not required to exhibit a sum of product of perpendicular diameters (SPD) of up to 6 index lesions of greater than or equal to 25 cm². Subjects also have relapsed or been refractory to at least two prior lines of systemic therapy for the disease, including a CD20-targeted agent and an anthracycline.

T cell compositions enriched for CD4⁺ and/or CD8⁺ cells from leukapheresis samples from individual subjects to be treated are administered to the subjects. The isolated compositions enriched for CD4⁺ and/or CD8⁺ T cells are separately activated with anti-CD3/anti-CD28 antibody reagents and independently transduced with a viral vector (e.g., lentiviral vector) encoding an anti-CD19 CAR, followed by separate expansion and cryopreservation of each of the engineered CD4+ and CD8+ cell populations. The CAR contains an anti-CD19 scFv derived from a murine antibody (variable region derived from FMC63, V_{L}-linker-V_{H} orientation), an immunoglobulin-derived spacer linking the antigen-binding domain to a transmembrane domain, a transmembrane domain derived from human CD28), a costimulatory region derived from human 4-1BB, and a human CD3-zeta intracellular signaling domain. The viral vector further contains sequences encoding a truncated receptor, which is separated from the CAR sequence by a T2A ribosome skip sequence and serves as a surrogate marker for CAR expression.

The cryopreserved cell compositions are separately thawed prior to intravenous administration. Subjects are administered a dose of 1×10⁸ total CAR-expressing T cells (e.g. via separate infusions of CD4⁺ CAR-expressing T cells and CD8⁺ CAR-expressing T cells provided at approximately a 1:1 ratio of CD4+ to CD8+ cells).

Subjects receive a lymphodepleting chemotherapy with fludarabine (Flu, 30 mg/m²) and cyclophosphamide (Cy, 300 mg/m²) for 3 days. The subjects receive the dose of CAR-expressing T cells (e.g. 1×10⁸ total CAR-expressing T cells) 2-7 days after lymphodepletion (e.g. 5-7 days after lymphodepletion), on Day 1. Optionally, subjects may receive bridging therapy between the leukapheresis sample being obtained and the lymphodepleting therapy being administered.

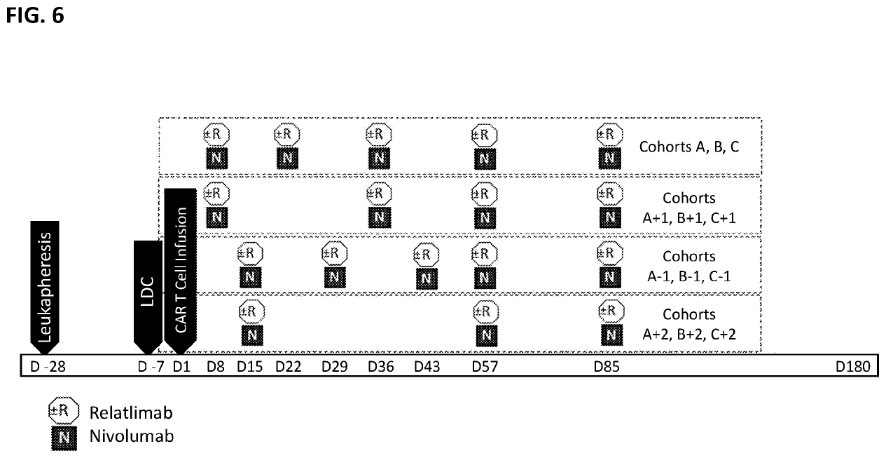

Subjects are assigned to a dosing cohort, selected from among A, B, C, A+1, B+1, C+1, A-1, B-1, C-1, A+2, B+2, and C+2 (FIG. 6 and Table E2).

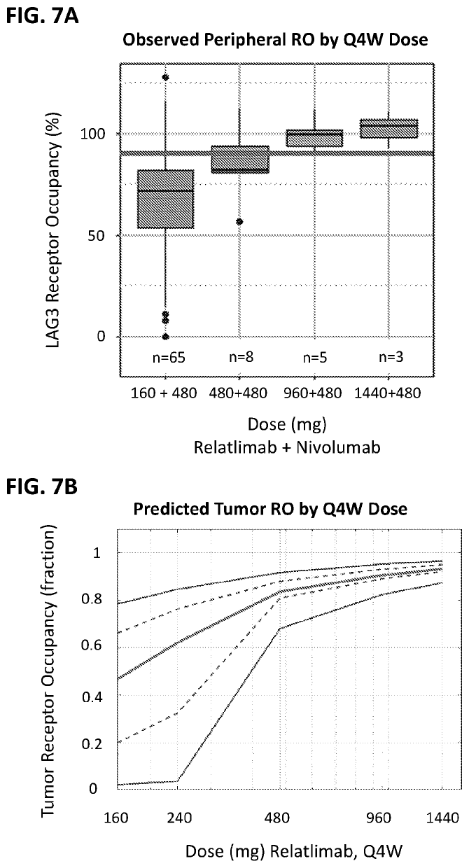

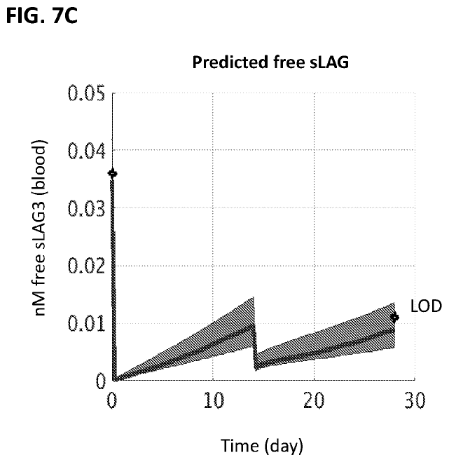

Dosing cohorts were designed based on analysis on LAG3 receptor occupancy and free soluble LAG3. In particular, dosing cohorts were designed to achieve saturation of peripheral blood and tumoral LAG3 receptor occupancy, and to minimize the free soluble LAG3 in blood. As shown in FIGS. 7A-C, these aspects were found or predicted to be optimized with higher doses of relatlimab, such as 960 mg relatlimab every four weeks (Q4W). Dosing cohorts were therefore designed to reach doses of 480 mg of nivolumab and 960 mg of relatlimab.

Cohort A subjects receive 240 mg nivolumab and 240 mg relatlimab on days 8, 22, and 36; and 480 mg nivolumab and 480 mg relatlimab on days 57 and 85. Cohort B subjects receive 240 mg nivolumab and 280 mg relatlimab on days 8, 22, and 36; and 480 mg nivolumab and 960 mg relatlimab on days 57 and 85. Cohort C subjects receive 240 mg nivolumab on days 8, 22, and 36; and 480 mg nivolumab on days 57 and 85 (no relatlimab is administered).

Cohort A+1 subjects receive 480 mg nivolumab and 480 mg relatlimab on days 8 and 36; and 480 mg nivolumab and 480 mg relatlimab on days 57 and 85. Cohort B+1 subjects receive 480 mg nivolumab and 960 mg relatlimab on days 8 and 36; and 480 mg nivolumab and 960 mg relatlimab on days 57 and 85. Cohort C+1 subjects receive 480 mg nivolumab on days 8 and 36; and 480 mg nivolumab on days 57 and 85 (no relatlimab is administered).

Cohort A-1 subjects receive 240 mg nivolumab and 240 mg relatlimab on days 15, 29, and 43; and 480 mg nivolumab and 480 mg relatlimab on days 57 and 85. Cohort B-1 subjects receive 240 mg nivolumab and 480 mg relatlimab on days 15, 29, and 43; and 480 mg nivolumab and 960 mg relatlimab on days 57 and 85. Cohort C-1 subjects receive 240 mg nivolumab on days 15, 29, and 43; and 480 mg nivolumab on days 57 and 85 (no relatlimab is administered).

Cohort A+2 subjects receive 480 mg nivolumab and 480 mg relatlimab on day 15; and 480 mg nivolumab and 480 mg relatlimab on days 57 and 85. Cohort B+2 subjects receive 480 mg nivolumab and 960 mg relatlimab on day 15; and 480 mg nivolumab and 960 mg relatlimab on days 57 and 85. Cohort C+2 subjects receive 480 mg nivolumab on day 15; and 480 mg nivolumab on days 57 and 85 (no relatlimab is administered).

Two subjects were enrolled in Cohort C and were treated with nivolumab only, as shown in Table E2 below.

TABLE E2
CAR T Cell and Checkpoint Inhibitor Combination Therapy Cohorts

| | | Nivolumab ± | Before Day 57 | | Days 57 and 85 | |
|---|---|---|---|---|---|---|
| | CAR T cell | Relatlimab | Nivolumab | Relatlimab | Nivolumab | Relatlimab |
| Cohort | dose | Schedule | Dose | Dose | Dose | Dose |
| A | 100 × 10⁶ | Days 8, 22, | 240 mg | 240 mg | 480 mg | 480 mg |
| B | CAR + | 36, 57, and 85 | | 480 mg | | 960 mg |
| C | T cells | | | Not given | | Not given |
| A + 1 | 100 × 10⁶ | Days 8, 36, | 480 mg | 480 mg | 480 mg | 480 mg |
| B + 1 | CAR T cells | 57, and 85 | | 960 mg | | 960 mg |
| C + 1 | | | | Not given | | Not given |
| A − 1 | 100 × 10⁶ | Days 15, 29, | 240 mg | 240 mg | 480 mg | 480 mg |
| B − 1 | CAR T cells | 43, 57, and 85 | | 480 mg | | 960 mg |
| C − 1 | | | | Not given | | Not given |
| A + 2 | 100 × 10⁶ | Days 15, 57, | 480 mg | 480 mg | 480 mg | 480 mg |
| B + 2 | CAR T cells | and 85 | | 960 mg | | 960 mg |
| C + 2 | | | | Not given | | Not given |

Subjects are monitored for response (e.g. complete response rate; CRR) and rates of dose-limiting toxicity (DLT). Other endpoints may include health-related quality of life (HRQoL); safety (e.g. adverse events; AEs); efficacy, including progression-free survival (PFS), overall survival (OS), overall response rate (ORR), duration of response (DOR), and event-free survival (EFS); pharmacokinetics (PK, e.g. Cₘₐₓ, Tₘₐₓ, and AUC); and pharmacodynamics (PD), which may be monitored for up to approximately 24 months or more (e.g. up to 42 months) post-CAR T cell infusion. Response may be assessed, such as by positron emission tomography (PET)-computed tomography (CT) and/or magnetic resonance imaging (MRI), at approximately 1, 3, 6, 9, 12, 18, and 24 months following CAR T cell treatment.

### Example 3: Alternative Protocol for Treatment of Relapsed/Refractory Non-Hodgkin Lymphoma with CD19-Targeting CAR T Cells, Nivolumab, and Relatlimab

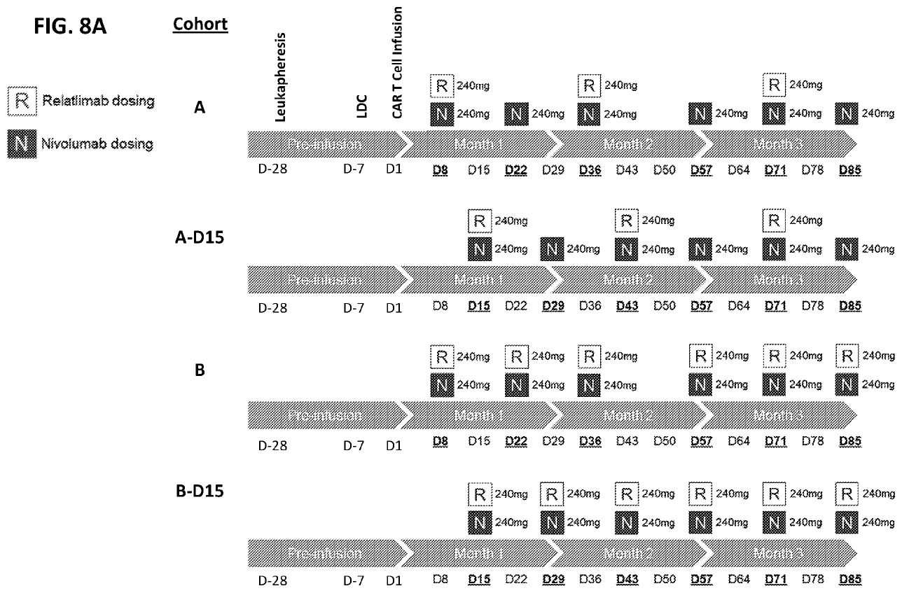

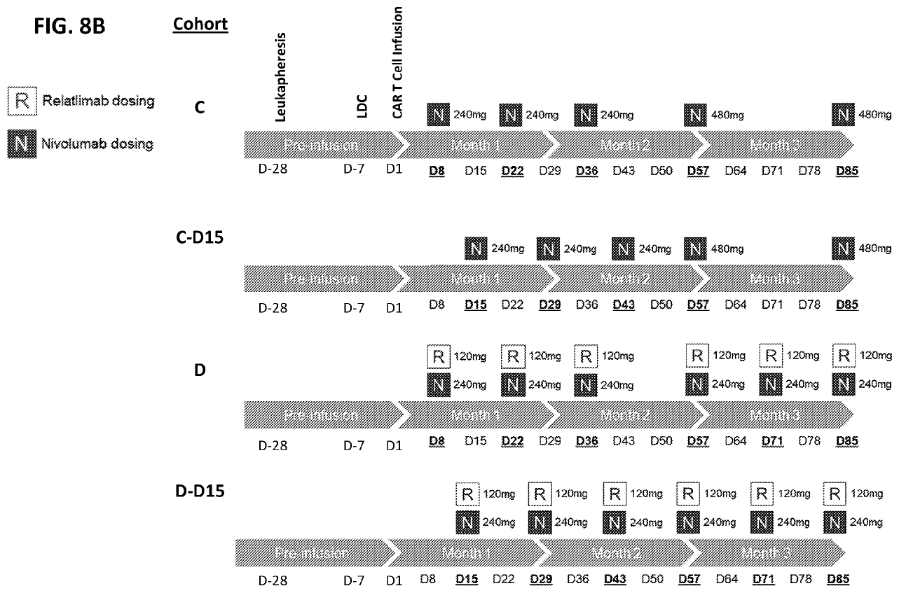

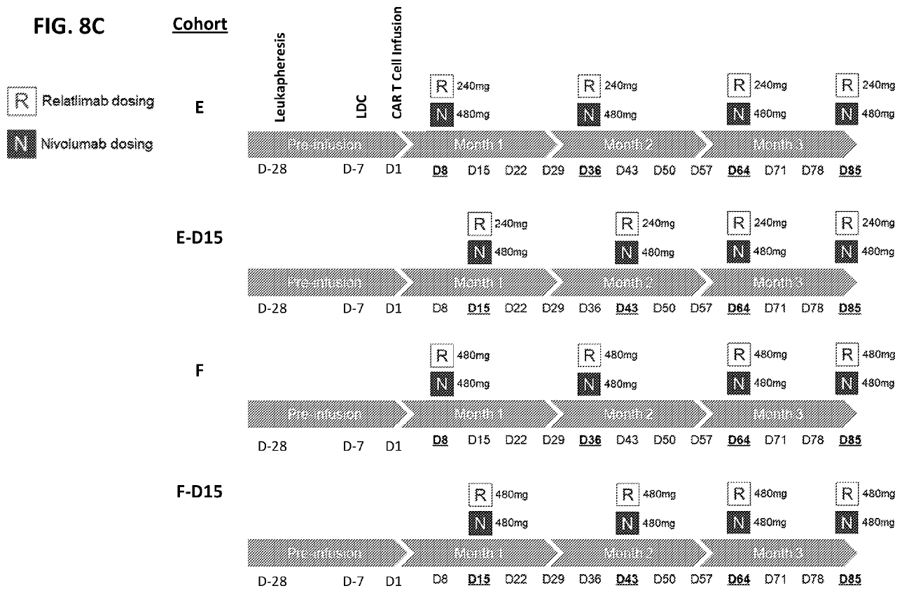

Therapeutic CAR T cell compositions containing autologous T cells expressing a chimeric antigen-receptor (CAR) specific for CD19 are administered as a combination therapy with nivolumab and/or relatlimab to subjects with relapsed/refractory (R/R) aggressive B-cell Non-Hodgkin lymphoma (NHL) as in Example 2, except that subjects are assigned to alternative dosing cohorts. In particular, the subjects are assigned to dosing cohorts selected from among A, B, C, D, E, F, A-D15, B-D15, C-D15, D-D15, E-D15, and F-D15 (FIGS. 8A-C and Table E3).

Cohort A subjects receive a 1:2 ratio of relatlimab to nivolumab, with relatlimab dosed half as frequently as nivolumab. Specifically, 240 mg nivolumab is provided on days 8, 22, 36, 57, 71, and 85; and 240 mg relatlimab is provided on days 8, 36, and 71.

Cohort A-D15 subjects receive a 1:2 ratio of relatlimab to nivolumab, with relatlimab dosed half as frequently as nivolumab. Specifically, 240 mg nivolumab is provided on days 15, 29, 43, 57, 71, and 85; and 240 mg relatlimab is provided on days 15, 43, and 71.

Cohort B subjects receive a 1:1 ratio of relatlimab to nivolumab, with relatlimab dosed with the same frequency as nivolumab. Specifically, 240 mg nivolumab is provided on days 8, 22, 36, 57, 71, and 85; and 240 mg relatlimab is provided on days 8, 22, 36, 57, 71, and 85.

Cohort B-D15 subjects receive a 1:1 ratio of relatlimab to nivolumab, with relatlimab dosed with the same frequency as nivolumab. Specifically, 240 mg nivolumab is provided on days 15, 29, 43, 57, 71, and 85; and 240 mg relatlimab is provided on days 15, 29, 43, 57, 71, and 85.

Cohort C subjects receive nivolumab without relatlimab. Specifically, 240 mg nivolumab is provided on days 8, 22, and 36; and 480 mg nivolumab is provided on days 57 and 85.

Cohort C-D15 subjects receive nivolumab without relatlimab. Specifically, 240 mg nivolumab is provided on days 15, 29, and 43; and 480 mg nivolumab is provided on days 57 and 85.

Cohort D subjects receive a 1:2 ratio of relatlimab to nivolumab, with relatlimab dosed with the same frequency as nivolumab. Specifically, 240 mg nivolumab is provided on days 8, 22, 36, 57, 71, and 85; and 120 mg relatlimab is provided on days 8, 22, 36, 57, 71, and 85.

Cohort D-D15 subjects receive a 1:2 ratio of relatlimab to nivolumab, with relatlimab dosed with the same frequency as nivolumab. Specifically, 240 mg nivolumab is provided on days 15, 29, 43, 57, 71, and 85; and 120 mg relatlimab is provided on days 15, 29, 43, 57, 71, and 85.

Cohort E subjects receive a 1:2 ratio of relatlimab to nivolumab, with relatlimab dosed with the same frequency as nivolumab. Specifically, 480 mg nivolumab is provided on days 8, 36, 64, and 85; and 240 mg relatlimab is provided on days 8, 36, 64, and 85.

Cohort E-D15 subjects receive a 1:2 ratio of relatlimab to nivolumab, with relatlimab dosed with the same frequency as nivolumab. Specifically, 480 mg nivolumab is provided on days 15, 43, 64, and 85; and 240 mg relatlimab is provided on days 15, 43, 64, and 85.

Cohort F subjects receive a 1:1 ratio of relatlimab to nivolumab, with relatlimab dosed with the same frequency as nivolumab. Specifically, 480 mg nivolumab is provided on days 8, 36, 64, and 85; and 480 mg relatlimab is provided on days 8, 36, 64, and 85.

Cohort F-D15 subjects receive a 1:1 ratio of relatlimab to nivolumab, with relatlimab dosed with the same frequency as nivolumab. Specifically, 480 mg nivolumab is provided on days 15, 43, 64, and 85; and 480 mg relatlimab is provided on days 15, 43, 64, and 85.

TABLE E3
CAR T Cell and Checkpoint Inhibitor Combination Therapy Cohorts

| | CAR T | Nivolumab | | Nivolumab | Relatlimab |
|---|---|---|---|---|---|
| Cohort | cell dose | Schedule | Relatlimab Schedule | Dose | Dose |
| A | 100 × | Day 8, 22, 36, 57, 71, | Day 8, 36, 71 | 240 mg | 240 mg |
| | 10⁶ | 85 | | | |
| A-D15 | CAR + | Day 15, 29, 43, 57, 71, | Day 15, 43, 71 | | |
| | T cells | 85 | | | |
| B | | Day 8, 22, 36, 57, 71, | Day 8, 22, 36, 57, 71, 85 | 240 mg | 240 mg |
| | | 85 | | | |
| B-D15 | | Day 15, 29, 43, 57, 71, | Day 15, 29, 43, 57, 71, | | |
| | | 85 | 85 | | |
| C | | Day 8, 22, 36, 57^{a}, 85^{a} | Not given | 240 or | Not given |
| C-D15 | | Day 15, 29, 43, 57^{a}, 85^{a} | | 480 mg^{a} | |
| D | | Day 8, 22, 36, 57, 71, | Day 8, 22, 36, 57, 71, 85 | 240 mg | 120 mg |
| | | 85 | | | |
| D-D15 | | Day 15, 29, 43, 57, 71, | Day 15, 29, 43, 57, 71, | | |
| | | 85 | 85 | | |
| E | | Day 8, 36, 64, 85 | Day 8, 36, 64, 85 | 480 mg | 240 mg |
| E-D15 | | Day 15, 43, 64, 85 | Day 15, 43, 64, 85 | | |
| F | | Day 8, 36, 64, 85 | Day 8, 36, 64, 85 | 480 mg | 480 mg |
| F-D15 | | Day 15, 43, 64, 85 | Day 15, 43, 64, 85 | | |

^{a}Dose for cohort C and C-D15 at Day 57 and 84 is doubled to 480 mg
In case of Grade ≥ 3 CRS or NT, nivolumab and relatlimab must be delayed until resolution to ≤ Grade 1
Abbreviations:
CAR = Chimeric Antigen Receptor;
CRS = Cytokine Release Syndrome;
NT = Neurotoxicity

Subjects are monitored for response (e.g. complete response rate; CRR) and rates of dose-limiting toxicity (DLT). Other endpoints may include health-related quality of life (HRQoL); safety (e.g. adverse events; AEs); efficacy, including progression-free survival (PFS), overall survival (OS), overall response rate (ORR), duration of response (DOR), and event-free survival (EFS); pharmacokinetics (PK, e.g. Cₘₐₓ, Tₘₐₓ, and AUC); and pharmacodynamics (PD), which may be monitored for up to approximately 24 months or more (e.g. up to 42 months) post-CAR T cell infusion. Response may be assessed, such as by positron emission tomography (PET)-computed tomography (CT) and/or magnetic resonance imaging (MRI), at approximately 1, 3, 6, 9, 12, 18, and 24 months following CAR T cell treatment.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer (IgG4hinge) |
| | | (aa) |
| 2 | GAATCTAAGTACGGACCGCCCTGCCCCCCTTGCCCT | spacer (IgG4hinge) |
| | | (nt) |
| 3 | ESKYGPPCPPCPGQPREPQVYTLPPSQEEMTKNQVSLTCLVKGFYPSD | Hinge-CH3 spacer |
| | IAVEWESNGQPENNYKTTPPVLDSDGSFFLYSRLTVDKSRWQEGNVFS | |
| | CSVMHEALHNHYTQKSLSLSLGK | |
| 4 | ESKYGPPCPPCPAPEFLGGPSVFLFPPKPKDTLMISRTPEVTCVVVDV | Hinge-CH2-CH3 |
| | SQEDPEVQFNWYVDGVEVHNAKTKPREEQFNSTYRVVSVLTVLHQDWL | spacer |
| | NGKEYKCKVSNKGLPSSIEKTISKAKGQPREPQVYTLPPSQEEMTKNQ | |
| | VSLTCLVKGFYPSDIAVEWESNGQPENNYKTTPPVLDSDGSFFLYSRL | |
| | TVDKSRWQEGNVFSCSVMHEALHNHYTQKSLSLSLGK | |
| 5 | RWPESPKAQASSVPTAQPQAEGSLAKATTAPATTRNTGRGGEEKKKEK | IgD-hinge-Fc |
| | EKEEQEERETKTPECPSHTQPLGVYLLTPAVQDLWLRDKATFTCFVVG | |
| | SDLKDAHLTWEVAGKVPTGGVEEGLLERHSNGSQSQHSRLTLPRSLWN | |
| | AGTSVTCTLNHPSLPPQRLMALREPAAQAPVKLSLNLLASSDPPEAAS | |
| | WLLCEVSGFSPPNILLMWLEDQREVNTSGFAPARPPPQPGSTTFWAWS | |
| | VLRVPAPPSPQPATYTCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGELLI | |
| | QAWPENRTDLHAFENLEIIRGRTKQHGQFSLAVVSLNITSLGLRSLKE | |
| | ISDGDVIISGNKNLCYANTINWKKLFGTSGQKTKIISNRGENSCKATG | |
| | QVCHALCSPEGCWGPEPRDCVSCRNVSRGRECVDKCNLLEGEPREFVE | |
| | NSECIQCHPECLPQAMNITCTGRGPDNCIQCAHYIDGPHCVKTCPAGV | |
| | MGENNTLVWKYADAGHVCHLCHPNCTYGCTGPGLEGCPTNGPKIPSIA | |
| | TGMVGALLLLLVVALGIGLFM | |
| 8 | FWVLVVVGGVLACYSLLVTVAFIIFWV | CD28 (amino acids |
| | | 153-179 of |
| | | Accession No. |
| | | P10747) |
| 9 | IEVMYPPPYLDNEKSNGTIIHVKGKHLCPSPLFPGPSKP | CD28 (amino acids |
| | FWVLVVVGGVLACYSLLVTVAFIIFWV | 114-179 of |
| | | Accession No. |
| | | P10747) |
| 10 | RSKRSRLLHSDYMNMTPRRPGPTRKHYQPYAPPRDFAAYRS | CD28 (amino acids |
| | | 180-220 of |
| | | Accession No. |
| | | P10747) |
| 11 | RSKRSRGGHSDYMNMTPRRPGPTRKHYQPYAPPRDFAAYRS | CD28 (LL to GG) |
| 12 | KRGRKKLLYIFKQPFMRPVQTTQEEDGCSCRFPEEEEGGCEL | 4-1BB (amino |
| | | acids 214-255 of |
| | | Accession No. |
| | | Q07011.1) |
| 13 | RVKFSRSADAPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 14 | RVKFSRSAEPPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 15 | RVKFSRSADAPAYKQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 16 | RKVCNGIGIGEFKDSLSINATNIKHFKNCTSISGDLHILPVAFRGDSF | tEGFR |
| | THTPPLDPQELDILKTVKEITGFLLIQAWPENRTDLHAFENLEIIRGR | |
| | TKQHGQFSLAVVSLNITSLGLRSLKEISDGDVIISGNKNLCYANTINW | |
| | KKLFGTSGQKTKIISNRGENSCKATGQVCHALCSPEGCWGPEPRDCVS | |
| | CRNVSRGRECVDKCNLLEGEPREFVENSECIQCHPECLPQAMNITCTG | |
| | RGPDNCIQCAHYIDGPHCVKTCPAGVMGENNTLVWKYADAGHVCHLCH | |
| | PNCTYGCTGPGLEGCPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)₅-P- wherein P is proline, G is | Linker |
| | glycine and S is serine | |
| 23 | GSADDAKKDAAKKDGKS | Linker |
| 24 | atgcttctcctggtgacaagccttctgctctgtgagttaccacaccca | GMCSFR alpha |
| | gcattcctcctgatccca | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha signal |
| | | peptide |
| 27 | Glu Pro Lys Ser Cys Asp Lys Thr His Thr Cys Pro | Hinge |
| | Pro Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEP | Hinge |
| | KSCDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro | Hinge |
| | Cys Pro | |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWL | VH |
| | GVIWGSETTYYNSALKSRLTIIKDNSKSQVFLKMNSLQTDDTAIYYCA | |
| | KHYYYGGSYAMDYWGQGTSVTVSS | |
| 42 | DIQMTQTTSSLSASLGDRVTISCRASQDISKYLNWYQQKPDGTVKLLI | VL |
| | YHTSRLHSGVPSRFSGSGSGTDYSLTISNLEQEDIATYFCQQGNTLPY | |
| | TFGGGTKLEIT | |
| 43 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | |
| | TFGGGTKLEITGSTSGSGKPGSGEGSTKGEVKLQESGPGLVAPSQSLS | |
| | VTCTVSGVSLPDYGVSWIRQPPRKGLEWLGVIWGSETTYYNSALKSRL | |
| | TIIKDNSKSQVFLKMNSLQTDDTAIYYCAKHYYYGGSYAMDYWGQGTS | |
| | VTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQGLEWI | VH |
| | GQIYPGDGDTNYNGKFKGQATLTADKSSSTAYMQLSGLTSEDSAVYFC | |
| | ARKTISSVVDFYFDYWGQGTTVTVSS | |
| 51 | DIELTQSPKFMSTSVGDRVSVTCKASQNVGTNVAWYQQKPGQSPKPLI | VL |
| | YSATYRNSGVPDRFTGSGSGTDFTLTITNVQSKDLADYFCQQYNRYPY | |
| | TSGGGTKLEIKR | |
| 52 | GGGGSGGGGSGGGGS | Linker |
| 53 | EVKLQQSGAELVRPGSSVKISCKASGYAFSSYWMNWVKQRPGQGLEWI | scFv |
| | GQIYPGDGDTNYNGKFKGQATLTADKSSSTAYMQLSGLTSEDSAVYFC | |
| | ARKTISSVVDFYFDYWGQGTTVTVSSGGGGGGGGSGGGGSDIELTQS | |
| | PKFMSTSVGDRVSVTCKASQNVGTNVAWYQQKPGQSPKPLIYSATYRN | |
| | SGVPDRFTGSGSGTDFTLTITNVQSKDLADYFCQQYNRYPYTSGGGTK | |
| | LEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggc | Sequence encoding |
| | gaccgggtgaccatcagctgccgggccagccaggacatcagcaagtac | scFv |
| | ctgaactggtatcagcagaagcccgacggcaccgtcaagctgctgatc | |
| | taccacaccagccggctgcacagcggcgtgcccagccggtttagcggc | |
| | agcggctccggcaccgactacagcctgaccatctccaacctggaacag | |
| | gaagatatcgccacctacttttgccagcagggcaacacactgccctac | |
| | acctttggcggcggaacaaagctggaaatcaccggcagcacctccggc | |
| | agcggcaagcctggcagcggcgagggcagcaccaagggcgaggtgaag | |
| | ctgcaggaaagcggccctggcctggtggcccccagccagagcctgagc | |
| | gtgacctgcaccgtgagcggcgtgagcctgcccgactacggcgtgagc | |
| | tggatccggcagccccccaggaagggcctggaatggctgggcgtgatc | |
| | tggggcagcgagaccacctactacaacagcgccctgaagagccggctg | |
| | accatcatcaaggacaacagcaagagccaggtgttcctgaagatgaac | |
| | agcctgcagaccgacgacaccgccatctactactgcgccaagcactac | |
| | tactacggcggcagctacgccatggactactggggccagggcaccagc | |
| | gtgaccgtgagcagc | |
| 58 | X₁PPX₂P | Hinge |
| | X₁ is glycine, cysteine or arginine | |
| | X₂ is cysteine or threonine | |
| 59 | GSTSGSGKPGSGEGSTKG | Linker |
| 60 | NSGMH | CDRH1 |
| 61 | VIWYDGSKRYYADSVKG | CDRH2 |
| 62 | NDDY | CDRH3 |
| 63 | RASQSVSSYLA | CDRL1 |
| 64 | DASNRAT | CDRL2 |
| 65 | QQSSNWPRT | CDRL3 |
| 66 | QVQLVESGGGVVQPGRSLRLDCKASGITFSNSGMHWVRQAPGKGLEWV | VH |
| | AVIWYDGSKRYYADSVKGRFTISRDNSKNTLFLQMNSLRAEDTAVYYC | |
| | ATNDDYWGQGTLVTVSS | |
| 67 | EIVLTQSPATLSLSPGERATLSCRASQSVSSYLAWYQQKPGQAPRLLI | VL |
| | YDASNRATGIPARFSGSGSGTDFTLTISSLEPEDFAVYYCQQSSNWPR | |
| | TFGQGTKVEIK | |
| 68 | DYYWN | CDR-H1 |
| 69 | EINHRGSTNSNPSLKS | CDR-H2 |
| 70 | GYSDYEYNWFDP | CDR-H3 |
| 71 | RASQSISSYLA | CDR-L1 |
| 72 | DASNRAT | CDR-L2 |
| 73 | QQRSNWPLT | CDR-L3 |
| 74 | QVQLQQWGAGLLKPSETLSLICAVYGGSFSDYYWNWIRQPPGKGLEWI | VH |
| | GEINHRGSINSNPSLKSRVILSLDISKNQFSLKLRSVTAADTAVYYCA | |
| | FGYSDYEYNWEDPWGQGTLVTVSS | |
| 75 | EIVLTQSPATLSLSPGERATLSCRASQSISSYLAWYQQKPGQAPRLLI | VL |
| | YDASNRATGIPARESGSGSGIDFILIISSLEPEDFAVYYCQQRSNWPL | |
| | TFGQGINLEIK | |

1. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering a PD-1 inhibitor to the subject; and
(3) administering a LAG3 inhibitor to the subject.

2. A method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1;
(2) administering a PD-1 inhibitor to the subject; and
(3) administering a LAG3 inhibitor to the subject.

3. The method of claim 1 or claim 2, wherein a first dose of the PD-1 inhibitor and a first dose of the LAG3 inhibitor are independently administered, each between Day 2 and Day 20, inclusive.

4. The method of claim 3, wherein the first dose of the PD-1 inhibitor and the first dose of the LAG3 inhibitor are administered on the same day.

5. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
(i) administration of a first dose of the PD-1 inhibitor is between Day 2 and Day 20, inclusive; and
(ii) each dose of the PD-1 inhibitor is between at or about 140 mg and at or about 580 mg, inclusive.

6. A method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1; and
(2) administering a PD-1 inhibitor to the subject in a dosing regimen comprising administration of at least two doses, wherein:
(i) administration of a first dose of the PD-1 inhibitor is between Day 2 and Day 20, inclusive; and
(ii) each dose of the PD-1 inhibitor is between at or about 140 mg and at or about 580 mg, inclusive.

7. The method of any of claims 1-6, wherein each subsequent dose of the PD-1 inhibitor is administered about two weeks, about three weeks, or about four weeks after the previous dose of the PD-1 inhibitor.

8. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
(i) administration of a first dose of the PD-1 inhibitor is between Day 2 and Day 20, inclusive; and
(ii) each subsequent dose of the PD-1 inhibitor is administered about two weeks, about three weeks, or about four weeks after the previous dose of the PD-1 inhibitor.

9. A method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1; and
(2) administering a PD-1 inhibitor to the subject in a dosing regimen comprising administration of at least two doses, wherein:
(i) administration of a first dose of the PD-1 inhibitor is between Day 2 and Day 20, inclusive; and
(ii) each subsequent dose of the PD-1 inhibitor is administered about two weeks, about three weeks, or about four weeks after the previous dose of the PD-1 inhibitor.

10. The method of any of claims 1-4 and 7-9, wherein each dose of the PD-1 inhibitor is between at or about 140 mg and at or about 580 mg, inclusive.

11. The method of any of claims 1-10, wherein a first dose of the PD-1 inhibitor is administered between Day 8 and Day 15, inclusive.

12. The method of any of claims 1-11, wherein a first dose of the PD-1 inhibitor is administered on Day 8.

13. The method of any of claims 1-11, wherein a first dose of the PD-1 inhibitor is administered on Day 15.

14. The method of any of claims 1-13, wherein the PD-1 inhibitor is administered for no longer than about three months.

15. The method of any of claims 1-14, wherein a final dose of the PD-1 inhibitor is administered between about Day 80 and about Day 90, optionally wherein the final dose of the PD-1 inhibitor is administered at about Day 85.

16. The method of any of claims 1-15, wherein each dose of the PD-1 inhibitor is between at or about 160 mg and 560 mg.

17. The method of any of claims 1-16, wherein each dose of the PD-1 inhibitor is at or about 240 mg, or at or about 480 mg.

18. The method of any of claims 1-17, wherein at least one dose of the PD-1 inhibitor is 240 mg, and at least one dose of the PD-1 inhibitor is 480 mg.

19. The method of any of claims 1-18, wherein at least four doses of the PD-1 inhibitor are administered.

20. The method of any of claims 1-19, wherein four doses, five doses, or six doses of the PD-1 inhibitor are administered.

21. The method of claim 19 or claim 20, wherein the first three doses of the PD-1 inhibitor are administered every two weeks (Q2W).

22. The method of any of claims 1-21, wherein each dose of the PD-1 inhibitor is administered every two weeks (Q2W).

23. The method of any of claims 19-21, wherein the fourth dose of the PD-1 inhibitor is administered about three weeks or about four weeks after the previous dose of the PD-1 inhibitor.

24. The method of any of claims 19-21 and 23, wherein five doses of the PD-1 inhibitor are administered, and the fifth dose of the PD-1 inhibitor is administered about four weeks after the fourth dose of the PD-1 inhibitor.

25. The method of any of claims 1-24, wherein about 240 mg of the PD-1 inhibitor is administered on each of Days 8, 22, and 36.

26. The method of any of claims 1-24, wherein about 240 mg of the PD-1 inhibitor is administered on each of Days 15, 29, and 43.

27. The method of any of claims 1-24, wherein about 480 mg of the PD-1 inhibitor is administered on each of Days 8, 36, 64, and 85.

28. The method of any of claims 1-24, wherein about 480 mg of the PD-1 inhibitor is administered on each of Days 15, 43, 64, and 85.

29. The method of any of claims 4-28, further comprising administering a LAG3 inhibitor to the subject.

30. The method of claim 29, wherein a first dose of the LAG3 inhibitor is administered between Day 2 and Day 20, inclusive.

31. The method of any of claims 1-3 and 30, wherein a first dose of the LAG3 inhibitor is administered between Day 8 and Day 15, inclusive.

32. The method of any of claims 1-3, 30, and 31, wherein a first dose of the LAG3 inhibitor is administered on Day 8.

33. The method of claim 1-3, 30, and 31, wherein a first dose of the LAG3 inhibitor is administered on Day 15.

34. The method of any of claims 1-3 and 29-33, wherein each dose of the LAG3 inhibitor is between about 60 mg and about 540 mg, inclusive.

35. The method of any of claims 1-3 and 29-34, wherein each dose of the LAG3 inhibitor is between about 120 mg and about 480 mg.

36. The method of any of claims 1-3 and 29-35, wherein each dose of the LAG3 inhibitor is about 120 mg.

37. The method of any of claims 1-3 and 29-35, wherein each dose of the LAG3 inhibitor is about 240 mg.

38. The method of any of claims 1-3 and 29-35, wherein each dose of the LAG3 inhibitor is about 480 mg.

39. The method of any of claims 1-3 and 29-38, wherein at least three doses of the LAG3 inhibitor are administered.

40. The method of any of claims 1-3 and 29-39, where three doses, four doses, or six doses of the LAG3 inhibitor are administered.

41. The method of claim 39 or claim 40, wherein the first three doses of the LAG3 inhibitor are administered every two weeks (Q2W).

42. The method of any of claims 1-3 and 29-41, wherein each dose of the LAG3 inhibitor is administered every two weeks (Q2W).

43. The method of any of claims 39-41, wherein the second dose of the LAG3 inhibitor is administered about four weeks after the first dose of the LAG3 inhibitor.

44. The method of any of claims 1-3 and 29-43, wherein doses of the PD-1 inhibitor and doses of the LAG3 inhibitor are administered with the same frequency.

45. The method of any of claims 1-3 and 29-44, wherein (i) each dose of the PD-1 inhibitor is administered on the same day as a dose of the LAG3 inhibitor; and/or (ii) each dose of the LAG3 inhibitor is administered on the same day as a dose of the PD-1 inhibitor.

46. The method of any of claims 1-3 and 29-43, wherein doses of the LAG3 inhibitor are administered half as frequently as doses of the PD-1 inhibitor.

47. The method of any of claims 1-3 and 29-46, wherein each dose of the PD-1 inhibitor is double the dose of the LAG3 inhibitor.

48. The method of any of claims 1-3 and 29-46, wherein each dose of the PD-1 inhibitor is the same as the dose of the LAG3 inhibitor.

49. The method of any of claims 1-3, 29-45, 47, and 48, wherein the PD-1 inhibitor and the LAG3 inhibitor are formulated in a single composition, optionally for intravenous administration.

50. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 8, 22, 36, 57, 71, and 85; and
(3) administering to the subject a LAG3 inhibitor in a dosing regimen comprising administration of 240 mg of the LAG3 inhibitor on Days 8, 36, and 71.

51. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 15, 29, 43, 57, 71, and 85; and
(3) administering to the subject a LAG3 inhibitor in a dosing regimen comprising administration of 240 mg on Days 15, 43, and 71.

52. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 8, 22, 36, 57, 71, and 85; and
(3) administering to the subject a LAG3 inhibitor in a dosing regimen comprising administration of 240 mg of the LAG3 inhibitor on Days 8, 22, 36, 57, 71, and 85.

53. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 15, 29, 43, 57, 71, and 85; and
(3) administering to the subject a LAG3 inhibitor in a dosing regimen comprising administration of 240 mg of the LAG3 inhibitor on Days 815, 29, 43, 57, 71, and 85.

54. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
(i) administration of a first amount of the PD-1 inhibitor, wherein the first amount is 240 mg and is administered on Days 8, 22, and 36; and
(ii) administration of a second amount of the PD-1 inhibitor, wherein the second amount is 480 mg and is administered on Days 57 and 85.

55. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering to the subject a PD-1 inhibitor in a dosing regimen comprising:
(i) administration of a first amount of the PD-1 inhibitor, wherein the first amount is 240 mg and is administered on Days 15, 29, and 43; and
(ii) administration of a second amount of the PD-1 inhibitor, wherein the second amount is 480 mg and is administered on Days 57 and 85.

56. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 8, 22, 36, 57, 71, and 85; and
(3) administering to the subject a LAG3 inhibitor in a dosing regimen comprising administration of 120 mg of the LAG3 inhibitor on Days 8, 22, 36, 57, 71, and 85.

57. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 240 mg of the PD-1 inhibitor on Days 15, 29, 43, 57, 71, and 85; and
(3) administering to the subject a LAG3 inhibitor in a dosing regimen comprising administration of 120 mg of the LAG3 inhibitor on Days 15, 29, 43, 57, 71, and 85.

58. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 8, 36, 64, and 85; and
(3) administering to the subject a LAG3 inhibitor in a dosing regimen comprising administration of 240 mg of the LAG3 inhibitor on Days 8, 36, 64, and 85.

59. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 15, 43, 64, and 85; and
(3) administering to the subject a LAG3 inhibitor in a dosing regimen comprising administration of 240 mg of the LAG3 inhibitor on Days 15, 43, 64, and 85.

60. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 8, 36, 64, and 85; and
(3) administering to the subject a LAG3 inhibitor in a dosing regimen comprising administration of 480 mg of the LAG3 inhibitor on Days 8, 36, 64, and 85.

61. A method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering to the subject a PD-1 inhibitor in a dosing regimen comprising administration of 480 mg of the PD-1 inhibitor on Days 15, 43, 64, and 85; and
(3) administering to the subject a LAG3 inhibitor in a dosing regimen comprising administration of 480 mg of the LAG3 inhibitor on Days 15, 43, 64, and 85.

62. The method of any of claims 1-61, wherein the PD-1 inhibitor is an anti-PD-1 antibody.

63. The method of claim 62, wherein the anti-PD-1 antibody comprises a heavy chain variable (VH) region comprising a CDR1, a CDR2, and a CDR3 comprising the amino acid sequences set forth in SEQ ID NOS: 60, 61, and 62 respectively, and a light chain variable (VL) region comprising a CDR1, a CDR2, and a CDR3 comprising the amino acid sequences set forth in SEQ ID NOS: 63, 64, and 65 respectively.

64. The method of claim 63, wherein the VH region comprises the amino acid sequence set forth in SEQ ID NO: 66, and the VL region comprises the amino acid sequence set forth in SEQ ID NO: 67.

65. The method of any of claims 62-64, wherein the anti-PD-1 antibody is nivolumab.

66. The method of any of claims 1-3 and 29-65, wherein the LAG3 inhibitor is an anti-LAG3 antibody.

67. The method of claim 66, wherein the anti-LAG3 antibody comprises a heavy chain variable (VH) region comprising a CDR1, a CDR2, and a CDR3 comprising the amino acid sequences set forth in SEQ ID NOS: 68, 69, and 70 respectively, and a light chain variable (VL) region comprising a CDR1, a CDR2, and a CDR3 comprising the amino acid sequences set forth in SEQ ID NOS: 71, 72, and 73 respectively.

68. The method of claim 67, wherein the VH region comprises the amino acid sequence set forth in SEQ ID NO: 74, and the VL region comprises the amino acid sequence set forth in SEQ ID NO: 75.

69. The method of any of claims 66-68, wherein the anti-LAG3 antibody is relatlimab.

70. The method of any of claims 1-69, further comprising administering a lymphodepleting therapy to the subject prior to administration of the dose of engineered T cells.

71. The method of claim 70, wherein the lymphodepleting therapy is completed within about 7 days prior to initiation of the administration of the dose of engineered T cells.

72. The method of claim 70 or claim 71, wherein the administration of the lymphodepleting therapy is completed within about 2 to 7 days prior to initiation of the administration of the dose of engineered T cells.

73. The method of any of claims 70-72, wherein the lymphodepleting therapy comprises the administration of fludarabine and/or cyclophosphamide.

74. The method of any of claims 70-73, wherein the lymphodepleting therapy comprises administration of cyclophosphamide at or about 200-400 mg/m², optionally at or about 300 mg/m², inclusive, and/or fludarabine at or about 20-40 mg/m², optionally 30 mg/m², daily for 2-4 days, optionally for 3 days.

75. The method of any one of claims 70-74, wherein the lymphodepleting therapy comprises administration of cyclophosphamide at or about 300 mg/m² and fludarabine at or about 30 mg/m² daily concurrently for 3 days.

76. The method of any of claims 1-75, wherein CD19 is human CD19.

77. The method of any of claims 1-76, wherein the chimeric antigen receptor (CAR) comprises an scFv comprising the variable heavy chain region and the variable light chain region of the antibody FMC63, a spacer that is 15 amino acids or less and contains an immunoglobulin hinge region or a modified version thereof, a transmembrane domain, and an intracellular signaling domain comprising a signaling domain of a CD3-zeta (CD3ζ) chain and a costimulatory signaling region that is a signaling domain of 4-1BB.

78. The method of claim 77, wherein the immunoglobulin hinge region or a modified version thereof comprises the formula X₁PPX₂P, wherein X₁ is glycine, cysteine or arginine and X₂ is cysteine or threonine (SEQ ID NO:58).

79. The method of claim 77 or claim 78, wherein the immunoglobulin hinge region or a modified version thereof is an IgG1 hinge or a modified version thereof.

80. The method of claim 77 or claim 78, wherein the immunoglobulin hinge region or a modified version thereof is an IgG4 hinge or a modified version thereof.

81. The method of any of claims 77-80, wherein the spacer comprises the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

82. The method of any of claims 77-81, wherein the spacer consists of the sequence set forth in SEQ ID NO: 1, SEQ ID NO: 30, SEQ ID NO: 31, SEQ ID NO: 32, SEQ ID NO: 33, or SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity to the sequence set forth in SEQ ID NO: 1, SEQ ID NO: 30, SEQ ID NO: 31, SEQ ID NO: 32, SEQ ID NO: 33, or SEQ ID NO: 34.

83. The method of any of claims 77-82, wherein the spacer is at or about 12 amino acids in length.

84. The method of any of claims 77-83, wherein the spacer comprises the sequence set forth in SEQ ID NO: 1.

85. The method of any of claims 77-84, wherein the spacer consists of the sequence set forth in SEQ ID NO: 1.

86. The method of any of claims 77-85, wherein the transmembrane domain is a transmembrane domain of CD28.

87. The method of any of claims 77-86, wherein the transmembrane domain comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 8.

88. The method of any of claims 77-87, wherein the costimulatory domain comprises the sequence set forth in SEQ ID NO: 12 or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 12.

89. The method of any of claims 77-88, wherein the signaling domain of a CD3-zeta (CD3ζ) chain comprises the sequence set forth in SEQ ID NO: 13, 14, or 15, or is a variant thereof having at least 85%, 86%, 87%, 88%, 89%, 90%, 91%, 92%, 93%, 94%, 95%, 96%, 97%, 98%, 99% or more sequence identity to the sequence set forth in SEQ ID NO: 13, 14, or 15.

90. The method of any of claims 77-89, wherein the scFv comprises a CDRL1 sequence of SEQ ID NO: 35, a CDRL2 sequence of SEQ ID NO: 55, and a CDRL3 sequence of SEQ ID NO: 56; and a CDRH1 sequence of SEQ ID NO: 38, a CDRH2 sequence of SEQ ID NO: 39, and a CDRH3 sequence of SEQ ID NO: 54.

91. The method of any of claims 77-90, wherein the scFv comprises a CDRL1 sequence of SEQ ID NO: 35, a CDRL2 sequence of SEQ ID NO: 36, and a CDRL3 sequence of SEQ ID NO: 37; and a CDRH1 sequence of SEQ ID NO: 38, a CDRH2 sequence of SEQ ID NO: 39, and a CDRH3 sequence of SEQ ID NO: 40.

92. The method of any of claims 77-91, wherein the scFv comprises, in order from N-terminus to C-terminus, a V_{L} comprising the sequence set forth in SEQ ID NO: 42, and a V_{H}, comprising the sequence set forth in SEQ ID NO: 41.

93. The method of any of claims 77-92, wherein the scFv comprises the sequence set forth in SEQ ID NO: 43.

94. The method of any of claims 77-93, wherein the CAR contains in order from N-terminus to C-terminus: an extracellular antigen-binding domain that is the scFv set forth in SEQ ID NO: 43, the spacer set forth in SEQ ID NO: 1, the transmembrane domain set forth in SEQ ID NO: 8, the 4-1BB costimulatory signaling domain set forth in SEQ ID NO: 12, and the signaling domain of a CD3-zeta (CD3ζ) chain set forth in SEQ ID NO: 13.

95. The method of any of claims 1-94, wherein the dose of the engineered T cells comprises CD4+ T CAR-expressing cells and CD8+ CAR-expressing T cells.

96. The method of any of claims 1-95, wherein the dose of engineered T cells comprises between about 5×10⁷ CAR-expressing T cells and about 1.1×10⁸ CAR-expressing T cells, inclusive of each.

97. The method of any of claims 1-96, wherein the dose of engineered T cells comprises about 5×10⁷ CAR-expressing T cells.

98. The method of any of claims 1-97, wherein the dose of engineered T cells comprises about 1×10⁸ CAR-expressing T cells.

99. The method of any of claims 95-98, wherein administration of the dose of engineered T cells comprises administering a plurality of separate compositions, wherein the plurality of separate compositions comprises a first composition comprising the CD8+ CAR-expressing T cells and a second composition comprising the CD4+ CAR-expressing T cells.

100. The method of claim 99, wherein:
the first composition and the second composition are administered 0 to 12 hours apart, 0 to 6 hours apart, or 0 to 2 hours apart, or wherein the administration of the first composition and the administration of the second composition are carried out on the same day, between about 0 and about 12 hours apart, between about 0 and about 6 hours apart, or between about 0 and 2 hours apart; and/or
the initiation of administration of the first composition and the initiation of administration of the second composition are carried out between about 1 minute and about 1 hour apart or between about 5 minutes and about 30 minutes apart.

101. The method of claim 99 or claim 100, wherein the first composition and the second composition are administered no more than 2 hours, no more than 1 hour, no more than 30 minutes, no more than 15 minutes, no more than 10 minutes, or no more than 5 minutes apart.

102. The method of any of claims 99-101, wherein the first composition and the second composition are administered less than 2 hours apart.

103. The method of any of claims 99-102, wherein the first composition comprising the CD8⁺ CAR-expressing T cells is administered prior to the second composition comprising the CD4⁺ CAR-expressing T cells.

104. The method of any of claims 1-103, wherein the cells of the dose of the engineered T cells are administered intravenously.

105. The method of any of claims 1-104, wherein the T cells are primary T cells obtained from a sample from the subject, optionally wherein the sample is a whole blood sample, an apheresis sample, or a leukapheresis sample.

106. The method of claim 105, wherein the sample is obtained from the subject prior to administration of the lymphodepleting therapy to the subject.

107. The method of any of claims 1-106, wherein the T cells are autologous to the subject.

108. The method of any of claims 1-107, wherein the subject is human.

109. The method of any of claims 1-108, wherein the CD19-expressing cancer is a B cell malignancy.

110. The method of any of claims 1-109, wherein the CD19-expressing cancer is a myeloma, a leukemia, or a lymphoma.

111. The method of any of claims 1-110, wherein the CD19-expressing cancer is an acute lymphoblastic leukemia (ALL), adult ALL, chronic lymphoblastic leukemia (CLL), a small lymphocytic lymphoma (SLL), non-Hodgkin lymphoma (NHL), or a large B cell lymphoma.

112. The method of any of claims 1-111, wherein the CD19-expressing cancer is a non-Hodgkin lymphoma (NHL).

113. The method of any claim 111 or claim 112, wherein the NHL is selected from the group consisting of: diffuse large B-cell lymphoma (DLBCL) not otherwise specified (NOS) including transformed indolent NHL, follicular lymphoma Grade 3B (FL3B), T cell/histiocyte-rich large B-cell lymphoma, Epstein-Barr virus (EBV) positive DLBCL NOS, primary mediastinal (thymic) large B-cell lymphoma, Richter's Transformation and high grade B-cell lymphoma with MYC and BCL2 and/or BCL6 rearrangements with DLBCL histology (double/triple-hit lymphoma).

114. The method of any of claims 111-113, wherein the NHL is a relapsed/refractory (R/R) NHL.

115. The method of any of claims 111-114, wherein the subject is relapsed or refractory to at least two prior lines of systemic therapy for the CD19-expressing cancer.

116. The method of claim 115, wherein at least one of the at least two prior lines of systemic therapy includes a CD20-targeted agent and an anthracycline.

117. The method of any of claims 1-116, wherein the subject has an ECOG performance status of 0 or 1.

118. The method of any of claims 1-117, wherein the subject has positron-emission tomography (PET)-positive disease.

119. The method of any of claims 1-118, wherein the subject has computed tomography (CT) measurable disease.

120. The method of any of claims 1-119, wherein the subject has a sum of product of perpendicular diameters (SPD) of up to 6 index lesions of greater than or equal to 25 cm², optionally by CT scan.

121. Use of a PD-1 inhibitor and a LAG3 inhibitor in the manufacture of a medicament for treating a subject having a CD19-expressing cancer, wherein the subject was previously administered a cell therapy comprising a dose of engineered cells comprising T cells expressing a chimeric antigen receptor (CAR) that binds cluster of differentiation 19 (CD19) on Day 1.

122. Use of a PD-1 inhibitor and a LAG3 inhibitor in the manufacture of a medicament for treating a subject having a CD19-expressing cancer, wherein the subject was previously administered a cell therapy comprising a dose of engineered cells comprising T cells expressing a chimeric antigen receptor (CAR) directed against the cancer on Day 1.

123. The use of claim 121 or claim 122, wherein the medicament is to be administered to the subject between Day 2 and Day 20.

124. Use of a PD-1 inhibitor in the manufacture of a medicament for treating a subject having a CD19-expressing cancer, wherein:
(1) at least two doses of the medicament are to be administered to the subject;
(2) a first dose of the medicament is to be administered to the subject between Day 2 and Day 20, inclusive;
(3) each dose of the medicament comprises between at or about 140 mg and at or about 580 mg of the PD-1 inhibitor, inclusive; and
(4) the subject was previously administered a cell therapy comprising a dose of engineered cells comprising T cells expressing a chimeric antigen receptor (CAR) that binds cluster of differentiation 19 (CD19) on Day 1.

125. Use of a PD-1 inhibitor in the manufacture of a medicament for treating a subject having a CD19-expressing cancer, wherein:
(1) at least two doses of the medicament are to be administered to the subject;
(2) a first dose of the medicament is to be administered to the subject between Day 2 and Day 20, inclusive;
(3) each dose of the medicament comprises between at or about 140 mg and at or about 580 mg of the PD-1 inhibitor, inclusive; and
(4) the subject was previously administered a cell therapy comprising a dose of engineered cells comprising T cells expressing a chimeric antigen receptor (CAR) directed against the cancer on Day 1.

126. The use of any of claims 121-125, wherein each subsequent dose of the medicament is to be administered about two weeks, about three weeks, or about four weeks after the previous dose of the medicament.

127. Use of a PD-1 inhibitor in the manufacture of a medicament for treating a CD19-expressing cancer, wherein:
(1) at least two doses of the medicament are to be administered to the subject;
(2) a first dose of the medicament is to be administered to the subject between Day 2 and Day 20, inclusive;
(3) each subsequent dose of the medicament is to be administered about two weeks, about three weeks, or about four weeks after the previous dose of the medicament; and
(4) the subject was previously administered a cell therapy comprising a dose of engineered cells comprising T cells expressing a chimeric antigen receptor (CAR) that binds cluster of differentiation 19 (CD19) on Day 1.

128. Use of a PD-1 inhibitor in the manufacture of a medicament for treating a CD19-expressing cancer, wherein:
(1) at least two doses of the medicament are to be administered to the subject;
(2) a first dose of the medicament is to be administered to the subject between Day 2 and Day 20, inclusive;
(3) each subsequent dose of the medicament is to be administered about two weeks, about three weeks, or about four weeks after the previous dose of the medicament; and
(4) the subject was previously administered a cell therapy comprising a dose of engineered cells comprising T cells expressing a chimeric antigen receptor (CAR) directed against the cancer on Day 1.

129. The use of any of claims 121-123 and 126-128, wherein each dose of the medicament comprises between at or about 140 mg and at or about 580 mg of the PD-1 inhibitor, inclusive.

130. The use of any of claims 124-129, wherein the subject is administered a LAG3 inhibitor following administration of the cell therapy.

131. A combination of a PD-1 inhibitor and a LAG3 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1;
(2) administering a PD-1 inhibitor to the subject; and
(3) administering a LAG3 inhibitor to the subject.

132. A combination of a PD-1 inhibitor and a LAG3 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1;
(2) administering a PD-1 inhibitor to the subject; and
(3) administering a LAG3 inhibitor to the subject.

133. The combination for use of claim 131 or claim 132, wherein a first dose of the PD-1 inhibitor and a first dose of the LAG3 inhibitor are independently administered, each between Day 2 and Day 20.

134. The combination for use of claim 133, wherein the first dose of the PD-1 inhibitor and the first dose of the LAG3 inhibitor are administered on the same day.

135. A PD-1 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
(i) administration of a first dose of the PD-1 inhibitor is between Day 2 and Day 20, inclusive; and
(ii) each dose of the PD-1 inhibitor is between at or about 140 mg and at or about 580 mg, inclusive.

136. A PD-1 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1; and
(2) administering a PD-1 inhibitor to the subject in a dosing regimen comprising administration of at least two doses, wherein:
(i) administration of a first dose of the PD-1 inhibitor is between Day 2 and Day 20, inclusive; and
(ii) each dose of the PD-1 inhibitor is between at or about 140 mg and at or about 580 mg, inclusive.

137. The combination for use of any of claims 131-134 or the PD-1 inhibitor for use of claim 135 or claim 136, wherein each subsequent dose of the PD-1 inhibitor is administered about two weeks, about three weeks, or about four weeks after the previous dose of the PD-1 inhibitor.

138. A PD-1 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) that binds cluster of differentiation 19 (CD19) on Day 1; and
(2) administering a PD-1 inhibitor to the subject in a dosing regimen comprising administration of at least two doses, wherein:
(i) a first dose of the PD-1 inhibitor is administered between Day 2 and Day 20, inclusive; and
(ii) each subsequent dose of the PD-1 inhibitor is administered about two weeks, about three weeks, or about four weeks after the previous dose of the PD-1 inhibitor.

139. A PD-1 inhibitor for use in a method of treating a cancer, the method comprising:
(1) administering to a subject having a CD19-expressing cancer a cell therapy comprising a dose of engineered cells comprising T cells expressing a chimeric antigen receptor (CAR) directed against the cancer on Day 1; and
(2) administering a PD-1 inhibitor to the subject in a dosing regimen comprising administration of at least two doses, wherein:
(i) a first dose of the PD-1 inhibitor is administered between Day 2 and Day 20, inclusive; and
(ii) each subsequent dose of the PD-1 inhibitor is administered about two weeks, about three weeks, or about four weeks after the previous dose of the PD-1 inhibitor.

140. The combination for use of any of claims 131-134 or the PD-1 inhibitor for use of claim 138 or claim 139, wherein each dose of the PD-1 inhibitor is between at or about 140 mg and at or about 580 mg, inclusive.

141. The PD-1 inhibitor for use of any of claims 135-140, wherein the method further comprises administering a LAG3 inhibitor to the subject.
